(19) United States
(12) Patent Application Publication
(10) Pub. No.: US 2024/0270759 A1
(43) Pub. Date: Aug. 15, 2024

(54) Preparation of SHP2 phosphatase inhibitors and its applications

(71) Applicant: Ya Therapeutics Inc, Shanghai (CN)

(72) Inventors: Yonghong LIANG, Shanghai (CN); Zhiyong XU, Shanghai (CN); Zhaosen ZENG, Shanghai (CN); Wenguang YAN, Shanghai (CN); Fangjun XIONG, Shanghai (CN)

(21) Appl. No.: 18/559,830

(22) PCT Filed: May 7, 2022
(86) PCT No.: PCT/CN2022/091425

§ 371 Date: Nov. 9, 2023

(30) Foreign Application Priority Data
May 12, 2021 (CN) 202110517521.4
May 14, 2021 (CN) 202110529037.3
May 31, 2021 (CN) 202110601920.9

(51) Int. Cl. C07D 519/00; A61K 31/495; A61K 31/501; A61K 31/506; A61K 31/517; A61K 31/519; A61K 31/53; C07D 401/04; C07D 471/10; C07D 475/02; C07D 491/107

(52) U.S. Cl. CPC C07D 519/00; A61K 31/495; A61K 31/501; A61K 31/506; A61K 31/517; A61K 31/519; A61K 31/53; C07D 401/04; C07D 471/10; C07D 475/02; C07D 491/107

(57) ABSTRACT

The present invention has disclosed SHP2 phosphatase inhibitors and its applications. Specifically, the present invention has disclosed the compounds shown in the general formula (I), methods of preparation thereof, and pharmaceutical compositions containing the compound, and their use as tyrosine phosphatase SHP-2 inhibitors in the treatment of leukemia, neuroblastoma, melanoma, acute myeloid leukemia, breast cancer, esophageal cancer, lung cancer, colon cancer, head cancer, pancreatic cancer, head and neck squamous cell carcinoma, stomach cancer, liver cancer, anaplastic large cell lymphoma, and glioblastoma, wherein each substituent in general formula (I)k is as defined in the specification.

### TECHNICAL FIELD

The present invention belongs to the field of drug synthesis, and specifically relates to a new SHP2 phosphatase inhibitor and its preparation method and its applications.

### BACKGROUND TECHNOLOGY

The present invention generally relates to novel compounds and methods for their preparation and use as SHP2 phosphatase inhibitors (e.g., for the treatment of cancer).

SHP2 is a non-receptor protein tyrosine phosphatase encoded by the PTPN11 gene, and contains two N-terminal Src homology 2 (SH2) structural domains, a protein tyrosine phosphatase (PTP) structural domain and a poorly sequenced C-terminus. X-ray crystallographic studies have shown that SHP2 inhibits its own phosphatase activity by using the N-terminal SH2 structural domain to block the proximity to the catalytic site on the PTP structural domain. Tyrosyl diphosphate proteins or peptides (e.g., IRS-1) have been shown to bind to the SH2 structural domains of SHP2, disrupting the interaction of N-terminal SH2-PTP domain. This binding allows substrate access to the catalytic site and activation of the phosphatase.

SHP2 is recruited by RTK to induce cell signaling and is involved in several intracellular oncogenic signaling cascade reaction such as Jak/STAT, PI3K/AKT, RAS/Raf/MAPK, PD-1/PD-L1, as well as mTOR pathways. Wherein, RAS, a key GTPase that transduces extracellular signals to the nucleus, is regulated by SHP2 (tyrosine dephosphorylation in the adaptor/scaffolding proteins) to be activated GTP-binding mode to exert oncogenic effects; and during acquired drug-resistance, activation of RAS signaling by SHP2 facilitates compensatory activation of signaling pathways (e.g., negative feedback regulation of MEK activates RTK, which activates SHP2 thereby activating downstream pathways), in which case the inhibitory effect on SHP2 could eliminate the reactivation of the RAS/Raf/ERK pathway, this mechanism can be considered as a potential therapeutic strategy for RTK pathway resistance.

Moreover, the germline or somatic mutations in PTPN11 that lead to SHP2 overactivation have been identified in a variety of pathophysiological states: e.g., developmental disorders such as Noonan syndrome, hematologic malignancies including juvenile myelomonocytic leukemia, myelodysplastic syndromes, B-cell acute lymphoblastic leukemia and acute myeloid leukemia, and low frequency solid tumors.

Therefore, SHP2 is one of the latest highly attractive targets of the new therapies for the treatment of various diseases.

SHP2 phosphatase inhibitors currently available in the clinical stage include TNO155 from Norvatis and JAB-3068 from JACOBIO, both of which are still in Phase II clinical trial stage. And there are no marketed products for this target. Therefore, it is important to develop more efficient inhibitors for this target.

### CONTENTS OF THE INVENTION

In order to solve the above problems, the present invention provides the compounds shown in general formula (I) and general formula (II), or their prodrugs, stable isotope derivatives, salts pharmaceutically usable, polymorphic substances or isomers thereof.

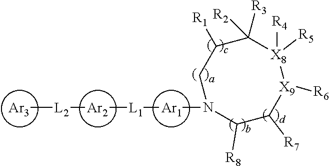

Wherein:
Each L₁ is, at each occurrence, independently selected from bond, O, CH₂, NH, CO, —S(O)ₘ—, or S;
Each L₂ is, at each occurrence, independently selected from bond, O, CH₂, NH, CONH₂, CO, —S(O)ₘ—, or S;
Each Ar₁, at each occurrence, is independently selected from a 6-membered heteroaryl or a 10-membered heteroaryl; Each Ar₁ at each occurrence is independently optionally substituted or unsubstituted by 1 or 2 R_{19S};
Each Ar₂ is, at each occurrence, independently selected from phenyl, naphthyl, 5-membered heteroaryl, 6-membered heteroaryl, 7-membered heteroaryl, 8-membered heteroaryl, 9-membered heteroaryl, or 10-membered heteroaryl, 3-membered to 10-membered cycloalkyl, 5-membered to 10-membered heterocyclic alkyl; and each heteroaryl and heterocyclic alkyl at each occurrence independently comprises, 1, 2, 3, or 4 heteroatoms selected from N, O, or S; Each Ar₂ at each occurrence is independently optionally substituted or unsubstituted by 1, 2, 3, 4, 5, or 6 R_{19S};
Each Ar₃ is, at each occurrence, independently selected from H, D, phenyl, naphthyl, 5-membered heteroaryl, 6-membered heteroaryl, 7-membered heteroaryl, 8-membered heteroaryl, 9-membered heteroaryl, or 10-membered heteroaryl, 3-membered to 10-membered cycloalkyl, 5-membered to 10-membered heterocyclic alkyl, and each heteroaryl and heterocyclic alkyl is, at each occurrence, independently comprises 1, 2, 3, or 4 heteroatoms selected from N, O, or S; Each Ar₃ is independently optionally substituted or unsubstituted by 1, 2, 3, 4, 5 or 6 R_{19S};
Each R₁₉ is, at each occurrence, independently selected from deuterium, halogen, oxo, —C₁₋₆ alkyl, —C₁₋₆ alkylidene-(halogen)₁₋₃, C₁₋₆ heteroalkyl, —CN, —OR₁₀, —C₁₋₆ alkylidene-(OR₁₀)₁₋₃, —O—C₁₋₆ alkylidene-(halogen)₁₋₃, —SR₁₀, —S—C₁₋₆ alkylidene-(halogen)₁₋₃, —NR₁₀R₁₁, —C₁₋₆ alkylidene-NR₁₀R₁₁, —C(═O)R₁₀, —C(═O)OR₁₀, —OC(═O)R₁₀, —C(═O)NR₁₀R₁₁, —NR₁₀C(═O)R₁₁, —S(O)₂NR₁₀R₁₁, or —C₃₋₆ carbocyclyl; Each R₁₉ is independently optionally substituted or unsubstituted by 1, 2, 3, 4, 5 or 6 substituents selected from deuterium, halogen, —C₁₋₆ alkyl, —C₁₋₆ alkoxy, oxo, —OR₁₀, —NR₁₀R₁₁, —CN, —C(═O)R₁₀, —C(═O)OR₁₀, —OC(═O)R₁₀, —C(═O)NR₁₀R₁₁, —NR₁₀C(═O)R₁₁, or —S(O)₂NR₆R₁₁;
Each R₁₀ and R₁₁ is, at each occurrence, independently selected from hydrogen, deuterium, or —C₁₋₆ alkyl, and each R₁₀ and R₁₁ is independently optionally substituted or unsubstituted by 1, 2, 3, 4, 5, or 6 substituents of R_{19S}; Or, R₁₀, R₁₁ together with the N atoms attached to R₁₀ and R₁₁, form a 3-membered and 10-membered heterocyclic ring, and the stated 3-membered and 10-membered heterocyclic ring can further comprise 1, 2, 3, or 4 heteroatoms selected from N, O, S, S(═O), S(═O), or S(═O)₂, and the stated 3-membered and 10-membered heterocyclic ring is independently optionally substituted or unsubstituted by 1, 2, 3, 4, 5, or 6 R_{20S};
Each R₂₀ is, at each occurrence, independently selected from deuterium, halogen, oxo, —C₁₋₆ alkyl, —C₁₋₆ alkylidene-(halogen)₁₋₃, C₁₋₆ heteroalkyl, —CN, —OC₁₋₆, —C₁₋₆ alkylidene-(OC₁₋₆)₁₋₃, —O—C₁₋₆ alkylidene-(halogen)₁₋₃, —SC₁₋₆, —S—C₁₋₆ alkylidene-(halogen)₁₋₃, or —C₃₋₆ carbocyclyl;
Each X₈ is, at each occurrence, independently selected from CR₄R₅, SiR₄R₅, NH, or O;
Each X₉ is, at each occurrence, independently selected from CR₆ or NH, wherein, one of X₇ and X₈ must be carbon atom;
Each R₁ is, at each occurrence, independently selected from H, deuterium, or —C₁₋₆ alkyl;
Each R₂ is, at each occurrence, independently selected from H, deuterium, OH, or CH₂NH₂;
Each R₃, R₇ and R₈ is, at each occurrence, independently selected from H or deuterium;
Each R₄ is, at each occurrence, independently selected from H, deuterium, OH, or C₀₋₃NR₁₂R₁₃;
Each R₅ is, at each occurrence, independently selected from H, deuterium, OH, or C₁₋₆ alkyl; C₁₋₆ alkyl comprises 1, 2, 3, 4, 5, or 6 deuteriums, OH, methyl, OCH₃, and 5-membered to 10-membered heteroaryl;
Each R₆ is, at each occurrence, independently selected from H, deuterium, or NH₂;
Two of R₁, R₂, R₃, R₄, R₅, R₆, R₇, and R₈ can connects each other in the following manner:
R₁ and R₂ can be connected in the form of CH₂NHCH₂ to form a fused bicyclic structure,
R₁ and R₆ can be connected by an alkylidene group to form a bridged bicyclic structure,
R₂ and R₃ can be connected by an NH₂-substituted alkylidene to form a spiro,
R₄ and R₅ can be connected to form a C₃₋₁₂ cycloalkyl, a C₃₋₁₂ heterocyclic alkyl, a C₃₋₁₂ bicyclic alkyl, or a C₃₋₁₂ heterobicyclic alkyl; wherein, the C₃₋₁₂ heterocyclic alkyl and the heterobicyclic alkyl of C₃₋₁₂, at each occurrence, independently comprises 1, 2, 3, or 4 heteroatoms selected from N, O, or S; and wherein each of C₃₋₁₂ cycloalkyl, C₃₋₁₂ heterocyclic alkyl, C₃₋₁₂ bicyclic alkyl, and C₃₋₁₂ heterobicyclic alkyl is, at each occurrence, independently optionally is substituted by deuterium, halogen, OH, CH₃, an OCH₃, an NH₂ to form a spiro.
R₁ and R₇ can form a bridged bicyclic structure through the connection of alkylidene, O and NH,
R₂ and R₆ can form a bridged bicyclic structure through the connection of alkylidenes,
R₂ and R₇ can form a bridged bicyclic structure through the connection of alkylidene and O,
R₄ and R₆ can connect with each other to form a fused bicyclic structure through NHCH₂ and NH₂-substituted C₃-12 cycloalkyl,
Each of a, b, c, and dis, at each occurrence, independently selected from 0, or 1;
In some embodiments, the compound of (I) described above, a pharmaceutically acceptable salt thereof, or a stereoisomer thereof, wherein each Ar₁ is, at each occurrence, independently selected from

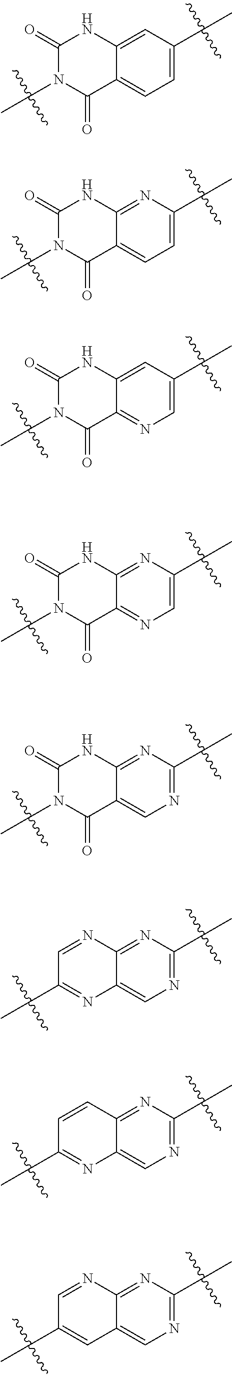

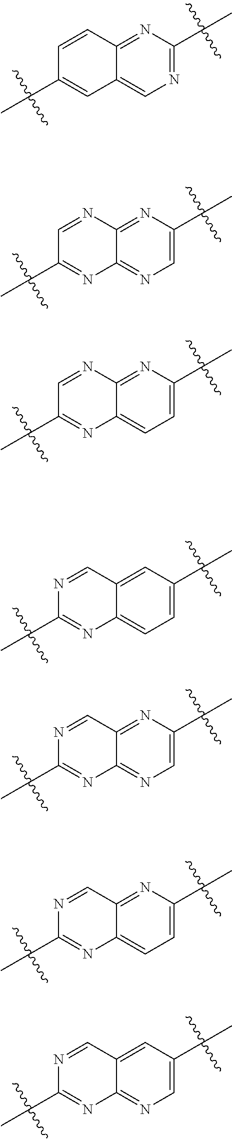

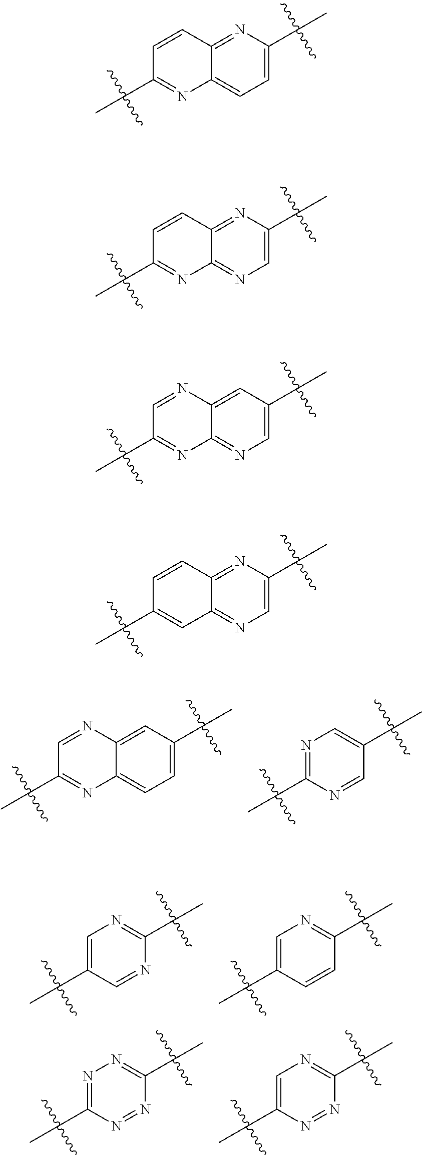

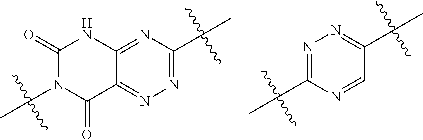 [SMILES: *c1nnc2c(=O)n(*)c(=O)[nH]c2n1 ; *c1cnc(*)nn1]

Each Ar₁ is, at each occurrence, independently optionally substituted or unsubstituted by 1 or 2 R_{19S};
Each R₁₉ is independently selected, at each occurrence, from deuterium, halogen, oxo, —C₁₋₆ alkyl, —C₁₋₆ alkylidene-(halogen)₁₋₃, C₁₋₆ heteroalkyl, —CN, —OR₁₀, —C₁₋₆ alkylidene-(OR₁₀)₁₋₃, —O—C₁₋₆ alkylidene-(halogen)₁₋₃, —SR₁₀, —S—C₁₋₆ alkylidene-(halogen)₁₋₃, —NR₁₀R₁₁, —C₁₋₆ alkylidene-NR₁₀R₁₁, —C(—O)R₁₀, —C(═O)OR₁₀, —OC(═O)R₁₀, —C(═O)NR₁₀R₁₁, —NR₁₀C(═O)R₁₁, —S(O)₂NR₁₀R₁₁, or —C₃₋₆ carbocyclic; Each R₁₉ is independently optionally substituted by 1, 2, 3, 4, 5 or 6 substituents selected from deuterium, halogen, —C₁₋₆ alkyl, —C₁₋₆ alkoxy, oxo, —OR₁₀, —NR₁₀R₁₁, —CN, —C(—O)R₁₀, —C(═O)OR₁₀, —OC(═O)R₁₀, —C(═O)NR₁₀R₁₁, —NR₁₀C(═O)R₁₁, or —S(O)₂NR₆R₁₁;
Each of R₁₀ and R₁₁ is independently selected, at each occurrence, from hydrogen, deuterium, or —C₁₋₆ alkyl, and each of R₁₀ and R₁₁ is independently optionally substituted or unsubstituted by 1, 2, 3, 4, 5, or 6 R₁₉ₛ; or R₁₀, R₁₁, and the atoms attached thereto form a 3-membered to 10-membered heterocyclic ring, and the stated 3-membered to 10-membered heterocyclic ring can further comprise 1, 2, 3, or 4 heteroatoms selected from N, O, S, S(═O), S(═O), or S(═O)₂, and the stated 3-membered to 10-membered heterocyclic ring is independently optionally substituted or unsubstituted by 1, 2, 3, 4, 5, or 6 R_{20S};
Each R₂₀ is independently selected, at each occurrence, from deuterium, halogen, oxo, —C₁₋₆ alkyl, —C₁₋₆ alkylidene-(halogen)₁₋₃, C₁₋₆ heteroalkyl, CN, —OC₁₋₆, —C₁₋₆ alkylidene-(OC₁₋₆)₁₋₃, —O—C₁₋₆ alkylidene-(halogen)₁₋₃, —SC 1-6, —S—C₁₋₆ alkylidene-(halogen)₁₋₃, or —C₃₋₆ carbocyclic;
In some embodiments, the compound of (I) described above, the pharmaceutically acceptable salt thereof, or the stereoisomer thereof, wherein the structure

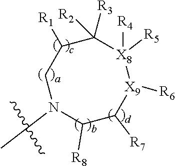

is selected from the following:

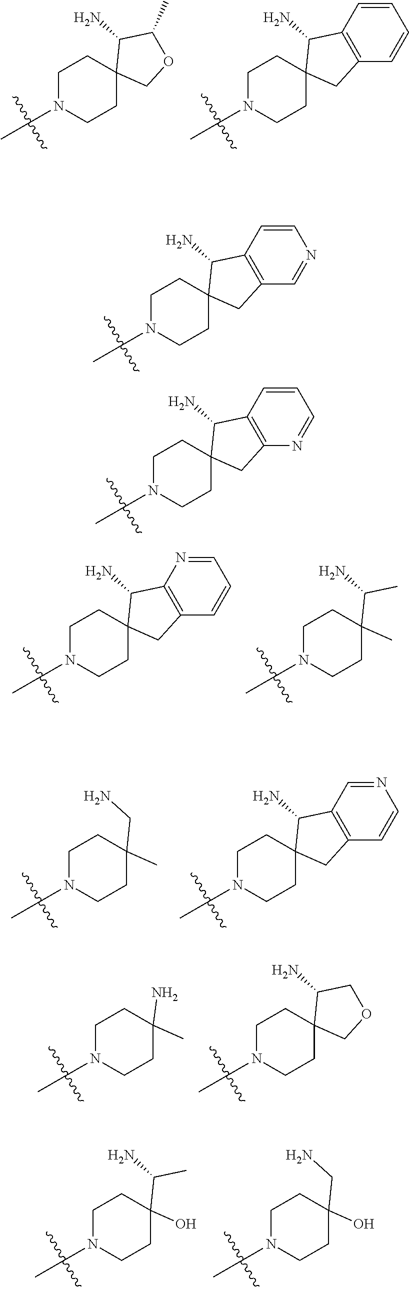

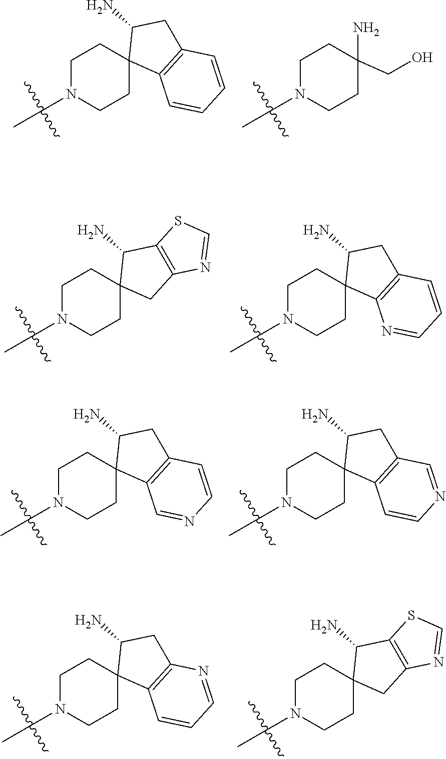

In some embodiments, the compounds of (I) described above, the pharmaceutically acceptable salts thereof, or the stereoisomers thereof, wherein, Ar₂ is independently selected, at each occurrence, from phenyl, naphthyl, 5-membered heteroaryl, 6-membered heteroaryl, 7-membered heteroaryl, 8-membered heteroaryl, 9-membered heteroaryl, or 10-membered heteroaryl, 3-membered to 10-membered cycloalkyl, 5-membered to 10-membered heterocyclic alkyl, and wherein, each of heteroaryl and heterocyclic alkyl at each occurrence independently comprises 1, 2, 3, or 4 heteroatoms selected from N, O, or S; Each Ar₃ is, at each occurrence, independently and optionally substituted or unsubstituted by 1, 2, 3, 4, 5, or 6 R₁₉;
Each Ar₃ is independently selected, at each occurrence, from phenyl, naphthyl, 5-membered heteroaryl, 6-membered heteroaryl, 7-membered heteroaryl, 8-membered heteroaryl, 9-membered heteroaryl, or 10-membered heteroaryl, 3-membered to 10-membered cycloalkyl, 5-membered to 10-membered heterocyclic alkyl, and wherein, each of heteroaryl and heterocyclic alkyl at each occurrence independently comprises 1, 2, 3, or 4 heteroatoms selected from N, O, or S; Each Ar₂ is, at each occurrence, independently and optionally substituted or unsubstituted by 1, 2, 3, 4, 5, or 6 R_{19S};
Each R₁₉ is independently selected, at each occurrence, from deuterium, halogen, oxo, —C₁₋₆ alkyl, —C₁₋₆ alkylidene-(halogen)₁₋₃, C₁₋₆ heteroalkyl, —CN, —OR₁₀, —C₁₋₆ alkylidene-(OR₁₀)₁₋₃, —O—C₁₋₆ alkylidene-(halogen)₁₋₃, —SR₁₀, —S—C₁₋₆ alkylidene-(halogen)₁₋₃, —NR₁₀R₁₁, —C₁₋₆ alkylidene-NR₁₀R₁₁, —C(═O)R₁₀, —C(═O)OR₁₀, —OC(═O)R₁₀, —C(—O)NR₁₀R₁₁, —NR₁₀C(═O)R₁₁, —S(O)₂NR₁₀R₁₁, or —C₃₋₆ carbocyclic; Each R₁₉ is independently optionally substituted by 1, 2, 3, 4, 5 or 6 substituents selected from deuterium, halogen, —C₁₋₆ alkyl, —C₁₋₆ alkoxy, oxo, —OR₁₀, —NR₁₀R₁₁, —CN, —C(═O)R₁₀, —C(═O)OR₁₀, —OC(═O)R₁₀, —C(═O)NR₁₀R₁₁, —NR₁₀C(═O)R₁₁, or —S(O)₂NR₆R₁₁;
Each of R₁₀ and R₁₁ is independently selected, at each occurrence, from hydrogen, deuterium, or —C₁₋₆ alkyl, and each of R₁₀ and R₁₁ is independently optionally substituted or unsubstituted by 1, 2, 3, 4, 5, or 6 R₁₉ₛ; or R₁₀, R₁₁, and the atoms attached thereto form a 3-membered to 10-membered heterocyclic ring, and the stated 3-membered to 10-membered heterocyclic ring can further comprise 1, 2, 3, or 4 heteroatoms selected from N, O, S, S(═O), S(═O), or S(═O)₂, and the stated 3-membered to 10-membered heterocyclic ring is independently optionally substituted or unsubstituted by 1, 2, 3, 4, 5, or 6 R_{20S};
Each R₂₀ is independently selected, at each occurrence, from deuterium, halogen, oxo, —C₁₋₆ alkyl, —C₁₋₆ alkylidene-(halogen)₁₋₃, C₁₋₆ heteroalkyl, CN, —OC₁₋₆, —C₁₋₆ alkylidene-(OC₁₋₆)₁₋₃, —O—C₁₋₆ alkylidene-(halogen)₁₋₃, —SC₁₋₆, —S—C₁₋₆ alkylidene-(halogen)₁₋₃, or —C₃₋₆ carbocyclic;
Further preferably, each

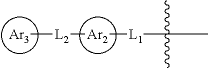

is selected from the following structure:

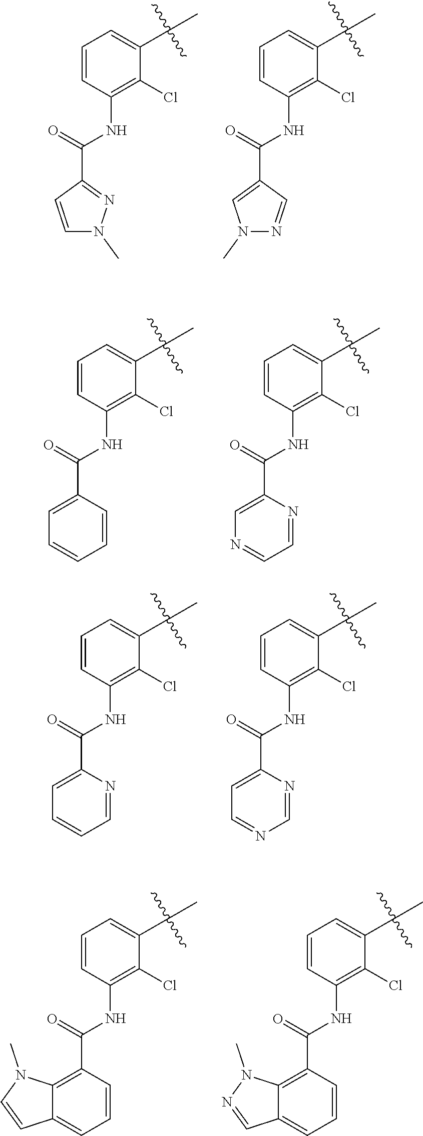

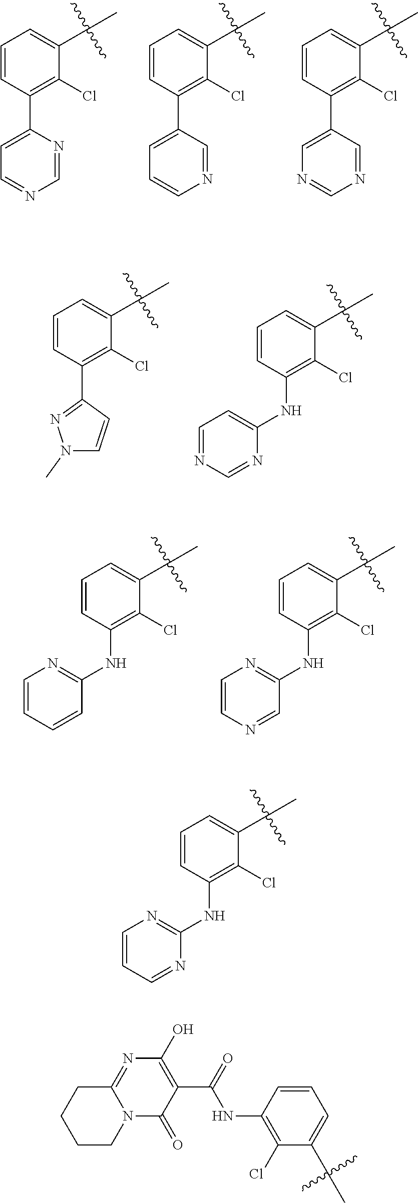

In some embodiments, the compound of (I) described above, the pharmaceutically acceptable salt thereof, or the stereoisomer thereof is selected from the following compounds:

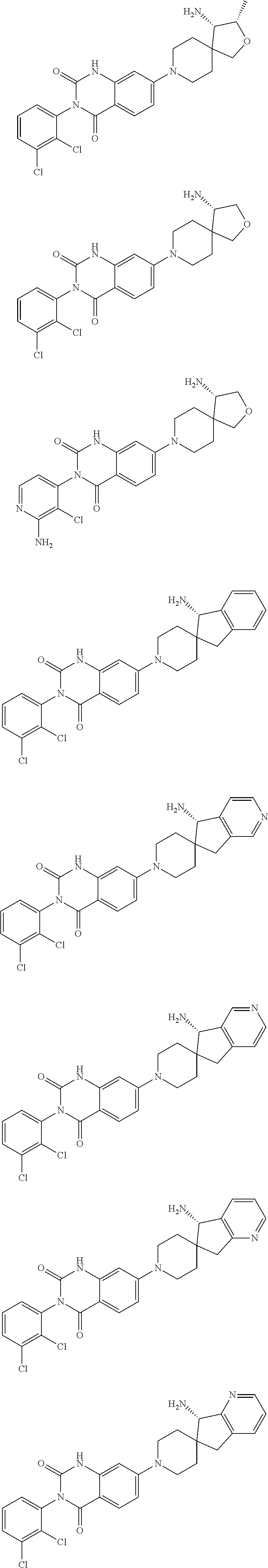

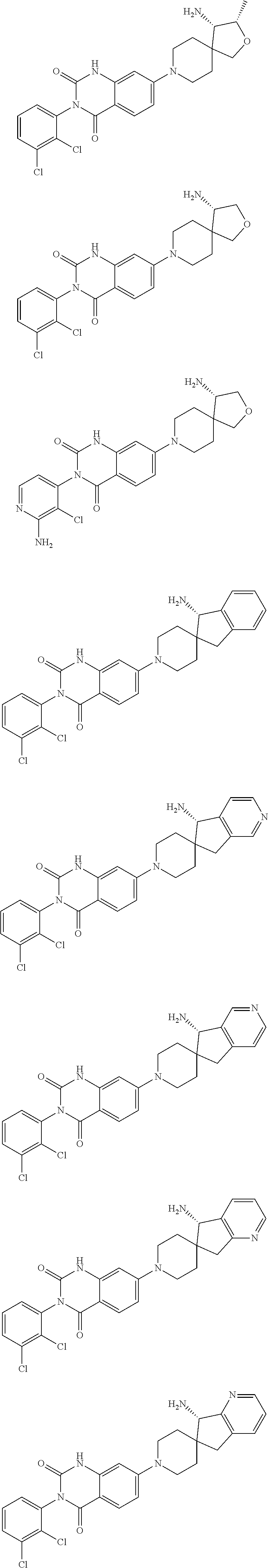

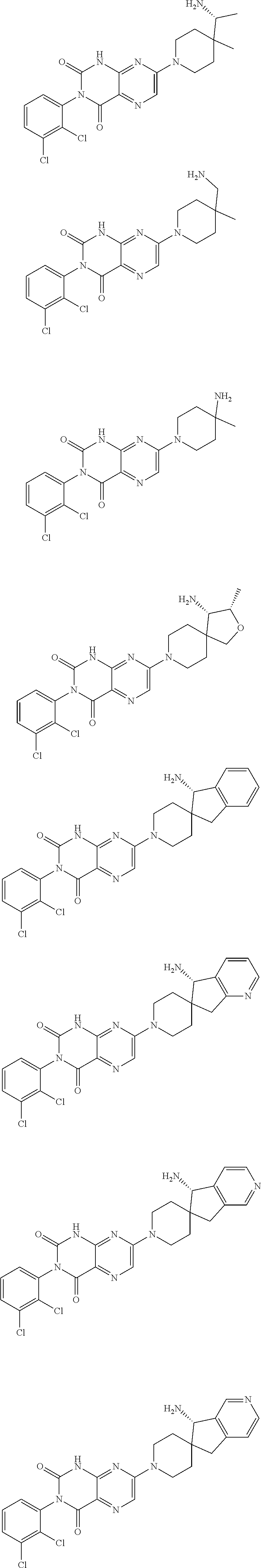

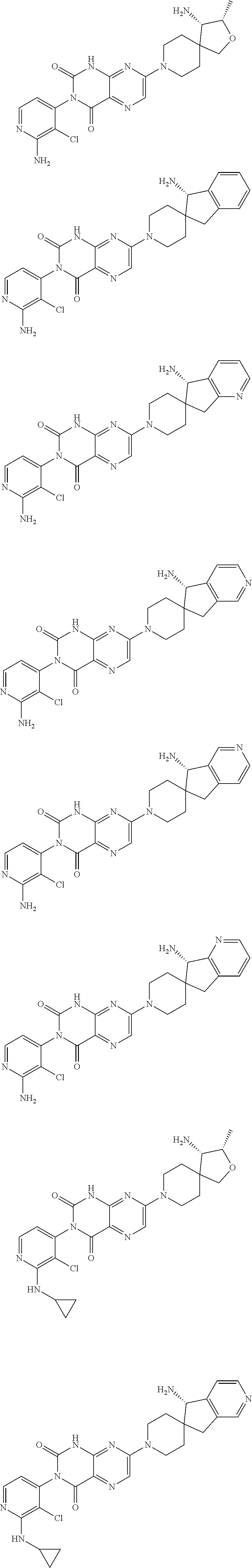

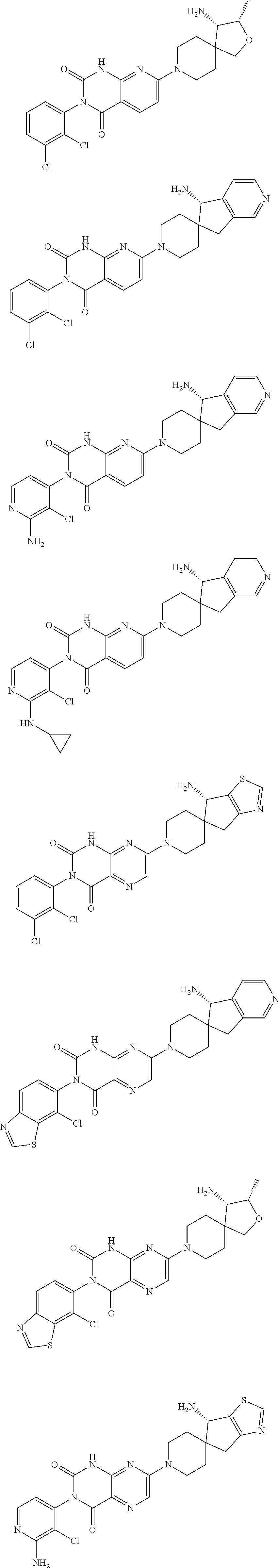

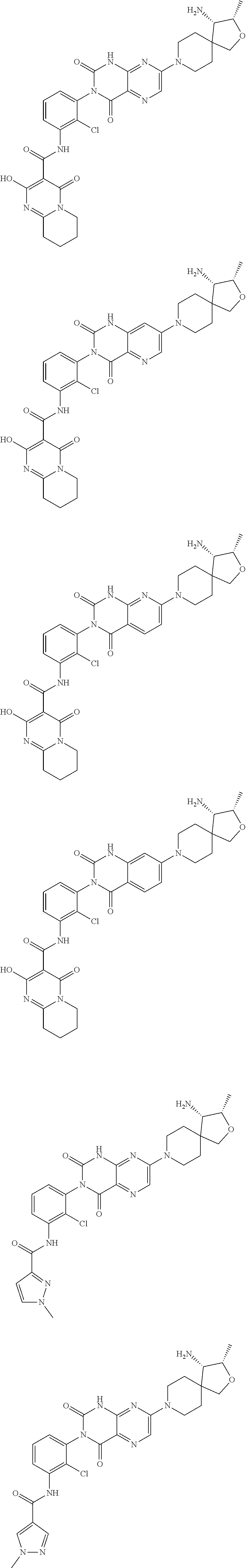

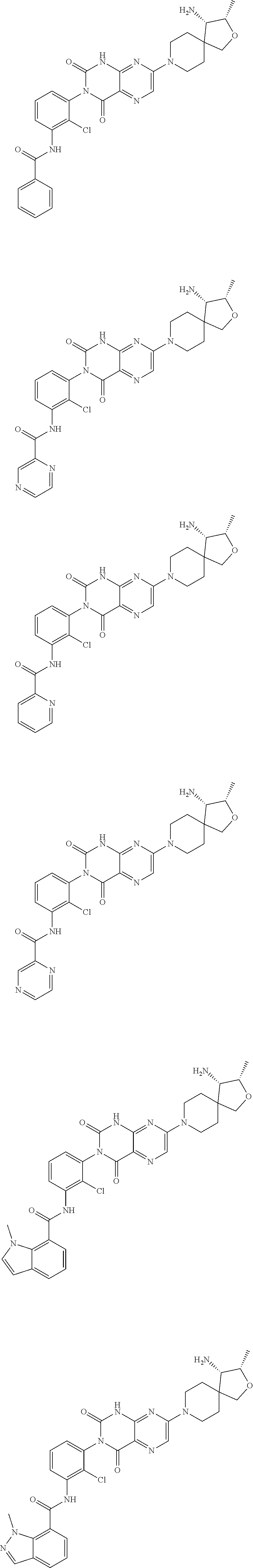

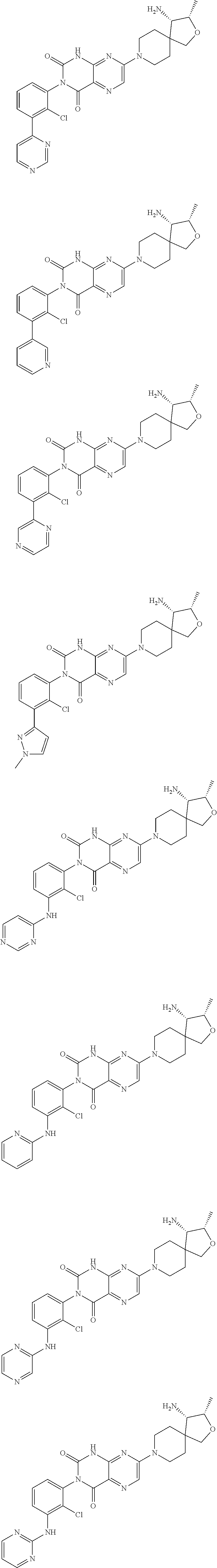

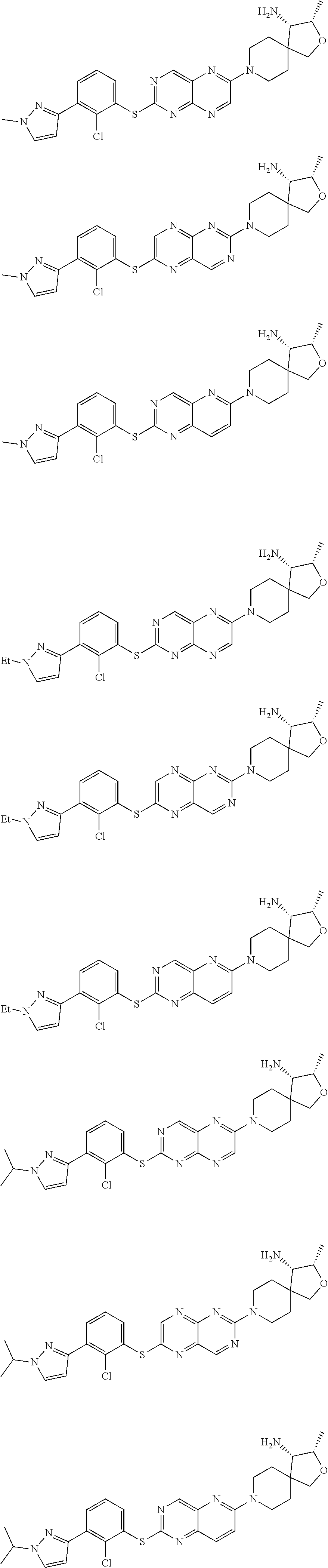

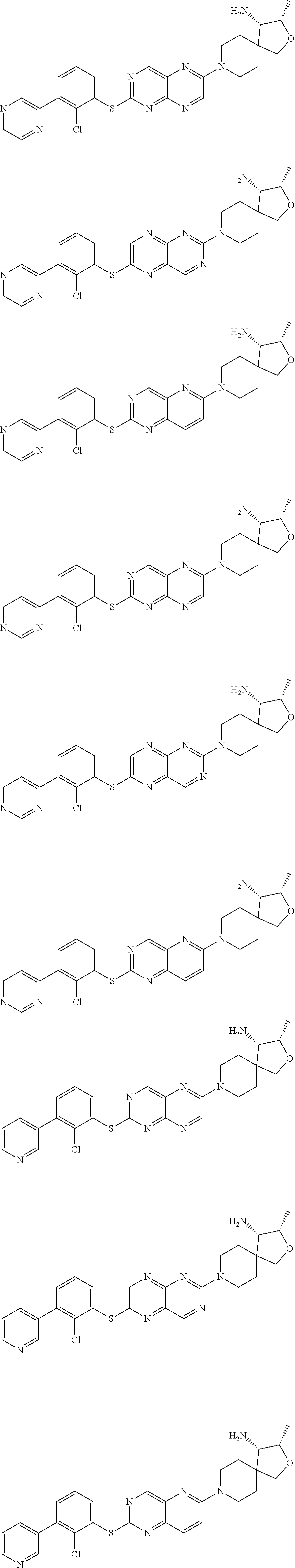

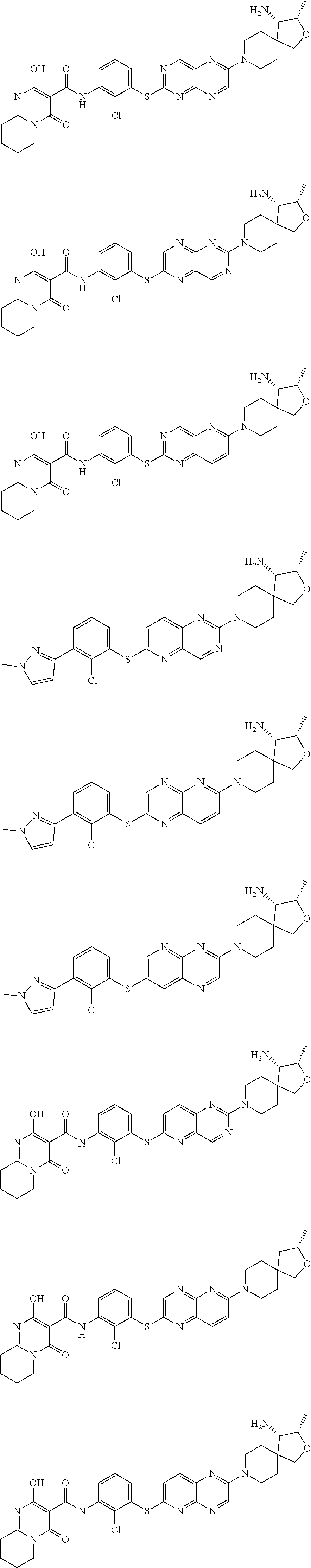

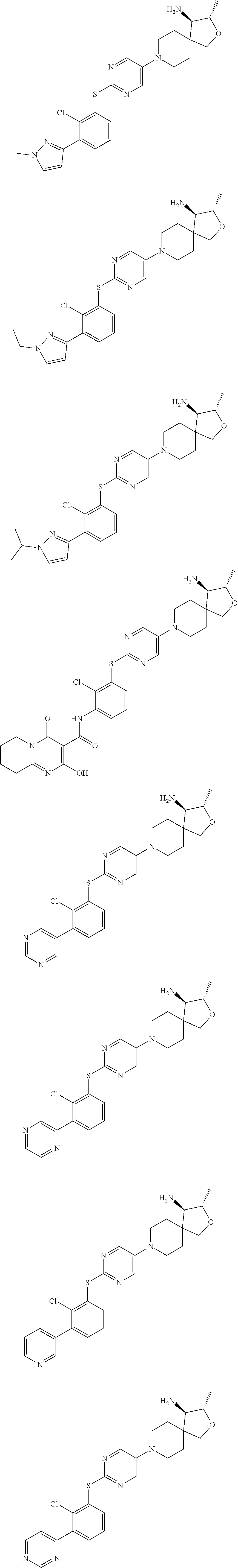

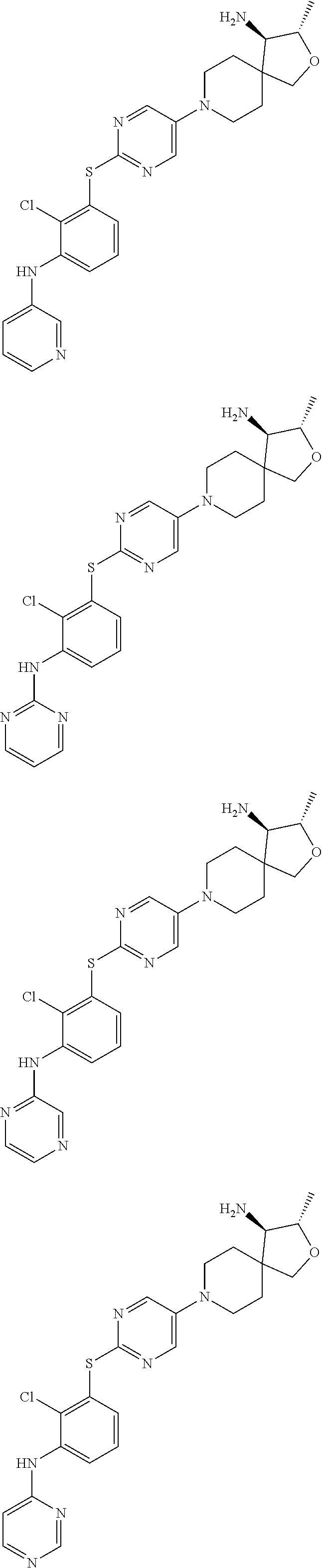 [SMILES: C[C@@H]1OCC2(CCN(c3cnc(Sc4cccc(Nc5cccnc5)c4Cl)nc3)CC2)[C@H]1N ; C[C@@H]1OCC2(CCN(c3cnc(Sc4cccc(Nc5ncccn5)c4Cl)nc3)CC2)[C@H]1N ; C[C@@H]1OCC2(CCN(c3cnc(Sc4cccc(Nc5cnccn5)c4Cl)nc3)CC2)[C@H]1N ; C[C@@H]1OCC2(CCN(c3cnc(Sc4cccc(Nc5ccncn5)c4Cl)nc3)CC2)[C@H]1N]

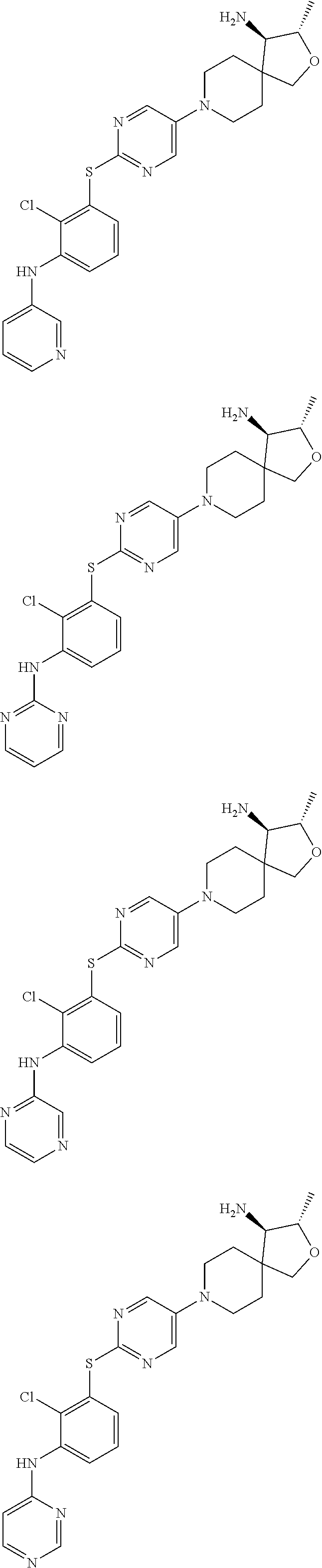 [SMILES: C[C@@H]1OCC2(CCN(c3cnc(Sc4cccc(Nc5cccnc5)c4Cl)nc3)CC2)[C@H]1N ; C[C@@H]1OCC2(CCN(c3cnc(Sc4cccc(Nc5ncccn5)c4Cl)nc3)CC2)[C@H]1N ; C[C@@H]1OCC2(CCN(c3cnc(Sc4cccc(Nc5cnccn5)c4Cl)nc3)CC2)[C@H]1N ; C[C@@H]1OCC2(CCN(c3cnc(Sc4cccc(Nc5ccncn5)c4Cl)nc3)CC2)[C@H]1N]

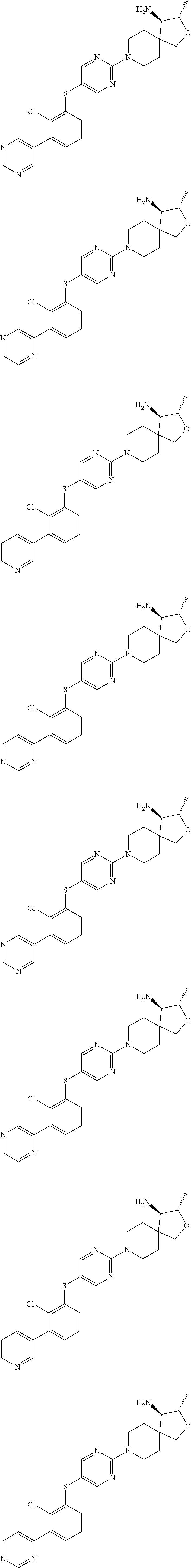

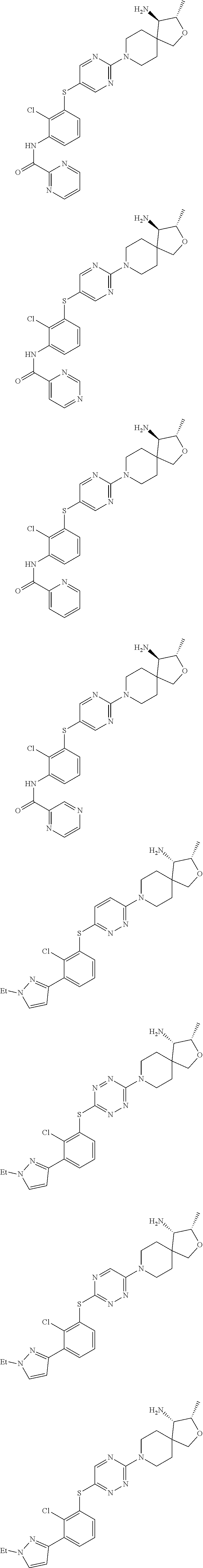

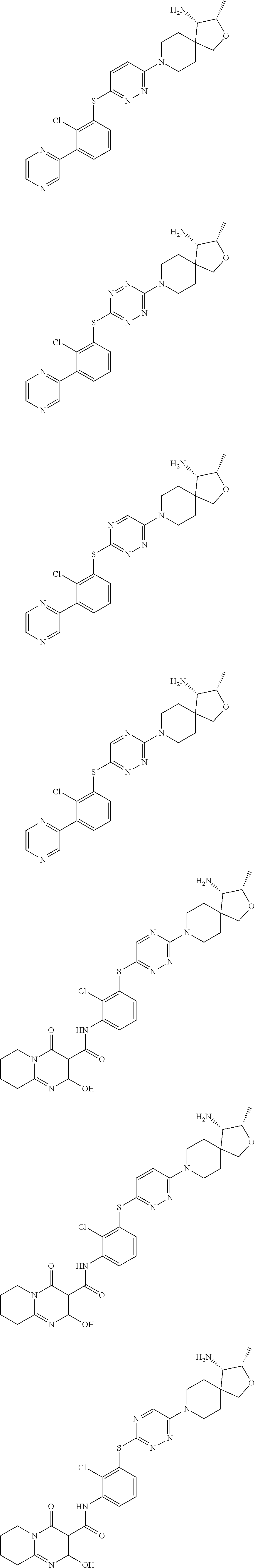

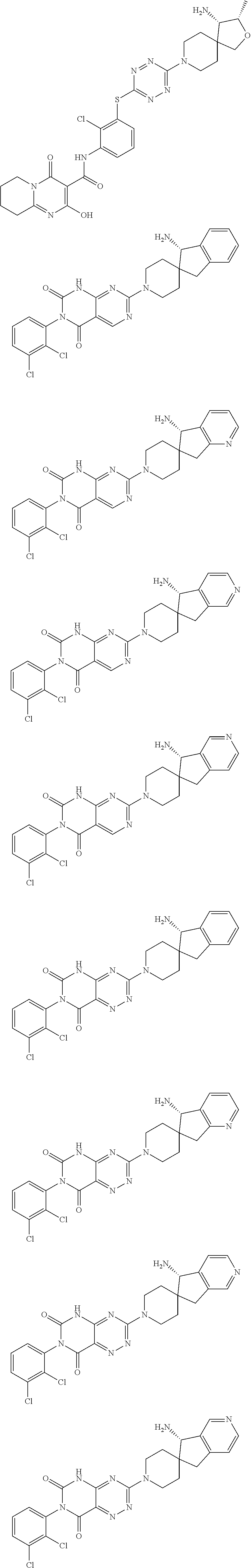

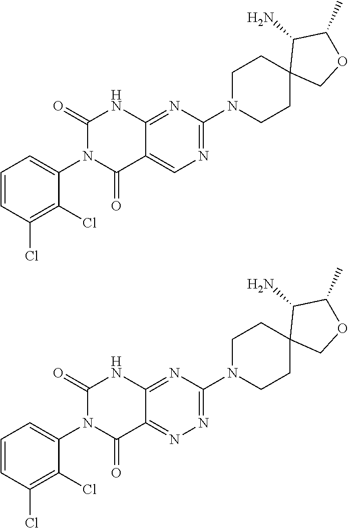 [SMILES: C[C@@H]1OCC2(CCN(c3ncc4c(=O)n(-c5cccc(Cl)c5Cl)c(=O)[nH]c4n3)CC2)[C@@H]1N ; C[C@@H]1OCC2(CCN(c3nnc4c(=O)n(-c5cccc(Cl)c5Cl)c(=O)[nH]c4n3)CC2)[C@@H]1N]

Another aspect of the present disclosure relates to a pharmaceutical composition comprising a therapeutically effective dose of a compound shown in general formula (I) and general formula (II), or a tautomer thereof, a mesomer, racemate, enantiomer, diastereoisomer, atropisomer or mixtures thereof, a pharmaceutically acceptable salt, and one or more pharmaceutically acceptable carriers, diluents, or excipients, wherein, in the present disclosure, the therapeutically effective dose can be selected from 0.1 to 2000 mg.

The present disclosure also relates to a method for preparing the stated pharmaceutical composition, which includes the method of mixing a pharmaceutically acceptable carrier, diluent or excipient with the following compounds: the compounds represented by general formula (I) and general formula (II), their tautomers, mesomers, racemates, enantiomers, diastereoisomers, atropisomers or the mixture thereof, the pharmaceutically acceptable salts thereof, also with the following compound: compounds represented by general formula, their tautomers, mesomers, racemates, enantiomers, diastereoisomers, atropisomers or the mixture thereof, the pharmaceutically acceptable salts thereof.

The present disclosure further relates to the use of the following compounds in the preparation of SHP2 inhibitors: the compounds represented by general formula (I) and general formula (II), or their tautomers, mesomers, racemates, enantiomers, diastereoisomers, atropisomers or mixtures thereof, or their pharmaceutically acceptable salts thereof, or the pharmaceutical compositions containing them.

The present disclosure further relates to the use of the following compounds in the preparation of the medicament for diseases or symptoms mediated by SHP2 activity: the compounds represented by general formula (I) and general formula (II), or their tautomers, mesomers, racemates, enantiomers, diastereoisomers, atropisomers or mixtures thereof, or their pharmaceutically acceptable salts thereof, or the pharmaceutical compositions containing them.

The present disclosure further relates to the use of the following compounds as an SHP2 inhibitor in the preparation of drugs for the prevention and/or treatment of tumors or cancer: the compounds represented by general formula (I) and general formula (II), or their tautomers, mesomers, racemates, enantiomers, diastereoisomers, atropisomers or mixtures thereof, or their pharmaceutically acceptable salts thereof, or the pharmaceutical compositions containing them.

The present disclosure further relates to the use of the following compounds in the preparation of drugs for the prevention and/or treatment of Noonan syndrome, Leopard syndrome, juvenile myelomonocytic leukemia, neuroblastoma, melanoma, acute myeloid leukemia, breast cancer, esophageal cancer, lung cancer, colon cancer, head cancer, pancreatic cancer, head and neck squamous cell carcinoma, gastric cancer, liver cancer, anaplastic large cell lymphoma, and glioblastoma: the compounds represented by general formula (I) and general formula (II), or their tautomers, mesomers, racemates, enantiomers, diastereoisomers, atropisomers or mixtures thereof, or their pharmaceutically acceptable salts thereof, or the pharmaceutical compositions containing them.

The present disclosure further relates to the following compounds as drugs: the compounds represented by general formula (I) and general formula (II), or their tautomers, mesomers, racemates, enantiomers, diastereoisomers, atropisomers or mixtures thereof, or their pharmaceutically acceptable salts thereof, or the pharmaceutical compositions containing them.

The present disclosure further relates to the following compounds as an SHP2 inhibitor: the compounds represented by general formula (I) and general formula (II), or their tautomers, mesomers, racemates, enantiomers, diastereoisomers, atropisomers or mixtures thereof, or their pharmaceutically acceptable salts thereof, or the pharmaceutical compositions containing them.

The present disclosure further relates to the use of the following compounds as a SHP2 inhibitor for the prevention and/or treatment of tumors or cancer: the compounds represented by general formula (I) and general formula (II), or their tautomers, mesomers, racemates, enantiomers, diastereoisomers, atropisomers or mixtures thereof, or their pharmaceutically acceptable salts thereof, or the pharmaceutical compositions containing them.

The present disclosure also relates to a method for preventing and/or treating tumors or cancers, the pharmaceutical composition of which are as follows: the compounds represented by the general formulas as an SHP2 inhibitor, or their tautomers, mesomers, racemates, enantiomers, diastereoisomers, atropisomers or mixtures thereof, or their pharmaceutically acceptable salts thereof, or the pharmaceutical compositions containing them; the pharmaceutical composition containing active ingredient may be in a form suitable for oral administration, such as tablet, trochiscus, lozenge, aqueous or oily suspensions, dispersible powder or granule, emulsion, hard or soft capsule, or syrup; this oral composition can be prepared according to any method known in the field for the preparation of pharmaceutical compositions, and such compositions can contain one or more ingredients as following: sweetener, corrigents, colorant and preservative, which is used to provide visually and taste-friendly pharmaceutical preparations, and non-toxic pharmaceutically acceptable excipient. These excipients can be inert excipients, granulation agents, disintegrants, binders, or lubricants. These tablets can be uncoated, or can be coated by known techniques that mask the taste of the drug or delay disintegration and absorption in the gastrointestinal tract.

Oral formulations can also be provided in soft gelatin capsules in which the active ingredient is mixed with an inert solid diluent or in which the active ingredient is mixed with a water-soluble carrier or oil-soluble medium.

Aqueous suspensions contain the active ingredient, and the mixed excipient suitable for preparing the aqueous suspension. Such excipients are suspending agent, dispersing agent or wetting agent. The aqueous suspension can also contain one or more preservatives, one or more colorants, one or more corrigents and one or more sweeteners.

Oil suspension can be prepared by suspending the active ingredient in vegetable oil, or mineral oil. The oil suspension can contain thickener. The sweeteners and colorants described above can be added to provide palate-friendly preparation. These compositions can be preserved by adding antioxidants.

The pharmaceutical compositions disclosed can also be in the form of an oil-in-water emulsion, wherein the oil phase can be a vegetable oil, or a mineral oil or the mixture thereof, and wherein the suitable emulsifier can be a naturally-existing phospholipid; and the emulsion can also contain sweetener, corrigent, preservative, and antioxidant. Such formulations can also contain moderator, preservative, colorant and antioxidant. The pharmaceutical compositions disclosed can be in the form of sterile injectable aqueous solutions. Acceptable media or solvents are water, ringer's solution and isotonic sodium chloride solution. The sterile injectable preparation can be a sterile injectable oil-in-water microemulsion in which the active ingredient is dissolved in the oil phase. The injectable solution or microemulsion can be administered into the blood circulation system of a patient by locally bulk injection or, preferably, the solution and microemulsion can be given in a manner that maintains a constant circulating concentration of the compounds disclosed in the present invention. To maintain this constant concentration, an example of a continuous intravenous drug delivery device is the Deltec CADD-plus.™. 5400 mode IV pump.

The pharmaceutical compositions in the present disclosure can be in the form of sterile injectable water or oil suspensions for intramuscular and subcutaneous administration. The mixture can be formulated by mixing those suitable dispersant or wetting agents with suspending agent as described above in accordance with known techniques, and the sterile injectable preparation can also be parenteral sterile injectable solution or suspension, which are prepared in non-toxic diluent or solvent, and in addition, sterile fixed oils can conveniently be used as solvents or suspending mediums, and for this purpose any blended fixed oils can be used. Additionally, fatty acids also can be used to prepare injectable formulations as well.

The compounds in the present disclosure can be administrated in the form of suppository for rectal use. These pharmaceutical compositions can be prepared by mixing the drug with a suitable non-irritating excipient that is solid at normal temperatures, but change to liquid in the rectum and thus will dissolve in the rectum to release the drug.

As is well known to those skilled in the field, the dosage of a drug to be administered is dependent on a variety of factors including, but not limited to, the following factors, e.g. the activity of the specific compound used, the age of the patient, the body weight of the patient, the health of the patient, the behavior of the patient, the diet of the patient, the time of administration, the way of administration, the rate of excretion, and the combination of drugs; in addition, the optimal treatment method such as the treatment mode, the daily dosage of the compounds in the general formulas (I) and (II) or the type of pharmaceutically available salt can be verified based on conventional treatment protocols.

### Certain Chemical Terms

Unless stated to the contrary, the following terms should be applied in the specification and claims.

The expression “C_{x-y}”, as used herein, describes a range of carbon atoms, wherein x and y are integers, e.g., C₃₋₈ cycloalkyl signifies the cycloalkyl with 3 to 8 carbon atoms, i.e., cycloalkyl with 3, 4, 5, 6, 7, or 8 carbon atoms. It should also be understood that the “C₃₋₈” also includes any of the subranges thereof, such as C₃₋₇, C₃₋₆, C₄₋₇, C₄₋₆, C₅₋₆, and the like.

The “alkyl” refers to a straight or branched alkyl group containing 1 to 20 carbon atoms, e.g., from 1 to 18 carbon atoms, from 1 to 12 carbon atoms, from 1 to 8 carbon atoms, from 1 to 6 carbon atoms, or from 1 to 4 carbon atoms. Non-limiting examples of alkyl groups include methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl and 2-ethylbutyl. The stated alkyl groups can be substituted or unsubstituted.

The “Alkenyl” refers to a straight or branched alkyl group containing at least one carbon-carbon double bond and carbon atoms ranging from 2 to 20, such as 2 to 8 carbon atoms, 2 to 6 carbon atoms or 2 to 4 carbon atoms. Non-limiting examples of alkenyl groups include vinyl, 1-propenyl, 2-propenyl, 1-butenyl, 2-butenyl, 3-butenyl, 2-methyl-2-propenyl, 1,4-pentadienyl, and 1,4-butadienyl. The stated alkenyl group can be substituted or unsubstituted.

The “alkynyl” refers to a straight or branched hydrocarbon group containing at least one carbon-carbon triple bond and carbon atoms ranging from 2 to 20, e.g., from 2 to 8 carbon atoms, from 2 to 6 carbon atoms or from 2 to 4 carbon atoms. Non-limiting examples of alkynyl groups include ethynyl, 1-propynyl, 2-propynyl, 1-butynyl, 2-butynyl and 3-butynyl. The stated alkynyl group can be substituted or unsubstituted.

“Cycloalkyl” refers to a saturated cyclic hydrocarbon substituent containing 3 to 14 carbocyclic atoms. The cycloalkyl group can be monocyclic, typically containing 3 to 7 carbocyclic atoms. Non-limiting examples of monocyclic cycloalkyl groups include cyclopropyl, cyclobutyl, cyclopentyl, cyclohexyl and cycloheptyl. Cycloalkyl can optionally be condensed bicyclic rings or tricyclic rings, such as a decahydronaphthyl group, and the stated cycloalkyl can be substituted or unsubstituted.

The “heterocyclyl”, “heterocyclic alkyl” and “heterocycle” refer to a stable 3-membered to 18-membered monovalent non-aromatic ring, comprising 2 to 12 carbon atoms, and 1 to 6 heteroatoms selected from nitrogen, oxygen and sulfur. Unless otherwise indicated, the heterocyclic group can be a monocyclic, bicyclic, tricyclic, or tetracyclic system, which can comprise condensed ring, spiro, or bridged ring system. The nitrogen, carbon, or sulfur on the heterocyclic group is optionally oxidized, the nitrogen atom is optionally quaternized, and the heterocyclic group can be partially or fully saturated. The carbons or heteroatoms in the heterocyclyl can be connected to the rest of the heterocyclyl by a single bond. Provided that it is atoms on the non-aromatic ring that are connected to the rest of a heterocyclyl, the heterocyclyl comprising a fused ring can contain one or more aromatic or heteroaromatic rings. For the purposes of the present application, the heterocyclic group is preferably a stabilized 4-membered to 11-membered monovalent non-aromatic monocyclic or bicyclic ring, which comprises 1 to 3 heteroatoms selected from nitrogen, oxygen or sulfur; and more preferably, it is a 4-membered to 8-membered monovalent non-aromatic monocyclic ring, which comprises 1 to 3 heteroatoms selected from nitrogen, oxygen or sulfur. Non-limiting examples of heterocyclic groups include azepanyl, azetidinyl, decahydroisoquinolinyl, dihydrofuranyl, dihydroindolyl, dioxolyl, 1,1-dioxo-morpholino-thiomorpholino, imidazolidinyl, imidazolidinyl, isothiazolidinyl, isoxazolidinyl, morpholiny, octahydroindolyl, octahydroisoindolyl, oxazinyl, piperazinyl, piperidinyl, 4-piperidinoneyl, pyranyl, pyrazolidinyl, pyrrolidinyl, quinuclidinyl, quinuclidine ringyl, tetrahydrofuranyl, and tetrahydropyranyl, etc.

The “spiro heterocycly” refers to 5-membered to 20-membered polycyclic heterocyclic group of which monocyclic rings share a single atom (called a spiro atom) each other, wherein, one or more of the ring atoms is selected from the heteroatoms of nitrogen, oxygen, or S(O)ₘ (wherein m is an integer from 0 to 2), and the remaining ring atom(s) is/are carbon. These electronic systems containing one or more double bonds, but none of the rings being fully completely conjugated are preferably 6-membered to 14-membered, more preferably 7-membered to 10-membered. Spiro cycloalkyls are categorized as mono-spiro cycloalkyl, double spiro heterocyclyl or polyspiro heterocyclyl based on the number of spiro atoms shared between the rings, preferably mono-spiro cycloalkyl and double spiro heterocyclyl. More preferred are 4-membered/4-membered, 4-membered/5-membered, 4-membered/6-membered, 5-membered/5-membered or 5-membered/6-membered mono-spiro cycloalkyls. Non-limiting examples of spiro heterocyclyl comprise:

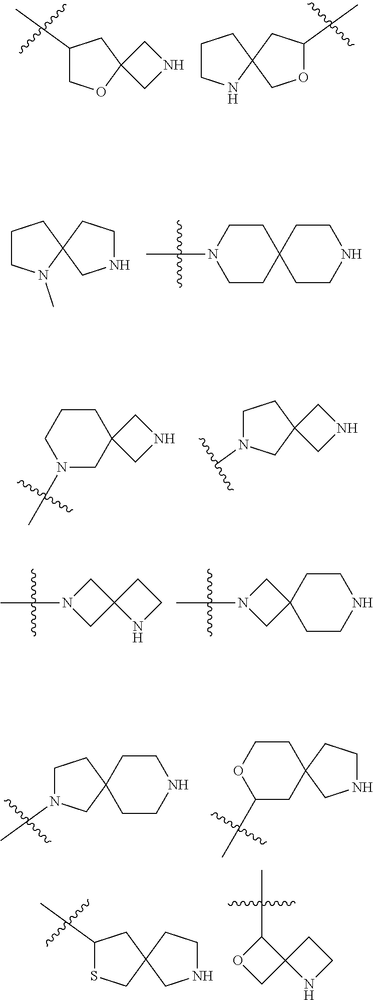

The “fused heterocyclyl” refers to 5-membered to 20-membered polycyclic heterocyclic group, wherein each ring thereof shares an adjoining pair of atoms with other rings, where one or more of the rings can contain one or more double bonds but none of the rings has a fully conjugated π-electronic system, wherein one or more of the ring atoms is selected from a heteroatom of nitrogen, oxygen, or S(O)ₘ (wherein m is an integer from 0 to 2) and the remaining ring atom(s) is/are carbon. Preferably are 6-membered to 14-membered, more preferably are 7-membered to 10-membered. Depending on the number of constituent rings, the fused heterocyclyl can be classified as bicyclic, tricyclic, tetracyclic or polycyclic fused heterocyclic alkyl, preferably bicyclic or tricyclic, more preferably 5-membered/5-membered or 5-membered/6-membered bicyclic fused heterocyclyl. Non-limiting examples of fused heterocyclic group comprise:

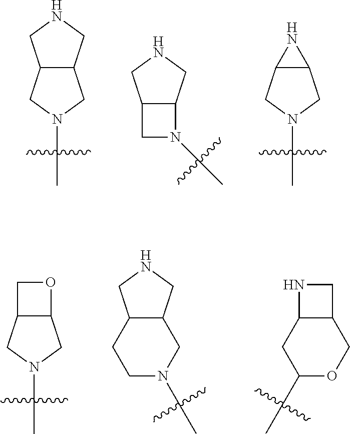 [SMILES: *N1CC2CNCC2C1 ; *N1CC2CNCC21 ; *N1CC2NC2C1 ; *N1CC2COC2C1 ; *N1CCC2CNCC2C1 ; *C1CC2NCC2CO1]

The “aryl” or “aromatic” refers to an aromatic monocyclic group or fused polycyclic group containing 6 to 14 carbon atoms, preferably 6-membered to 10-membered, such as phenyl and naphthyl, more preferably phenyl. The stated aryl ring can be condensed bound to heteroaryl, heterocyclyl or cycloalkyl rings, wherein the ring which is attached to the parent structure is an aryl ring.

The “heteroaryl” or “heteroaromatic” refers to 5-membered to 16-membered ring system comprising 1 to 15 carbon atoms, preferably 1 to 10 carbon atoms, 1 to 4 heteroatoms selected from nitrogen, oxygen and sulfur, and at least one aromatic ring. Unless otherwise indicated, the heteroaryl group can be monocyclic, bicyclic, tricyclic or tetracyclic systems, which can contain fused or bridged ring systems, provided that the site of attachment to the rest of the molecule is an aryl atom, that the nitrogen, carbon and sulphur atoms on the heteroaryl ring can be selectively oxidized and the nitrogen atoms can be selectively quaternized. For the purposes of the present invention, the heteroaryl group is preferably a stabilized 4-membered to 11-membered monoaromatic ring comprising 1 to 3 heteroatoms selected from nitrogen, oxygen and sulfur, and more preferably a stabilized 5-membered to 8-membered monoaromatic ring comprising 1 to 3 heteroatoms selected from nitrogen, oxygen and sulfur. Non-limiting examples of heteroaryl group include acridinyl, azepinenyl, benzimidazolyl, benzindolyl, benzodioxinyl, benzodioxolyl, benzofuranonyl, benzo Furyl, benzonaphthofuryl, benzopyrone, benzopyranyl, benzopyrazolyl, benzothiadiazolyl, benzothiazolyl, benzotriazolyl, furyl, imidazolyl, indazolyl, indolyl, oxazolyl, purinyl, pyrazinyl, pyrazolyl, pyridazinyl, pyridyl, pyrimidinyl, pyrrolyl, quinazolinyl, quinolinyl, quinine Base, tetrazolyl, thiadiazolyl, thiazolyl, thienyl, triazinyl, and triazolyl. In the present application, the heteroaryl group is preferably a 5-membered to 8-membered heteroaryl group comprising 1 to 3 heteroatoms selected from nitrogen, oxygen and sulfur, and more preferably pyridinyl, pyrimidinyl, and thiazolyl. The stated heteroaryl group can be substituted or unsubstituted.

The “halogen” refers to fluorine, chlorine, bromine or iodine.

The “hydroxyl” refers to —OH, “amino” refers to —NH₂, “acylamino” refers to —NHCO—, “cyano group” refers to —CN, “nitro” refers to —NO₂, “isocyano” refers to —NC, and “trifluoromethyl” refers to —CF₃.

The term “heteroatom” or “hetero” as used herein, alone or as part of other compositions, refers to atoms other than carbon and hydrogen, the heteroatom is independently selected from, but not limited to, oxygen, nitrogen, sulfur, phosphorus, silicon, selenium, and tin; in embodiments where two or more heteroatoms are present, the stated two or more heteroatoms can be identical to each other or some or all of the stated two or more heteroatoms can be different.

The terms “fused” or “fused ring” as used herein, alone or in combination, refer to a cyclic structure in which two or more rings share one or more bonds.

The terms “spiro” or “spiro ring” as used herein, alone or in combination, refer to a cyclic structure in which two or more rings share one or more atoms.

The “optional” or “optionally” means that the subsequently described event or circumstance may but not necessary to occur, and that the description includes instances where the event or circumstance occurs or does not occur; for example, “heterocyclic group optionally substituted by alkyl” means that the alkyl group may but not necessary to be present, and this description includes both cases where the heterocyclic group is substituted by an alkyl group and cases where the heterocyclic group is not substituted by an alkyl group.

The “substituted” refers to that one or more atoms of the group, preferably 5, more preferably 1 to 3 atoms, are substituted independently by a corresponding number of substituents. It is self-evident that the substituents are in their possible chemical positions and that the person skilled in the field is able to determine (experimentally or theoretically) possible or impossible substitutions without undue effort. For example, carbon atoms with free amine or hydroxyl groups may be unstable when bonded to carbon atoms with unsaturated (e.g., alkenyl) bonds. The stated substituents include, but are not limited to, hydroxyl, amine, halogen, cyano group, C₁₋₆ alkyl, C₁₋₆ alkoxy, C₂₋₆ alkenyl, C₂₋₆ alkynyl, and C₃₋₈ cycloalkyl, etc.

The “pharmaceutical composition” refers to a composition containing one or more of compounds described herein, pharmaceutically acceptable salts or prodrugs thereof, or other ingredients such as pharmaceutically acceptable carriers and excipients. Pharmaceutical compositions are intended to facilitate the administration to an organism, and the absorption of the active ingredient(s), and thus biological activity is developed.

The “isomer” refers to the compounds that have the same molecular formula but a different nature or order of atomic bonding, or a different spatial arrangement of atoms thereof, and an isomer with a different spatial arrangement of atoms is called a “stereoisomer”. The stereoisomers include optical isomer, geometric isomer and conformational isomer. The compounds of the present invention can present in the form of optical isomer. Depending on the configuration of the substituents around the chiral carbon atom, these optical isomers are either “R” or “S” configurations. Optical isomers include enantiomer and diastereomer, and methods for preparing and isolating optical isomer are known in the field.

The compounds of the present invention can also present as geometrical isomer. The present invention contemplates various geometrical isomers and mixtures thereof resulted from the distribution of substituents around carbon-carbon double bonds, carbon-nitrogen double bonds, cycloalkyl or heterocyclic groups. The substituents around carbon-carbon double bonds or carbon-nitrogen bonds are designated as Z or E configurations, and the substituents around cycloalkyl or heterocyclic groups are designated as cis or trans configurations.

The compounds of the present invention can also present with tautomerism, such as keto-enol tautomerism.

It can be understood that the present invention includes any of the tautomeric or stereoisomeric forms and mixtures thereof, and is not limited to any one of the tautomeric or stereoisomeric forms used in the nomenclature or chemical conjugation formulae of the compounds.

The “isotope” refers to all isotopes of the atoms appear in the compounds of the present invention. Isotope include those atoms having the same atomic number but different mass numbers. Examples of isotopes suitable for incorporation into the compounds of the present invention are hydrogen, carbon, nitrogen, oxygen, phosphorus, fluorine and chlorine, such as, but not limited to, ²H, ³H, ¹³C, ¹⁴C, ¹⁵N, ¹⁸O, ³¹P, ³²P, ³⁵S, ¹⁸F and ³⁶Cl. The isotopically labeled compounds of the invention can, using an appropriate isotopically labeled reagent, generally replace non-isotopically labeled preparation with conventional techniques known to those skilled in the field or methods analogous to those described in the appended examples. Such compounds have a variety of potential uses, such as specimens and reagents in the determination of biological activity. In the case of stable isotopes, such compounds have the potential to favorably modify biological, pharmacological or pharmacokinetic properties.

The “prodrug” refers to that a compound of the invention can be given in the form of a prodrug. A prodrug is a derivative of a biologically active compound of the invention that is converted under physiological conditions in vivo, e.g., by oxidation, reduction, hydrolysis, etc. (each of which is carried out using an enzyme or without an enzyme). Examples of prodrugs are compounds as follows: wherein the amine groups in the compounds of the present invention are acylated, alkylated or phosphorylated, such as eicosanoylamino, alanylamide, pivaloyloxymethylamine; Or, wherein, the hydroxyl groups are acylated, alkylate, phosphorylated, or converted into borates, such as acetoxy, palmitoyloxy, pivaloyloxy, succinyloxy, fumaryloxy, alanyloxy; Or, wherein the carboxyl groups are esterified or aminated; Or, wherein, the sulfhydryl group forms a disulfide bridge with a carrier molecule (e.g., a peptide) that selectively delivers drugs to the cytosol of cells, these compounds can be prepared from the compounds of the present invention according to known methods.

The “pharmaceutically usable salt” or “pharmaceutically acceptable” refers to pharmaceutically usable base or acid, which are made from inorganic base or acid, and organic base or acid. In cases of when the compounds of the invention contain one or more acidic or basic groups, the invention also comprises their corresponding pharmaceutically usable salts. Thus, the compounds of the invention containing acidic groups can be present in the form of salts and can be used according to the invention as alkali metal salt, alkaline-earth metal or ammonium salt. More specific examples of such salts include sodium salt, potassium salt, calcium salt, magnesium salt, amine or organic amine such as primary amine, secondary amine, tertiary amine, and cyclic amine, e.g. ammonia, isopropylamine, trimethylamine, diethylamine, triethylamine, tripropylamine, ethanolamine, diethanolamine, ethanolamine, diethanolamine, dicyclohexylamine, ethylenediamine, purine, piperazine, piperidine, choline and caffein, etc.; particularly preferred organic bases are isopropylamine, diethylamine, ethanolamine, trimethylamine, dicyclohexylamine, choline, and caffeine salt. The compounds of the invention containing basic groups can be present in the form of salts and can be used according to the invention in the form of their addition to inorganic or organic acids. Examples of suitable acids include hydrochloric acid, hydrobromic acid, phosphoric acid, sulfuric acid, phosphoric acid, methylsulfonic acid, p-toluenesulfonic acid, naphthalene disulfonic acid, oxalic acid, acetic acid, dihydroxysuccinic acid, lactic acid, salicylic acid, benzoic acid, formic acid, propionic acid, valproic acid, malonic acid, succinic acid, heptanedioic acid, fumaric acid, maleic acid, malic acid, aminosulfonic acid, phenylpropionic acid, gluconic acid, ascorbic acid, isonicotinic acid, citric acid, adipic acid, and other acids known to those skilled in the field. If the compounds of the present invention contain both acidic and basic groups, the invention also includes, in addition to the salt forms mentioned, inner salts or betaines. The respective salts are obtained by conventional methods known to those skilled in the field, for example by contacting these with organic or inorganic acids or bases in solvents or dispersants or by anion-exchange or cation-exchange with other salts.

Accordingly, in this application, reference to “the compounds”, “the compounds of the invention” or “the compounds of the present invention” includes all the described compound forms, such as their prodrugs, stable isotope derivatives, pharmaceutically usable salts, isomer, mesomere and racemate, enantiomer, diastereomer and mixtures thereof.

As used herein, the term “cancer” refers to both benign and malignant tumors (e.g., cancer).

As used herein, the term “cancer” includes a variety of malignancies in which SHP2 phosphatase is involved, including, but not limited to, non-small cell lung cancer, esophageal cancer, melanoma, rhabdomyosarcoma, cellular carcinoma, multiple myeloma, breast cancer, ovarian cancer, uterine membrane cancer, cervical cancer, gastric cancer, node cancer, bladder cancer, pancreatic cancer, lung cancer, breast cancer, prostate cancer, and liver cancer (e.g., hepatocellular carcinoma), and more specifically liver cancer, gastric cancer, and bladder cancer.

As used herein, the term “effective dose”, “therapeutically effective dose” or “pharmaceutically effective dose” refers to the dose of at least one agentia or compound that, when administered, is sufficient to provide some relief of one or more symptoms of the disease or condition being treated. The results can be an abatement and/or relief of the sign, symptom or cause of disease or any other desired improvement of the biological system. For example, an “effective dose” for therapeutic use is the dose of the compositions of the compounds disclosed herein required to provide clinically significant relief from diseases. An effective dose suitable for use in any individual case can be determined using techniques such as dose-escalation testing.

The term of “polymorphic substance” or “polycrystalline type (phenomenon)” as used in the present invention refers to that the compounds of the present invention have multiple crystalline lattice forms, some compounds of the present invention can have more than one crystalline form, and that the present invention encompasses all of the polymorphic states or mixtures thereof.

Intermediate compounds of the compounds of the present invention and their polymorphs are also within the present invention.

Crystallization often produces solvates of the compounds of the present invention, and the term “solvate” as used herein refers to a combination of one or more compound molecules of the present invention with one or more solvent molecules.

The solvent can be water, in which case the solvate is a hydrate, alternatively an organic solvent. Thus, the compounds of the present invention can exist as hydrates, including monohydrates, dihydrates, hemihydrates, trihydrates, tetrahydrates, and the like, and the corresponding solvated forms. The compounds of the present invention can be true solvates, but in some other cases, the compounds of the present invention can also only incidentally retain water or mixtures of water with some other solvents. The compounds of the present invention can be reacted in one solvent or precipitated or crystallized in one solvent. The solvates of the compounds of the present invention are also included within the invention.

The term “acceptable” as used herein related to preparation, composition or ingredient refers to no sustained deleterious effect on the general health of the subject to be treated.

The term “pharmaceutically acceptable XX” as used herein refers to a substance (e.g., a carrier or diluent) that does not interfere with the biological activity or properties of the compounds of the present invention and is relatively non-toxic, i.e., the substance can be administered to an individual without causing an adverse biological reaction or interacting adversely with any of the components contained in the composition.

“Pharmaceutically acceptable carriers” include, but are not limited to, adjuvants, carriers, excipients, auxiliaries, deodorizers, diluents, preservatives, dyes/colorants, flavor enhancers, surfactants, wetting agents, dispersants, suspending agents and stabilizers, isotonizing agents, solvent or emulsifiers, etc, which have been approved for use in human beings and in domesticated animals by the relevant governmental administration.

The terms “subject”, “patient”, “object”, or “individual” as used herein refer to an individual suffering from a disease, disorder, or symptom, etc., and include both mammals and non-mammals. Examples of mammals include, but are not limited to, any member of the class Mammalia: humans, non-human primates (e.g., chimpanzees and other apes, and monkeys); livestock, such as cows, horses, sheep, goats, and swine; domesticated animals, such as rabbits, dogs, and cats; laboratory animals, including rodents such as rats, mice, and guinea pigs, etc. Examples of non-human mammals include, but are not limited to, birds and fishes, etc. In one embodiment of the methods and compositions provided herein, the stated mammal is human being.

The term “treatment” as used herein refers to the treatment of relevant diseases and syndromes in mammals, especially in human beings, including
(i) Preventing the disease and syndrome in mammals, especially mammals that have been previously exposed to a disease or syndrome but have not been diagnosed with the disease or syndrome;
(ii) Inhibiting a disease or syndrome, i.e., controlling its progress;
(iii) Alleviating the disease or syndrome, i.e., causing the disease or syndrome to subside;
(iv) Alleviating the symptoms caused by a disease or syndrome.

The terms “disease” and “syndrome” as used herein can be interchangeable or have different meanings, since certain specific diseases or syndromes have no known causative factor (and therefore no known cause) and therefore cannot be recognized as diseases but only as unwanted symptoms or syndromes. The stated syndromes have more or less specific symptoms that have been confirmed by clinical researchers.

The term “administration” as used herein, refers to the methods that enable delivery of a compound or composition to a desired site of biological action, including, but not limited to, oral route, transduodenal route, parenteral injection (including intravenous, subcutaneous, intraperitoneal, intramuscular, intraarterial injection or infusion), local delivery, and transrectal administration. In preferred embodiments, the compounds and compositions discussed herein are administered orally.

### Specific Embodiments

The present invention also provides methods of preparing the stated compounds. The preparation of the compounds described in general formula (I) and general formula (II) of the present invention can be done by the following exemplary methods and embodiments, which however, should not be considered in any way as limiting the scope of the present invention. The compounds described herein can also be synthesized by synthetic techniques known to those skilled in the field, or by a combination of methods known in the field and methods described herein. The product that should be given from each step is obtained by separation techniques known in the field, including but not limited to extraction, filtration, distillation, crystallization, and chromatographic separation, etc. The starting materials and chemical reagents required for the syntheses can be conventionally synthesized or purchased according to literature (the Reaxys).

The pyrimidine heterocyclic compounds described in general formula (IIa) and general formula (IIb) of the present invention can be synthesized according to the following route

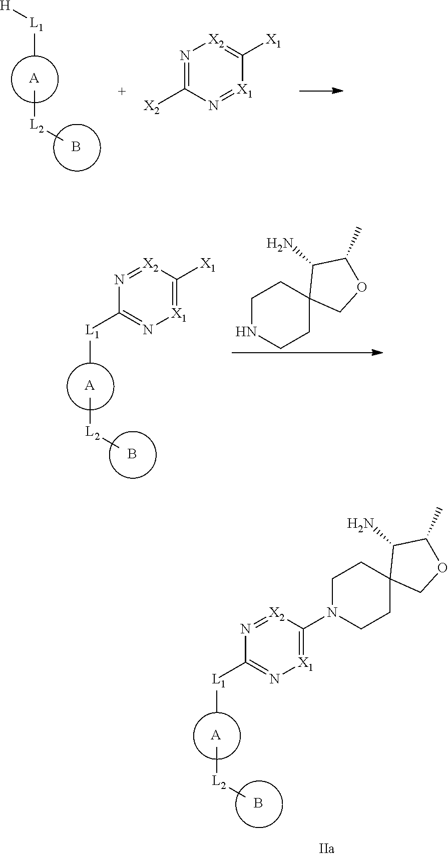

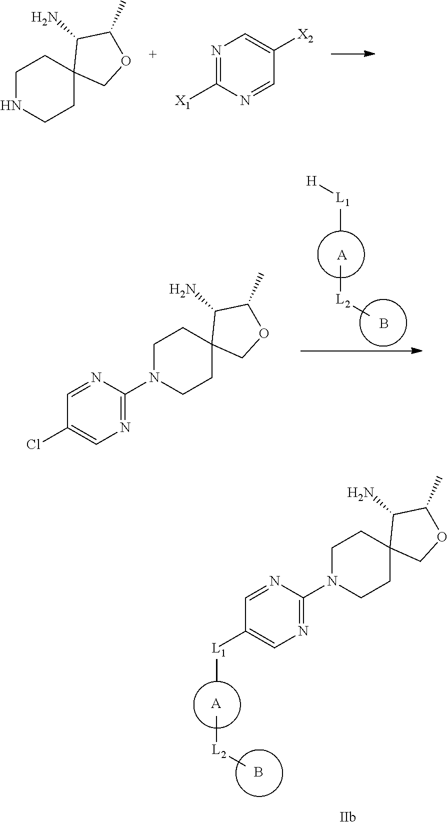

Temperature is expressed as Celsius unless otherwise indicated. Reagents are purchased from commercial suppliers such as Chem blocks Inc, Astatech Inc or McLean, etc., and, these reagents are used directly without further purification unless otherwise indicated.

Unless otherwise indicated, the following reactions are carried out at room temperature, in anhydrous solvents, under positive pressure of nitrogen or argon or using drying tubes and, glassware is dried and/or heated to dryness.

Unless otherwise indicated, 200-300 mesh silica gel from Qingdao Ocean Chemical Factory is used for column chromatography purification; thin-layer chromatography silica gel prefabricated plates (HSGF254) produced by Yantai Chemical Industry Research Institute are used for the preparative thin-layer chromatography; liquid chromatograph mass spectrometer of Thermo LCD Fleet mode is used for MS determination.

Nuclear magnetic data (¹H NMR) is collected with Bruker Avance-400 MHz or Varian Oxford-400 Hz NMR equipment, the solvents used for nuclear magnetic data are CDCl₃, CD₃OD, D₂O, DMSO-d₆, etc.; based on tetramethylsilane (0.000 ppm) or residual solvent (CDCl₃: 7.26 ppm; CD₃OD: 3.31 ppm; D₂O: 4.79 ppm; DMSO-d₆: 2.50 ppm), when there are virous peak shapes, the following abbreviations represent different peak shapes: s (singlet), d (doublet), t (triplet), q (quartet), m (multiplet), br (broad), dd (double doublet), dt (double triplet). If coupling constants are given, they are in Hertz (Hz).

The following embodiments can further describe the invention; however, these embodiments should not be seen as limiting the scope of the invention.

### Preparation of Intermediates

### Preparation of the Intermediate (S)-1,3-dihydrospiro[indene-2,4′-piperidine]-1-amine

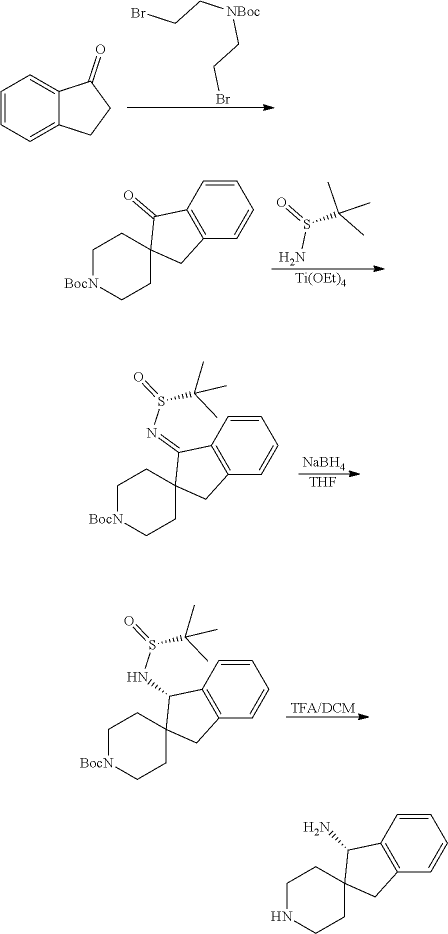

Dissolve the compound 1-indanone (2.64 g, 20 mmol) in 30 mL N,N-dimethylformamide, add NaH (2.40 g, 60 mmol, 60%) at room temperature, stir the reaction mixture for 30 min. Add N—BOC-N,N-bis(2-bromomethyl)amine (7.28 g, 22 mmol), heat to 50° C., and stir, react for 12 hours. Cool to room temperature, the reaction solution is quenched with water and, extracted with ethyl acetate. The resulting organic phase is then washed with saturated brine, dried with anhydrous sodium sulfate, and the organic phase is evaporated to dryness under reduced pressure. The residue is purified by column chromatography to give the compound tert-butyl 1-oxo-1,3-dihydrospiro[indene-2,4′-piperidine]-1′-carboxylate (2.11 g, 35% yield). LC/MS (ESI): m/z=202.1[M+H]⁺.

Dissolve the compound tert-butyl 1-oxo-1,3-dihydrospiro[indene-2,4′-piperidine]-1′-carboxylate (1.0 g, 3.32 mmol) and compound (R)-tert-butylsulfinamide (1.21 g, 10.0 mmol) in 10 mL of tetrahydrofuran, add ethyl titanate (4.87 mL, 23.23 mmol), heat to 65° C., and stir for 48 hours, then cool to room temperature, add ethyl acetate and water, stir for 15 minutes, the resulting solid is removed by filtration. The organic phase is dried with anhydrous sodium sulfate, filtered and evaporated under reduced pressure to give the crude product (R,E)-1-(tert-butylsulfinylimino)-1,3-dihydrospiro[indene-2,4′-piperidine]-1′-carboxylic acid tert-butyl ester, which is used directly in the next step.

Dissolve the compound (R,E)-1-(tert-butylsulfinylimino)-1,3-dihydrospiro[indene-2,4′-piperidine]-1′-carboxylic acid tert-butyl ester (1.21 g, 3 mmol) in 15 mL tetrahydrofuran solution and, cool to −45° C., add sodium borohydride (0.17 g, 4.5 mmol). Stir the mixture and let the temperature resume to room temperature, react for 18 hours, quench with ice water and, extract with dichloromethane. The resulting organic phase is then washed with saturated brine, dried with anhydrous sodium sulfate, and the organic phase is evaporated to dryness under reduced pressure. The residue is purified by column chromatography to give the compound (S,R)-1-(tert-butylsulfinylamino)-1,3-dihydrospirocyclo[indene-2,4′-piperidine]-1′-carbox ylic acid tert-butyl ester (485 mg, 40% yield).

Dissolve the compound (S,R)-1-(tert-butylsulfinamido)-1,3-dihydrospiro[indene-2,4′-piperidine]-1′-carboxylic acid tert-butyl ester (485 mg, 1.19 mmol) in 1 mL of dichloromethane, add 1 mL of trifluoroacetic acid, stir, and react for 1 hour. The reaction solution is concentrated under reduced pressure. The residue is purified by reverse preparative column chromatography to give compound (S)-1,3-dihydrospiro[indene-2,4′-piperidine]-1-amine (229 mg, 95% yield). LC/MS (ESI): m/z=203.1[M+H]⁺.

### Preparation of the Intermediate (S)-5,7-dihydrospiro[cyclopenta[b]pyridine-6,4′-piperidine]-5-amine

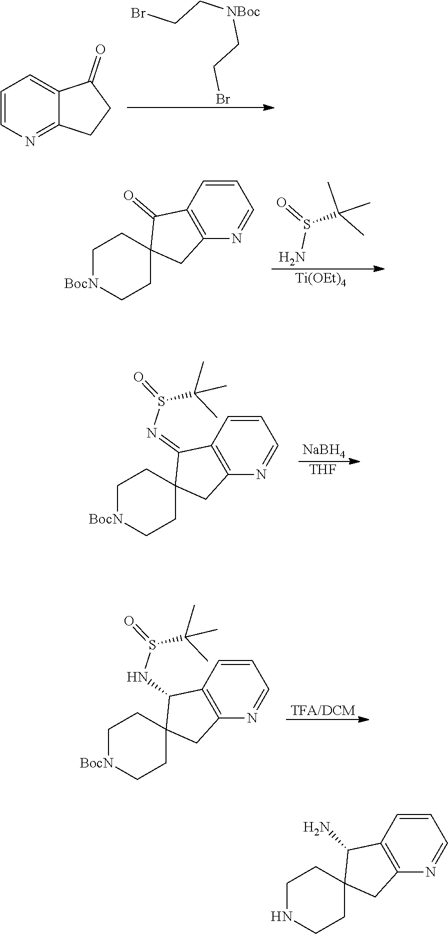

The compound (S)-5,7-dihydrospiro[cyclopenta[b]pyridine-6,4′-piperidine]-5-amine is obtained by a method intermediate similar to that of (S)-1,3-dihydrospiro[indene-2,4′-piperidine]-1-amine(raw material is changed to 6,7-dihydro-5H-cyclopenta[b]pyridine-5-one). LC/MS (ESI): m/z=204.1[M+H]⁺.

### Preparation of the Intermediate (S)-5,7-dihydrospiro[cyclopenta[c]pyridine-6,4′-piperidine]-5-amine

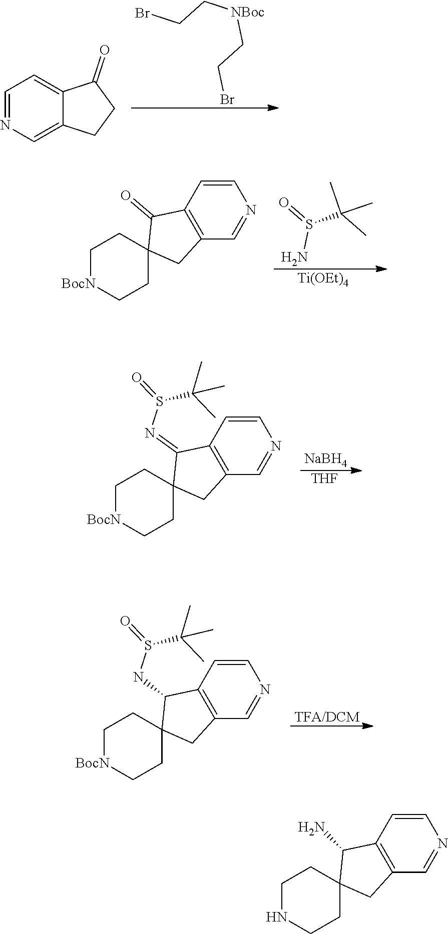

The compound (S)-5,7-dihydrospiro[cyclopenta[c]pyridine-6,4′-piperidine]-5-amine is obtained by a method similar to that of intermediate (S)-1,3-dihydrospiro[indene-2,4′-piperidine]-1-amine (the raw material is changed to 6,7-dihydro-5H-cyclopenta[c]pyridine-5-ketone). LC/MS (ESI): m/z=204.1 [M+H]⁺.

### Preparation of the Intermediate (S)-5,7-dihydrospiro[cyclopenta[c]pyridine-6,4′-piperidine]-7-amine

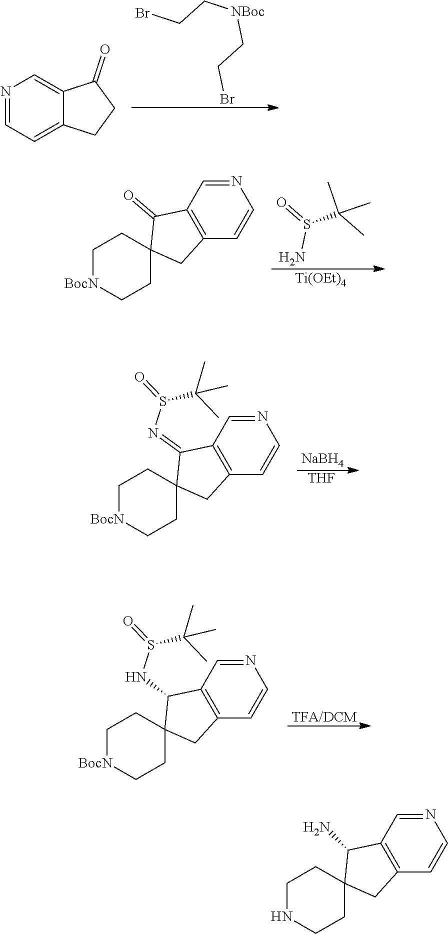

The compound (S)-5,7-dihydrospiro[cyclopenta[c]pyridine-6,4′-piperidine]-7-amine is obtained by a method similar to that of intermediate (S)-1,3-dihydrospiro[indene-2,4′-piperidine]-1-amine (the raw material is changed to 5H-cyclopenta[c]pyridine-7(6H)-ketone). LC/MS (ESI): m/z=204.1[M+H]⁺.

### Preparation of the Intermediate (S)-5,7-dihydrospiro[cyclopenta[b]pyridine-6,4′-piperidine]-7-amine

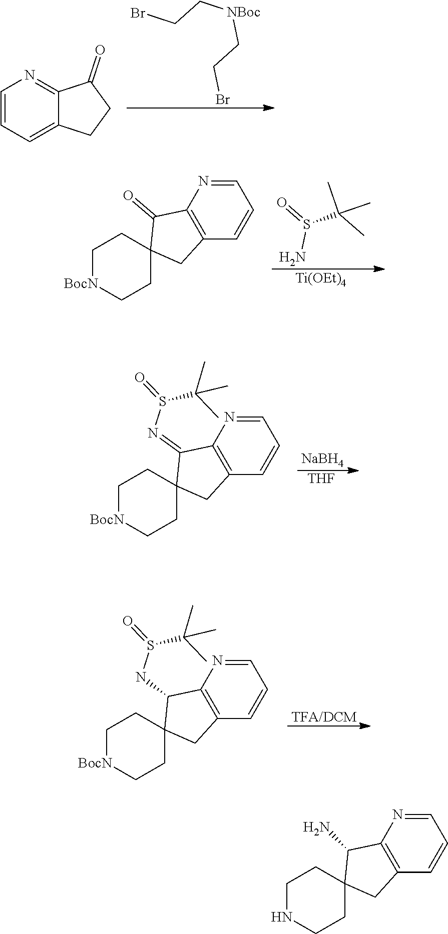

The compound (S)-5,7-dihydrospiro[cyclopenta[b]pyridine-6,4′-piperidine]-7-amine is obtained by a method similar to that of intermediate (S)-1,3-dihydrospiro[indene-2,4′-piperidine]-1-amine (the raw material is changed to 5H-cyclopenta[b]pyridine-7(6H)-ketone). LC/MS (ESI): m/z=204.1[M+H]⁺.

### Preparation of the Intermediate (R)-1-(4-methylpiperidine-4-yl)ethylamine

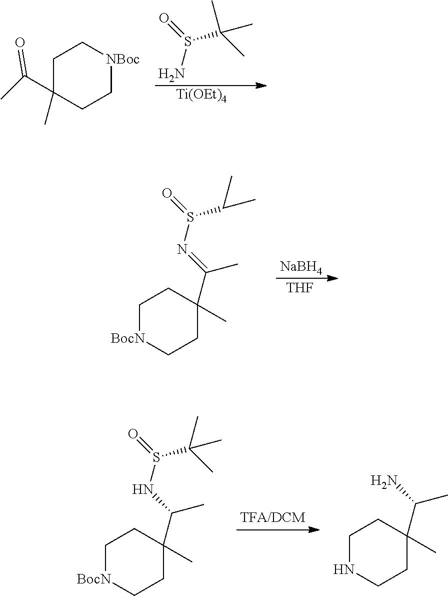

The compound (R)-1-(4-methylpiperidine-4-yl)ethylamine is obtained by a preparation similar to that of intermediate (S)-1,3-dihydrospiro[indene-2,4′-piperidine]-1-amine (the raw material is changed to tert-butyl-4-acyl-4-methyl-piperidine-1-carboxylate). LC/MS (ESI): m/z=143.1[M+H]⁺.

### Preparation of the Intermediate (4-methylpiperidine-4-yl)methylamine

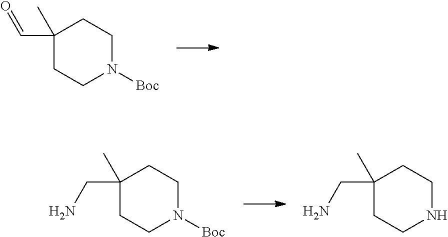

Dissolve the compound 1-BOC-4-formyl-4-methylpiperidine (0.68 g, 3 mmol) and ammonium acetate (1.21 g, 30.0 mmol) in 10 mL of methanol, add sodium cyanoborohydride (0.25 g, 4 mmol), stir, and react at room temperature for 1 hour. The resulting mixture is diluted by chloroform, washed with 3N sodium hydroxide solution and saturated brine, the organic phase is dried with anhydrous sodium sulfate, filtered, and evaporated under reduced pressure to give the compound 4-(aminomethyl)-4-methylpiperidine-1-carboxylic acid tert-butyl ester (513 mg, 75% yield). LC/MS (ESI): m/z=129.1[M+H]⁺.

Dissolve the compound 4-(aminomethyl)-4-methylpiperidine-1-carboxylic acid tert-butyl ester (456 mg, 2 mmol) in 1 mL of methanol, add 1,4-dioxane solution in HCl (1 mL, 4M), stir, and react at room temperature for 1 hour. The reaction solution is concentrated under reduced pressure. The residue is purified by a reverse preparative column chromatography to give the compound (4-methylpiperidine-4-yl) methylamine (240 mg, 93% yield). LC/MS(ESI): m/z=129.1[M+H]⁺.

### Preparation of the Intermediate (S)-4,6-dihydrospiro[cyclopenta[b]thiazol-5,4′-piperidine]-6-amine

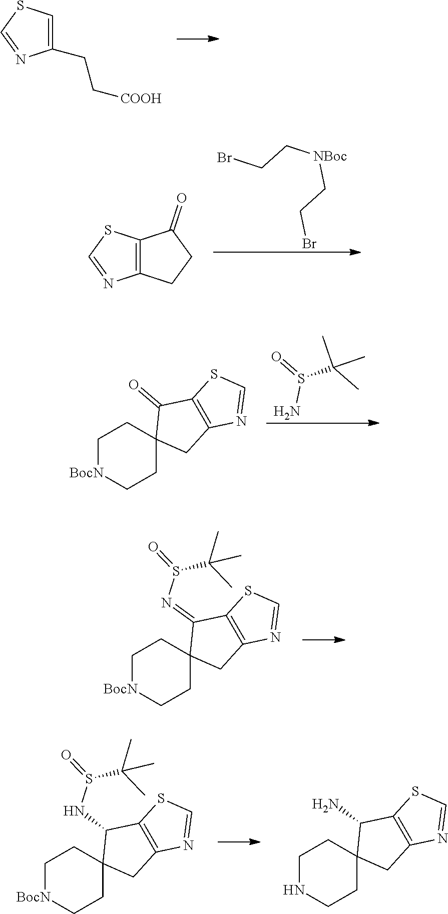

To a 100 mL suspension of phosphorus pentoxide (25.4 g, 179.0 mmol) in methanesulfonic acid, add 3-(1,3-thiazol-4-yl)propanoic acid (5.0 g, 32.0 mmol, stir and react at room temperature for 1 hour. The reaction solution is concentrated under reduced pressure and, the residue is purified by column chromatography to give the compound 4H-cyclopenta[b]thiazol-6(5H)-ketone (1.2 g, 27% yield). LC/MS (ESI): m/z=140.0[M+H]⁺.

In the subsequent steps, a preparation method similar to that of the intermediate (S)-1,3-dihydrospiro[indene-2,4′-piperidine]-1-amine (the raw material is changed to 4H-cyclopenta[b]thiazole-6(5H)-ketone) is used to obtain compound (S)-4,6-dihydrospiro[cyclopenta[b]thiazole-5,4′-piperidine]-6-amine. LC/MS (ESI): m/z=210.1 [M+H]⁺.

### Preparation of the Intermediate 7-bromo-3-(2,3-dichlorophenyl)quinazoline-2,4(1H,3H)-dione

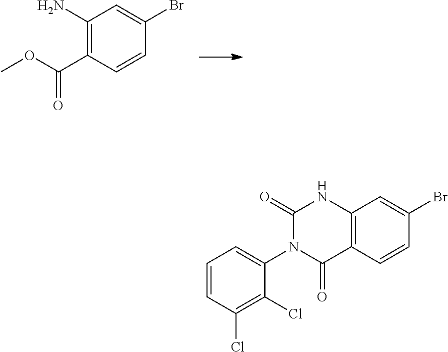 [SMILES: COC(=O)c1ccc(Br)cc1N ; O=c1[nH]c2cc(Br)ccc2c(=O)n1-c1cccc(Cl)c1Cl]

Dissolve the compound methyl 2-amino-4-bromobenzoate (1.15 g, 5 mmol) in 25 mL of anhydrous 1,4-dioxane, add 2,3-dichlorophenylisocyanate (1.13 g, 6 mmol) and triethylamine (101 mg, 1 mmol). The reaction is carried out at 90° C. for 48 h under stirring. Cool to room temperature, the reaction solution is evaporated under reduced pressure and, the residue is purified by column chromatography to give the intermediate 7-bromo-3-(2,3-dichlorophenyl)quinazoline-2,4(1H,3H)-dione (1.5 g, 78% yield). LC/MS (ESI): m/z=384.9[M+H]⁺.

### Preparation of Intermediate 7-chloro-3-(2,3-dichlorophenyl)pteridine-2,4(1H,3H)-dione

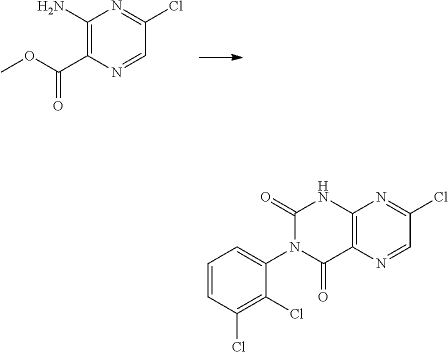 [SMILES: COC(=O)c1ncc(Cl)nc1N ; O=c1[nH]c2nc(Cl)cnc2c(=O)n1-c1cccc(Cl)c1Cl]

The preparation method similar to that of intermediate 7-bromo-3-(2,3-dichlorophenyl)quinazoline-2,4(1H,3H)-dione (the raw material is replaced by 3-amino-5-chloro pyrazine-2-carboxylic acid methyl ester) to obtain the compound 7-chloro-3-(2,3-dichlorophenyl)pteridine-2,4(1H,3H)-dione. LC/MS (ESI): m/z=342.9[M+H]⁺.

### Preparation of the Intermediate 7-chloro-3-(2,3-dichlorophenyl)pyrido[2,3-d]pyrimidine-2,4(1H,3H)-dione

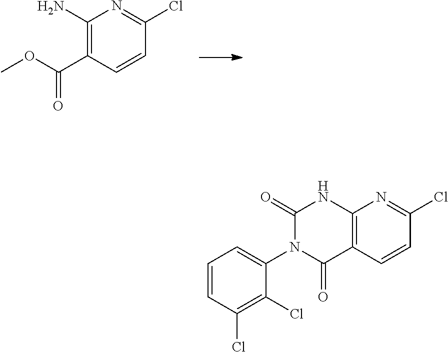 [SMILES: COC(=O)c1ccc(Cl)nc1N ; O=c1[nH]c2nc(Cl)ccc2c(=O)n1-c1cccc(Cl)c1Cl]

The compound 7-chloro-3-(2,3-dichlorophenyl)pyrido[2,3-d]pyrimidine-2,4(1H,3H)-dione is obtained by a preparation method similar to that of the intermediate 7-bromo-3-(2,3-dichlorophenyl)quinazoline-2,4(1H,3H)-dione (raw material is changed to 2-amino-6-chloronicotinic acid methyl ester). LC/MS(ESI): m/z=342.0[M+H]⁺.

### Preparation of the Intermediate 7-chloro-3-(7-chlorobenzo[d]thiazol-6-yl)pteridine-2,4(1H,3H)-dione

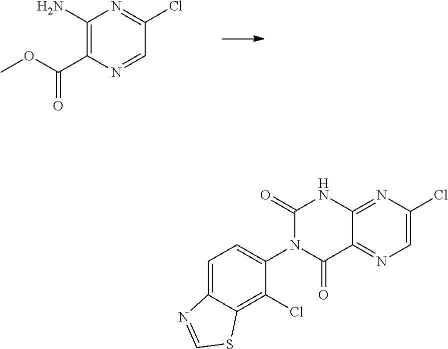 [SMILES: COC(=O)c1ncc(Cl)nc1N ; O=c1[nH]c2nc(Cl)cnc2c(=O)n1-c1ccc2ncsc2c1Cl]

The compound 7-chloro-3-(7-chlorobenzo[d]thiazol-6-yl)pteridine-2,4(1H,3H)-dione is obtained by a method similar to the intermediate 7-bromo-3-(2,3-dichlorophenyl)quinazoline-2,4(1H,3H)-dione (raw material is changed to methyl 3-amino-5-chloropyrazine-2-carboxylate and 7-chlorobenzo[d]thiazol-6-amine). LC/MS (ESI): m/z=366.0[M+H]⁺.

### Preparation of 7-bromo-3-(2-amino-3-chloropyridine-4-yl)quinazoline-2,4(1H,3H)-dione

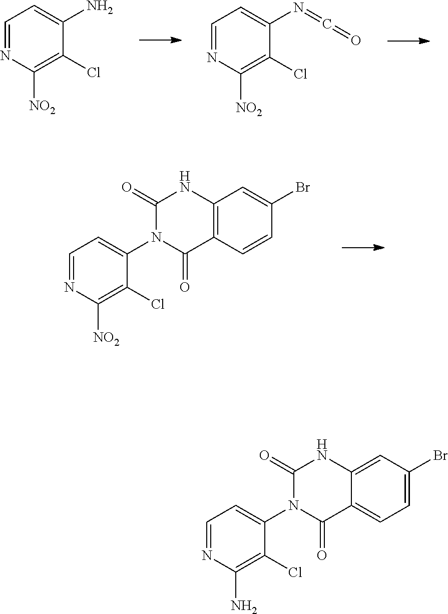 [SMILES: Nc1ccnc([N+](=O)[O-])c1Cl ; O=C=Nc1ccnc([N+](=O)[O-])c1Cl ; O=c1[nH]c2cc(Br)ccc2c(=O)n1-c1ccnc([N+](=O)[O-])c1Cl ; Nc1nccc(-n2c(=O)[nH]c3cc(Br)ccc3c2=O)c1Cl]

Dissolve the compound 2-nitro-3-chloro-4-aminopyridine (0.87 g, 5 mmol) in 25 mL of anhydrous dichloromethane and add triethylamine(1.52 g, 15 mmol). In an ice water bath, add triphosgene (1.48 g, 5 mmol) in portions, resume to room temperature naturally, and react for 2 h. The reaction is quenched with water and extracted with dichloromethane. The resulting organic phase is then washed with saturated brine, dried with anhydrous sodium sulfate and, the organic phase is evaporated under reduced pressure. The crude product 2-nitro-3-chloro-4-isocyanatopyridine is obtained and used directly in the next step.

7-bromo-3-(2-nitro-3-chloropyridine-4-yl)quinazoline-2,4(1H,3H)-dione is obtained by a method similar to that of the intermediate 7-bromo-3-(2,3-dichlorophenyl)quinazoline-2,4(1H,3H)-dione (raw material is changed to 2-nitro-3-chloro-4-isocyanato pyridine). LC/MS (ESI): m/z=396.9[M+H]⁺.

Dissolve the compound 7-bromo-3-(2-nitro-3-chloropyridine-4-yl)quinazoline-2,4(1H,3H)-dione (1.19 g, 3 mmol) in 15 mL of methanol. Hydrogen substitution is performed for 3 times, then Raney Ni (119 mg) is added. Stir the reaction at room temperature and react for 12 hours under a hydrogen atmosphere. The reaction solution is filtered and evaporated under reduced pressure to give the intermediate 7-bromo-3-(2-amino-3-chloropyridine-4-yl)quinazoline-2,4(1H,3H)-dione (1.05 g, 95% yield). LC/MS(ESI): m/z=367.0[M+H]⁺.

### Preparation of the Intermediate 3-(2-amino-3-chloropyridine-4-yl)-7-chloropteridine-2,4(1H,3H)-dione

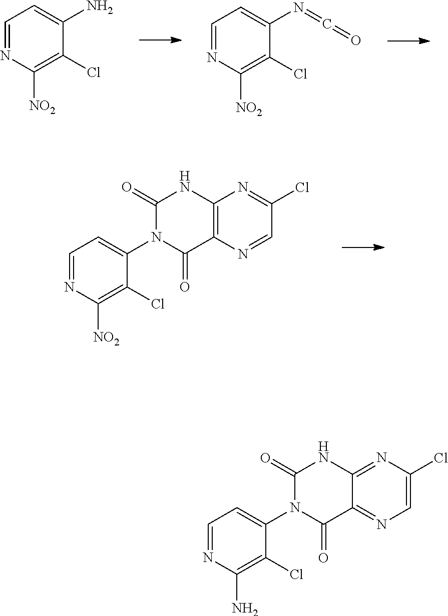 [SMILES: Nc1ccnc([N+](=O)[O-])c1Cl ; O=C=Nc1ccnc([N+](=O)[O-])c1Cl ; O=c1[nH]c2nc(Cl)cnc2c(=O)n1-c1ccnc([N+](=O)[O-])c1Cl ; Nc1nccc(-n2c(=O)[nH]c3nc(Cl)cnc3c2=O)c1Cl]

The compound 3-(2-amino-3-chloropyridine-4-yl)-7-chloropteridine-2,4(1H,3H)-dione is obtained by a method similar to that of intermediate 7-bromo-3-(2-amino-3-chloropyridine-4-yl)quinazoline-2,4(1H,3H)-dione (the intermediate is changed to methyl 3-amino-5-chloropyrazine-2-carboxylate). LC/MS (ESI): m/z=325.0[M+H]⁺.

### Preparation of the Intermediate 3-(2-amino-3-chloropyridine-4-yl)-7-chloropyrido[2,3-d]pyrimidine-2,4(1H,3H)-dion e

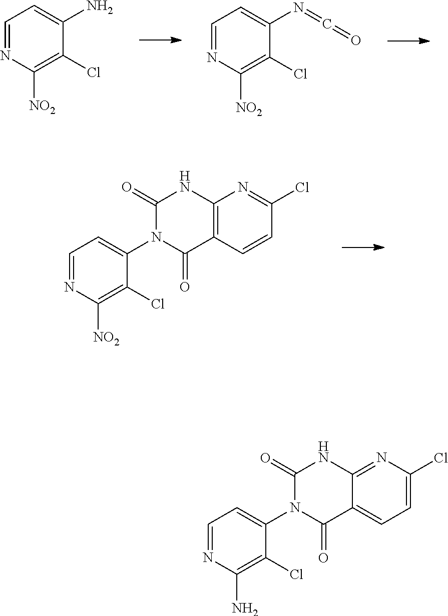 [SMILES: Nc1ccnc([N+](=O)[O-])c1Cl ; O=C=Nc1ccnc([N+](=O)[O-])c1Cl ; O=c1[nH]c2nc(Cl)ccc2c(=O)n1-c1ccnc([N+](=O)[O-])c1Cl ; Nc1nccc(-n2c(=O)[nH]c3nc(Cl)ccc3c2=O)c1Cl]

3-(2-amino-3-chloropyridine-4-yl)-7-chloropyrido[2,3-d]pyrimidine-2,4(1H,3H)-dione is obtained by a method similar to that of the intermediate 7-bromo-3-(2-amino-3-chloropyridine-4-yl)quinazoline-2,4(1H,3H)-dione (the intermediate is changed to 2-amino-6-chloronicotinicacid methyl ester). LC/MS(ESI): m/z=324.0[M+H]⁺.

### Preparation of the Intermediate 7-chloro-3-(3-chloro-2-(cyclopropylamino)-4-yl)pteridine-2,4(1H,3H)-dione

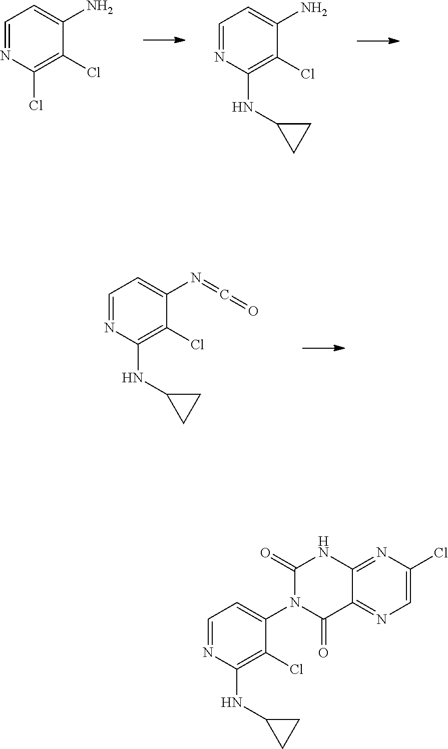 [SMILES: Nc1ccnc(Cl)c1Cl ; Nc1ccnc(NC2CC2)c1Cl ; O=C=Nc1ccnc(NC2CC2)c1Cl ; O=c1[nH]c2nc(Cl)cnc2c(=O)n1-c1ccnc(NC2CC2)c1Cl]

Dissolve the compound 2,3-dichloropyridine-4-amine(1.63 g, 10 mmol) in 20 mL of cyclopropylamine. Heat to 120° C. by microwave, and react for 30 min. Cool to room temperature, the reaction solution is evaporated under reduced pressure and, the residue is purified by column chromatography to give the intermediate 3-chloro-N²-cyclopropylpyridine-2,4-diamine (1.06 g, 65% yield). LC/MS(ESI): m/z=163.0[M+H]⁺.

Dissolve the compound 3-chloro-N²-cyclopropylpyridine-2,4-diamine (0.82 g, 5 mmol) in 25 mL of anhydrous dichloromethane and add triethylamine (1.52 g, 15 mmol). In an ice water bath, triphosgene (1.48 g, 5 mmol) is added in portions. Let the mixture resume to room temperature naturally, react for 4 h, quench with water and, extract with dichloromethane. The resulting organic phase is then washed with saturated brine, dried with anhydrous sodium sulfate, and the organic phase is evaporated to dryness under reduced pressure. The crude product 3-chloro-N-cyclopropyl-4-isocyanato pyridine-2-diamine is obtained and used directly in the next step.

Dissolve the compound 3-amino-5-chloropyrazine-2-carboxylic acid methyl ester (0.94 g,5 mmol) in 20 mL of anhydrous 1,4-dioxane, add 3-chloro-N-cyclopropyl-4-isocyanato pyridine-2-diamine (0.84 g, 4 mmol) and triethylamine (101 mg, 1 mmol). The reaction is carried out at 90° C. for 72 h with stirring. Cool to room temperature, the reaction solution is evaporated to dryness under reduced pressure and, the residue is purified by column chromatography to give the intermediate 7-chloro-3-(3-chloro-2-(cyclopropylamino)pyridine-4-yl)pteridine-2,4(1H,3H)-dione (1.5 g, 69% yield). LC/MS (ESI): m/z=365.0[M+H]⁺.

### Preparation of the Intermediate 7-chloro-3-(3-chloro-2-(cyclopropylamino)pyridine-4-yl)pyrido[2,3-d]pyrimidine-2,4 (1H,3H)-dione

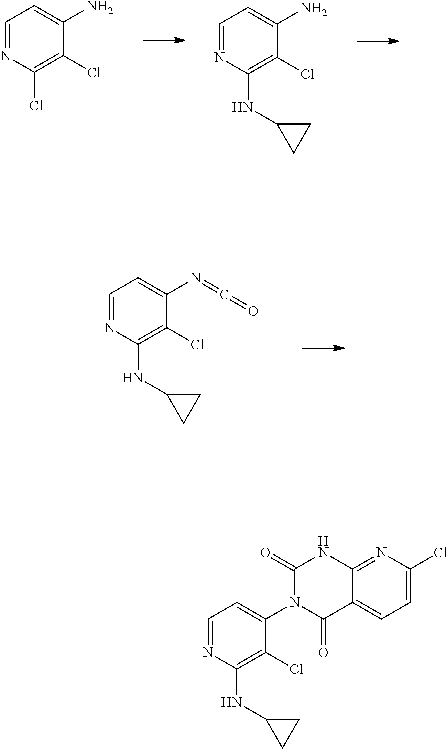 [SMILES: Nc1ccnc(Cl)c1Cl ; Nc1ccnc(NC2CC2)c1Cl ; O=C=Nc1ccnc(NC2CC2)c1Cl ; O=c1[nH]c2nc(Cl)ccc2c(=O)n1-c1ccnc(NC2CC2)c1Cl]

The compound 7-chloro-3-(3-chloro-2-(cyclopropylamino)pyridine-4-yl)pyridinyl[2,3-d]pyrimidine-2,4(1H,3H)-dione is obtained by a method similar to that of the intermediate 7-chloro-3-(3-chloro-2-(cyclopropylamino)pyridine-4-yl) pteridine-2,4(1H,3H)-dione (the intermediate is changed to 2-amino-6-chloronicotinic acid methyl ester). LC/MS(ESI): m/z=364.0[M+H]⁺.

### Preparation of the Intermediate N-(3-amino-2-chlorophenyl)-2-hydroxy-4-oxa-6,7,8,9-tetrahydro-4H-pyrido[1,2-a]py rimidine-3-carboxamide

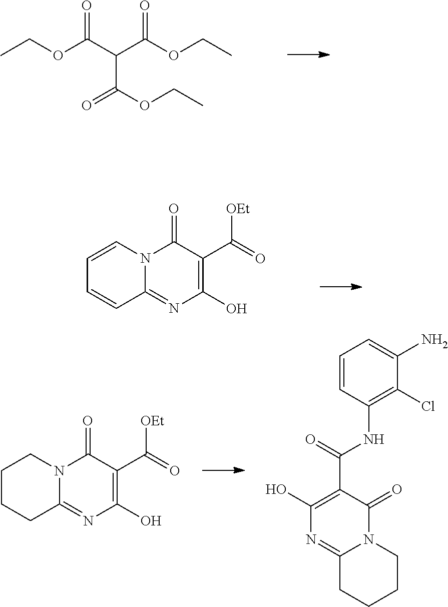 [SMILES: CCOC(=O)C(C(=O)OCC)C(=O)OCC ; CCOC(=O)c1c(O)nc2ccccn2c1=O ; CCOC(=O)c1c(O)nc2n(c1=O)CCCC2 ; Nc1cccc(NC(=O)c2c(O)nc3n(c2=O)CCCC3)c1Cl]

Dissolve the compound triethyl methane tricarboxylate (9.28 g, 40 mmol) and 2-aminopyridine (1.88 g, 20 mmol) in 70 mL of xylene, heat to 120° ° C. and stir, react for 24 hours. Cool to room temperature, filter. The filter cake is washed 3 times with methanol and dried to give the compound ethyl 2-hydroxy-4-oxo-pyrido[1,2-a]pyrimidine-3-carboxylate(1.16 g, 25% yield). LC/MS (ESI): m/z=235.1[M+H]⁺.

Under nitrogen protection, dissolve the compound ethyl 2-hydroxy-4-oxo-pyrido[1,2-a]pyrimidine-3-carboxylate (936 mg, 4 mmol) in 10 mL of methanol, add 80 mg of palladium-carbon and perform hydrogen substitution for 3 times. Stir and react at room temperature for 2 hours. The reaction solution is filtered and concentrated under reduced pressure to give the compound 2-hydroxyl-4-oxo-6,7,8,9-tetrahydro-4H-pyridinyl[1,2-a]pyrimidine-3-carboxylic acid ethyl ester (904 mg, yield 95%). LC/MS (ESI): m/z=239.1[M+H]⁺.

Dissolve the compound 2-chlorobenzene-1,3-diamine(542 mg,3.8 mmol) and ethyl 2-hydroxy-4-oxo-6,7,8,9-tetrahydro-4H-pyrido[1,2-a]pyrimidine-3-carboxylate (904 mg, 3.8 mmol) in 20 mL of chlorobenzene. Heat and reflux for 3 hourH; cool to room temperature, filter, after drying, the compound N-(3-amino-2-chlorophenyl)-2-hydroxy-4-oxa-6,7,8,9-tetrahydro-4H-pyrido[1,2-a]pyrimi dine-3-carboxamide (661 mg, 52% yield) is obtained. LC/MS (ESI): m/z=335.1[M+H]⁺.

### Preparation of the Intermediate N-(2-chloro-3-(7-chloro-2,4-dioxa-1,2-dihydropteridine-3(4H)-yl)phenyl)-2-hydroxy-4-oxa-6,7,8,9-tetrahydro-4H-pyrido[1,2-a]pyrimidine-3-carboxamide

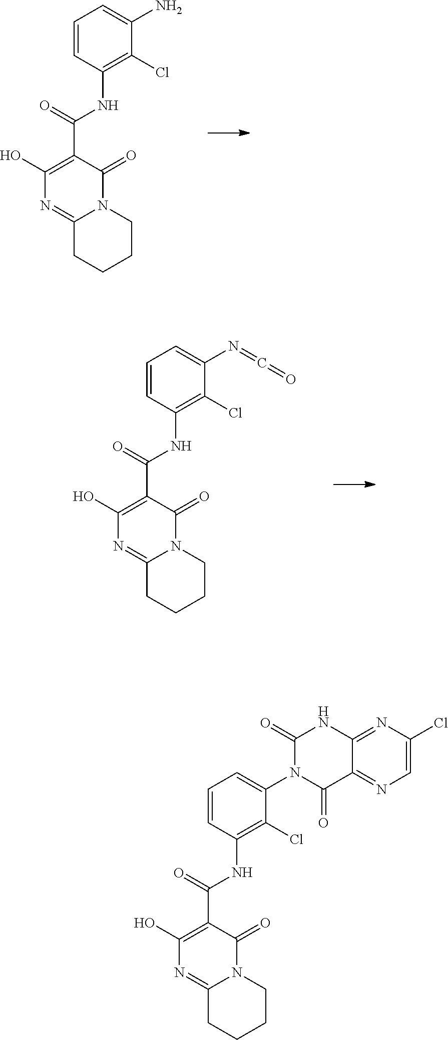 [SMILES: Nc1cccc(NC(=O)c2c(O)nc3n(c2=O)CCCC3)c1Cl ; O=C=Nc1cccc(NC(=O)c2c(O)nc3n(c2=O)CCCC3)c1Cl ; O=C(Nc1cccc(-n2c(=O)[nH]c3nc(Cl)cnc3c2=O)c1Cl)c1c(O)nc2n(c1=O)CCCC2]

A preparation method similar to the first two steps of preparation of the intermediate 7-bromo-3-(2-amino-3-chloropyridine-4-yl)quinazoline-2,4(1H,3H)-dione is used to obtain the compound N-(2-chloro-3-(7-chloro-2,4-dioxa-1,2-dihydropteridine-3(4H)-yl)phenyl)-2-hydroxy-4-o xa-6,7,8,9-tetrahydro-4H-pyrido[1,2-a]pyrimidine-3-carboxamide. LC/MS(ESI): m/z=516.1[M+H]⁺.

### Preparation of the Intermediate N-(3-(7-bromo-2,4-dioxa-1,2-dihydropyrido[3,2-d]pyrimidine-3(4H)-yl)-2-chlorophe nyl)-2-hydroxy-4-oxa-6,7,8,9-tetrahydro-4H-pyrido[1,2-a]pyrimidine-3-carboxamide

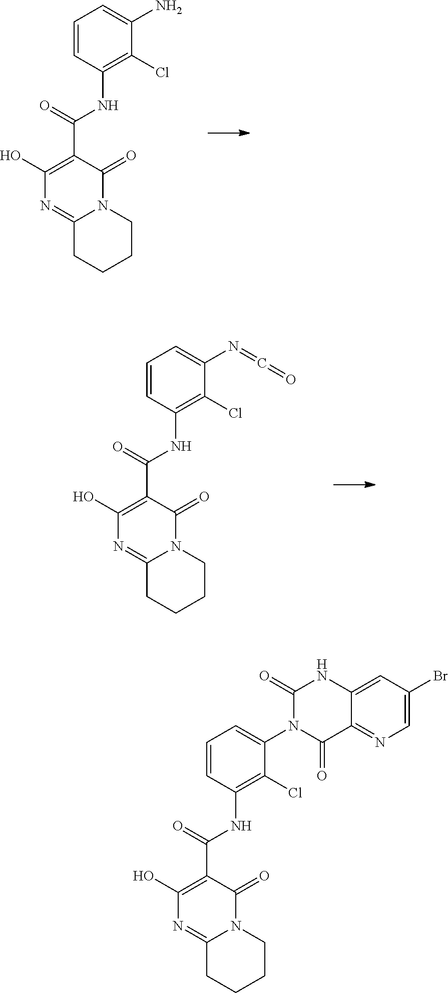 [SMILES: Nc1cccc(NC(=O)c2c(O)nc3n(c2=O)CCCC3)c1Cl ; O=C=Nc1cccc(NC(=O)c2c(O)nc3n(c2=O)CCCC3)c1Cl ; O=C(Nc1cccc(-n2c(=O)[nH]c3cc(Br)cnc3c2=O)c1Cl)c1c(O)nc2n(c1=O)CCCC2]

The compound N-(3-(7-bromo-2,4-dioxa-1,2-dihydropyrido[3,2-d]pyrimidine-3(4H)-yl)-2-chlorophenyl)-2-hydroxy-4-oxa-6,7,8,9-tetrahydro-4H-pyrido[1,2-a]pyrimidine-3-carboxamide is obtained using a similar synthetic method as the two preceding steps for the intermediate 7-bromo-3-(2-amino-3-chloropyridine-4-yl)quinazolin-2,4(1H,3H)-dione. LC/MS(ESI): m/z=559.0[M+H]⁺.

### Preparation of the Intermediate N-(2-chloro-3-(7-chloro-2,4-dioxa-1,2-dihydropyrido[2,3-d]pyrimidine-3(4H)-yl)phe nyl)-2-hydroxy-4-oxa-6,7,8,9-tetrahydro-4H-pyrido[1,2-a]pyrimidine-3-carboxamide

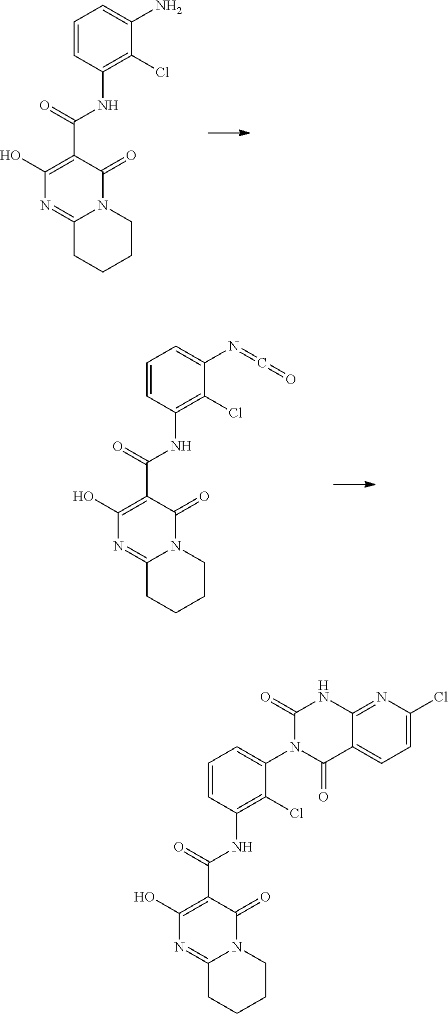 [SMILES: Nc1cccc(NC(=O)c2c(O)nc3n(c2=O)CCCC3)c1Cl ; O=C=Nc1cccc(NC(=O)c2c(O)nc3n(c2=O)CCCC3)c1Cl ; O=C(Nc1cccc(-n2c(=O)[nH]c3nc(Cl)ccc3c2=O)c1Cl)c1c(O)nc2n(c1=O)CCCC2]

The compound N-(2-chloro-3-(7-chloro-2,4-dioxo-1,2-dihydropyrido[2,3-d]pyrimidine-3(4H)-yl)phenyl)-2-hydroxy-4-oxo-6,7,8,9-tetrahydro-4H-pyrido[1,2-a]pyrimidine-3-carboxamide is obtained using a similar synthetic method as the two preceding steps for the intermediate 7-bromo-3-(2-amino-3-chloropyridine-4-yl)quinazolin-2,4(1H,3H)-dione. LC/MS(ESI): m/z=515.1[M+H]⁺.

### Preparation of the Intermediate N-(3-(7-bromo-2,4-dioxa-1,2-dihydroquinazolin-3(4H)-yl)-2-chlorophenyl)-2-hydrox y-4-oxa-6,7,8,9-tetrahydro-4H-pyrido[1,2-a]pyrimidine-3-carboxamide

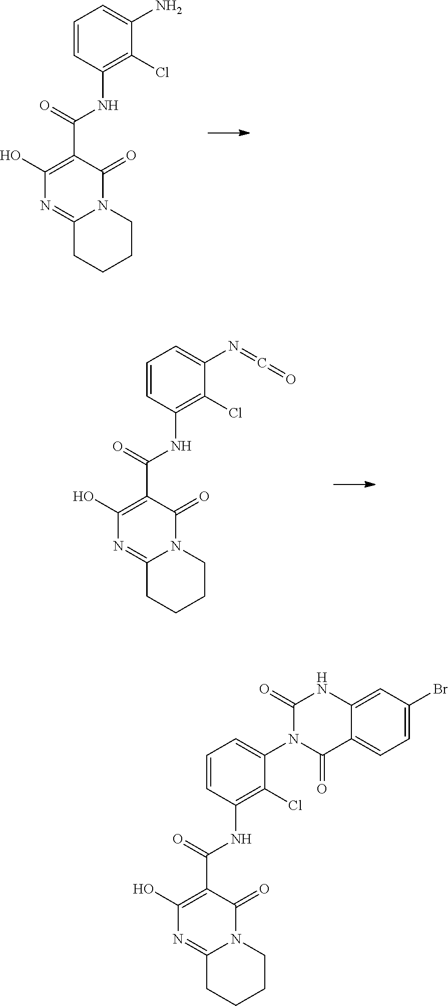 [SMILES: Nc1cccc(NC(=O)c2c(O)nc3n(c2=O)CCCC3)c1Cl ; O=C=Nc1cccc(NC(=O)c2c(O)nc3n(c2=O)CCCC3)c1Cl ; O=C(Nc1cccc(-n2c(=O)[nH]c3cc(Br)ccc3c2=O)c1Cl)c1c(O)nc2n(c1=O)CCCC2]

The compound N-(2-chloro-3-(7-chloro-2,4-dioxa-1,2-dihydropyrido[2,3-d]pyrimidine-3(4H)-yl)phenyl)-2-hydroxy-4-oxa-6,7,8,9-(tetrahydro-4H-pyridine-6,7,8,9-yl)-3-carboxamide is obtained by the method of preparation similar to that for the first two steps of the intermediate 7-bromo-3-(2-amino-3-chloropyridine-4-yl)quinazoline-2,4(1H,3H)-dione. LC/MS(ESI): m/z=558.0[M+H]⁺.

### Preparation of the Intermediate N-(2-chloro-3-(7-chloro-2,4-dioxa-1,2-dihydropteridine-3(4H)-yl)phenyl)-1-methyl-1H-pyrazole-3-carboxamide

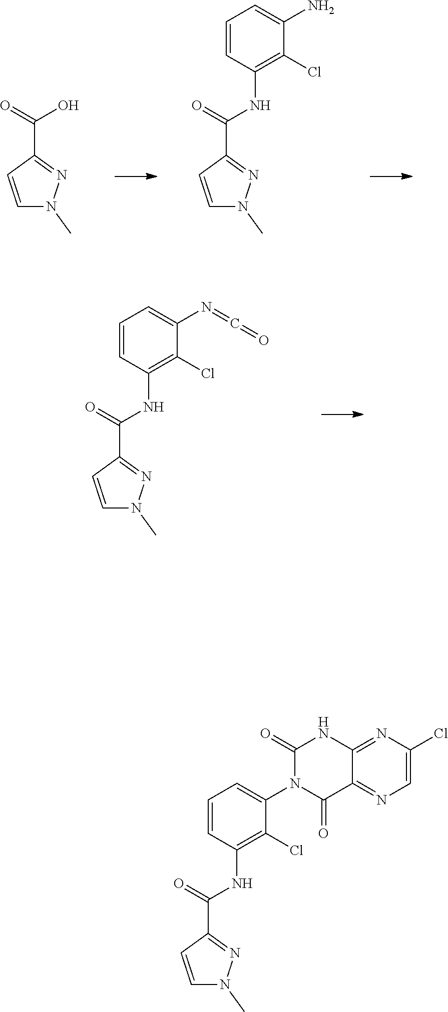

Add 1-Methylpyrazole-3-carboxylic acid (630 mg, 5 mmol) to 10 mL of sulfuryl chloride, and the mixture is refluxed with stirring for 2 hours. Then, the mixture is concentrated under reduced pressure to give the acyl chloride. 15 mL of dichloromethane is added to the acyl chloride, followed by pyridine (593 mg, 7.5 mmol), 2-chloro-1,3-phenylenediamine (357 mg, 2.5 mmol), and N,N-dimethyl-4-aminopyridine (153 mg, 1.25 mmol). The reaction mixture is stirred at room temperature for 2 hours, quenched with water, extracted with dichloromethane. The resulting organic phase is then washed with saturated brine, dried with anhydrous sodium sulfate and, the organic phase is evaporated to dryness under reduced pressure. The residue is purified by column chromatography to give the compound N-(3-amino-2-chlorophenyl)-1-methyl-1H-pyrazole-3-carboxamide (520 mg, 83% yield). LC/MS (ESI): m/z=251.1[M+H]⁺.

The compound N-(2-chloro-3-(7-chloro-2,4-dioxo-1,2-dihydropteridine-3(4H)-yl)phenyl)-1-methyl-1H-p yrazole-3-carboxamide is obtained using a similar synthetic method as the two preceding steps for the intermediate 7-bromo-3-(2-amino-3-chloropyridine-4-yl)quinazolin-2,4(1H,3H)-dione (the raw material is changed to N-(3-amino-2-chlorophenyl)-1-methyl-1H-pyrazole-3-carboxamide). LC/MS(ESI): m/z=432.0[M+H]⁺.

### Preparation of the Intermediate N-(2-chloro-3-(7-chloro-2,4-dioxa-1,2-dihydropteridine-3(4H)-yl)phenyl)-1-methyl-1H-pyrazole-4-carboxamide

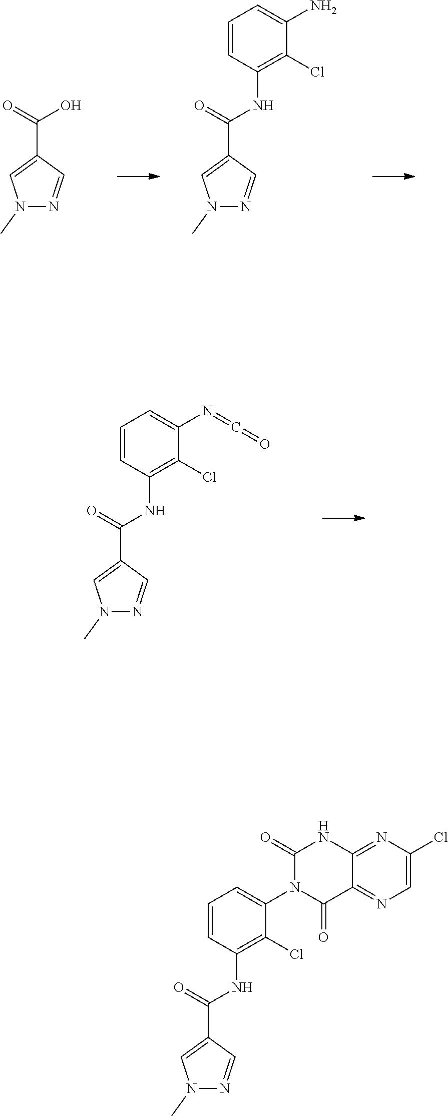

The compound N-(2-chloro-3-(7-chloro-2,4-dioxo-1,2-dihydropteridine-3(4H)-yl)phenyl)-1-methyl-1H-p yrazole-4-carboxamide is obtained using a similar synthetic method as the intermediate N-(2-chloro-3-(7-chloro-2,4-dioxo-1,2-dihydropteridine-3(4H)-yl)phenyl)-1-methyl-1H-p yrazole-3-carboxamid (the raw material is changed to 1-methylpyrazole-4-carboxylic acid. LC/MS (ESI): m/z=432.0[M+H]⁺.

### Preparation of the Intermediate N-(2-chloro-3-(7-chloro-2,4-dioxa-1,2-dihydropteridine-3(4H)-yl)phenyl)benzamide

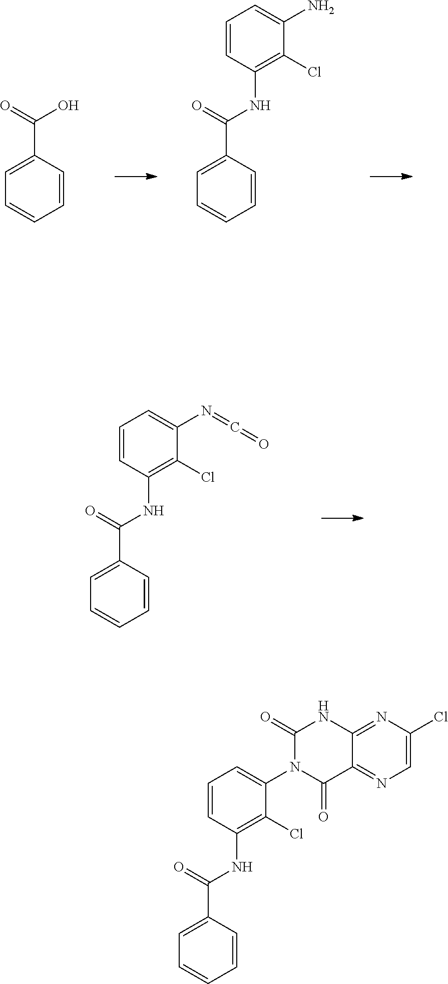 [SMILES: O=C(O)c1ccccc1 ; Nc1cccc(NC(=O)c2ccccc2)c1Cl ; O=C=Nc1cccc(NC(=O)c2ccccc2)c1Cl ; O=C(Nc1cccc(-n2c(=O)[nH]c3nc(Cl)cnc3c2=O)c1Cl)c1ccccc1]

The compound N-(2-chloro-3-(7-chloro-2,4-dioxo-1,2-dihydropteridine-3(4H)-yl)phenyl)benzamide is obtained using a similar synthetic method as the intermediate N-(2-chloro-3-(7-chloro-2,4-dioxo-1,2-dihydropteridine-3(4H)-yl)phenyl)-1-methyl-1H-p yrazole-3-carboxamide (the raw material is changed to benzonic acid). LC/MS(ESI): m/z=428.0[M+H]⁺.

### Preparation of the Intermediate N-(2-chloro-3-(7-chloro-2,4-dioxa-1,2-dihydropteridine-3(4H)-yl)phenyl)pyrazine-2-carboxamide

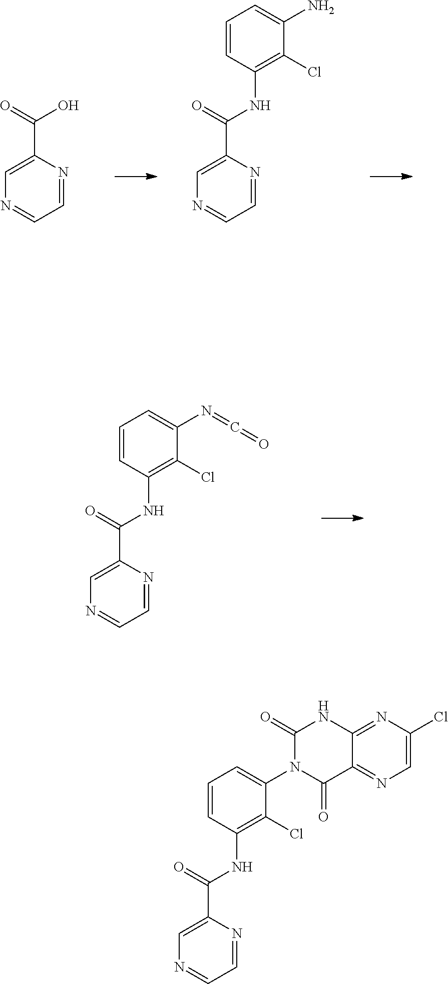 [SMILES: O=C(O)c1cnccn1 ; Nc1cccc(NC(=O)c2cnccn2)c1Cl ; O=C=Nc1cccc(NC(=O)c2cnccn2)c1Cl ; O=C(Nc1cccc(-n2c(=O)[nH]c3nc(Cl)cnc3c2=O)c1Cl)c1cnccn1]

The compound N-(2-chloro-3-(7-chloro-2,4-dioxo-1,2-dihydropteridine-3(4H)-yl)phenyl)pyrazine-2-carb oxamide is obtained using a similar synthetic method as the intermediate N-(2-chloro-3-(7-chloro-2,4-dioxo-1,2-dihydropteridine-3(4H)-yl)phenyl)-1-methyl-1H-p yrazole-3-carboxamide (the raw material is changed to 2-pyrazinecarboxylic acid). LC/MS(ESI): m/z=430.0[M+H]⁺.

### Preparation of the Intermediate N-(2-chloro-3-(7-chloro-2,4-dioxa-1,2-dihydropteridine-3(4H)-yl)phenyl)pyridine-2-c arboxamide

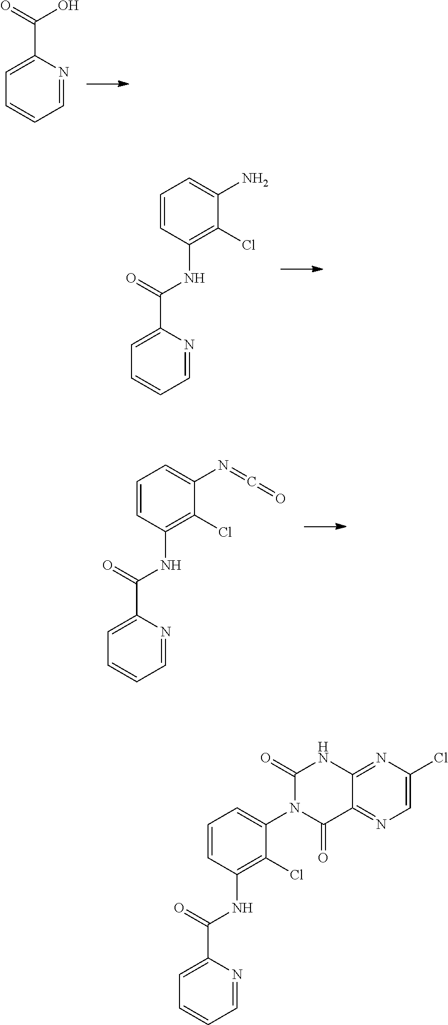 [SMILES: O=C(O)c1ccccn1 ; Nc1cccc(NC(=O)c2ccccn2)c1Cl ; O=C=Nc1cccc(NC(=O)c2ccccn2)c1Cl ; O=C(Nc1cccc(-n2c(=O)[nH]c3nc(Cl)cnc3c2=O)c1Cl)c1ccccn1]

The compound N-(2-chloro-3-(7-chloro-2,4-dioxo-1,2-dihydropteridine-3(4H)-yl)phenyl)pyridine-2-carb oxamide is synthesized using a similar method as the intermediate N-(2-chloro-3-(7-chloro-2,4-dioxo-1,2-dihydropteridine-3(4H)-yl)phenyl)-1-methyl-1H-p yrazole-3-carboxamide (the raw material is changed to 2-pyridinecarboxylic acid). LC/MS(ESI): m/z=429.0[M+H]⁺.

### Preparation of the Intermediate N-(2-chloro-3-(7-chloro-2,4-dioxa-1,2-dihydropteridine-3(4H)-yl)phenyl)pyrimidine-4-carboxamide

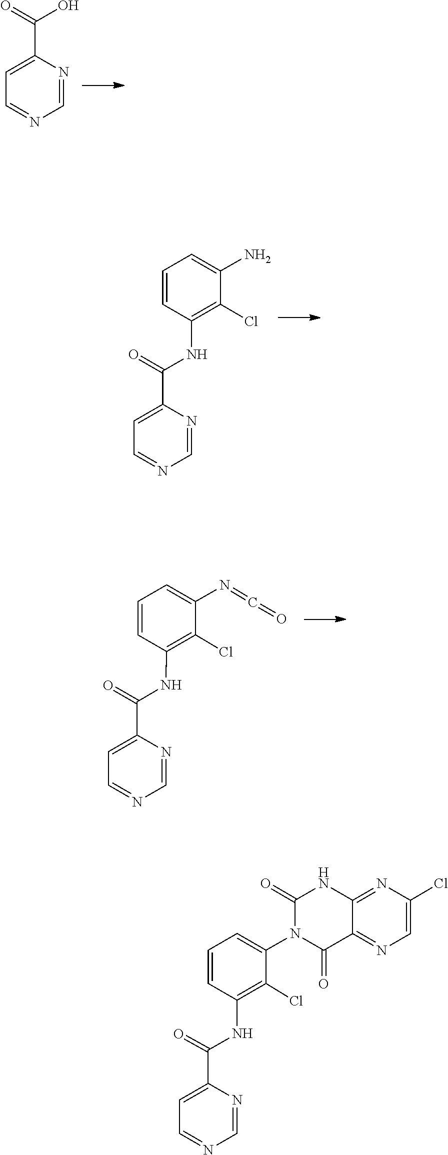 [SMILES: O=C(O)c1ccncn1 ; Nc1cccc(NC(=O)c2ccncn2)c1Cl ; O=C=Nc1cccc(NC(=O)c2ccncn2)c1Cl ; O=C(Nc1cccc(-n2c(=O)[nH]c3nc(Cl)cnc3c2=O)c1Cl)c1ccncn1]

The compound N-(2-chloro-3-(7-chloro-2,4-dioxo-1,2-dihydropteridine-3(4H)-yl)phenyl)pyrimidine-4-ca rboxamide is obtained using a similar synthetic method as the intermediate N-(2-chloro-3-(7-chloro-2,4-dioxo-1,2-dihydropteridine-3(4H)-yl)phenyl)-1-methyl-1H-p yrazole-3-carboxamide (the starting material is changed to 4-pyrimidinecarboxylic acid). LC/MS(ESI): m/z=430.0[M+H]⁺.

### Preparation of the Intermediate N-(2-chloro-3-(7-chloro-2,4-dioxa-1,2-dihydropteridine-3(4H)-yl)phenyl)-1-methyl-1H-indole-7-carboxamide

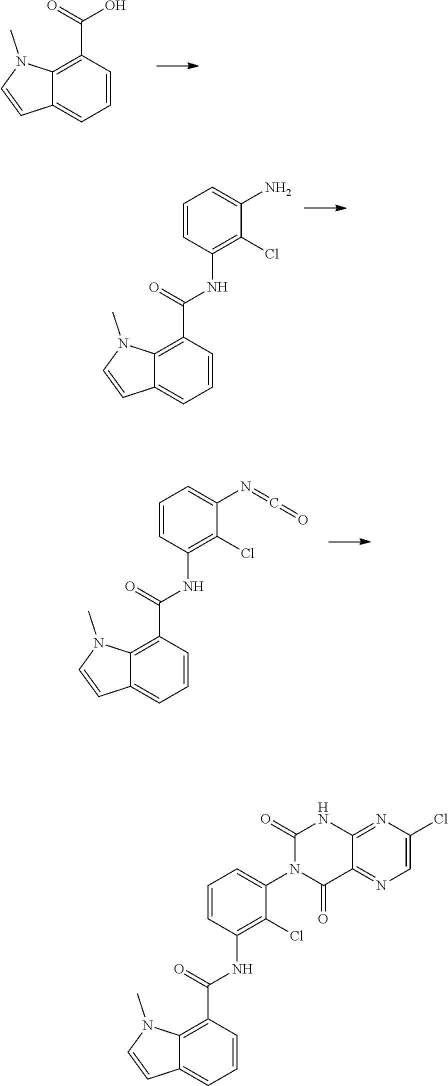 [SMILES: Cn1ccc2cccc(C(=O)O)c21 ; Cn1ccc2cccc(C(=O)Nc3cccc(N)c3Cl)c21 ; Cn1ccc2cccc(C(=O)Nc3cccc(N=C=O)c3Cl)c21 ; Cn1ccc2cccc(C(=O)Nc3cccc(-n4c(=O)[nH]c5nc(Cl)cnc5c4=O)c3Cl)c21]

The compound N-(2-chloro-3-(7-chloro-2,4-dioxo-1,2-dihydropteridine-3(4H)-yl)phenyl)-1-methyl-1H-i ndole-7-carboxamide is obtained using a similar synthetic method as the intermediate N-(2-chloro-3-(7-chloro-2,4-dioxo-1,2-dihydropteridine-3(4H)-yl)phenyl)-1-methyl-1H-p yrazole-3-carboxamide (the raw material is changed to 1-methyl-7-indolecarboxylic acid). LC/MS(ESI): m/z=481.1[M+H]⁺.

### Preparation of the Intermediate N-(2-chloro-3-(7-chloro-2,4-dioxa-1,2-dihydropteridine-3(4H)-yl)phenyl)-1-methyl-1H-indazole-7-carboxamide

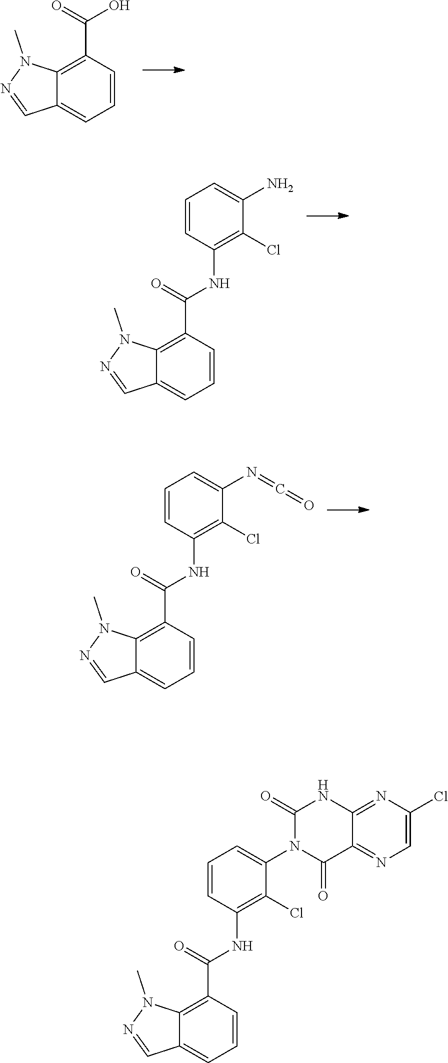 [SMILES: Cn1ncc2cccc(C(=O)O)c21 ; Cn1ncc2cccc(C(=O)Nc3cccc(N)c3Cl)c21 ; Cn1ncc2cccc(C(=O)Nc3cccc(N=C=O)c3Cl)c21 ; Cn1ncc2cccc(C(=O)Nc3cccc(-n4c(=O)[nH]c5nc(Cl)cnc5c4=O)c3Cl)c21]

The compound N-(2-chloro-3-(7-chloro-2,4-dioxo-1,2-dihydropteridine-3(4H)-yl)phenyl)-1-methyl-1H-i ndazole-7-carboxamide is obtained using a similar synthetic method as the intermediate N-(2-chloro-3-(7-chloro-2,4-dioxo-1,2-dihydropteridine-3(4H)-yl)phenyl)-1-methyl-1H-p yrazole-3-carboxamide (the raw material is changed to 1-methyl-7-indazolylcarboxylic acid). LC/MS(ESI): m/z=482.0[M+H]⁺.

### Preparation of the Intermediate 7-chloro-3-(2-chloro-3-(pyrimidine-4-yl)phenyl)pteridine-2,4(1H,3H)-dione

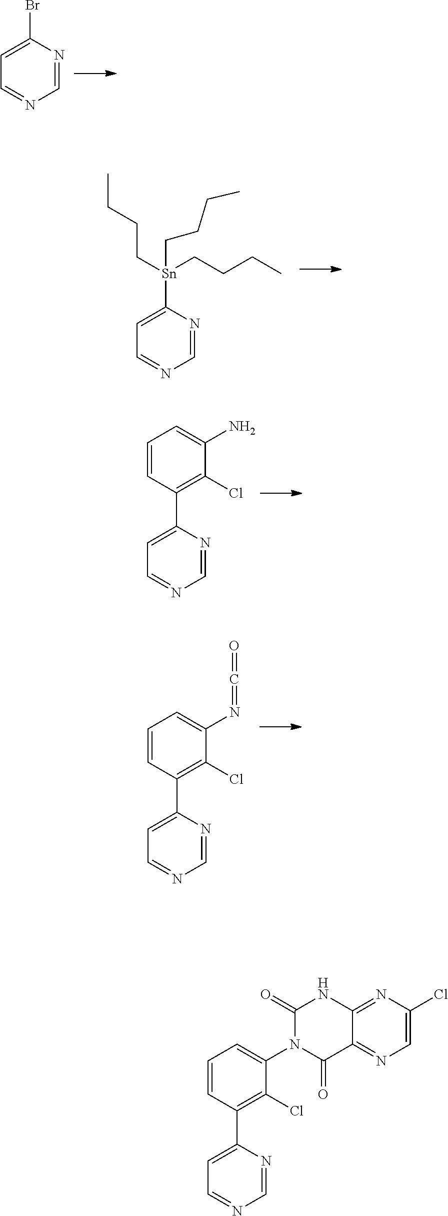 [SMILES: Brc1ccncn1 ; CCC[CH2][Sn]([CH2]CCC)([CH2]CCC)[c]1ccncn1 ; Nc1cccc(-c2ccncn2)c1Cl ; O=C=Nc1cccc(-c2ccncn2)c1Cl ; O=c1[nH]c2nc(Cl)cnc2c(=O)n1-c1cccc(-c2ccncn2)c1Cl]

Dissolve 4-Bromopyrimidine (4.97 g, 31.25 mmol) in 80 mL of toluene, add hexabutylditin (18.05 g, 31.25 mmol) and tetrakis (triphenylphosphine)palladium (1.79 g, 1.56 mmol). The mixture is heated to 120° C. and stirred for 6 hours. After cooled to room temperature, the reaction solution is evaporated under reduced pressure. The residue is purified by column chromatography to give the compound 4-(tributylstannyl)pyrimidine (3.23 g, 28% yield).

Dissolve the compound 4-(tributylstannyl)pyrimidine (1.85 g, 5 mmol) in 40 mL of xylene, add the compound 2-chloro-3-iodoaniline (1.27 g, 5 mmol) and tetrakis(triphenylphosphine)palladium (289 mg, 0.25 mmol), heat to 120° C. and stir for 3 hours. Cool to room temperature and the reaction solution is evaporated under reduced pressure. The residue is purified by column chromatography to give the compound 2-chloro-3-(pyrimidine-4-yl)aniline (0.67 g, 65% yield). LC/MS (ESI): m/z=206.0[M+H]⁺.

The compound 7-chloro-3-(2-chloro-3-(pyridine-4-yl)phenyl)pteridine-2,4(1H,3H)-dione is prepared using a similar synthetic method as the previous two steps for the intermediate 7-bromo-3-(2-amino-3-chloropyridine-4-yl)quinazolin-2,4(1H,3H)-dione. LC/MS(ESI): m/z=387.0[M+H]⁺.

### Preparation of the Intermediate 7-chloro-3-(2-chloro-3-(pyridine-3-yl)phenyl)pteridine-2,4(1H,3H)-dione

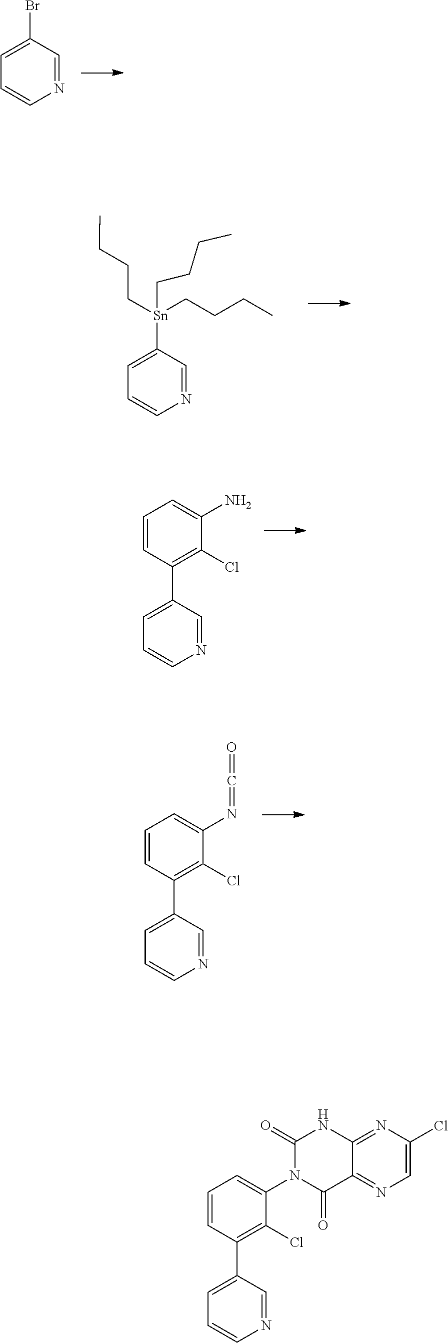 [SMILES: Brc1cccnc1 ; CCC[CH2][Sn]([CH2]CCC)([CH2]CCC)[c]1cccnc1 ; Nc1cccc(-c2cccnc2)c1Cl ; O=C=Nc1cccc(-c2cccnc2)c1Cl ; O=c1[nH]c2nc(Cl)cnc2c(=O)n1-c1cccc(-c2cccnc2)c1Cl]

The compound 7-chloro-3-(2-chloro-3-(pyridine-3-yl)phenyl)pteridine-2,4(1H,3H)-dione is obtained using a similar synthetic method as the intermediate 7-chloro-3-(2-chloro-3-(pyrimidine-4-yl)phenyl)pteridine-2,4(1H,3H)-dione (the raw material is changed to 3-bromopyridine). LC/MS (ESI): m/z=386.0[M+H]⁺.

### Preparation of the Intermediate 7-chloro-3-(2-chloro-3-(pyrazine-2-yl)phenyl)pteridine-2,4(1H,3H)-dione

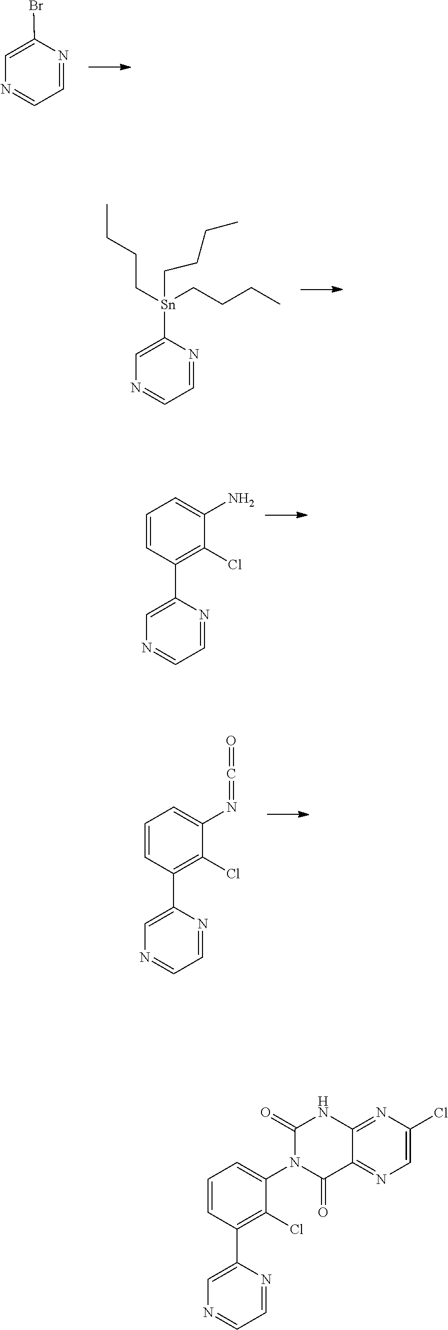 [SMILES: Brc1cnccn1 ; CCC[CH2][Sn]([CH2]CCC)([CH2]CCC)[c]1cnccn1 ; Nc1cccc(-c2cnccn2)c1Cl ; O=C=Nc1cccc(-c2cnccn2)c1Cl ; O=c1[nH]c2nc(Cl)cnc2c(=O)n1-c1cccc(-c2cnccn2)c1Cl]

The compound 7-chloro-3-(2-chloro-3-(pyrimidine-2-yl)phenyl)pteridine-2,4(1H,3H)-dione is obtained using a similar synthetic method as the intermediate 7-chloro-3-(2-chloro-3-(pyrimidine-4-yl)phenyl)pteridine-2,4(1H,3H)-dione (the raw material is changed to 2-bromopyrazine). LC/MS(ESI): m/z=387.0[M+H]⁺.

### Preparation of the Intermediate 7-chloro-3-(2-chloro-3-(pyrazine-2-yl)phenyl)pteridine-2,4(1H,3H)-dione

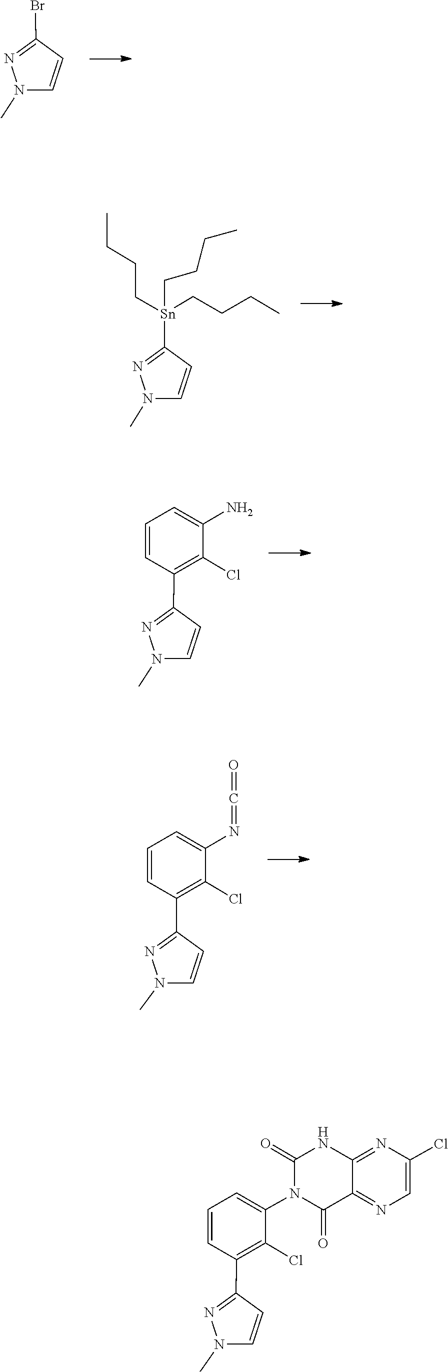

The compound 7-chloro-3-(2-chloro-3-(1-methyl-1H-pyrazole-3-yl)phenyl)pteridine-2,4(1H,3H)-dione is obtained a similar method the using synthetic as intermediate 7-chloro-3-(2-chloro-3-(pyrimidine-4-yl)phenyl)pteridine-2,4(1H,3H)-dione (the raw material is changed to 3-bromo-1-methylpyrrole). LC/MS(ESI): m/z=389.0[M+H]⁺.

### Preparation of the Intermediate 7-chloro-3-(2-chloro-3-(pyrimidine-4-amino)phenyl)pteridine-2,4(1H,3H)-dione

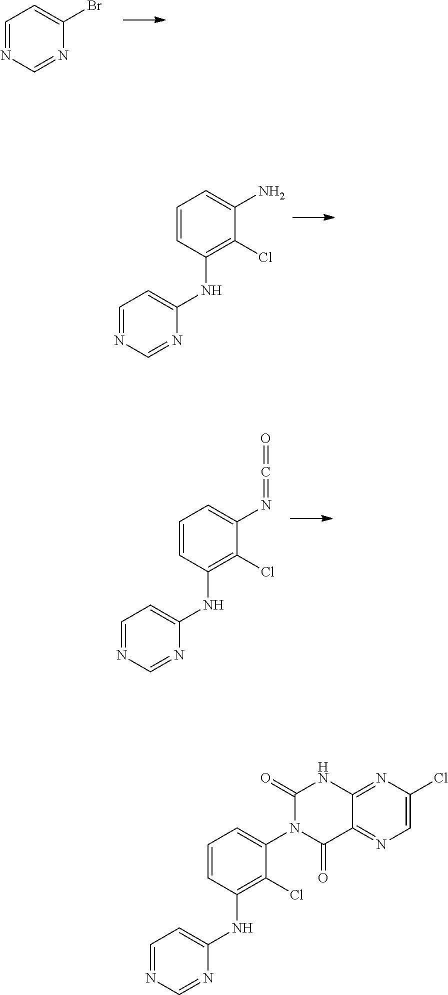 [SMILES: Brc1ccncn1 ; Nc1cccc(Nc2ccncn2)c1Cl ; O=C=Nc1cccc(Nc2ccncn2)c1Cl ; O=c1[nH]c2nc(Cl)cnc2c(=O)n1-c1cccc(Nc2ccncn2)c1Cl]

Dissolve the compound 4-bromopyrimidine (1.59 g, 10 mmol) in toluene (15 mL), add sodium t-butoxide (1.15 g, 12 mmol), and 4,5-bisdiphenylphosphine-9,9-dimethylxanthene (60 mg). The nitrogen substitution is performed three times. Add tris(dibenzylideneacetone)dipalladium (85 mg) to the mixed solution, and the mixture is stirred and refluxed for 3 hours. Cool to room temperature, the reaction solution is diluted with water and extracted with ethyl acetate. The resulting organic phase is washed with saturated brine, dried with anhydrous sodium sulfate, and the organic phase is evaporated to dryness under reduced pressure. The residue is purified by column chromatography to give the compound 2-chloro-N¹-(pyrimidine-4-yl)benzene-1,3-diamine (1.52 g, 52% yield). LC/MS(ESI): m/z=221.1[M+H]⁺.

The compound 7-chloro-3-(2-chloro-3-(pyrimidine-4-amino)phenyl)-2,4(1H,3H)-dione is obtained in the subsequent two steps using a similar synthetic method as the first two steps of the intermediate 7-bromo-3-(2-amino-3-chloropyridine-4-yl)quinazolin-2,4(1H,3H)-dione. LC/MS (ESI): m/z=402.0[M+H]⁺.

### Preparation of the Intermediate 7-chloro-3-(2-chloro-3-(pyridine-2-ylamino)phenyl)pteridine-2,4(1H,3H)-dione

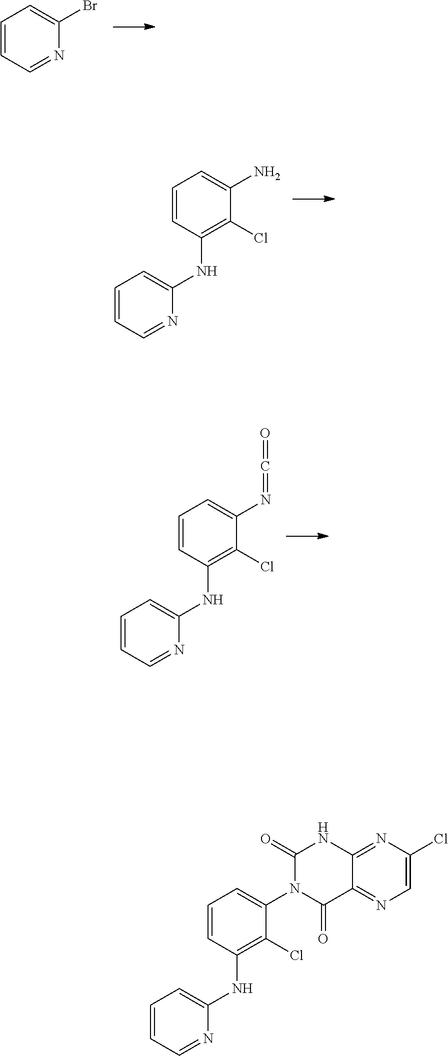 [SMILES: Brc1ccccn1 ; Nc1cccc(Nc2ccccn2)c1Cl ; O=C=Nc1cccc(Nc2ccccn2)c1Cl ; O=c1[nH]c2nc(Cl)cnc2c(=O)n1-c1cccc(Nc2ccccn2)c1Cl]

The compound 7-chloro-3-(2-chloro-3-(pyrimidine-4-yl)phenyl)pteridine-2,4(1H,3H)-dione is obtained by similar the a preparation to intermediate 7-chloro-3-(2-chloro-3-(pyrimidine-2-ylamino)phenyl)pteridine-2,4(1H,3H)-dione (the raw material is changed to 2-bromopyridine). LC/MS (ESI): m/z=401.0[M+H]⁺.

### Preparation of the Intermediate 7-chloro-3-(2-chloro-3-(pyrazine-2-ylamino)phenyl)pteridine-2,4(1H,3H)-dione

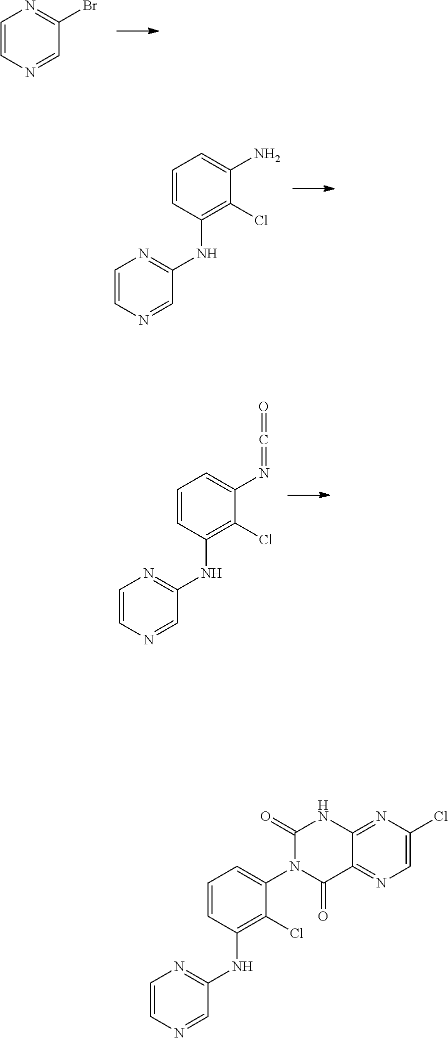 [SMILES: Brc1cnccn1 ; Nc1cccc(Nc2cnccn2)c1Cl ; O=C=Nc1cccc(Nc2cnccn2)c1Cl ; O=c1[nH]c2nc(Cl)cnc2c(=O)n1-c1cccc(Nc2cnccn2)c1Cl]

The compound 7-chloro-3-(2-chloro-3-(pyrazine-2-ylamino)phenyl)pteridine-2,4(1H,3H)-dione is obtained by a preparation similar to the intermediate 7-chloro-3-(2-chloro-3-(pyrimidine-4-yl)phenyl)pteridine-2,4(1H,3H)-dione (the raw material is changed to 2-bromopyrazine). LC/MS(ESI): m/z=402.0[M+H]⁺.

### Preparation of the Intermediate 7-chloro-3-(2-chloro-3-(pyrimidine-2-amino)phenyl)pteridine-2,4(1H,3H)-dione

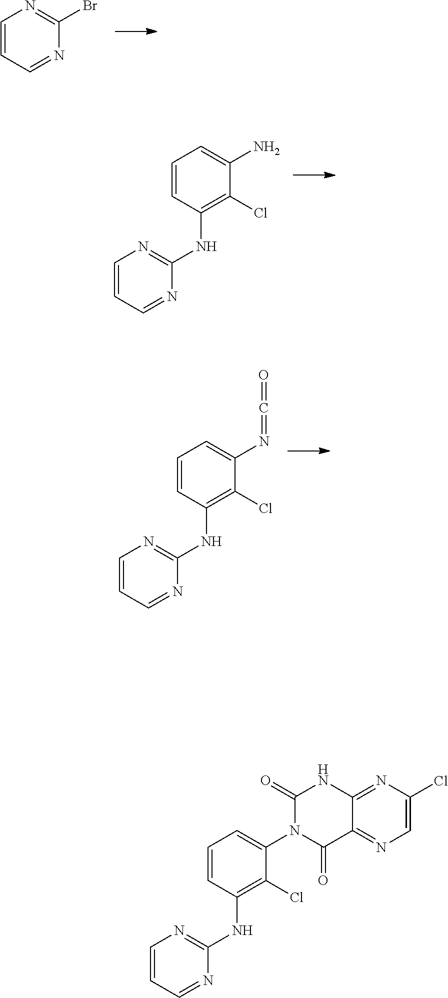 [SMILES: Brc1ncccn1 ; Nc1cccc(Nc2ncccn2)c1Cl ; O=C=Nc1cccc(Nc2ncccn2)c1Cl ; O=c1[nH]c2nc(Cl)cnc2c(=O)n1-c1cccc(Nc2ncccn2)c1Cl]

The compound 7-chloro-3-(2-chloro-3-(pyrimidine-2-amino)phenyl)pteridine-2,4(1H,3H)-dione is obtained by a preparation method similar to that of the intermediate 7-chloro-3-(2-chloro-3-(pyrimidine-4-yl)phenyl)pteridine-2,4(1H,3H)-dione (the raw material is changed to 2-bromopyrimidine). LC/MS (ESI): m/z=402.0[M+H]⁺.

### Preparation of the Intermediate 3-(1-methylpyrazole-4-oxy)-2-chloro-benzenethiol

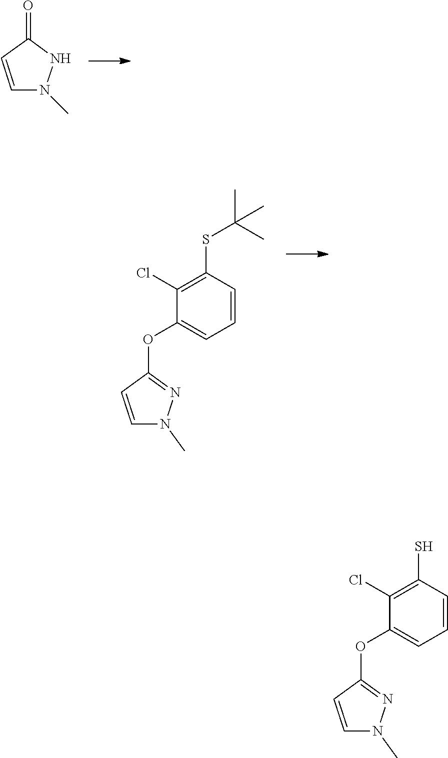

Dissolve the compound 1-methyl-1H-pyrazole-3(2H)-ketone (0.49 g, 5 mmol) in 8 mL of N, N-dimethylformamide, add 3-(tert-butylmercapto)-2-chloro-1-iodobenzene (1.79 g, 5.5 mmol) and cesium carbonate (3.26 g, 10 mmol). The reaction mixture is heated to 50° C. and stirred for 6 hours. Cool to room temperature, the reaction solution is diluted with water and extracted with ethyl acetate. The resulting organic phase is then washed with saturated brine, dried with anhydrous sodium sulfate, and the organic phase is evaporated to dryness under reduced pressure. The residue is purified by column chromatography to give the compound 3-(3-(tert-butylmercapto)-2-chlorophenoxy)-1-methyl-1H-pyrazole (1.11 g, 75% yield). LC/MS (ESI): m/z=297.1[M+H]⁺.

Dissolve the compound 3-(3-(tert-butylmercapto)-2-chlorophenoxy)-1-methyl-1H-pyrazole (890 mg, 3 mmol) in 30 mL of concentrated hydrochloric acid, react for 2 hours at 50° C. After cooling to room temperature, the reaction mixture is neutralized with sodium bicarbonate and the aqueous phase is extracted with ethyl acetate. The resulting organic phase is then washed with saturated brine, dried with anhydrous sodium sulfate, and the organic phase is evaporated to dryness under reduced pressure. The residue is purified by column chromatography to give the compound 3-(1-methylpyrazole-4-oxy)-2-chloro-benzenethiol (440 mg, 61% yield). LC/MS (ESI): m/z=241.0[M+H]⁺.

### Preparation of the Intermediate 2,6-dichloropyrido[3,2-d]pyrimidine

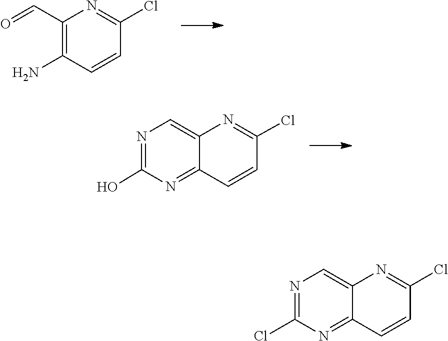 [SMILES: Nc1ccc(Cl)nc1C=O ; Oc1ncc2nc(Cl)ccc2n1 ; Clc1ccc2nc(Cl)ncc2n1]

Dissolve the compound 3-amino-6-chloropyridine aldehyde (1.57 g, 10 mmol) in 15 mL of urea, heat to 130° C., and react for 2 hours with stirring. After cooling to room temperature, the reaction mixture is filtered, and the filter cake is washed with water and dried to give the compound 2-hydroxy-6-chloropyrido[3,2-d]pyrimidine (1.63 g, 90% yield). LC/MS(ESI): m/z=182.0[M+H]⁺.

Dissolve the compound 2-hydroxy-6-chloropyrido[3,2-d]pyrimidine (0.91 g, 5 mmol) in 20 mL of phosphorous trichloride, heat to 105° C., and react for 3 hours with stirring. After cooling to room temperature, most of the solvent is removed under reduced pressure, the residue is poured into ice water and extracted with dichloromethane, the resulting organic phase is then washed with saturated sodium bicarbonate solution and saturated brine, dried with anhydrous sodium sulfate, and the organic phase is evaporated under reduced pressure. The residue is purified by column chromatography to give the compound 2,6-dichloropyrido[3,2-d]pyrimidine(0.62 g, 62% yield). LC/MS (ESI): m/z=200.0[M+H]⁺.

### Preparation of the Intermediate 2,6-dichloropyrido[2,3-b]pyrazine

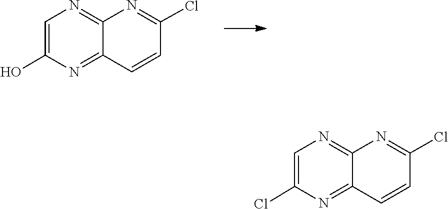 [SMILES: Oc1cnc2nc(Cl)ccc2n1 ; Clc1cnc2nc(Cl)ccc2n1]

Dissolve the compound 2-hydroxy-6-chloropyrido[3,2-d]pyrimidine (0.91 g, 5 mmol) in 10 mL of toluene, and add thionyl chloride (5.1 mL, 55 mmol). Heat the reaction mixture to reflux, let react for 24 hours with stirring, then cool to room temperature. The residue is poured into ice water, extracted with dichloromethane. The resulting organic phase is then washed with saturated sodium bicarbonate solution and saturated brine, dried with anhydrous sodium sulfate, the organic phase is evaporated under reduced pressure. The residue is purified by column chromatography to give the compound 2,6-dichloropyrido[2,3-b]pyrazine(0.85 g, 85% yield). LC/MS (ESI): m/z=200.0[M+H]⁺.

### Preparation of the Intermediate 2,6-dichloropteridine

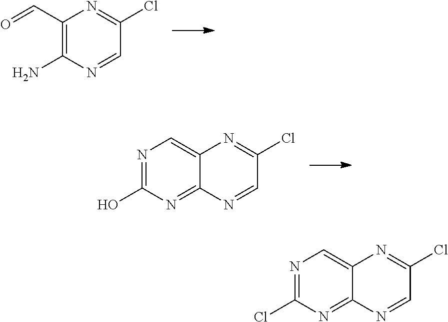 [SMILES: Nc1ncc(Cl)nc1C=O ; Oc1ncc2nc(Cl)cnc2n1 ; Clc1cnc2nc(Cl)ncc2n1]

The compound 2,6-dichloropteridine is obtained by a similar method to the intermediate 2,6-dichloropyrido[3,2-d]pyrimidine (the raw material is changed to 3-amino-6-chloropyrazine-2-aldehyde). LC/MS(ESI): m/z=201.0[M+H]⁺.

### Preparation of the Intermediate 1-methyl-3-(tributylstannyl)-1H-pyrazole

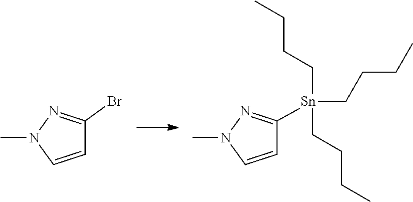

Dissolve the compound 1-methyl-3-bromopyrazole (5 g, 31.25 mmol) in 80 mL of toluene, add hexa-n-butylditin (18.05 g. 31.25 mmol), and tetrakis(triphenylphosphine)palladium (1.79 g, 1.56 mmol), heat the mixture to 120° C., and react for 5 hours with stirring, then cool to room temperature. The reaction solution is evaporated under reduced pressure. The residue is purified by column chromatography to give the compound 1-methyl-3-(tributylstannyl)-1H-pyrazole (2.9 g, 25% yield).

### Preparation of the Intermediate 3-(1-methyl-1H-pyrazole-3-yl)-2-chloro-benzenethiol

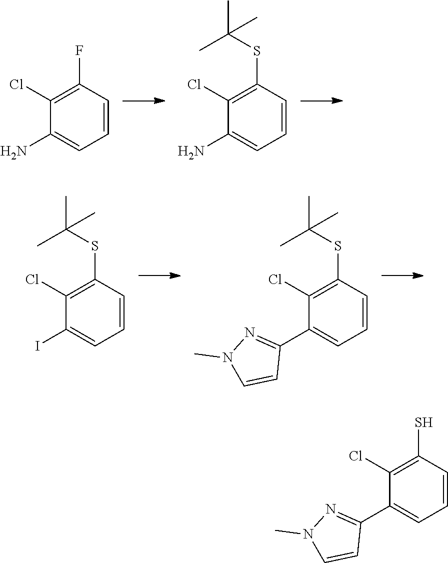 [SMILES: Nc1cccc(F)c1Cl ; CC(C)(C)Sc1cccc(N)c1Cl ; CC(C)(C)Sc1cccc(I)c1Cl ; Cn1ccc(-c2cccc(SC(C)(C)C)c2Cl)n1 ; Cn1ccc(-c2cccc(S)c2Cl)n1]

Dissolve the compound 3-fluoro-2-chloroaniline (5 g, 34.35 mmol) in 80 mL of N,N-dimethylformamide, add tert-butyl mercaptan (9.29 g, 103 mmol) and cesium carbonate (16.79 g, 51.53 mmol), heat the mixture to 120° C., react for 24 with stirring, then cool to room temperature. The reaction solution is poured into 150 mL of saturated ammonium chloride solution and, extracted with ethyl acetate. The resulting organic phase is then washed with saturated brine, dried with anhydrous sodium sulfate, and the organic phase is evaporated to dryness under reduced pressure. The residue is purified by column chromatography to give the compound 3-(tert-butylmercapto)-2-chloroaniline (5.85 g, 79% yield). LC/MS (ESI): m/z=216.1[M+H]⁺.

Dissolve the compound 3-(tert-butylmercapto)-2-chloroaniline (5 g, 23.25 mmol) in 15 mL of concentrated hydrochloric acid, drop in 40 mL of aqueous solution of sodium nitrite (1.25 g, 26.3 mmol) at −5° C., stir for 1 h, then drop in 40 mL of aqueous solution of potassium iodide (5.4 g, 46.5 mmol) at −5° C., stir for 30 min. The reaction solution is extracted with ethyl acetate, and the resulting organic phase is then washed with water and saturated brine, dried with anhydrous sodium sulfate, and the organic phase is evaporated to dryness under reduced pressure. The residue is purified by column chromatography to give the compound 3-(tert-butylmercapto)-2-chloro-1-iodobenzene (4.93 g, 65% yield). LC/MS (ESI): m/z=326.9[M+H]⁺.

Dissolve the compound 1-methyl-3-(tributylstannyl)-1H-pyrazole (2 g, 5.39 mmol) in 50 mL of xylene, add compound 3-(tert-butylmercapto)-2-chloro-1-iodobenzene (1.76 g, 5.39 mmol) and tetrakis(triphenylphosphine)palladium (312 mg, 0.27 mmol). Heat the reaction mixture to reflux, stir for 2 hours. After cooling to room temperature, the reaction solution is evaporated under reduced pressure. The residue is purified by column chromatography to give the compound (3-(3-(tert-butylmercapto)-2-chlorophenyl)-1-methyl-1H-pyrazole (1.14 g, 75% yield). LC/MS (ESI): m/z=281.1 [M+H]⁺.

Dissolve the compound 3-(3-(tert-butylmercapto)-2-chlorophenyl)-1-methyl-1H-pyrazole (1.12 g, 4 mmol) in 25 mL of toluene, add anhydrous aluminum trichloride (2.13 g, 16 mmol), and the mixture is stirred at room temperature for 1 hour under nitrogen protection, quenched with ice water and, extracted with ethyl acetate. The resulting organic phase is then washed with saturated brine, dried with anhydrous sodium sulfate, and the organic phase is evaporated under reduced pressure. The crude product 3-(1-methyl-1H-pyrazole-3-yl)-2-chloro-benzenethiol (0.89 g, yield 99%) is obtained, which is directly used in the next reaction. LC/MS(ESI): m/z=225.0[M+H]⁺.

### Preparation of the Intermediate 3-(1-ethyl-1H-pyrazole-3-yl)-2-chloro-benzenethiol

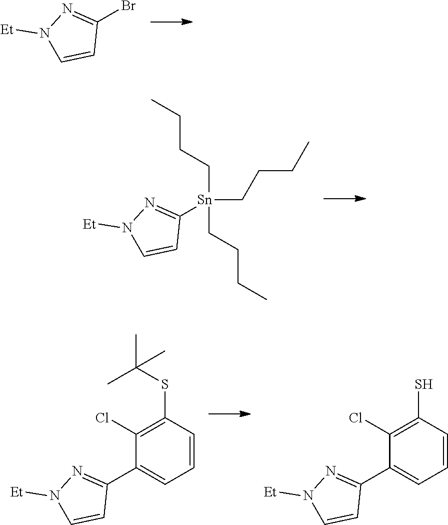 [SMILES: CCn1ccc(Br)n1 ; CCC[CH2][Sn]([CH2]CCC)([CH2]CCC)[c]1ccn(CC)n1 ; CCn1ccc(-c2cccc(SC(C)(C)C)c2Cl)n1 ; CCn1ccc(-c2cccc(S)c2Cl)n1]

The compound 3-(1-ethyl-1H-pyrazole-3-yl)-2-chloro-benzenethiol is obtained by a preparation method similar to that of the intermediate 3-(1-methyl-1H-pyrazole-3-yl)-2-chloro-benzenethiol (raw material is changed to 3-bromo-1-ethylpyrazole). LC/MS(ESI): m/z=240.0[M+H]⁺.

### Preparation of the Intermediate 3-(1-isopropyl-1H-pyrazole-3-yl)-2-chloro-benzenethiol

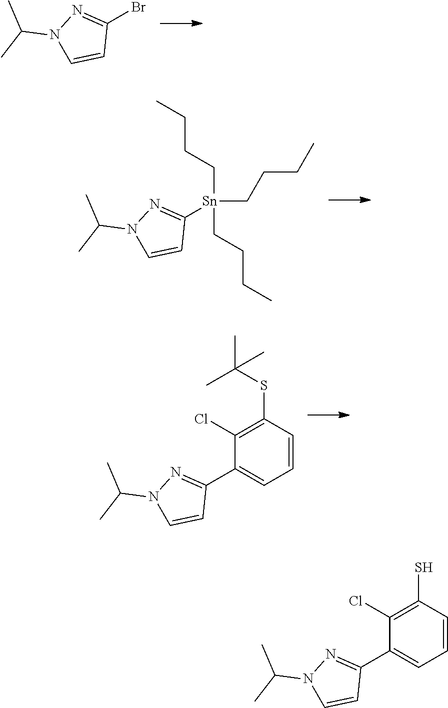

The compound 3-(1-isopropyl-1H-pyrazole-3-yl)-2-chloro-benzenethiol is obtained by a preparation method similar to that of the intermediate 3-(1-methyl-1H-pyrazole-3-yl)-2-chloro-benzenethiol (the raw material is changed to 3-bromo-1-isopropylpyrazole). LC/MS(ESI): m/z=254.0[M+H]⁺.

### Preparation of the Intermediate 3-(pyrimidine-5-yl)-2-chloro-benzenethiol

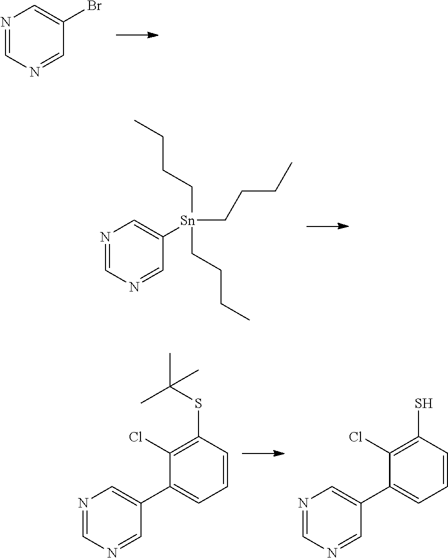 [SMILES: Brc1cncnc1 ; CCC[CH2][Sn]([CH2]CCC)([CH2]CCC)[c]1cncnc1 ; CC(C)(C)Sc1cccc(-c2cncnc2)c1Cl ; Sc1cccc(-c2cncnc2)c1Cl]

The compound 3-(pyrimidine-5-yl)-2-chloro-benzenethiol is obtained by a preparation method similar to that of the intermediate 3-(1-methyl-1H-pyrazole-3-yl)-2-chloro-benzenethiol (the raw material is changed to 3-bromo-1-isopropylpyrazole). LC/MS(ESI): m/z=224.0[M+H]⁺.

### Preparation of the Intermediate 3-(pyrazine-2-yl)-2-chloro-benzenethiol

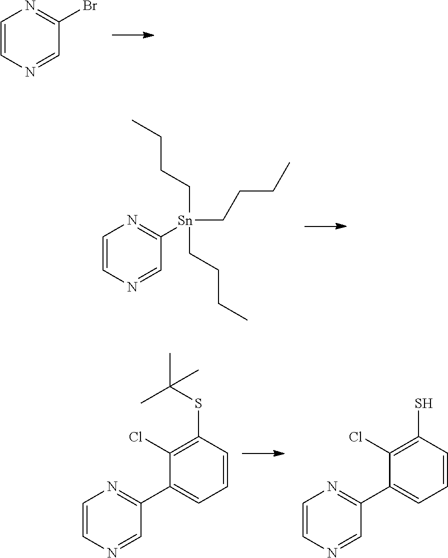 [SMILES: Brc1cnccn1 ; CCC[CH2][Sn]([CH2]CCC)([CH2]CCC)[c]1cnccn1 ; CC(C)(C)Sc1cccc(-c2cnccn2)c1Cl ; Sc1cccc(-c2cnccn2)c1Cl]

The compound 3-(pyrazine-2-yl)-2-chloro-benzenethiol is obtained by a preparation method similar to that of the intermediate 3-(1-methyl-1H-pyrazole-3-yl)-2-chloro-benzenethiol (the raw material is changed to 3-bromo-1-isopropylpyrazole). LC/MS(ESI): m/z=224.0[M+H]⁺.

### Preparation of the Intermediate 3-(pyridine-3-yl)-2-chloro-benzenethiol

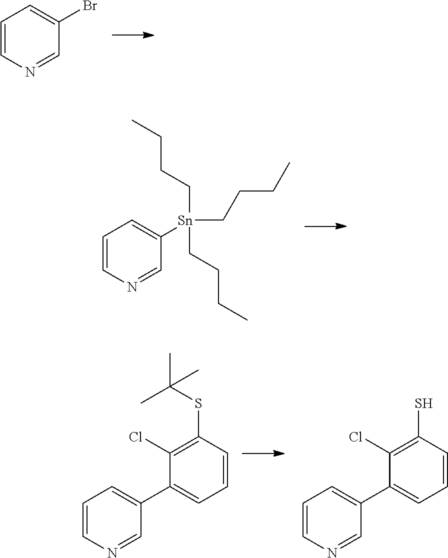 [SMILES: Brc1cccnc1 ; CCC[CH2][Sn]([CH2]CCC)([CH2]CCC)[c]1cccnc1 ; CC(C)(C)Sc1cccc(-c2cccnc2)c1Cl ; Sc1cccc(-c2cccnc2)c1Cl]

The compound 3-(pyridine-2-yl)-2-chloro-benzenethiol is obtained by a similar preparation to that of the intermediate 3-(1-methyl-1H-pyrazole-3-yl)-2-chloro-benzenethiol (the raw material is changed to 3-bromo-1-isopropylpyrazole). LC/MS(ESI): m/z=222.0[M+H]⁺.

### Preparation of the Intermediate 3-(pyrimidine-4-yl)-2-chloro-benzenethiol

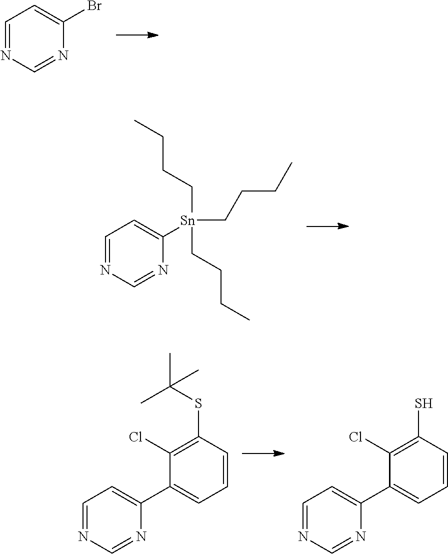 [SMILES: Brc1ccncn1 ; CCC[CH2][Sn]([CH2]CCC)([CH2]CCC)[c]1ccncn1 ; CC(C)(C)Sc1cccc(-c2ccncn2)c1Cl ; Sc1cccc(-c2ccncn2)c1Cl]

The compound 3-(pyrimidine-4-yl)-2-chloro-benzenethiol is obtained by a preparation method similar to that of the intermediate 3-(1-methyl-1H-pyrazole-3-yl)-2-chloro-benzenethiol (the raw material is changed to 3-bromo-1-isopropylpyrazole). LC/MS(ESI): m/z=224.0[M+H]⁺.

### Preparation of the Intermediate 2-hydroxy-4-oxo-6,7,8,9-tetrahydro-4H-pyrido[1,2-a]pyrimidine-3-carboxylic acid ethyl ester

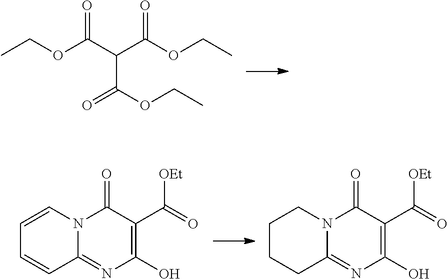 [SMILES: CCOC(=O)C(C(=O)OCC)C(=O)OCC ; CCOC(=O)c1c(O)nc2ccccn2c1=O ; CCOC(=O)c1c(O)nc2n(c1=O)CCCC2]

Dissolve the compound 4c (4.64 g, 20 mmol) and 2-aminopyridine (0.94 g, 10 mmol) in 40 mL of xylene, heat the mixture to 120° C., stir for 16 hours, then cool to room temperature, filter. The filter cake is washed 3 times with methanol and, dried to give the compound ethyl 2-hydroxy-4-oxo-pyrido[1,2-a]pyrimidine-3-carboxylate(0.58 g, 25% yield). LC/MS (ESI): m/z=235.1[M+H]⁺.

Under nitrogen protection, dissolve the compound 2-hydroxy-4-oxo-pyrido[1,2-a]pyrimidine-3-carboxylicacid ethyl ester (468 mg, 2 mmol) in 4 mL of methanol, then add palladium-carbon (40 mg), and perform hydrogen substitution for 3 times. Stir the reaction mixture at room temperature for 2 hours, filter, and concentrate under reduced pressure, to give the compound ethyl 2-hydroxy-4-oxo-6,7,8,9-tetrahydro-4H-pyrido[1,2-a]pyrimidine-3-carboxylate (452 mg, 95% yield). LC/MS(ESI): m/z=239.1[M+H]⁺.

### Preparation of the Intermediate 3-amino-2-chloro-benzenethiol

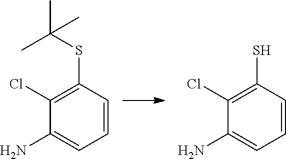

Dissolve the compound 3-(3-(tert-butylmercapto)-2-chloroaniline (10 g, 46.4 mmol) in 100 mL of concentrated hydrochloric acid, heat to 55° C. and stir overnight. Cool to room temperature, quench to neutrality with sodium bicarbonate, the aqueous phase is extracted with ethylacetate. The resulting organic phase is then washed with saturated brine, dried with anhydrous sodium sulfate, and the organic phase is evaporated to dryness under reduced pressure. The residue is purified by column chromatography to give the compound 3-amino-2-chloro-benzenethiol (4.9 g, 66% yield). LC/MS(ESI): m/z=160.0[M+H]⁺.

### Preparation of the Intermediate 3-(pyridine-4-amino)-2-chloro-benzenethiol

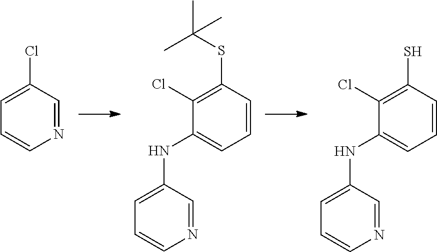 [SMILES: Clc1cccnc1 ; CC(C)(C)Sc1cccc(Nc2cccnc2)c1Cl ; Sc1cccc(Nc2cccnc2)c1Cl]

Dissolve the compound 3-chloropyridine (1.14 g, 10 mmol) in 15 mL of toluene, add sodium tert-butoxide (1.15 g, 12 mmol), and 4,5-bis(diphenylene)lyn-9,9-dimethyloxanthene (60 mg). Perform nitrogen substitution for 3 times. Add 85 mg of tris(dibenzylideneindeneacetketone)dipalladium and the reaction mixture is refluxed and stirred for 2 hours. Cool to room temperature, dilute with water and extract with ethyl acetate. The resulting organic phase is then washed with saturated brine, dried with anhydrous sodium sulfate, and the organic phase is evaporated to dryness under reduced pressure. The residue is purified by column chromatography to give the compound N-(3-tert-butylmercapto)-2-chlorophenylpyridine-3-amine (1.52 g, 52% yield). LC/MS (ESI): m/z=293.1[M+H]⁺.

Dissolve the compound N-(3-tert-butylmercapto)-2-chlorophenylpyridine-3-amine (1.17 g, 4 mmol) in 30 mL of concentrated hydrochloric acid and, heat to 50° C. and react for 2 hours, cool to room temperature, quench to neutrality with sodium bicarbonate. The aqueous phase is extracted with ethyl acetate. The resulting organic phase is then washed with saturated brine, dried with anhydrous sodium sulfate, and the organic phase is evaporated to dryness under reduced pressure. The residue is purified by column chromatography to give the compound 3-(pyridine-4-amino)-2-chloro-benzenethiol (0.66 g, 70% yield). LC/MS (ESI): m/z=237.0[M+H]⁺.

### Preparation of the Intermediate 3-(pyrimidine-2-amino)-2-chloro-benzenethiol

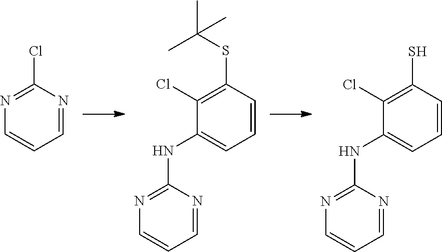 [SMILES: Clc1ncccn1 ; CC(C)(C)Sc1cccc(Nc2ncccn2)c1Cl ; Sc1cccc(Nc2ncccn2)c1Cl]

The compound 3-(pyrimidine-2-amino)-2-chloro-benzenethiol is obtained by a preparation method similar to that of the intermediate 3-(pyridine-4-amino)-2-chloro-benzenethiol (the raw material is changed to 2-chloropyrimidine). LC/MS(ESI): m/z=239.0[M+H]⁺.

### Preparation of the Intermediate 3-(pyrazine-4-amino)-2-chloro-benzenethiol

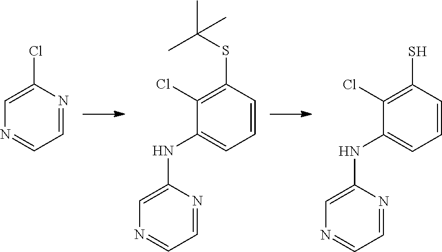 [SMILES: Clc1cnccn1 ; CC(C)(C)Sc1cccc(Nc2cnccn2)c1Cl ; Sc1cccc(Nc2cnccn2)c1Cl]

The compound 3-(pyrazine-2-amino)-2-chloro-benzenethiol is obtained by a preparation method similar to that of the intermediate 3-(pyrazine-4-amino)-2-chloro-benzenethiol (the raw material is changed to 2-chloropyrazine). LC/MS(ESI): m/z=239.0[M+H]⁺.

### Preparation of the Intermediate 3-(pyrimidine-4-amino)-2-chloro-benzenethiol

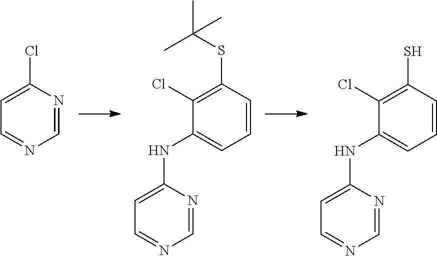 [SMILES: Clc1ccncn1 ; CC(C)(C)Sc1cccc(Nc2ccncn2)c1Cl ; Sc1cccc(Nc2ccncn2)c1Cl]

The compound 3-(pyrimidine-4-amino)-2-chloro-benzenethiol is obtained by a preparation method similar to that of the intermediate 3-(pyrimidine-4-amino)-2-chloro-benzenethiol (the raw material is changed to 4-chloropyrimidine). LC/MS(ESI): m/z=239.0[M+H]⁺.

### Preparation of the Intermediate (3S,4S)-8-(5-chloropyrimidine-2-yl)-3-methyl-2-oxa-8-azaspiro[4.5]decan-4-amine

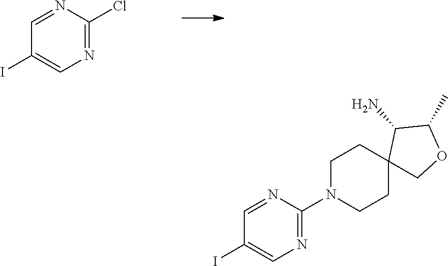 [SMILES: Clc1ncc(I)cn1 ; C[C@@H]1OCC2(CCN(c3ncc(I)cn3)CC2)[C@@H]1N]

Dissolve 2-Chloro-5-iodopyrimidine (7.2 g, 30 mmol) in 50 mL of dichloromethane, add (3S,4S)-4-amino-3-methyl-2-oxa-8-azaspiro[4.5]decane dihydrochloride (7.3 g, 30 mmol) and triethylamine (9.11 g, 90 mmol). Stir the reaction mixture at room temperature and react overnight. The reaction solution is diluted with dichloromethane, washed with saturated sodium bicarbonate, dried with anhydrous sodium sulfate, and the organic phase is evaporated to dryness under reduced pressure. The residue is purified by column chromatography to give the compound (3S,4S)-8-(5-chloropyrimidine-2-yl)-3-methyl-2-oxa-8-azaspiro[4.5]decan-4-amine (5.34 g, 63% yield). LC/MS(ESI): m/z=283.1[M+H]⁺.

### Preparation of the Intermediate N-(2-chloro-3-mercaptophenyl)pyrimidine-2-carboxamide

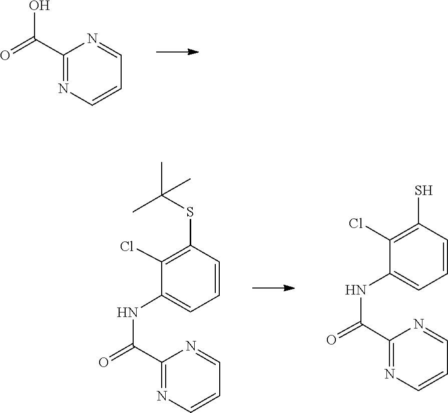 [SMILES: O=C(O)c1ncccn1 ; CC(C)(C)Sc1cccc(NC(=O)c2ncccn2)c1Cl ; O=C(Nc1cccc(S)c1Cl)c1ncccn1]

Add 2-oxypyrimidine (620 mg, 5 mmol) to 10 mL of thionyl chloride, stir and reflux for 2 hours. The mixture is concentrated under reduced pressure to give acyl chloride. Add 15 mL of dichloromethane, pyridine (593 mg, 7.5 mmol), 3-(3-(tert-butylthio)-2-chlorophenyl)aniline (539 mg, 2.5 mmol), and 4-dimethylaminopyridine (153 mg, 1.25 mmol). Stir at room temperature for 2 hours, then quench with water, and extract with dichloromethane. The resulting organic phase is then washed with saturated brine, dried with anhydrous sodium sulfate and, the organic phase is evaporated to dryness under reduced pressure. The residue is purified by column chromatography to give the compound N-(2-chloro-3-(3-(tert-butylmercapto)phenyl)pyrimidine-2-carboxamide (627 mg, 78% yield). LC/MS (ESI): m/z=322.1[M+H]⁺.

Dissolve the compound N-(2-chloro-3-(3-(tert-butylmercapto)phenyl)pyrimidine-2-carboxamide (578 mg, 1.8 mmol) in 30 mL of concentrated hydrochloric acid, react at 50° C. for 2 hours, cool to room temperature, and quench to neutrality with sodium bicarbonate. The aqueous phase is extracted with ethylacetate. The resulting organic phase is then washed with saturated brine, dried with anhydrous sodium sulfate, and the organic phase is evaporated to dryness under reduced pressure. The residue is purified by column chromatography to give the compound N-(2-chloro-3-mercaptophenyl)pyrimidine-2-carboxamide (0.27 g, 56% yield). LC/MS (ESI): m/z=267.0[M+H]⁺.

### Preparation of the Intermediate N-(2-chloro-3-mercaptophenyl)pyrimidine-4-carboxamide

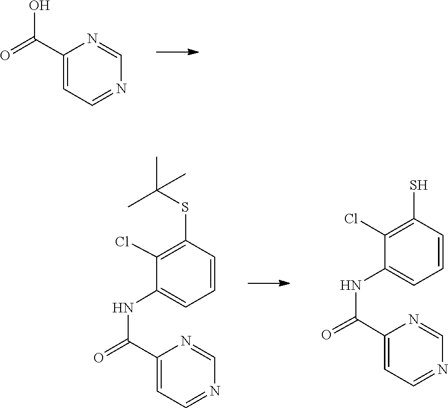 [SMILES: O=C(O)c1ccncn1 ; CC(C)(C)Sc1cccc(NC(=O)c2ccncn2)c1Cl ; O=C(Nc1cccc(S)c1Cl)c1ccncn1]

compound N-(2-chloro-3-mercaptophenyl)pyrimidine-4-carboxamide is obtained by a similar method to that of the intermediate N-(2-chloro-3-mercaptophenyl)pyrimidine-2-carboxamide (the raw material is changed to pyrimidine-4-carboxylicacid). LC/MS(ESI): m/z=267.0[M+H]⁺.

### Preparation of the Intermediate N-(2-chloro-3-mercaptophenyl)-2-hydroxy-4-oxo-6,7,8,9-tetrahydro-4H-pyrido[1,2-a]pyrimidine-3-carboxamide

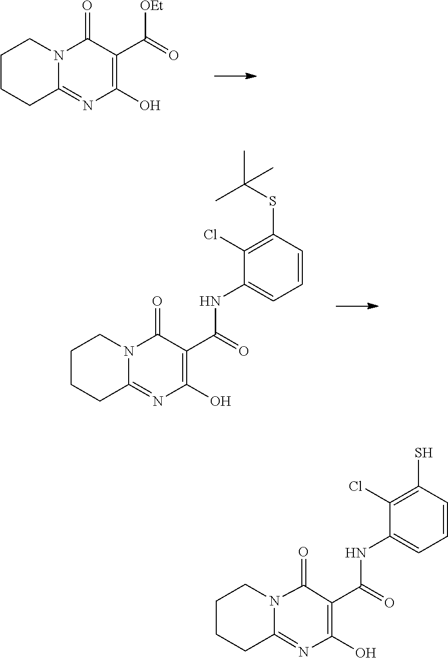 [SMILES: CCOC(=O)c1c(O)nc2n(c1=O)CCCC2 ; CC(C)(C)Sc1cccc(NC(=O)c2c(O)nc3n(c2=O)CCCC3)c1Cl ; O=C(Nc1cccc(S)c1Cl)c1c(O)nc2n(c1=O)CCCC2]

Dissolve the compound 3-(3-(tert-butylmercapto)-2-chloroaniline (1.08 g, 5 mmol) and the compound 2-hydroxy-4-oxo-6,7,8,9-tetrahydro-4H-pyrido[1,2-a]pyrimidine-3-carboxylicacid ethyl ester (1.43 g, 6 mmol) in 20 mL of chlorobenzene, heat to reflux with stirring for 3 hours, cool to room temperature, filter. After drying, compound N-(3-(tert-butylmercapto)-2-chlorophenyl)-2-hydroxy-4-oxo-6,7,8,9-tetrahydro-4H-pyrid o[1,2-a]pyrimidine-3-carboxamide (1.18 g, 58% yield) is obtained. LC/MS (ESI): m/z=408.1[M+H]⁺.

Dissolve the compound N-(3-(tert-butylmercapto)-2-chlorophenyl)-2-hydroxy-4-oxo-6,7,8,9-tetrahydro-4H-pyrid o[1,2-a]pyrimidine-3-carboxamide (1.02 g,2.5 mmol) in 10 mL of concentrated hydrochloric acid, heat to 50° C. and stir for 3 hours, cool to room temperature, and quench to neutrality with sodium bicarbonate. The aqueous phase is extracted with ethyl acetate. The resulting organic phase is then washed with saturated brine, dried with anhydrous sodium sulfate, and the organic phase is evaporated to dryness under reduced pressure. The residue is purified by column chromatography to give the compound N-(2-chloro-3-mercaptophenyl)-2-hydroxy-4-oxo-6,7,8,9-tetrahydro-4H-pyrido[1,2-a]pyr imidine-3-carboxamide(0.57 g, 65% yield). LC/MS (ESI): m/z=353.0[M+H]⁺.

### EXAMPLE 1

### Preparation of 7-((3S,4S)-4-amino-3-methyl-2-oxa-8-azaspiro[4.5]decane-8-yl)-3-(2,3-dichloropheny l)quinazoline-2,4(1H,3H)-dione

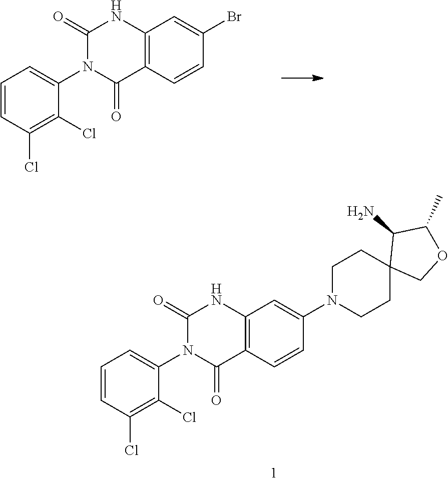 [SMILES: O=c1[nH]c2cc(Br)ccc2c(=O)n1-c1cccc(Cl)c1Cl ; C[C@@H]1OCC2(CCN(c3ccc4c(=O)n(-c5cccc(Cl)c5Cl)c(=O)[nH]c4c3)CC2)[C@H]1N]

Dissolve the compound 7-bromo-3-(2,3-dichlorophenyl)quinazoline-2,4(1H,3H)-dione (772 mg, 2 mmol) in 8 mL of toluene, add (3S,4S)-4-amino-3-methyl-2-oxa-8-azaspiro[4.5]decane dihydrochloride (584 mg, 2.4 mmol), cesium carbonate (1.95 g, 6.0 mmol), and bis(dibenzylideneacetone)palladium (12 mg, 0.02 mmol), 2-dicyclohexylphosphonium-2′,6′-diisopropoxy-1,1′-biphenyl (19 mg, 0.04 mmol).

Perform nitrogen substitution is 3 times, and react at 90° C. for 6 hours with stirring. After cooling to room temperature, the reaction solution is passed through a silica gel short column, rinse the column with ethyl acetate, and evaporate the solution to dryness under reduced pressure. The residue is purified by column chromatography to give the target product 1 (494 mg, yield 52%). 1H NMR (400 MHZ, DMSO-d₆) δ: 11.51 (s, 1H), 7.83 (d, 1H), 7.51-7.45 (m, 2H), 7.31-7.26 (m, 2H), 6.83 (d, 1H), 4.10-4.07 (m, 1H), 3.89-3.45 (m, 4H), 3.32-3.25 (m, 2H), 2.91 (d, 1H), 1.77-1.32 (m, 6H), 1.13 (d, 3H); LC/MS(ESI): m/z=475.1[M+H]⁺.

### Embodiment 2

### Preparation of (S)-7-(4-amino-2-oxa-8-azaspiro[4.5]decane-8-yl)-3-(2,3-dichlorophenyl)quinazoline-2,4(1H,3H)-dione

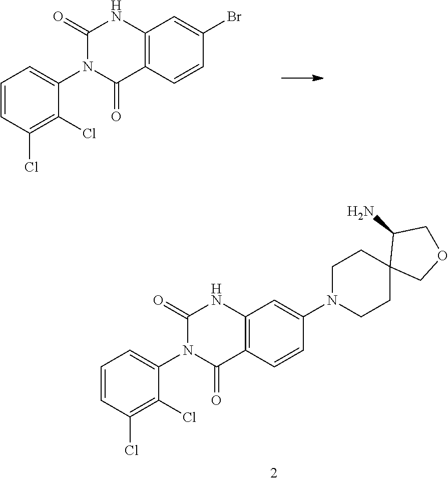 [SMILES: O=c1[nH]c2cc(Br)ccc2c(=O)n1-c1cccc(Cl)c1Cl ; N[C@H]1COCC12CCN(c1ccc3c(=O)n(-c4cccc(Cl)c4Cl)c(=O)[nH]c3c1)CC2]

Compound 2 (415 mg, yield 45%) is obtained in a similar manner to Embodiment 1. LC/MS(ESI): m/z=461.1[M+H]⁺.

### Embodiment 3

### Preparation of (S)-3-(2-amino-3-chloropyridine-4-yl)-7-(4-amino-2-oxa-8-azaspiro[4.5]decane-8-yl)q uinazoline-2,4(1H,3H)-dione

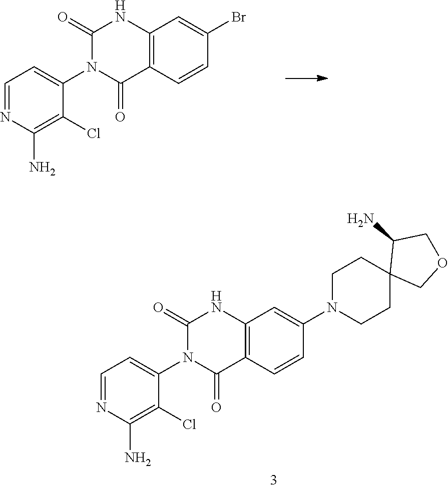 [SMILES: Nc1nccc(-n2c(=O)[nH]c3cc(Br)ccc3c2=O)c1Cl ; Nc1nccc(-n2c(=O)[nH]c3cc(N4CCC5(CC4)COC[C@@H]5N)ccc3c2=O)c1Cl]

Compound 3 (222 mg, yield 25%) is obtained in a similar manner to Embodiment 1. ¹H NMR (400 MHZ, DMSO-d₆) δ: 11.23 (s, 1H), 7.82 (d, 1H), 7.51-7.45 (m, 1H), 7.30-7.26 (m, 1H), 6.85 (d, 1H), 6.64 (d, 1H), 5.87 (s, 2H), 4.12-4.08 (m, 1H), 3.89-3.45 (m, 5H), 3.32-3.25 (m, 2H), 2.82-2.74 (m, 1H), 1.74-1.29 (m, 6H); LC/MS(ESI): m/z=443.2[M+H]⁺.

### Embodiment 4

### Preparation of (S)-7-(1-amino-1,3-dihydrospiro[indene-2,4′-piperidine]-1-yl)-3-(2,3-dichlorophenyl) quinazoline-2,4(1H,3H)-dione

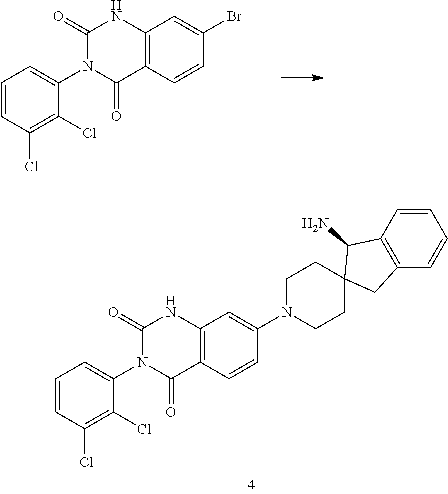 [SMILES: O=c1[nH]c2cc(Br)ccc2c(=O)n1-c1cccc(Cl)c1Cl ; N[C@H]1c2ccccc2CC12CCN(c1ccc3c(=O)n(-c4cccc(Cl)c4Cl)c(=O)[nH]c3c1)CC2]

Compound 4 (548 mg, yield 54%) is obtained in a similar manner to Embodiment 1. ¹H NMR (400 MHz, DMSO-d₆) δ: 11.28 (s, 1H), 7.83 (d, 1H), 7.51-7.45 (m, 2H), 7.31-7.23 (m, 6H), 6.83 (d, 1H), 4.08-4.03 (m, 1H), 3.53-3.45 (m, 2H), 3.32-3.25 (m, 2H), 2.91-2.79 (m, 2H), 1.65-1.30 (m, 6H); LC/MS(ESI): m/z=507.1[M+H]⁺.

### Embodiment 5

### Preparation of (S)-7-(5-Amino-5,7-dihydrospiro[cyclopenta[c]pyridine-6,4′-piperidine]-1′-yl)-3-(2,3-dichlorophenyl)auinazoline-2.4(1H,3H)-dione

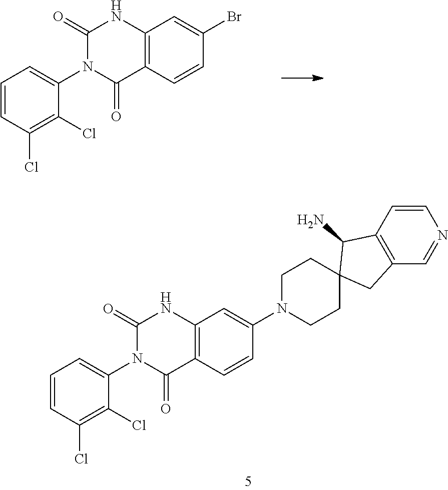 [SMILES: O=c1[nH]c2cc(Br)ccc2c(=O)n1-c1cccc(Cl)c1Cl ; N[C@H]1c2ccncc2CC12CCN(c1ccc3c(=O)n(-c4cccc(Cl)c4Cl)c(=O)[nH]c3c1)CC2]

Compound 5 (498 mg, yield 49%) is obtained in a similar manner to Embodiment 1. ¹H NMR (400 MHZ, DMSO-d₆) δ: 11.50 (s, 1H), 8.28-8.23 (m, 2H), 7.83 (d, 1H), 7.51-7.45 (m, 2H), 7.31-7.23 (m, 3H), 6.83 (d, 1H), 4.03-3.95 (m, 1H), 3.52-3.45 (m, 2H), 3.32-3.25 (m, 2H), 2.71-2.63 (m, 2H), 1.61-1.29 (m, 6H); LC/MS(ESI): m/z=508.1[M+H]⁺.

### Embodiment 6

### Preparation of (S)-7-(7-Amino-5,7-dihydrospiro [cyclopenta[c]pyridine-6,4′-piperidine]-1′-yl)-3-(2,3-dichlorophenyl)quinazoline-2,4(1H,3H)-dione

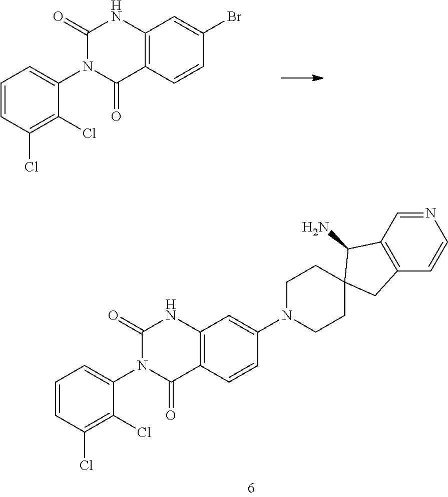 [SMILES: O=c1[nH]c2cc(Br)ccc2c(=O)n1-c1cccc(Cl)c1Cl ; N[C@H]1c2cnccc2CC12CCN(c1ccc3c(=O)n(-c4cccc(Cl)c4Cl)c(=O)[nH]c3c1)CC2]

Compound 6 (437 mg, yield 43%) is obtained in a similar manner to Embodiment 1. LC/MS(ESI): m/z=508.1[M+H]⁺.

### Embodiment 7

### Preparation of (S)-7-(5-Amino-5,7-dihydrospiro [cyclopenta[b]pyridine-6,4′-piperidine]-1′-yl)-3-(2,3-dichlorophenyl)quinazoline-2,4(1H,3H)-dione

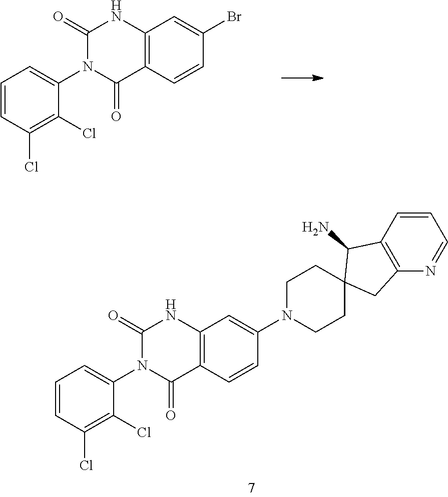 [SMILES: O=c1[nH]c2cc(Br)ccc2c(=O)n1-c1cccc(Cl)c1Cl ; N[C@H]1c2cccnc2CC12CCN(c1ccc3c(=O)n(-c4cccc(Cl)c4Cl)c(=O)[nH]c3c1)CC2]

Compound 7 (528 mg, yield 52%) is obtained in a similar manner to Embodiment 1. LC/MS(ESI): m/z=508.1[M+H]⁺.

### Embodiment 8

### Preparation of (S)-7-(7-Amino-5,7-dihydrospiro[cyclopenta[b]pyridine-6,4′-piperidine]-1′-yl)-3-(2,3-dichlorophenyl)quinazoline-2,4(1H,3H)-dione

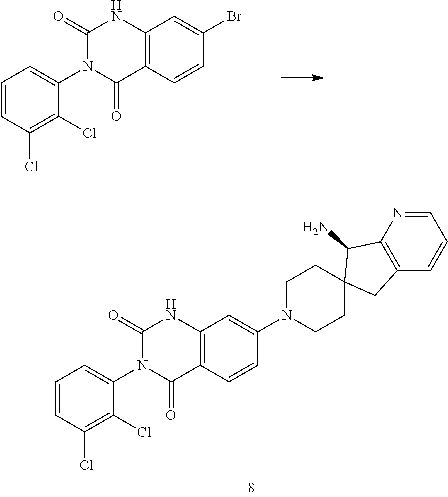 [SMILES: O=c1[nH]c2cc(Br)ccc2c(=O)n1-c1cccc(Cl)c1Cl ; N[C@H]1c2ncccc2CC12CCN(c1ccc3c(=O)n(-c4cccc(Cl)c4Cl)c(=O)[nH]c3c1)CC2]

Compound 8 (416 mg, yield 41%) is obtained in a similar manner to Embodiment 1. LC/MS(ESI): m/z=508.1[M+H]⁺.

### Embodiment 9

### Preparation of (R)-7-(4-(1-aminoethyl)-4-methylpiperidine-1-yl)-3-(2,3-dichlorophenyl)quinazoline-2,4(1H,3H)-dione

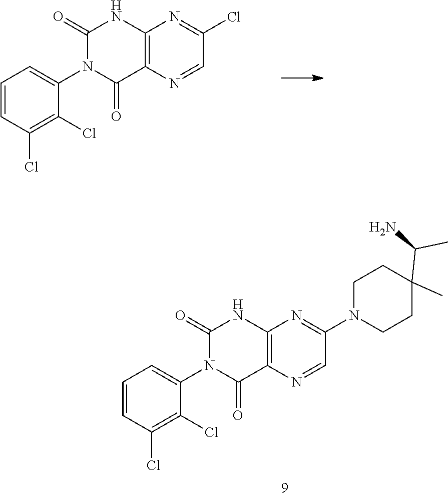 [SMILES: O=c1[nH]c2nc(Cl)cnc2c(=O)n1-c1cccc(Cl)c1Cl ; C[C@H](N)C1(C)CCN(c2cnc3c(=O)n(-c4cccc(Cl)c4Cl)c(=O)[nH]c3n2)CC1]

Compound 9 (394 mg, yield 44%) is obtained in a similar manner to Embodiment 1. LC/MS(ESI): m/z=449.1[M+H]⁺.

### Embodiment 10

### Preparation of 7-(4-(aminoethyl)-4-methylpiperidine-1-yl)-3-(2,3-dichlorophenyl)quinazoline-2,4(1 H,3H)-dione

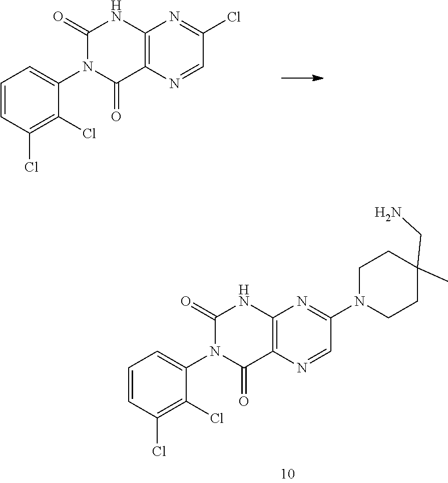 [SMILES: O=c1[nH]c2nc(Cl)cnc2c(=O)n1-c1cccc(Cl)c1Cl ; CC1(CN)CCN(c2cnc3c(=O)n(-c4cccc(Cl)c4Cl)c(=O)[nH]c3n2)CC1]

Compound 10 (328 mg, yield 38%) is obtained in a similar manner to Embodiment 1. ¹H NMR (400 MHZ, DMSO-d₆) δ: 11.42 (s, 1H), 7.93 (s, 1H), 7.51-7.45 (m, 2H), 7.26 (d, 1H), 3.48-3.39 (m, 2H), 3.34-3.26 (m, 2H), 2.63 (s, 2H), 1.72-1.45 (m, 4H), 1.12 (s, 3H); LC/MS(ESI): m/z=433.1[M+H]⁺.

### Embodiment 11

### Preparation of 7-(4-amino-4-methylpiperidine-1-yl)-3-(2,3-dichlorophenyl)quinazoline-2,4(1H,3H)-dione

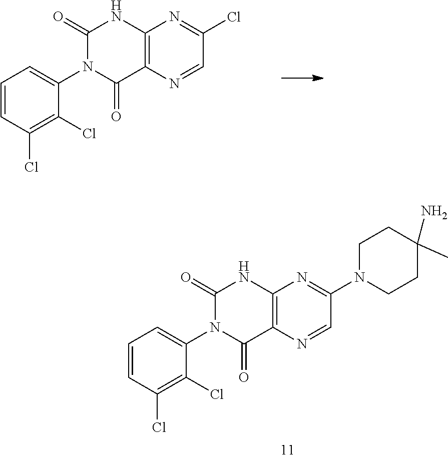 [SMILES: O=c1[nH]c2nc(Cl)cnc2c(=O)n1-c1cccc(Cl)c1Cl ; CC1(N)CCN(c2cnc3c(=O)n(-c4cccc(Cl)c4Cl)c(=O)[nH]c3n2)CC1]

Compound 11 (361 mg, yield 43%) is obtained in a similar manner to Embodiment 1. ¹H NMR (400 MHZ, DMSO-d₆) δ: 11.42 (s, 1H), 7.93 (s, 1H), 7.51-7.45 (m, 2H), 7.26 (d, 1H), 3.45-3.37 (m, 2H), 3.32-3.25 (m, 2H), 1.82-1.65 (m, 4H), 1.24 (s, 3H); LC/MS(ESI): m/z=421.1[M+H]⁺.

### Embodiment 12

### Preparation of 7-((3S,4S)-4-amino-3-methyl-2-oxa-8-azaspiro[4.5]decane-8-yl)-3-(2,3-dichloropheny l)pteridine-2,4(1H,3H)-dione

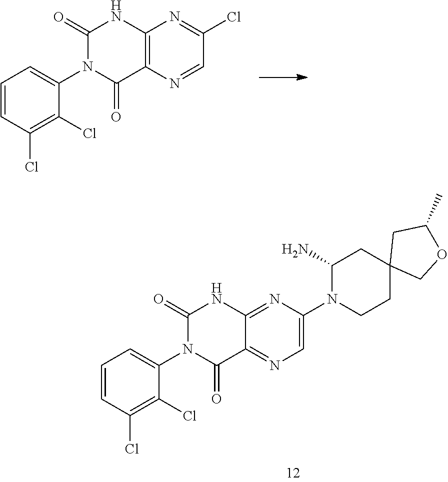 [SMILES: O=c1[nH]c2nc(Cl)cnc2c(=O)n1-c1cccc(Cl)c1Cl ; C[C@H]1CC2(CCN(c3cnc4c(=O)n(-c5cccc(Cl)c5Cl)c(=O)[nH]c4n3)[C@H](N)C2)CO1]

Compound 12 (485 mg, yield 51%) is obtained in a similar manner to Embodiment 1. LC/MS(ESI): m/z=477.1[M+H]⁺.

### Embodiment 13

### Preparation of (S)-7-(1-amino-1,3-dihydrospiro[indene-2,4′-piperidine]-1′-yl)-3-(2,3-dichlorophenyl) pteridine-2,4(1H,3H)-dione

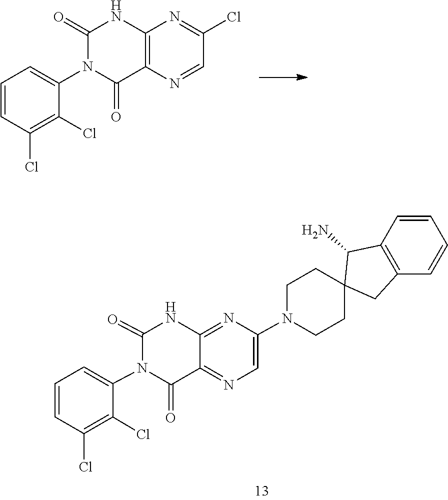 [SMILES: O=c1[nH]c2nc(Cl)cnc2c(=O)n1-c1cccc(Cl)c1Cl ; N[C@@H]1c2ccccc2CC12CCN(c1cnc3c(=O)n(-c4cccc(Cl)c4Cl)c(=O)[nH]c3n1)CC2]

Compound 13 (436 mg, yield 43%) is obtained in a similar manner to Embodiment 1. ¹H NMR (400 MHZ, DMSO-d₆) δ: 11.42 (s, 1H), 7.95 (s, 1H), 7.49-7.43 (m, 2H), 7.25 (d, 1H), 4.08-4.03 (m, 1H), 3.57-3.48 (m, 2H), 3.33-3.25 (m, 2H), 2.91-2.77 (m, 2H), 1.65-1.31 (m, 4H); LC/MS(ESI): m/z=509.1[M+H]⁺.

### Embodiment 14

### Preparation of (S)-7-(5-Amino-5,7-dihydrospiro [cyclopenta[b]pyridine-6,4′-piperidine]-1′-yl)-3-(2,3-dichlorophenyl)pteridine-2,4(1 H,3H)-dione

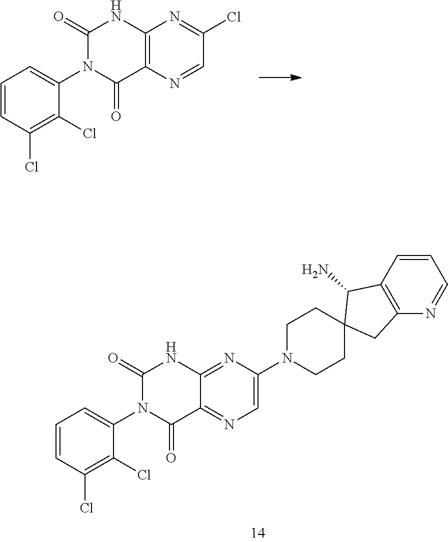 [SMILES: O=c1[nH]c2nc(Cl)cnc2c(=O)n1-c1cccc(Cl)c1Cl ; N[C@@H]1c2cccnc2CC12CCN(c1cnc3c(=O)n(-c4cccc(Cl)c4Cl)c(=O)[nH]c3n1)CC2]

Compound 14 (395 mg, yield 39%) is obtained in a similar manner to Embodiment 1. LC/MS(ESI): m/z=510.1[M+H]⁺.

### Embodiment 15

### Preparation of (S)-7-(5-Amino-5,7-dihydrospiro [cyclopenta[c]pyridine-6,4′-piperidine]-1′-yl)-3-(2,3-dichlorophenyl)pteridine-2,4(1H,3H)-dione

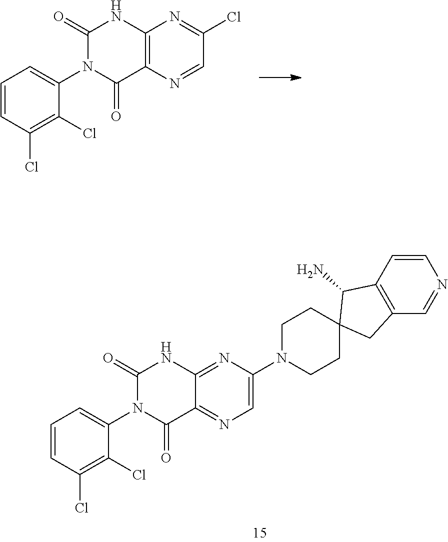 [SMILES: O=c1[nH]c2nc(Cl)cnc2c(=O)n1-c1cccc(Cl)c1Cl ; N[C@@H]1c2ccncc2CC12CCN(c1cnc3c(=O)n(-c4cccc(Cl)c4Cl)c(=O)[nH]c3n1)CC2]

Compound 15 (506 mg, yield 50%) is obtained in a similar manner to Embodiment 1. LC/MS(ESI): m/z=510.1[M+H]⁺.

### Embodiment 16

### Preparation of (S)-7-(7-Amino-5,7-dihydrospiro [cyclopenta[c]pyridine-6,4′-piperidine]-1′-yl)-3-(2,3-dichlorophenyl)pteridine-2,4(1H,3H)-dione

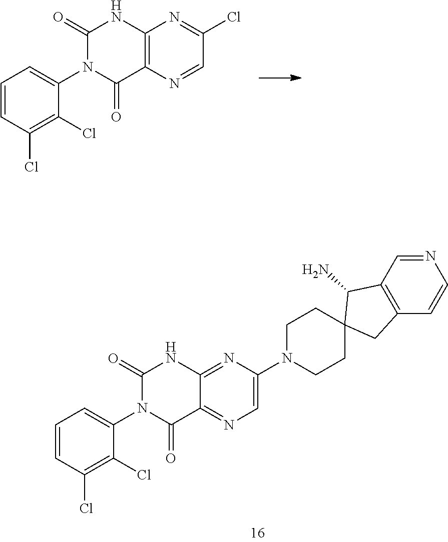 [SMILES: O=c1[nH]c2nc(Cl)cnc2c(=O)n1-c1cccc(Cl)c1Cl ; N[C@@H]1c2cnccc2CC12CCN(c1cnc3c(=O)n(-c4cccc(Cl)c4Cl)c(=O)[nH]c3n1)CC2]

Compound 16 (425 mg, yield 42%) is obtained in a similar manner to Embodiment 1. LC/MS(ESI): m/z=510.1[M+H]⁺.

### Embodiment 17

### Preparation of 3-(2-amino-3-chloropyridine-4-yl)-7-((3S,4S)-4-amino-3-methyl-2-oxa-8-azaspiro[4.5]decane-8-yl)pteridine-2,4(1H,3H)-dione

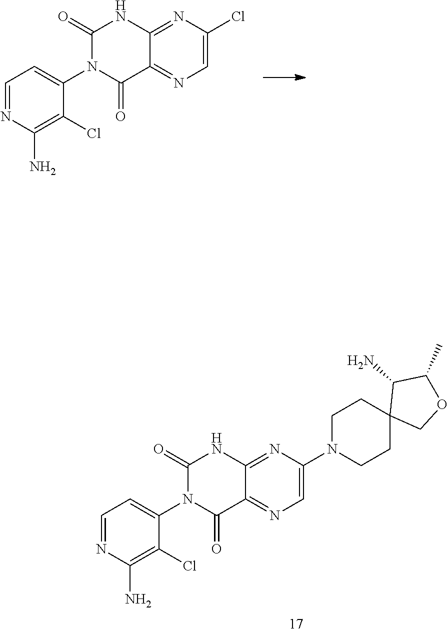 [SMILES: Nc1nccc(-n2c(=O)[nH]c3nc(Cl)cnc3c2=O)c1Cl ; C[C@@H]1OCC2(CCN(c3cnc4c(=O)n(-c5ccnc(N)c5Cl)c(=O)[nH]c4n3)CC2)[C@@H]1N]

Compound 17 (222 mg, yield 29%) is obtained in a similar manner to Embodiment 1. ¹H NMR (400 MHZ, DMSO-d₆) δ: 11.35 (s, 1H), 7.95 (s, 1H), 7.83 (d, 1H), 7.30-7.26 (m, 1H), 5.93 (s, 2H), 4.10-4.07 (m, 1H), 3.89-3.45 (m, 4H), 3.32-3.24 (m, 2H), 2.92 (d, 1H), 1.77-1.32 (m, 6H), 1.14 (d, 3H); LC/MS(ESI): m/z=459.2[M+H]⁺.

### Embodiment 18

### Preparation of (S)-7-(1-amino-1,3-dihydrospiro[indene-2,4′-piperidine]-1′-yl)-3-(2-amino-3-chlorop yridine-4-yl)pteridine-2,4(1H,3H)-dione

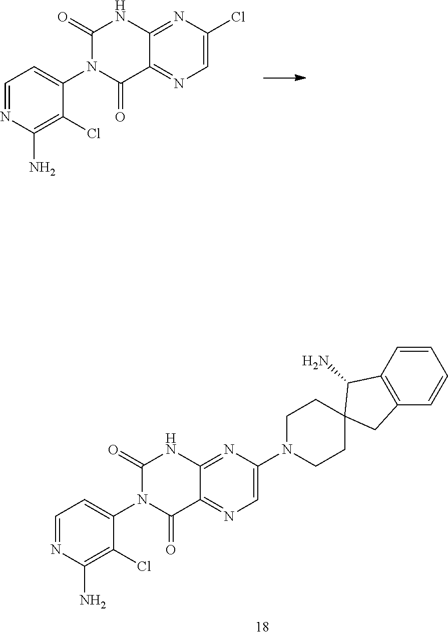 [SMILES: Nc1nccc(-n2c(=O)[nH]c3nc(Cl)cnc3c2=O)c1Cl ; Nc1nccc(-n2c(=O)[nH]c3nc(N4CCC5(CC4)Cc4ccccc4[C@H]5N)cnc3c2=O)c1Cl]

Compound 18 (222 mg, yield 21%) is obtained in a similar manner to Embodiment 1. LC/MS(ESI): m/z=491.2[M+H]⁺.

### Embodiment 19

### Preparation of (S)-3-(2-amino-3-chloropyridine-4-yl)-7-(5-amino-5,7-dihydrospiro[cyclopenta[b]pyr idine-6,4′-piperidine]-1′-yl)pteridine-2,4(1H,3H)-dione

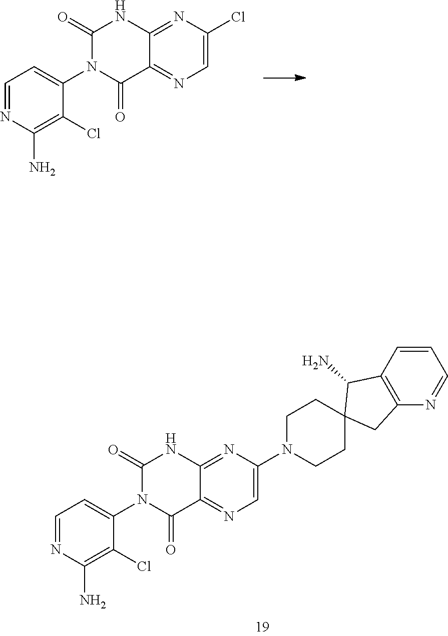 [SMILES: Nc1nccc(-n2c(=O)[nH]c3nc(Cl)cnc3c2=O)c1Cl ; Nc1nccc(-n2c(=O)[nH]c3nc(N4CCC5(CC4)Cc4ncccc4[C@H]5N)cnc3c2=O)c1Cl]

Compound 19 (244 mg, yield 23%) is obtained in a similar manner to Embodiment 1. LC/MS(ESI): m/z=492.2[M+H]⁺.

### Embodiment 20

### Preparation of (S)-3-(2-amino-3-chloropyridine-4-yl)-7-(5-amino-5,7-dihydrospiro[cyclopenta[c]pyr idine-6,4′-piperidine]-1′-yl)pteridine-2,4(1H,3H)-dione

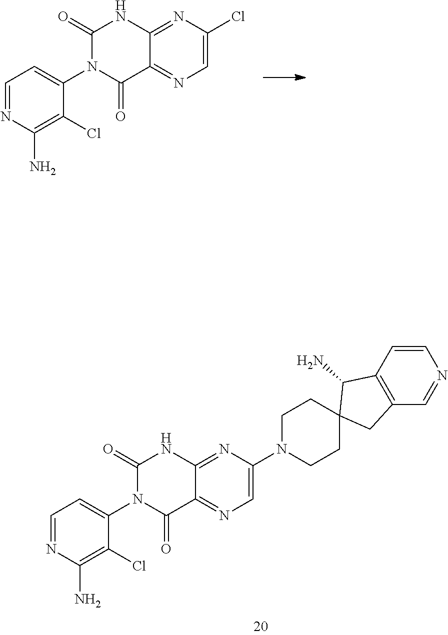 [SMILES: Nc1nccc(-n2c(=O)[nH]c3nc(Cl)cnc3c2=O)c1Cl ; Nc1nccc(-n2c(=O)[nH]c3nc(N4CCC5(CC4)Cc4cnccc4[C@H]5N)cnc3c2=O)c1Cl]

Compound 20 (202 mg, yield 19%) is obtained in a similar manner to Embodiment 1. ¹H NMR (400 MHZ, DMSO-d₆) δ: 11.50 (s, 1H), 7.95 (s, 1H), 7.83 (d, 1H), 7.30-7.26 (m, 1H), 5.84 (s, 2H), 4.03-3.96 (m, 1H), 3.52-3.45 (m, 2H), 3.32-3.25 (m, 2H), 2.73-2.65 (m, 2H), 1.61-1.30 (m, 6H); LC/MS(ESI): m/z=492.2[M+H]⁺.

### Embodiment 21

### Preparation of (S)-3-(2-amino-3-chloropyridine-4-yl)-7-(7-amino-5,7-dihydrospiro[cyclopenta[c]pyr idine-6,4′-piperidine]-1′-yl)pteridine-2,4(1H,3H)-dione

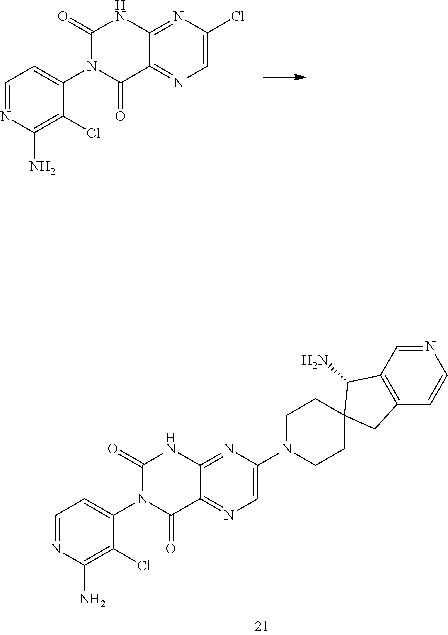 [SMILES: Nc1nccc(-n2c(=O)[nH]c3nc(Cl)cnc3c2=O)c1Cl ; Nc1nccc(-n2c(=O)[nH]c3nc(N4CCC5(CC4)Cc4ccncc4[C@H]5N)cnc3c2=O)c1Cl]

Compound 21 (266 mg, yield 25%) is obtained in a similar manner to Embodiment 1. LC/MS(ESI): m/z=492.2[M+H]⁺.

### Embodiment 22

### Preparation of (S)-3-(2-amino-3-chloropyridine-4-yl)-7-(7-amino-5,7-dihydrospiro[cyclopenta[b]pyr idine-6,4′-piperidine]-1′-yl)pteridine-2,4(1H,3H)-dione

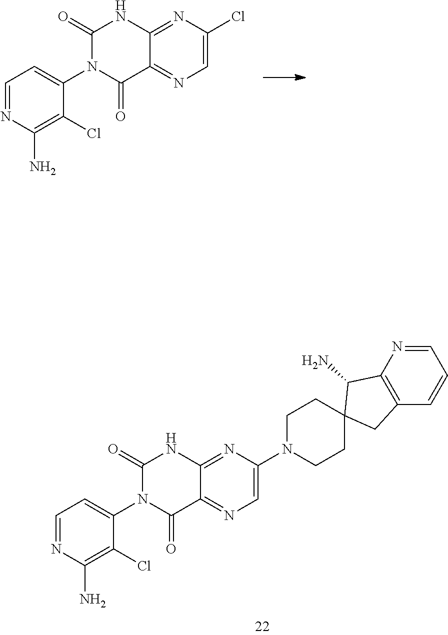 [SMILES: Nc1nccc(-n2c(=O)[nH]c3nc(Cl)cnc3c2=O)c1Cl ; Nc1nccc(-n2c(=O)[nH]c3nc(N4CCC5(CC4)Cc4cccnc4[C@H]5N)cnc3c2=O)c1Cl]

Compound 22 (223 mg, yield 21%) is obtained in a similar manner to Embodiment 1. LC/MS(ESI): m/z=492.2[M+H]⁺.

### Embodiment 23

### Preparation of 3-(2-amino-3-chloropyridine-4-yl)-7-((3S,4S)-4-amino-3-methyl-2-oxa-8-azaspiro[4.5]decane-8-yl)pteridine-2,4(1H,3H)-dione

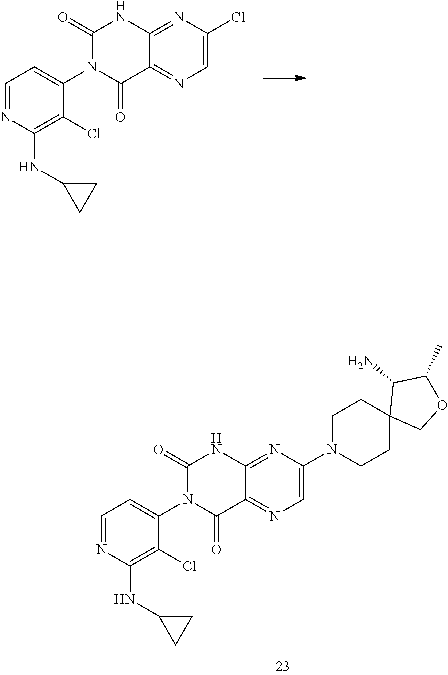 [SMILES: O=c1[nH]c2nc(Cl)cnc2c(=O)n1-c1ccnc(NC2CC2)c1Cl ; C[C@@H]1OCC2(CCN(c3cnc4c(=O)n(-c5ccnc(NC6CC6)c5Cl)c(=O)[nH]c4n3)CC2)[C@@H]1N]

Compound 23 (280 mg, yield 26%) is obtained in a similar manner to Embodiment 1. ¹H NMR (400 MHZ, DMSO-d₆) δ: 11.35 (s, 1H), 7.95 (s, 1H), 7.88 (d, 1H), 7.30-7.26 (m, 1H), 5.23 (s, 1H), 4.10-4.07 (m, 1H), 3.89-3.45 (m, 4H), 3.32-3.24 (m, 2H), 2.92 (d, 1H), 2.53-2.46 (m, 1H), 1.77-1.32 (m, 6H), 1.14 (d, 3H), 0.81-0.77 (m, 2H), 0.57-0.52 (m, 2H); LC/MS(ESI): m/z=499.2[M+H]⁺.

### Embodiment 24

### Preparation of (S)-7-(5-Amino-5,7-dihydrospiro[cyclopenta[c]pyridine-6,4′-piperidine-1′-yl)-3-(3-ch loro-2-(cyclopropylamino)pyridine-4-yl)-pteridine-2,4(1H,3H)-dione

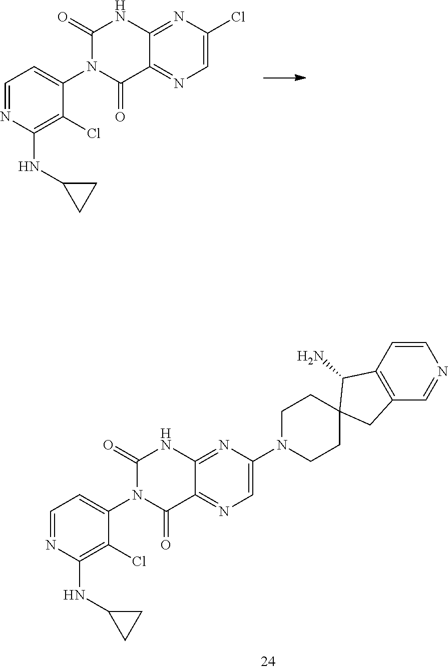 [SMILES: O=c1[nH]c2nc(Cl)cnc2c(=O)n1-c1ccnc(NC2CC2)c1Cl ; N[C@@H]1c2ccncc2CC12CCN(c1cnc3c(=O)n(-c4ccnc(NC5CC5)c4Cl)c(=O)[nH]c3n1)CC2]

Compound 24 (356 mg, yield 31%) is obtained in a similar manner to Embodiment 1. LC/MS(ESI): m/z=532.2[M+H]⁺.

### Embodiment 25

### Preparation of 7-((3S,4S)-4-amino-3-methyl-2-oxa-8-azaspiro[4.5]decane-8-yl)-3-(2,3-dichloropheny l)pyridinyl[2,3-d]pyrimidine-2,4(1H,3H)-dione

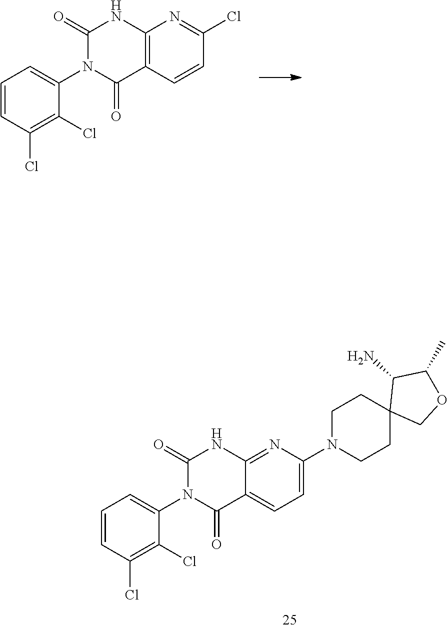 [SMILES: O=c1[nH]c2nc(Cl)ccc2c(=O)n1-c1cccc(Cl)c1Cl ; C[C@@H]1OCC2(CCN(c3ccc4c(=O)n(-c5cccc(Cl)c5Cl)c(=O)[nH]c4n3)CC2)[C@@H]1N]

Compound 25 (443 mg, yield 43%) is obtained in a similar manner to Embodiment 1. LC/MS(ESI): m/z=477.1[M+H]⁺.

### Embodiment 26

### Preparation of (S)-7-(5-Amino-5,7-dihydrospiro [cyclopenta[c]pyridine-6,4′-piperidine]-1′-yl)-3-(2,3-dichlorophenyl)pyridinyl[2,3-d]pyrimidine-2,4(1H,3H)-dione

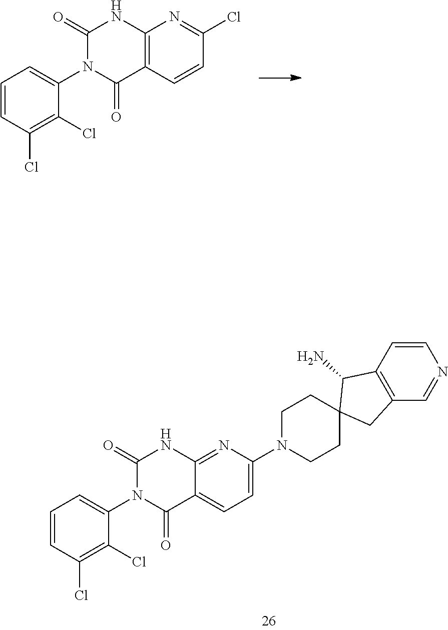 [SMILES: O=c1[nH]c2nc(Cl)ccc2c(=O)n1-c1cccc(Cl)c1Cl ; N[C@@H]1c2ccncc2CC12CCN(c1ccc3c(=O)n(-c4cccc(Cl)c4Cl)c(=O)[nH]c3n1)CC2]

Compound 26 (560 mg, yield 51%) is obtained in a similar manner to Embodiment 1. ¹H NMR (400 MHZ, DMSO-d₆) δ: 11.35 (s, 1H), 8.28-8.23 (m, 2H), 7.56-7.45 (m, 3H), 7.31-7.23 (m, 2H), 6.55 (d, 1H), 4.05-3.96 (m, 1H), 3.52-3.45 (m, 2H), 3.31-3.25 (m, 2H), 2.72-2.63 (m, 2H), 1.64-1.29 (m, 6H); LC/MS(ESI): m/z=509.1[M+H]⁺.

### Embodiment 27

### Preparation of (S)-3-(2-amino-3-chloropyridine-4-yl)-7-(5-amino-5,7-dihydrospiro[cyclopenta[c]pyr idine-6,4′-piperidine]-1′-yl) pyridinyl[2,3-d]pyrimidine-2,4(1H,3H)-dione

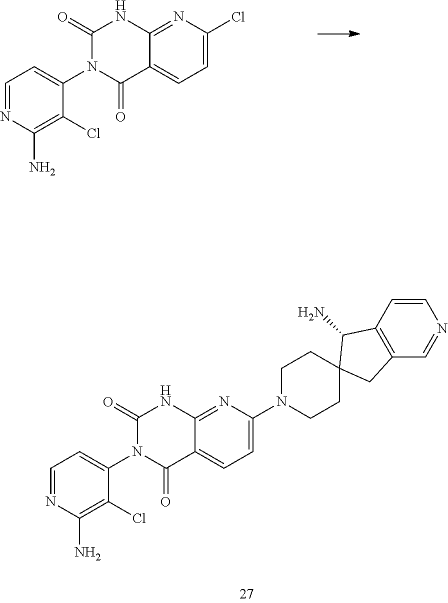 [SMILES: Nc1nccc(-n2c(=O)[nH]c3nc(Cl)ccc3c2=O)c1Cl ; Nc1nccc(-n2c(=O)[nH]c3nc(N4CCC5(CC4)Cc4cnccc4[C@H]5N)ccc3c2=O)c1Cl]

Compound 27 (265 mg, yield 25%) is obtained in a similar manner to Embodiment 1. LC/MS(ESI): m/z=491.2[M+H]⁺.

### Embodiment 28

### Preparation of (S)-7-(5-Amino-5,7-dihydrospiro[cyclopenta[c]pyridine-6,4′-piperidine-1′-yl)-3-(3-ch loro-2-(cyclopropylamino)pyridine-4-yl)pyridinyl[2,3-d]pyrimidine-2,4(1H,3H)-dion e

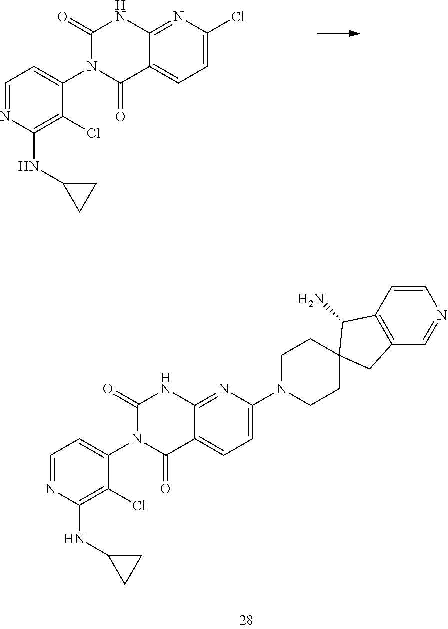 [SMILES: O=c1[nH]c2nc(Cl)ccc2c(=O)n1-c1ccnc(NC2CC2)c1Cl ; N[C@@H]1c2ccncc2CC12CCN(c1ccc3c(=O)n(-c4ccnc(NC5CC5)c4Cl)c(=O)[nH]c3n1)CC2]

Compound 28 (344 mg, yield 30%) is obtained in a similar manner to Embodiment 1. LC/MS(ESI): m/z=531.2[M+H]⁺.

### Embodiment 29

### Preparation of (S)-7-(6-Amino-4,6-dihydrospiro[cyclopenta[b]thiazole-5,4′-piperidine]-1′-yl)-3-(2,3-dichlorophenyl)pteridine-2,4(1H,3H)-dione

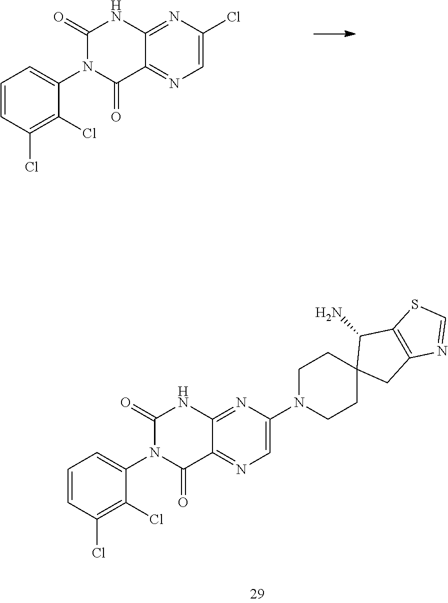 [SMILES: O=c1[nH]c2nc(Cl)cnc2c(=O)n1-c1cccc(Cl)c1Cl ; N[C@@H]1c2scnc2CC12CCN(c1cnc3c(=O)n(-c4cccc(Cl)c4Cl)c(=O)[nH]c3n1)CC2]

Compound 29 (457 mg, yield 41%) is obtained in a similar manner to Embodiment 1. LC/MS(ESI): m/z=531.2[M+H]⁺.

### Embodiment 30

### Preparation of (S)-7-(5-Amino-5,7-dihydrospiro[cyclopenta[c]pyridine-6,4′-piperidine-1′-yl)-3-(7-ch lorobenzo[d]thiazol-6-yl)pteridine-2,4(1H,3H)-dione

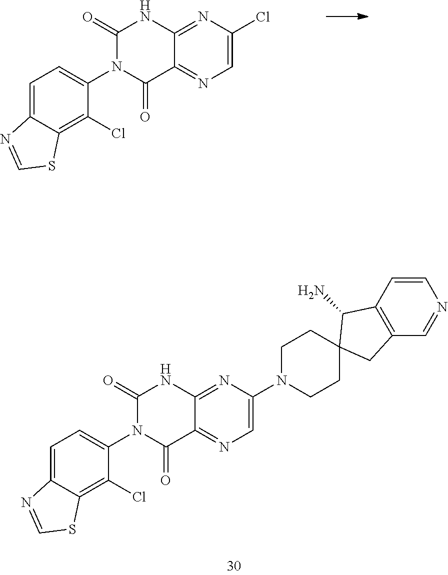 [SMILES: O=c1[nH]c2nc(Cl)cnc2c(=O)n1-c1ccc2ncsc2c1Cl ; N[C@@H]1c2ccncc2CC12CCN(c1cnc3c(=O)n(-c4ccc5ncsc5c4Cl)c(=O)[nH]c3n1)CC2]

Compound 30 (449 mg, yield 39%) is obtained in a similar manner to Embodiment 1. LC/MS(ESI): m/z=533.1[M+H]⁺.

### Embodiment 31

### Preparation of 7-((3S,4S)-4-amino-3-methyl-2-oxa-8-azaspiro[4.5]decane-8-yl)-3-(7-chlorobenzo[d]t hiazol-6-yl)pteridine-2,4(1H,3H)-dione

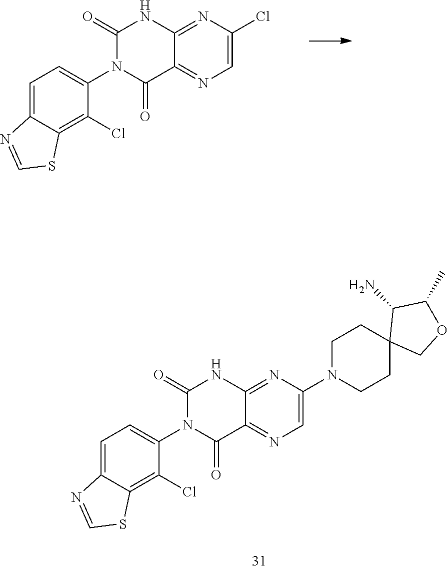 [SMILES: O=c1[nH]c2nc(Cl)cnc2c(=O)n1-c1ccc2ncsc2c1Cl ; C[C@@H]1OCC2(CCN(c3cnc4c(=O)n(-c5ccc6ncsc6c5Cl)c(=O)[nH]c4n3)CC2)[C@@H]1N]

Compound 31 (367 mg, yield 34%) is obtained in a similar manner to Embodiment 1. ¹H NMR (400 MHZ, DMSO-d₆) δ: 11.35 (s, 1H), 8.92 (s, 1H), 7.95 (s, 1H), 7.85 (d, 1H), 7.53 (d, 1H), 4.12-4.09 (m, 1H), 3.85-3.47 (m, 4H), 3.32-3.24 (m, 2H), 2.92 (d, 1H), 1.75-1.30 (m, 6H), 1.14 (d, 3H); LC/MS(ESI): m/z=500.1[M+H]⁺.

### Embodiment 32

### Preparation of (S)-3-(2-amino-3-chloropyridine-4-yl)-7-(6-amino-4,6-dihydrospiro[cyclopenta[b]thi azole-5,4′-piperidine]-1′-yl)pteridine-2,4(1H,3H)-dione

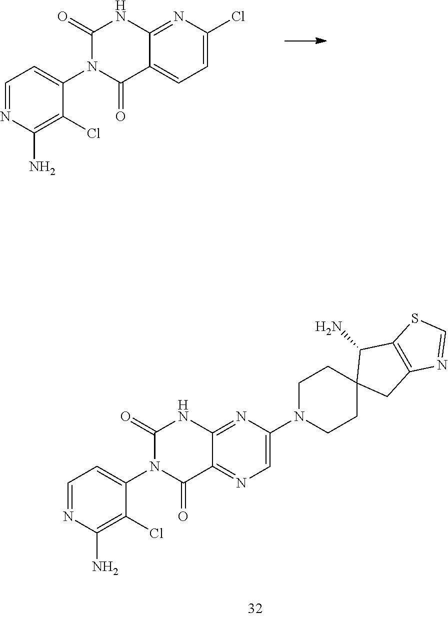 [SMILES: Nc1nccc(-n2c(=O)[nH]c3nc(Cl)ccc3c2=O)c1Cl ; Nc1nccc(-n2c(=O)[nH]c3nc(N4CCC5(CC4)Cc4ncsc4[C@H]5N)cnc3c2=O)c1Cl]

Compound 32 (247 mg, yield 23%) is obtained in a similar manner to Embodiment 1. LC/MS(ESI): m/z=498.1[M+H]⁺.

### Embodiment 33

### Preparation of N-(3-(7-((3S,4S)-4-amino-3-methyl-2-oxa-8-azaspiro[4.5]decane-8-yl)-2,4-dioxa-1,2-d ihydropteridine-3(4H)-yl)-2-chlorophenyl)-2-hydroxy-4-oxa-6,7,8,9-tetrahydro-4H-p yridinyl[1,2-a]pyrimidine-3-carboxamide

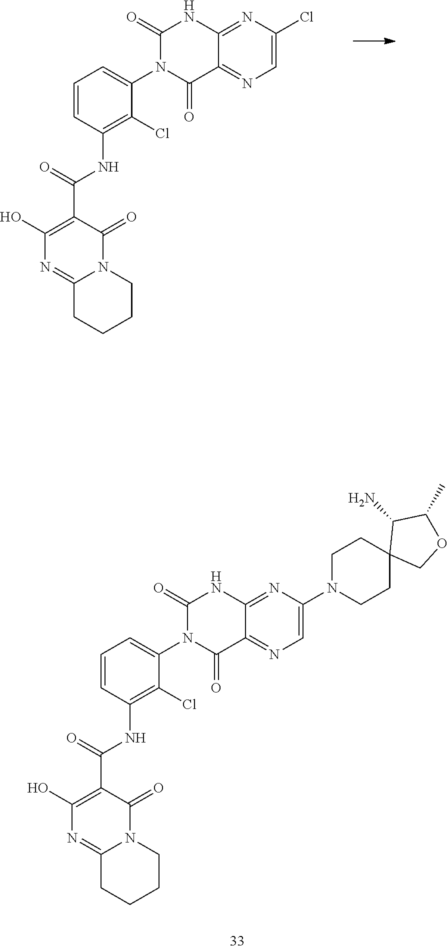 [SMILES: O=C(Nc1cccc(-n2c(=O)[nH]c3nc(Cl)cnc3c2=O)c1Cl)c1c(O)nc2n(c1=O)CCCC2 ; C[C@@H]1OCC2(CCN(c3cnc4c(=O)n(-c5cccc(NC(=O)c6c(O)nc7n(c6=O)CCCC7)c5Cl)c(=O)[nH]c4n3)CC2)[C@@H]1N]

Compound 33 (448 mg, yield 32%) is obtained in a similar manner to Embodiment 1. ¹H NMR (400 MHZ, DMSO-d₆) δ: 12.31 (br s, 1H), 11.42 (s, 1H), 7.97 (s, 1H), 7.63-7.52 (m, 2H), 7.28-7.25 (m, 1H), 5.51 (br s, 3H), 4.13-4.10 (m, 1H), 3.93-3.45 (m, 8H), 2.95 (d, 1H), 2.74 (s, 2H), 1.85-1.41 (m, 8H), 1.14 (d, 3H); LC/MS(ESI): m/z=650.2[M+H]⁺.

### Embodiment 34

### Preparation of N-(3-(7-((3S,4S)-4-amino-3-methyl-2-oxa-8-azaspiro[4.5]decane-8-yl)-2,4-dioxa-1,2-d ihydropyridinyl[3,2-d]pyrimidine-3(4H)-yl)-2-chlorophenyl)-2-hydroxy-4-oxa-6,7,8,9-tetrahydro-4H-pyridinyl[1,2-a]pyrimidine-3-carboxamide

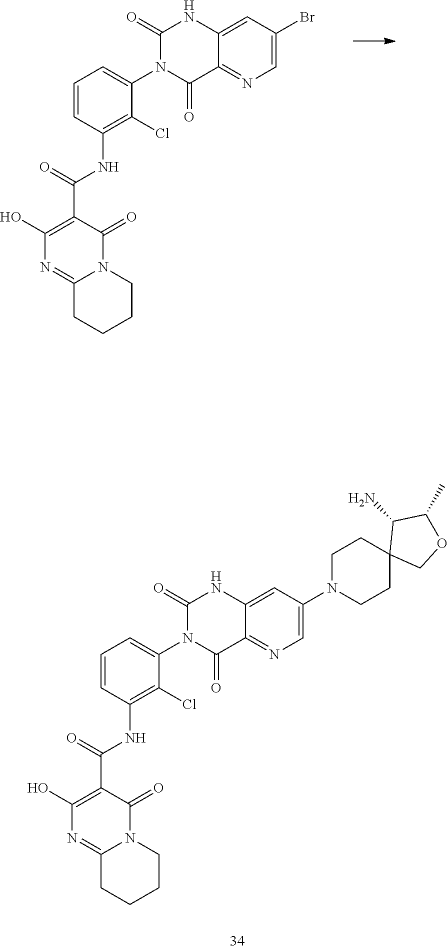 [SMILES: O=C(Nc1cccc(-n2c(=O)[nH]c3cc(Br)cnc3c2=O)c1Cl)c1c(O)nc2n(c1=O)CCCC2 ; C[C@@H]1OCC2(CCN(c3cnc4c(=O)n(-c5cccc(NC(=O)c6c(O)nc7n(c6=O)CCCC7)c5Cl)c(=O)[nH]c4c3)CC2)[C@@H]1N]

Compound 34 (406 mg, yield 29%) is obtained in a similar manner to Embodiment 1. LC/MS(ESI): m/z=649.2[M+H]⁺.

### Embodiment 35

### Preparation of N-(3-(7-((3S,4S)-4-amino-3-methyl-2-oxa-8-azaspiro[4.5]decane-8-yl)-2,4-dioxa-1,2-d ihydropyridinyl[2,3-d]pyrimidine-3(4H)-yl)-2-chlorophenyl)-2-hydroxy-4-oxa-6,7,8,9-tetrahydro-4H-pyridinyl[1,2-a]pyrimidine-3-carboxamide

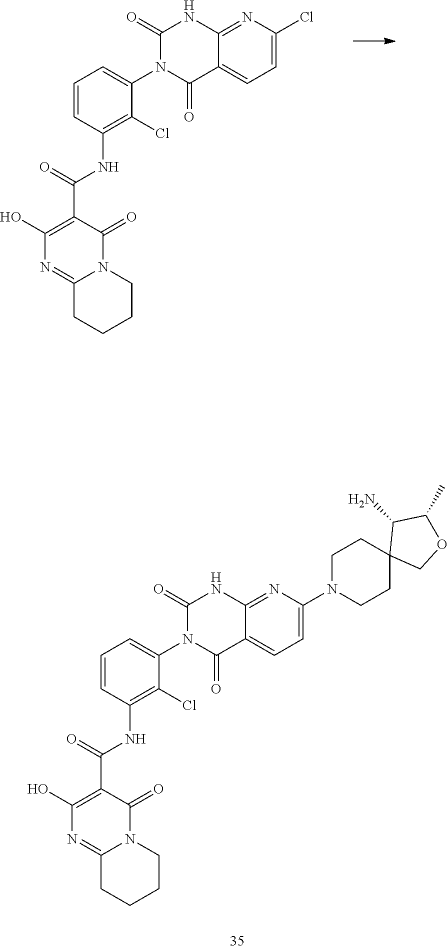 [SMILES: O=C(Nc1cccc(-n2c(=O)[nH]c3nc(Cl)ccc3c2=O)c1Cl)c1c(O)nc2n(c1=O)CCCC2 ; C[C@@H]1OCC2(CCN(c3ccc4c(=O)n(-c5cccc(NC(=O)c6c(O)nc7n(c6=O)CCCC7)c5Cl)c(=O)[nH]c4n3)CC2)[C@@H]1N]

Compound 35 (574 mg, yield 41%) is obtained in a similar manner to Embodiment 1. LC/MS(ESI): m/z=649.2[M+H]⁺.

### Embodiment 36

### Preparation of N-(3-(7-((3S,4S)-4-amino-3-methyl-2-oxa-8-azaspiro[4.5]decane-8-yl)-2,4-dioxa-1,2-d ihydroquinazolin-3(4H)-yl)-2-chlorophenyl)-2-hydroxy-4-oxa-6,7,8,9-tetrahydro-4H-pyridinyl[1,2-a]pyrimidine-3-carboxamide

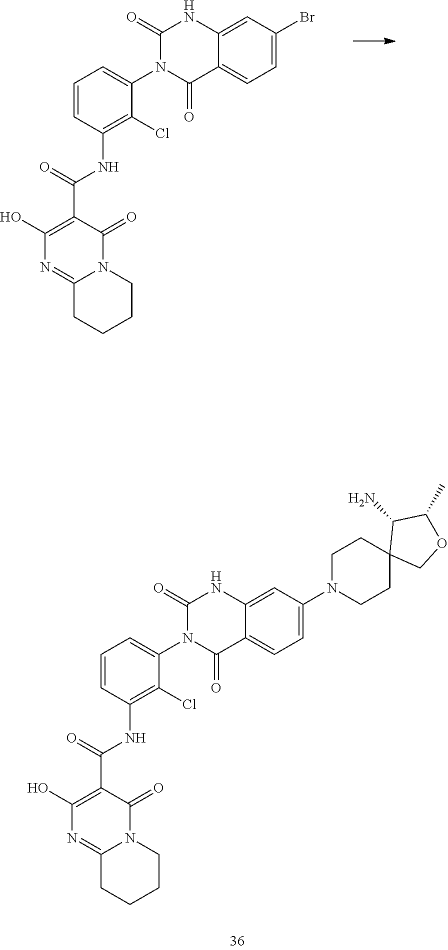 [SMILES: O=C(Nc1cccc(-n2c(=O)[nH]c3cc(Br)ccc3c2=O)c1Cl)c1c(O)nc2n(c1=O)CCCC2 ; C[C@@H]1OCC2(CCN(c3ccc4c(=O)n(-c5cccc(NC(=O)c6c(O)nc7n(c6=O)CCCC7)c5Cl)c(=O)[nH]c4c3)CC2)[C@@H]1N]

Compound 36 (531 mg, yield 38%) is obtained in a similar manner to Embodiment 1. LC/MS(ESI): m/z=648.2[M+H]⁺.

### Embodiment 37

### Preparation of N-(3-(7-((3S,4S)-4-amino-3-methyl-2-oxa-8-azaspiro[4.5]decane-8-yl)-2,4-dioxa-1,2-d ihydropteridine-3(4H)-yl)-2-chlorophenyl)-1-methyl-1H-pyrazole-3-carboxamide

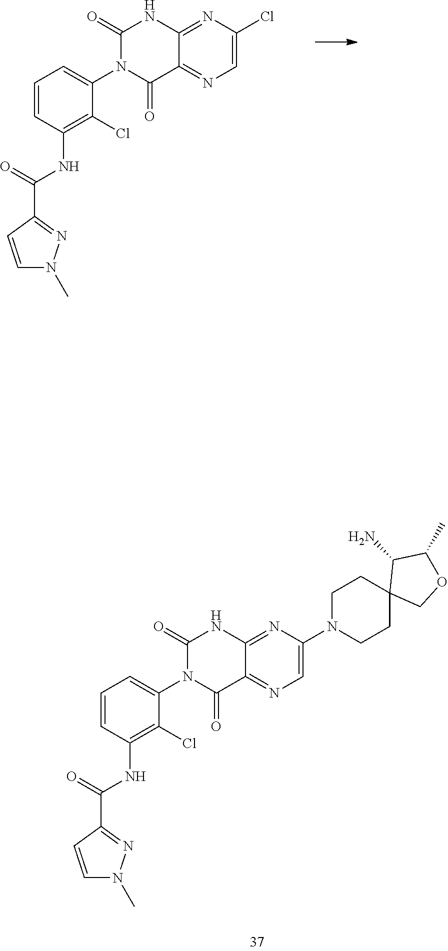 [SMILES: Cn1ccc(C(=O)Nc2cccc(-n3c(=O)[nH]c4nc(Cl)cnc4c3=O)c2Cl)n1 ; C[C@@H]1OCC2(CCN(c3cnc4c(=O)n(-c5cccc(NC(=O)c6ccn(C)n6)c5Cl)c(=O)[nH]c4n3)CC2)[C@@H]1N]

Compound 37 (586 mg, yield 48%) is obtained in a similar manner to Embodiment 1. ¹H NMR (400 MHZ, DMSO-d₆) δ: 11.50 (s, 1H), 8.45 (d, 1H), 7.95 (s, 1H), 7.63-7.52 (m, 3H), 7.33-7.28 (m, 1H), 6.65 (d, 1H), 4.18 (s, 1H), 4.12-4.09 (m, 1H), 3.85-3.47 (m, 4H), 3.32-3.24 (m, 2H), 2.92 (d, 1H), 1.75-1.30 (m, 6H), 1.14 (d, 3H); LC/MS(ESI): m/z=566.2[M+H]⁺.

### Embodiment 38

### Preparation of N-(3-(7-((3S,4S)-4-amino-3-methyl-2-oxa-8-azaspiro[4.5]decane-8-yl)-2,4-dioxa-1,2-d ihydropteridine-3(4H)-yl)-2-chlorophenyl)-1-methyl-1H-pyrazole-4-carboxamide

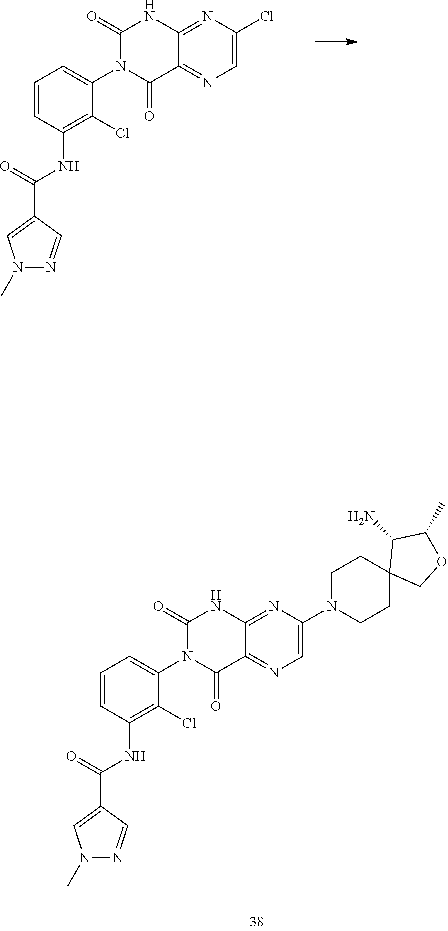

Compound 38 (525 mg, yield 43%) is obtained in a similar manner to Embodiment 1. LC/MS(ESI): m/z=566.2[M+H]⁺.

### Embodiment 39

### Preparation of N-(3-(7-((3S,4S)-4-amino-3-methyl-2-oxa-8-azaspiro[4.5]decane-8-yl)-2,4-dioxa-1,2-d ihydropteridine-3(4H)-yl)-2-chlorophenyl) benzamide

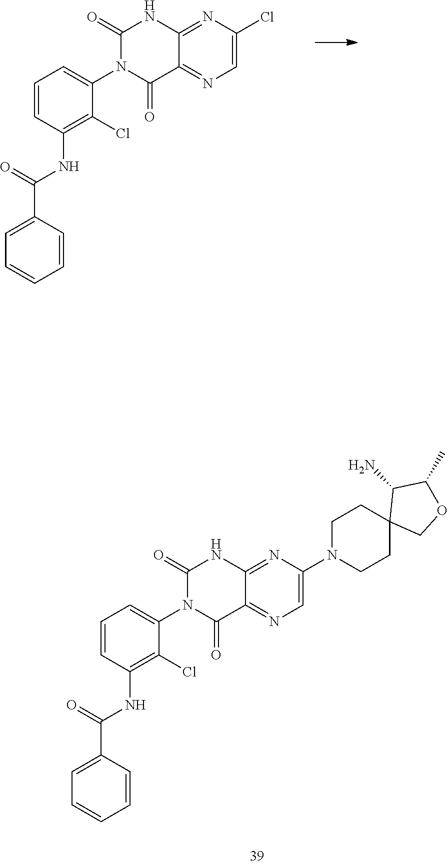 [SMILES: O=C(Nc1cccc(-n2c(=O)[nH]c3nc(Cl)cnc3c2=O)c1Cl)c1ccccc1 ; C[C@@H]1OCC2(CCN(c3cnc4c(=O)n(-c5cccc(NC(=O)c6ccccc6)c5Cl)c(=O)[nH]c4n3)CC2)[C@@H]1N]

Compound 39 (448 mg, yield 37%) is obtained in a similar manner to Embodiment 1. LC/MS(ESI): m/z=562.2[M+H]⁺.

### Embodiment 40

### Preparation of N-(3-(7-((3S,4S)-4-amino-3-methyl-2-oxa-8-azaspiro[4.5]decane-8-yl)-2,4-dioxa-1,2-d ihydropteridine-3(4H)-yl)-2-chlorophenyl)pyrazine-2-carboxamide

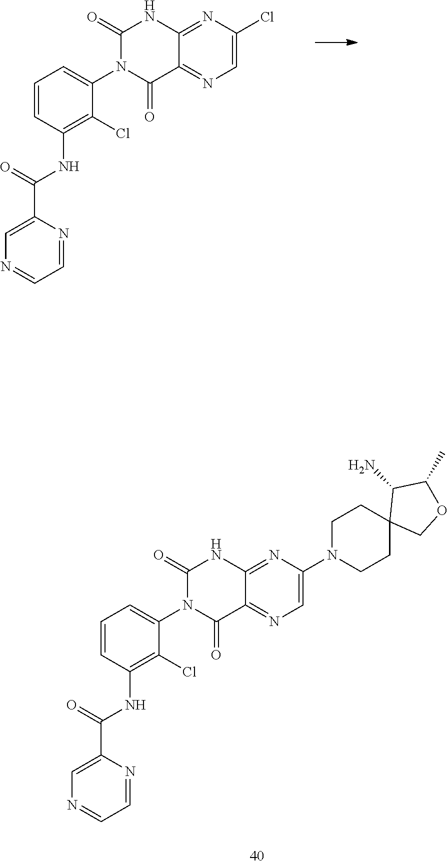 [SMILES: O=C(Nc1cccc(-n2c(=O)[nH]c3nc(Cl)cnc3c2=O)c1Cl)c1cnccn1 ; C[C@@H]1OCC2(CCN(c3cnc4c(=O)n(-c5cccc(NC(=O)c6cnccn6)c5Cl)c(=O)[nH]c4n3)CC2)[C@@H]1N]

Compound 40 (499 mg, yield 41%) is obtained in a similar manner to Embodiment 1. ¹H NMR (400 MHZ, DMSO-d₆) δ: 11.43 (s, 1H), 9.26 (s, 1H), 8.75 (d, 1H), 8.56 (s, 1H), 8.34 (d, 1H), 7.95 (s, 1H), 7.63-7.52 (m, 2H), 7.33-7.28 (m, 1H), 4.12-4.09 (m, 1H), 3.83-3.47 (m, 4H), 3.32-3.24 (m, 2H), 2.92 (d, 1H), 1.75-1.30 (m, 6H), 1.14 (d, 3H); LC/MS(ESI): m/z=564.2[M+H]⁺.

### Embodiment 41

### Preparation of N-(3-(7-((3S,4S)-4-amino-3-methyl-2-oxa-8-azaspiro[4.5]decane-8-yl)-2,4-dioxa-1,2-d ihydropteridine-3(4H)-yl)-2-chlorophenyl) pyridine-2-carboxamide

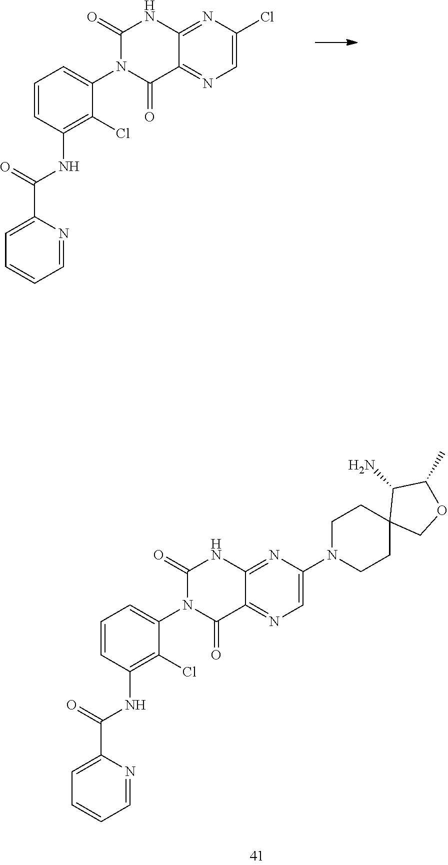 [SMILES: O=C(Nc1cccc(-n2c(=O)[nH]c3nc(Cl)cnc3c2=O)c1Cl)c1ccccn1 ; C[C@@H]1OCC2(CCN(c3cnc4c(=O)n(-c5cccc(NC(=O)c6ccccn6)c5Cl)c(=O)[nH]c4n3)CC2)[C@@H]1N]

Compound 41 (558 mg, yield 46%) is obtained in a similar manner to Embodiment 1. LC/MS(ESI): m/z=563.2[M+H]⁺.

### Embodiment 42

### Preparation of N-(3-(7-((3S,4S)-4-amino-3-methyl-2-oxa-8-azaspiro[4.5]decane-8-yl)-2,4-dioxa-1,2-d ihydropteridine-3(4H)-yl)-2-chlorophenyl)pyrimidine-4-carboxamide

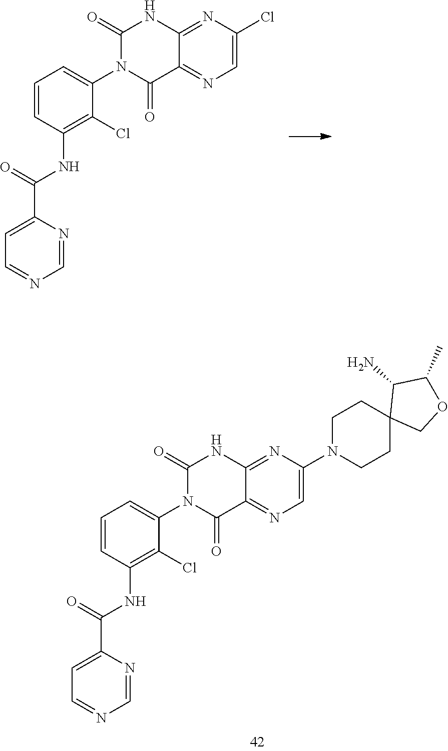 [SMILES: O=C(Nc1cccc(-n2c(=O)[nH]c3nc(Cl)cnc3c2=O)c1Cl)c1ccncn1 ; C[C@@H]1OCC2(CCN(c3cnc4c(=O)n(-c5cccc(NC(=O)c6ccncn6)c5Cl)c(=O)[nH]c4n3)CC2)[C@@H]1N]

Compound 42 (389 mg, yield 32%) is obtained in a similar manner to Embodiment 1. LC/MS(ESI): m/z=564.2[M+H]⁺.

### Embodiment 43

### Preparation of N-(3-(7-((3S,4S)-4-amino-3-methyl-2-oxa-8-azaspiro[4.5]decane-8-yl)-2,4-dioxa-1,2-d ihydropteridine-3(4H)-yl)-2-chlorophenyl)-1-methyl-1H-indole-7-carboxamide

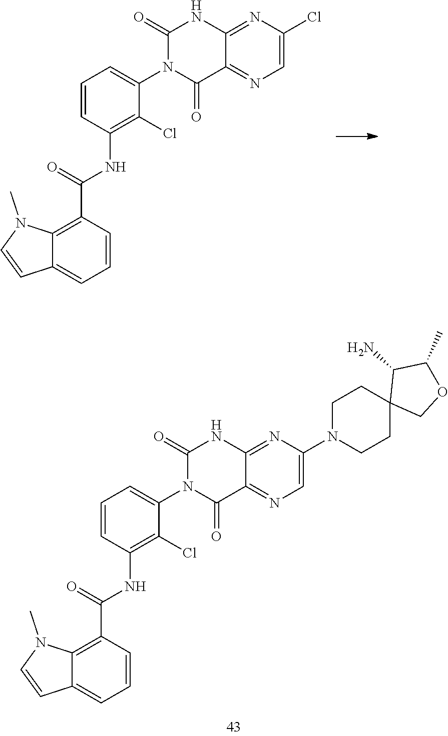 [SMILES: Cn1ccc2cccc(C(=O)Nc3cccc(-n4c(=O)[nH]c5nc(Cl)cnc5c4=O)c3Cl)c21 ; C[C@@H]1OCC2(CCN(c3cnc4c(=O)n(-c5cccc(NC(=O)c6cccc7ccn(C)c67)c5Cl)c(=O)[nH]c4n3)CC2)[C@@H]1N]

Compound 43 (477 mg, yield 36%) is obtained in a similar manner to Embodiment 1. LC/MS(ESI): m/z=615.2[M+H]⁺.

### Embodiment 44

### Preparation of N-(3-(7-((3S,4S)-4-amino-3-methyl-2-oxa-8-azaspiro[4.5]decane-8-yl)-2,4-dioxa-1,2-d ihydropteridine-3(4H)-yl)-2-chlorophenyl)-1-methyl-1H-indazole-7-carboxamide

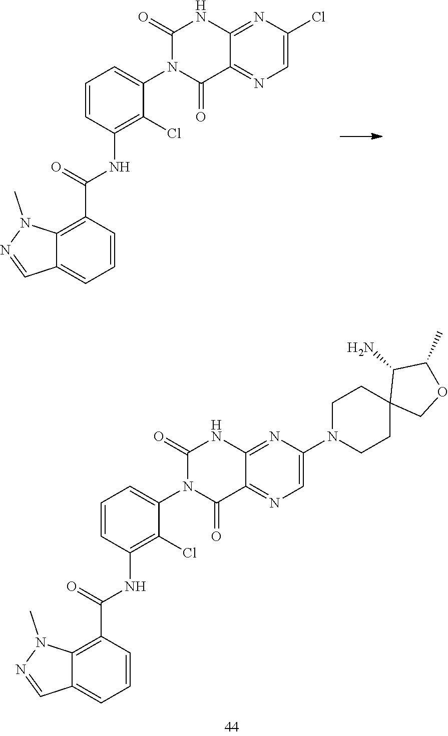 [SMILES: Cn1ncc2cccc(C(=O)Nc3cccc(-n4c(=O)[nH]c5nc(Cl)cnc5c4=O)c3Cl)c21 ; C[C@@H]1OCC2(CCN(c3cnc4c(=O)n(-c5cccc(NC(=O)c6cccc7cnn(C)c67)c5Cl)c(=O)[nH]c4n3)CC2)[C@@H]1N]

Compound 44 (545 mg, yield 41%) is obtained in a similar manner to Embodiment 1. LC/MS(ESI): m/z=616.2[M+H]⁺.

### Embodiment 45

### Preparation of 7-((3S,4S)-4-amino-3-methyl-2-oxa-8-azaspiro[4.5]decane-8-yl)-3-(2-chloro-3-(pyrim idine-4-yl)phenyl)pteridine-2,4(1H,3H)-dione

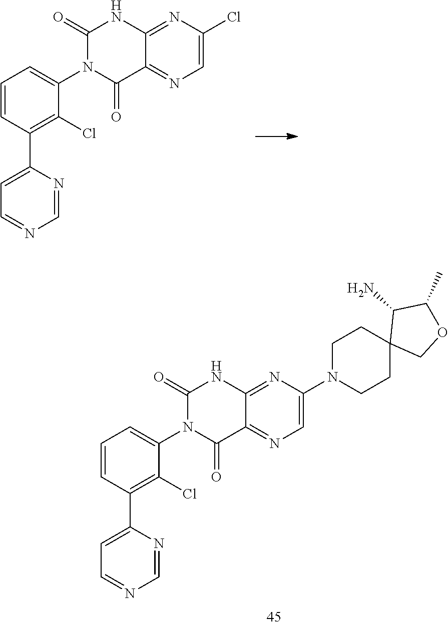 [SMILES: O=c1[nH]c2nc(Cl)cnc2c(=O)n1-c1cccc(-c2ccncn2)c1Cl ; C[C@@H]1OCC2(CCN(c3cnc4c(=O)n(-c5cccc(-c6ccncn6)c5Cl)c(=O)[nH]c4n3)CC2)[C@@H]1N]

Compound 45 (371 mg, yield 33%) is obtained in a similar manner to Embodiment 1. ¹H NMR (400 MHZ, DMSO-d₆) δ: 11.43 (s, 1H), 9.26 (s, 1H), 8.75 (d, 1H), 8.56 (s, 1H), 8.34 (d, 1H), 7.95 (s, 1H), 7.63-7.52 (m, 2H), 7.33-7.28 (m, 1H), 4.12-4.09 (m, 1H), 3.83-3.47 (m, 4H), 3.32-3.24 (m, 2H), 2.92 (d, 1H), 1.75-1.30 (m, 6H), 1.14 (d, 3H); LC/MS(ESI): m/z=521.2[M+H]⁺.

### Embodiment 46

### Preparation of 7-((3S,4S)-4-amino-3-methyl-2-oxa-8-azaspiro[4.5]decane-8-yl)-3-(2-chloro-3-(pyridi ne-3-yl)phenyl)pteridine-2,4(1H,3H)-dione

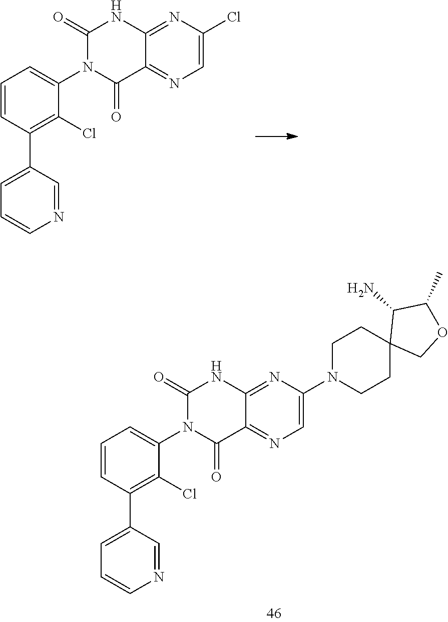 [SMILES: O=c1[nH]c2nc(Cl)cnc2c(=O)n1-c1cccc(-c2cccnc2)c1Cl ; C[C@@H]1OCC2(CCN(c3cnc4c(=O)n(-c5cccc(-c6cccnc6)c5Cl)c(=O)[nH]c4n3)CC2)[C@@H]1N]

Compound 46 (460 mg, yield 41%) is obtained in a similar manner to Embodiment 1. LC/MS(ESI): m/z=520.2[M+H]⁺.

### Embodiment 47

### Preparation of 7-((3S,4S)-4-amino-3-methyl-2-oxa-8-azaspiro[4.5]decane-8-yl)-3-(2-chloro-3-(pyrazi ne-2-yl)phenyl)pteridine-2,4(1H,3H)-dione

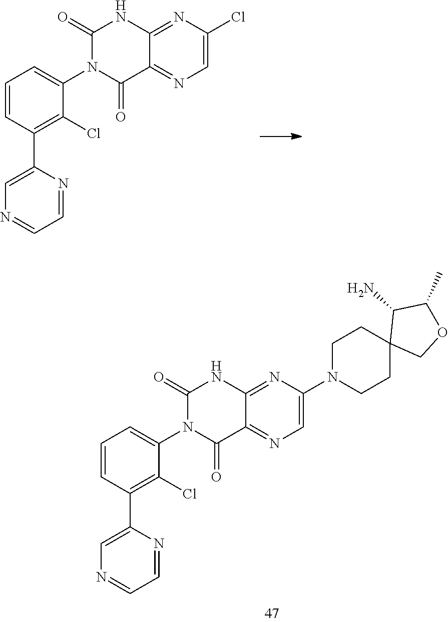 [SMILES: O=c1[nH]c2nc(Cl)cnc2c(=O)n1-c1cccc(-c2cnccn2)c1Cl ; C[C@@H]1OCC2(CCN(c3cnc4c(=O)n(-c5cccc(-c6cnccn6)c5Cl)c(=O)[nH]c4n3)CC2)[C@@H]1N]

Compound 47 (483 mg, yield 43%) is obtained in a similar manner to Embodiment 1. LC/MS(ESI): m/z=521.2[M+H]⁺.

### Embodiment 48

### Preparation of 7-((3S,4S)-4-amino-3-methyl-2-oxa-8-azaspiro[4.5]decane-8-yl)-3-(2-chloro-3-(1-met hyl-1H-pyrazole-3-yl)phenyl)pteridine-2,4(1H,3H)-dione

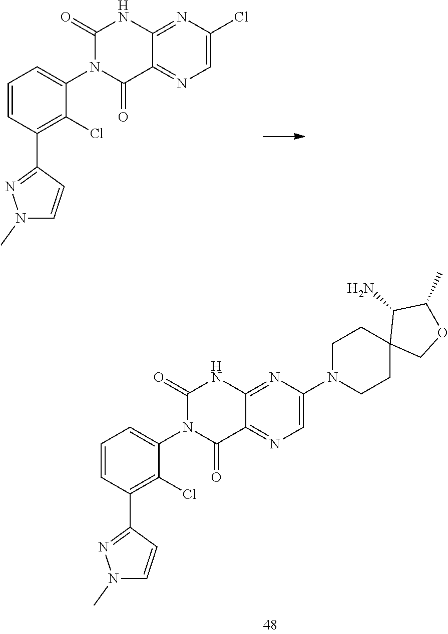 [SMILES: Cn1ccc(-c2cccc(-n3c(=O)[nH]c4nc(Cl)cnc4c3=O)c2Cl)n1 ; C[C@@H]1OCC2(CCN(c3cnc4c(=O)n(-c5cccc(-c6ccn(C)n6)c5Cl)c(=O)[nH]c4n3)CC2)[C@@H]1N]

Compound 48 (327 mg, yield 29%) is obtained in a similar manner to Embodiment 1. LC/MS(ESI): m/z=523.2[M+H]⁺.

### Embodiment 49

### Preparation of 7-((3S,4S)-4-amino-3-methyl-2-oxa-8-azaspiro[4.5]decane-8-yl)-3-(2-chloro-3-(pyrim idine-4-amino)phenyl)pteridine-2,4(1H,3H)-dione

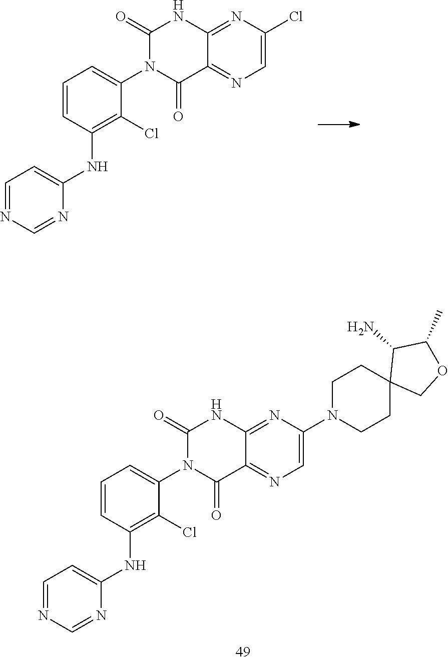 [SMILES: O=c1[nH]c2nc(Cl)cnc2c(=O)n1-c1cccc(Nc2ccncn2)c1Cl ; C[C@@H]1OCC2(CCN(c3cnc4c(=O)n(-c5cccc(Nc6ccncn6)c5Cl)c(=O)[nH]c4n3)CC2)[C@@H]1N]

Compound 49 (347 mg, yield 30%) is obtained in a similar manner to Embodiment 1. ¹H NMR (400 MHZ, DMSO-d₆) δ: 11.36 (s, 1H), 8.95 (s, 1H), 8.35 (d, 1H), 7.97-7.95 (m, 2H), 7.43-7.32 (m, 2H), 7.13-7.08 (m, 1H), 5.93 (s, 1H), 4.12-4.09 (m, 1H), 3.81-3.45 (m, 4H), 3.30-3.22 (m, 2H), 2.89 (d, 1H), 1.73-1.29 (m, 6H), 1.14 (d, 3H); LC/MS(ESI): m/z=536.2[M+H]⁺.

### Embodiment 50

### Preparation of 7-((3S,4S)-4-amino-3-methyl-2-oxa-8-azaspiro[4.5]decane-8-yl)-3-(2-chloro-3-(pyridi ne-2-amino)phenyl)pteridine-2,4(1H,3H)-dione

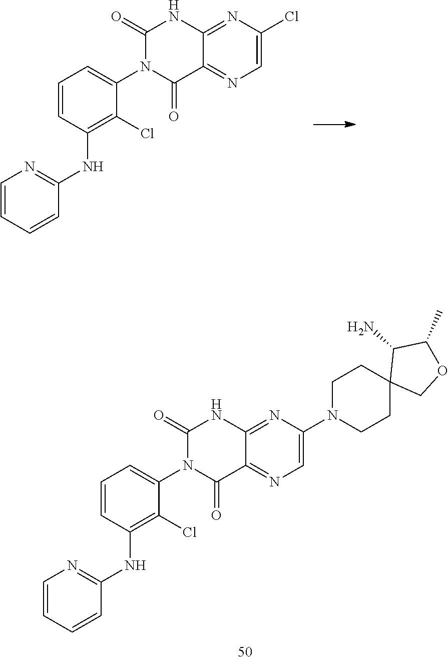 [SMILES: O=c1[nH]c2nc(Cl)cnc2c(=O)n1-c1cccc(Nc2ccccn2)c1Cl ; C[C@@H]1OCC2(CCN(c3cnc4c(=O)n(-c5cccc(Nc6ccccn6)c5Cl)c(=O)[nH]c4n3)CC2)[C@@H]1N]

Compound 50 (311 mg, yield 27%) is obtained in a similar manner to Embodiment 1. LC/MS(ESI): m/z=535.2[M+H]⁺.

### Embodiment 51

### Preparation of 7-((3S,4S)-4-amino-3-methyl-2-oxa-8-azaspiro[4.5]decane-8-yl)-3-(2-chloro-3-(pyrazi ne-2-amino)phenyl)pteridine-2,4(1H,3H)-dione

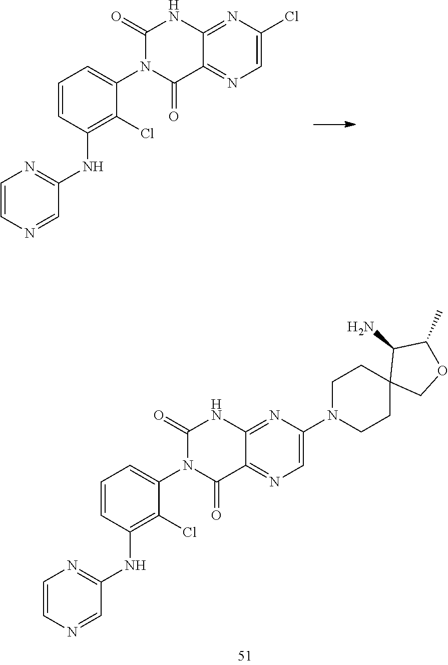 [SMILES: O=c1[nH]c2nc(Cl)cnc2c(=O)n1-c1cccc(Nc2cnccn2)c1Cl ; C[C@@H]1OCC2(CCN(c3cnc4c(=O)n(-c5cccc(Nc6cnccn6)c5Cl)c(=O)[nH]c4n3)CC2)[C@H]1N]

Compound 51 (405 mg, yield 35%) is obtained in a similar manner to Embodiment 1. LC/MS(ESI): m/z=536.2[M+H]⁺.

### Embodiment 52

### Preparation of 7-((3S,4S)-4-amino-3-methyl-2-oxa-8-azaspiro[4.5]decane-8-yl)-3-(2-chloro-3-(pyrim idine-2-amino)phenyl)pteridine-2,4(1H,3H)-dione

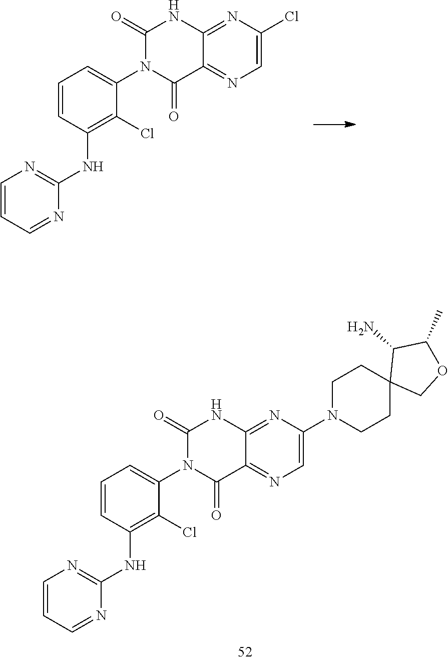 [SMILES: O=c1[nH]c2nc(Cl)cnc2c(=O)n1-c1cccc(Nc2ncccn2)c1Cl ; C[C@@H]1OCC2(CCN(c3cnc4c(=O)n(-c5cccc(Nc6ncccn6)c5Cl)c(=O)[nH]c4n3)CC2)[C@@H]1N]

Compound 52 (474 mg, yield 41%) is obtained in a similar manner to Embodiment 1. LC/MS(ESI): m/z=536.2[M+H]⁺.

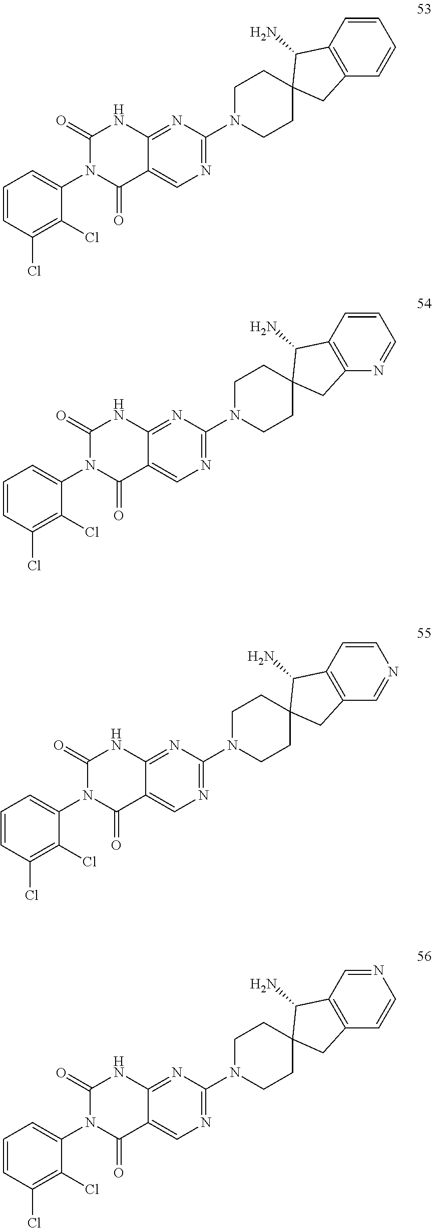 [SMILES: N[C@@H]1c2ccccc2CC12CCN(c1ncc3c(=O)n(-c4cccc(Cl)c4Cl)c(=O)[nH]c3n1)CC2 ; N[C@@H]1c2cccnc2CC12CCN(c1ncc3c(=O)n(-c4cccc(Cl)c4Cl)c(=O)[nH]c3n1)CC2 ; N[C@@H]1c2ccncc2CC12CCN(c1ncc3c(=O)n(-c4cccc(Cl)c4Cl)c(=O)[nH]c3n1)CC2 ; N[C@@H]1c2cnccc2CC12CCN(c1ncc3c(=O)n(-c4cccc(Cl)c4Cl)c(=O)[nH]c3n1)CC2]

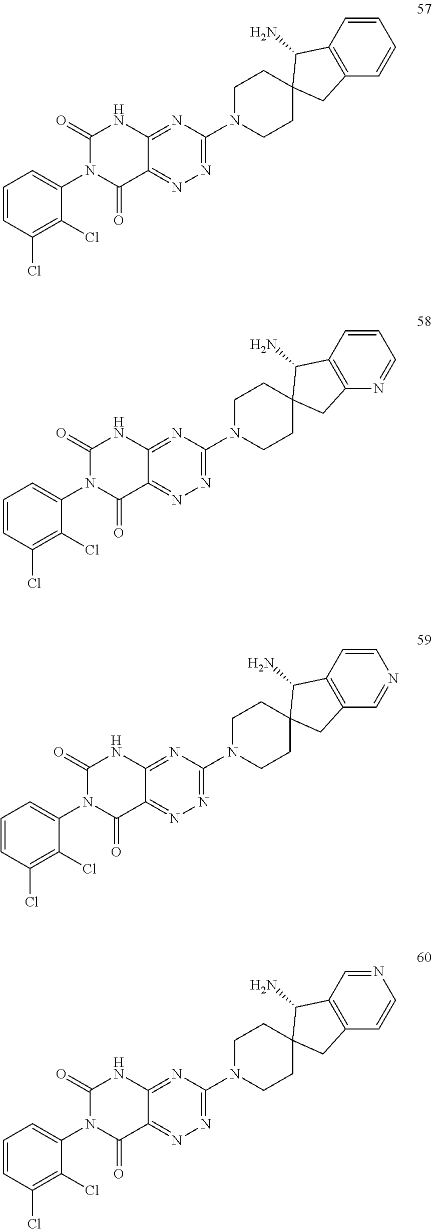 [SMILES: N[C@@H]1c2ccccc2CC12CCN(c1nnc3c(=O)n(-c4cccc(Cl)c4Cl)c(=O)[nH]c3n1)CC2 ; N[C@@H]1c2cccnc2CC12CCN(c1nnc3c(=O)n(-c4cccc(Cl)c4Cl)c(=O)[nH]c3n1)CC2 ; N[C@@H]1c2ccncc2CC12CCN(c1nnc3c(=O)n(-c4cccc(Cl)c4Cl)c(=O)[nH]c3n1)CC2 ; N[C@@H]1c2cnccc2CC12CCN(c1nnc3c(=O)n(-c4cccc(Cl)c4Cl)c(=O)[nH]c3n1)CC2]

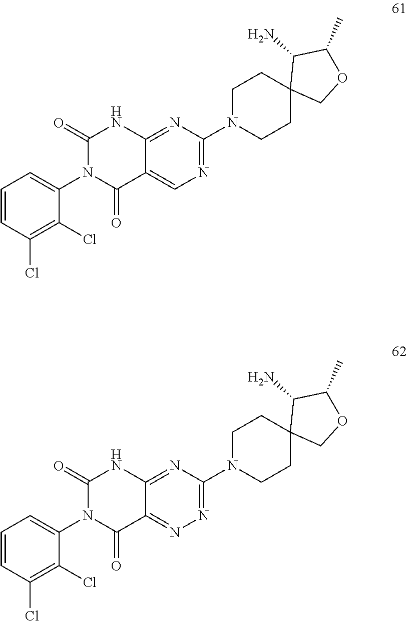 [SMILES: C[C@@H]1OCC2(CCN(c3ncc4c(=O)n(-c5cccc(Cl)c5Cl)c(=O)[nH]c4n3)CC2)[C@@H]1N ; C[C@@H]1OCC2(CCN(c3nnc4c(=O)n(-c5cccc(Cl)c5Cl)c(=O)[nH]c4n3)CC2)[C@@H]1N]

### Embodiment 63

### Preparation of (3S,4S)-8-(2-((2-chloro-3-(1-methyl-1H-pyrazole-3-yl)phenyl)mercapto)pteridine-6-y 1)-3-methyl-2-oxa-8-azaspiro[4.5]decane-4-amine

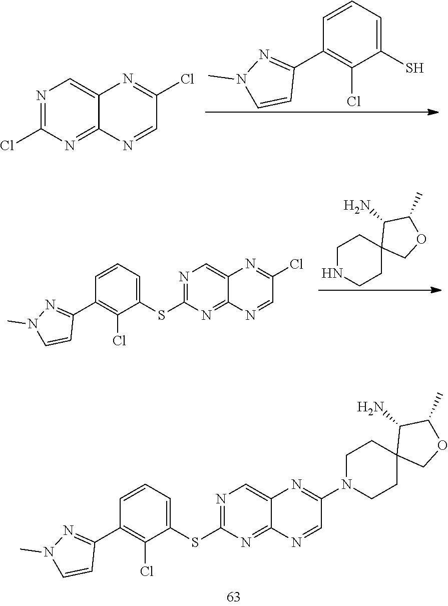

Dissolve the compound 3-(1-methyl-1H-pyrazole-3-yl)-2-chloro-thiophenol (674 mg, 3 mmol) in 8 mL of N,N-dimethylacetamide, add 2,6-dichloropteridine (720 mg, 3.6 mmol), potassium hydroxide (336 mg, 6.0 mmol) and cuprous oxide (215 mg, 1.5 mmol). Perform nitrogen substitution 3 times, and react at 60ºC for 8 hours with stirring. After cooling to room temperature, the reaction solution is diluted with water and, extracted with ethyl acetate. The obtained organic phase is washed with water and saturated brine, dried with anhydrous sodium sulfate, and the organic phase is evaporated to dryness under reduced pressure to give the intermediate 6-chloro-(2-((2-chloro-3-(1-methyl-1H-pyrazole-3-yl)phenyl)mercapto)pteridine (619 mg, 53% yield). LC/MS(ESI): m/z=389.0[M+H]⁺.

Dissolve the compound 6-chloro-(2-((2-chloro-3-(1-methyl-1H-pyrazole-3-yl)phenyl)mercapto)pteridine (584 mg, 1.5 mmol) in 10 mL of N,N-dimethylformamide, add (3S,4S)-4-amino-3-methyl-2-oxa-8-azaspiro[4.5]decane dihydrochloride (438 mg, 1.8 mmol) and cesium carbonate (1.95 g, 6.0 mmol), react at 120° C. for 12 hours under stirring. After cooling to room temperature, the reaction solution is diluted with water and extracted with ethyl acetate. The obtained organic phase is washed with water and saturated brine, dried with anhydrous sodium sulfate, and the organic phase is evaporated to dryness under reduced pressure. The residue is purified by column chromatography to give the target product 63 (282 mg, 36% yield). 1H NMR (400 MHZ, DMSO-d₆) δ: 8.41 (s, 1H), 8.34 (s, 1H), 7.78 (d, 1H), 7.51 (d, 1H), 7.23 (t, 1H), 6.84 (d, 1H), 6.60 (d, 1H), 4.13-4.09 (m, 1H), 3.95-3.45 (m, 7H), 3.32-3.25 (m, 2H), 2.90 (d, 1H), 1.75-1.30 (m, 6H), 1.14 (d, 3H); LC/MS(ESI): m/z=523.2[M+H]⁺.

### Embodiment 64

### Preparation of (3S,4S)-8-(6-((2-chloro-3-(1-methyl-1H-pyrazole-3-yl)phenyl)mercapto)pteridine-2-y 1)-3-methyl-2-oxa-8-azaspiro[4.5]decane-4-amine

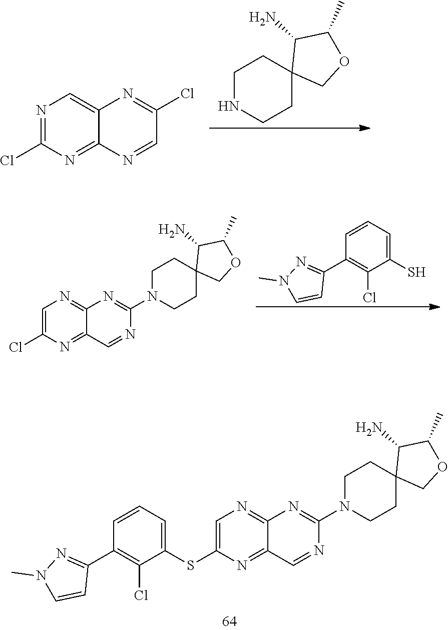

Dissolve 2,6-dichloropteridine (0.72 g, 3.6 mmol) in 10 mL of dichloromethane, add (3S,4S)-4-amino-3-methyl-2-oxa-8-azaspiro[4.5]decane dihydrochloride (0.73 g, 3 mmol) and triethylamine (0.91 g, 9 mmol), stir overnight at room temperature. The reaction solution is diluted with dichloromethane, washed with saturated sodium bicarbonate solution, dried with anhydrous sodium sulfate, and the organic phase is evaporated to dryness under reduced pressure. The residue is purified by column chromatography to give the compound (3S,4S)-8-(6-chloropteridine-2-yl)-3-methyl-2-oxa-8-azaspiro[4.5]decane-4-amine (0.65 g, 65% yield). LC/MS(ESI): m/z=335.1[M+H]⁺.

Dissolve the compound (3S,4S)-8-(6-chloropteridine-2-yl)-3-methyl-2-oxa-8-azaspiro[4.5]decane-4-amine (502 mg, 1.5 mmol) in 8 mL of 1,4-dioxane, add 3-(1-methyl-1H-pyrazole-3-yl)-2-chloro-thiophenol (405 mg, 1.8 mmol), potassium tert-butoxide (335 mg, 3.0 mmol), and cuprous iodide (29 mg, 0.15 mmol), perform nitrogen substitution 3 times, and reflux for 16 hours under stirring. After cooling to room temperature, the reaction solution is passed through a silica gel short column, rinse the column with ethyl acetate, and evaporate the solution to dryness under reduced pressure. The residue is purified by column chromatography to give the target product 64 (377 mg, 48% yield). 1H NMR (400 MHZ, DMSO-d₆) δ: 8.71 (s, 1H), 8.43 (s, 1H), 7.78 (d, 1H), 7.51 (d, 1H), 7.23 (t, 1H), 6.85 (d, 1H), 6.58 (d, 1H), 4.13-4.09 (m, 1H), 3.95-3.44 (m, 7H), 3.34-3.25 (m, 2H), 2.92 (d, 1H), 1.75-1.31 (m, 6H), 1.15 (d, 3H); LC/MS(ESI): m/z=523.2[M+H]⁺.

### Embodiment 65

### Preparation of (3S,4S)-8-(2-((2-chloro-3-(1-methyl-1H-pyrazole-3-yl)phenyl)mercapto)pyridinyl[3,2-d]pyrimidine-6-yl)-3-methyl-2-oxa-8-azaspiro[4.5]decane-4-amine

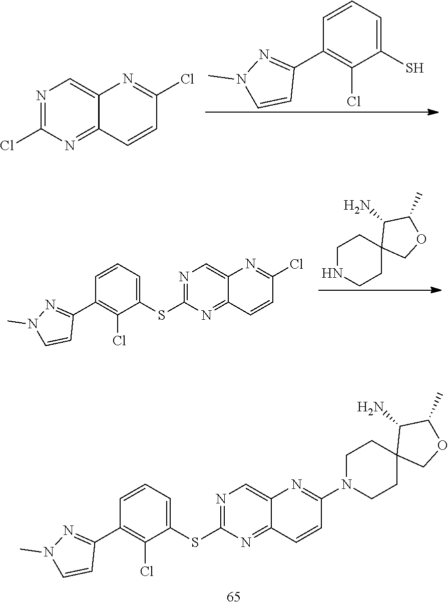

Compound 65 (328 mg, 42% yield, which is the yield of the last step, the same below) is obtained in a similar manner to that of Embodiment 63. ¹H NMR (400 MHZ, DMSO-d₆) δ: 8.42 (s, 1H), 7.93 (d, 1H), 7.78 (d, 1H), 7.51 (d, 1H), 7.23 (t, 1H), 6.88-6.75 (m, 2H), 6.59 (d, 1H), 4.13-4.09 (m, 1H), 3.94-3.45 (m, 7H), 3.31-3.25 (m, 2H), 2.91 (d, 1H), 1.75-1.30 (m, 6H), 1.15 (d, 3H); LC/MS(ESI): m/z=522.2[M+H]⁺.

### Embodiment 66

### Preparation of (3S,4S)-8-(2-((2-chloro-3-(1-ethyl-1H-pyrazole-3-yl)phenyl)mercapto)pteridine-6-yl)-3-methyl yl-2-oxa-8-azaspiro[4.5]decane-4-amine

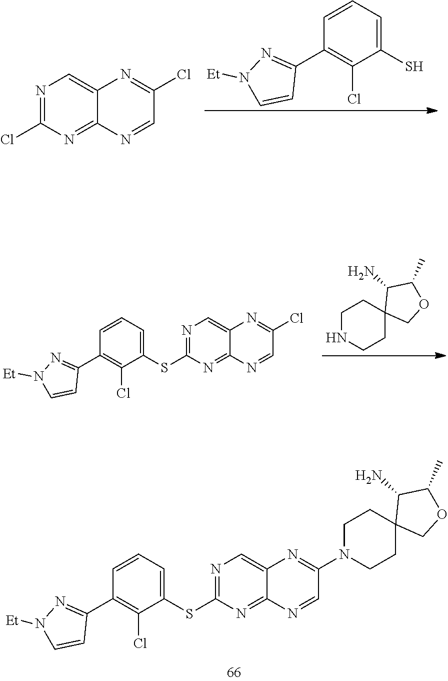

Compound 66 (322 mg, yield 40%) is obtained in a similar manner to Embodiment 63. ¹H NMR (400 MHZ, DMSO-d₆) δ: 8.42 (s, 1H), 8.34 (s, 1H), 7.75 (d, 1H), 7.51 (d, 1H), 7.23 (t, 1H), 6.83 (d, 1H), 6.56 (d, 1H), 4.13-4.09 (m, 1H), 3.95-3.44 (m, 6H), 3.32-3.25 (m, 2H), 2.90 (d, 1H), 1.73-1.31 (m, 6H), 1.28 (t, 3H), 1.13 (d, 3H); LC/MS(ESI): m/z=537.2[M+H]⁺.

### Embodiment 67

### Preparation of (3S,4S)-8-(6-((2-chloro-3-(1-ethyl-1H-pyrazole-3-yl)phenyl)mercapto)pteridine-2-yl)-3-methyl yl-2-oxa-8-azaspiro[4.5]decane-4-amine

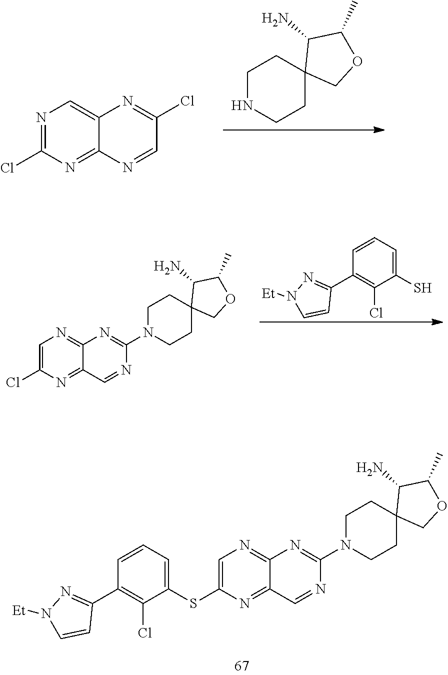

Compound 67 (362 mg, yield 45%) is obtained in a similar manner to Embodiment 64. LC/MS(ESI): m/z=537.2[M+H]⁺.

### Embodiment 68

### Preparation of (3S,4S)-8-(2-((2-chloro-3-(1-ethyl-1H-pyrazole-3-yl)phenyl)mercapto)pyridinyl[3,2-d]pyrimidine-6-yl)-3-methyl-2-oxa-8-azaspiro[4.5]decane-4-amine

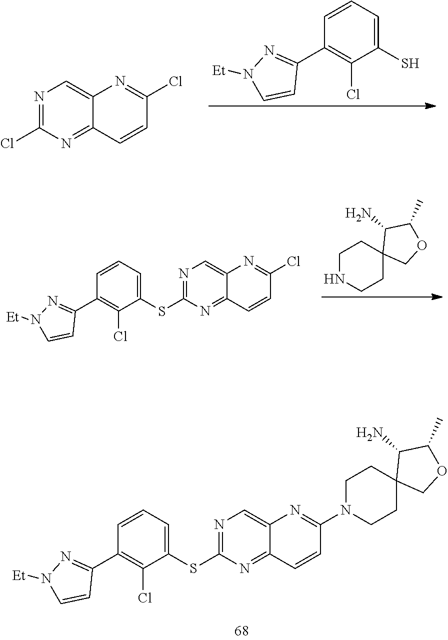 [SMILES: Clc1ccc2nc(Cl)ncc2n1 ; CCn1ccc(-c2cccc(S)c2Cl)n1 ; CCn1ccc(-c2cccc(Sc3ncc4nc(Cl)ccc4n3)c2Cl)n1 ; C[C@@H]1OCC2(CCNCC2)[C@@H]1N ; CCn1ccc(-c2cccc(Sc3ncc4nc(N5CCC6(CC5)CO[C@@H](C)[C@H]6N)ccc4n3)c2Cl)n1]

Compound 68 (305 mg, yield 38%) is obtained in a similar manner to Embodiment 63. LC/MS(ESI): m/z=536.2[M+H]⁺.

### Embodiment 69

### Preparation of (3S,4S)-8-(2-((2-chloro-3-(1-isopropyl-1H-pyrazole-3-yl)phenyl)mercapto)pteridine-6-yl)-3-methyl-2-oxa-8-azaspiro[4.5]decane-4-amine

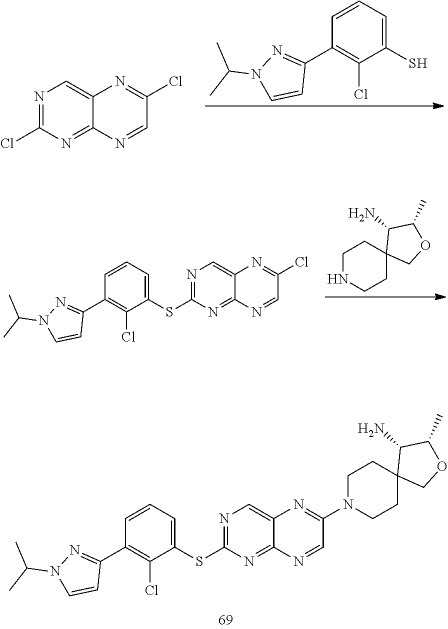

Compound 69 (305 mg, yield 37%) is obtained in a similar manner to Embodiment 63. ¹H NMR (400 MHZ, DMSO-d₆) δ: 8.42 (s, 1H), 8.34 (s, 1H), 7.76 (s, 1H), 7.51 (d, 1H), 7.23 (t, 1H), 6.83 (d, 1H), 6.55 (d, 1H), 4.14-4.07 (m, 2H), 3.93-3.45 (m, 4H), 3.32-3.25 (m, 2H), 2.90 (d, 1H), 1.73-1.30 (m, 12H), 1.14 (d, 3H); LC/MS(ESI): m/z=551.2[M+H]⁺.

### Embodiment 70

### Preparation of (3S,4S)-8-(6-((2-chloro-3-(1-isopropyl-1H-pyrazole-3-yl)phenyl)mercapto)pteridine-2-yl)-3-methyl-2-oxa-8-azaspiro[4.5]decane-4-amine

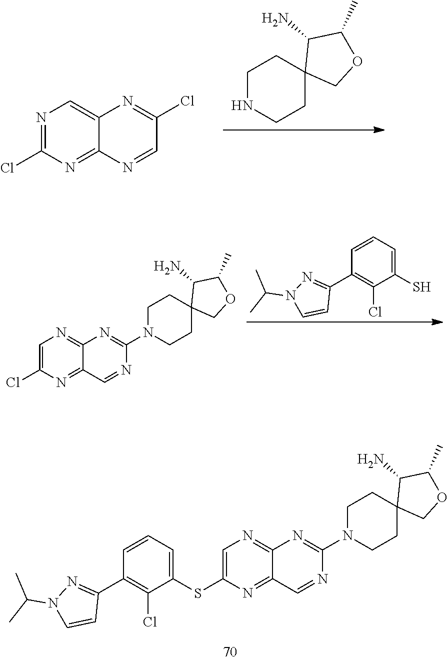

Compound 70 (380 mg, yield 46%) is obtained in a similar manner to Embodiment 64. LC/MS(ESI): m/z=551.2[M+H]⁺.

### Embodiment 71

### Preparation of (3S,4S)-8-(2-((2-chloro-3-(1-isopropyl-1H-pyrazole-3-yl)phenyl)mercapto)pyridinyl[3,2-d]pyrimidine-6-yl)-3-methyl-2-oxa-8-azaspiro[4.5]decane-4-amine

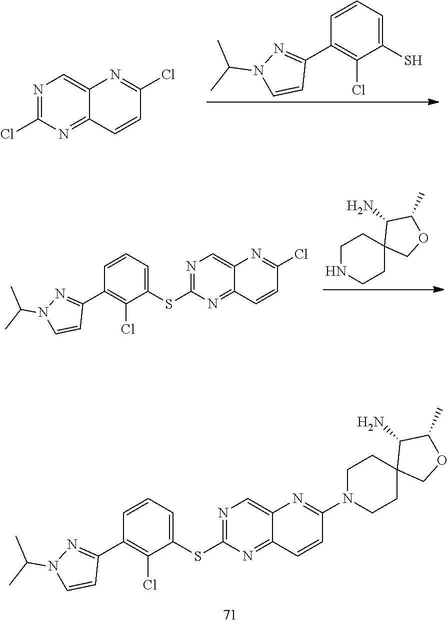 [SMILES: Clc1ccc2nc(Cl)ncc2n1 ; CC(C)n1ccc(-c2cccc(S)c2Cl)n1 ; CC(C)n1ccc(-c2cccc(Sc3ncc4nc(Cl)ccc4n3)c2Cl)n1 ; C[C@@H]1OCC2(CCNCC2)[C@@H]1N ; CC(C)n1ccc(-c2cccc(Sc3ncc4nc(N5CCC6(CC5)CO[C@@H](C)[C@H]6N)ccc4n3)c2Cl)n1]

Compound 71 (288 mg, yield 35%) is obtained in a similar manner to Embodiment 63. LC/MS(ESI): m/z=550.2[M+H]⁺.

### Embodiment 72

### Preparation of (3S,4S)-8-(2-((2-chloro-3-(pyrazine-2-yl)phenyl)mercapto)pteridine-6-yl)-3-methyl-2-oxa-8-azaspiro[4.5]decane-4-amine

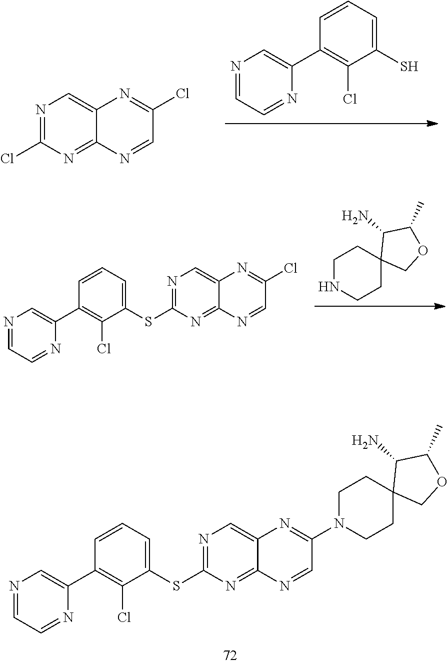 [SMILES: Clc1cnc2nc(Cl)ncc2n1 ; Sc1cccc(-c2cnccn2)c1Cl ; Clc1cnc2nc(Sc3cccc(-c4cnccn4)c3Cl)ncc2n1 ; C[C@@H]1OCC2(CCNCC2)[C@@H]1N ; C[C@@H]1OCC2(CCN(c3cnc4nc(Sc5cccc(-c6cnccn6)c5Cl)ncc4n3)CC2)[C@@H]1N]

Compound 72 (273 mg, yield 35%) is obtained in a similar manner to Embodiment 63. ¹H NMR (400 MHZ, DMSO-d₆) δ: 8.81 (s, 1H), 8.75 (s, 2H), 8.43 (s, 1H), 8.34 (s, 1H), 7.51 (d, 1H), 7.21 (t, 1H), 6.86 (d, 1H), 4.13-4.09 (m, 1H), 3.94-3.75 (m, 2H), 3.65 (d, 1H), 3.47 (d, 1H), 3.32-3.25 (m, 2H), 2.90 (d, 1H), 1.75-1.33 (m, 6H), 1.14 (d, 3H); LC/MS(ESI): m/z=521.2[M+H]⁺.

### Embodiment 73

### Preparation of (3S,4S)-8-(6-((2-chloro-3-(pyrazine-2-yl)phenyl)mercapto)pteridine-2-yl)-3-methyl-2-oxa-8-azaspiro[4.5]decane-4-amine

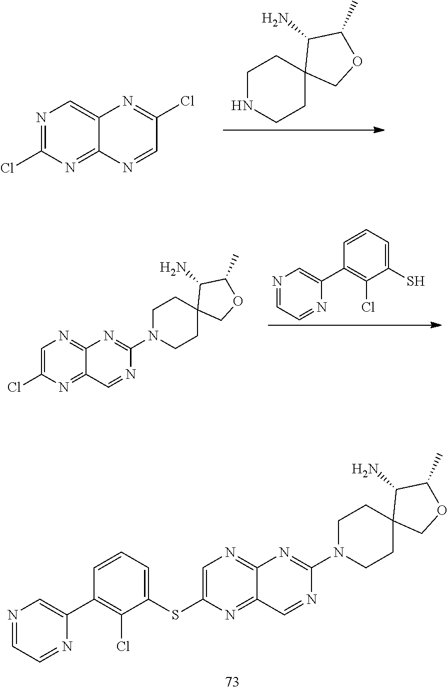

Compound 73 (328 mg, yield 42%) is obtained in a similar manner to Embodiment 64. ¹H NMR (400 MHZ, DMSO-d₆) δ: 8.82-8.80 (m, 2H), 8.75 (s, 2H), 8.43 (s, 1H), 7.51 (d, 1H), 7.23 (t, 1H), 7.05 (d, 1H), 4.13-4.09 (m, 1H), 3.94-3.43 (m, 4H), 3.32-3.23 (m, 2H), 2.91 (d, 1H), 1.79-1.33 (m, 6H), 1.15 (d, 3H); LC/MS(ESI): m/z=521.2[M+H]⁺.

### Embodiment 74

### Preparation of (3S,4S)-8-(2-((2-chloro-3-(pyrazine-2-yl)phenyl)mercapto)pyridinyl[3,2-d]pyrimidin e-6-yl)-3-methyl-2-oxa-8-azaspiro[4.5]decane-4-amine

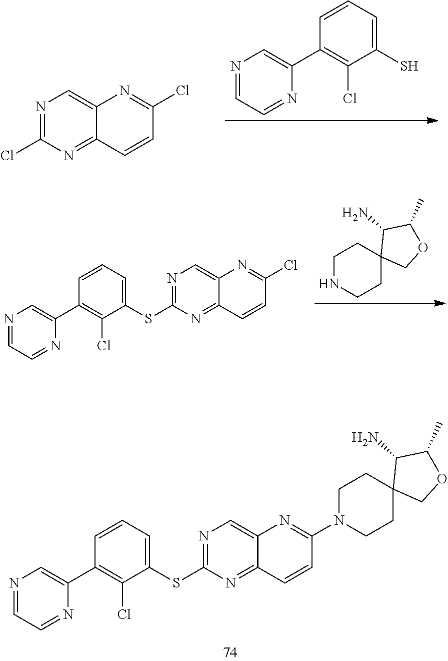 [SMILES: Clc1ccc2nc(Cl)ncc2n1 ; Sc1cccc(-c2cnccn2)c1Cl ; Clc1ccc2nc(Sc3cccc(-c4cnccn4)c3Cl)ncc2n1 ; C[C@@H]1OCC2(CCNCC2)[C@@H]1N ; C[C@@H]1OCC2(CCN(c3ccc4nc(Sc5cccc(-c6cnccn6)c5Cl)ncc4n3)CC2)[C@@H]1N]

Compound 74 (288 mg, yield 37%) is obtained in a similar manner to Embodiment 63. ¹H NMR (400 MHZ, DMSO-d₆) δ: 8.81 (s, 1H), 8.75 (s, 2H), 8.43 (s, 1H), 7.91 (d, 1H), 7.51 (d, 1H), 7.21 (t, 1H), 6.86-6.79 (m, 2H), 4.13-4.09 (m, 1H), 3.94-3.75 (m, 2H), 3.65 (d, 1H), 3.47 (d, 1H), 3.32-3.25 (m, 2H), 2.90 (d, 1H), 1.75-1.33 (m, 6H), 1.14 (d, 3H); LC/MS(ESI): m/z=520.2[M+H]⁺.

### Embodiment 75

### Preparation of (3S,4S)-8-(2-((2-chloro-3-(pyrimidine-4-yl)phenyl)mercapto)pteridine-6-yl)-3-methyl-2-oxa-8-azaspiro[4.5]decane-4-amine

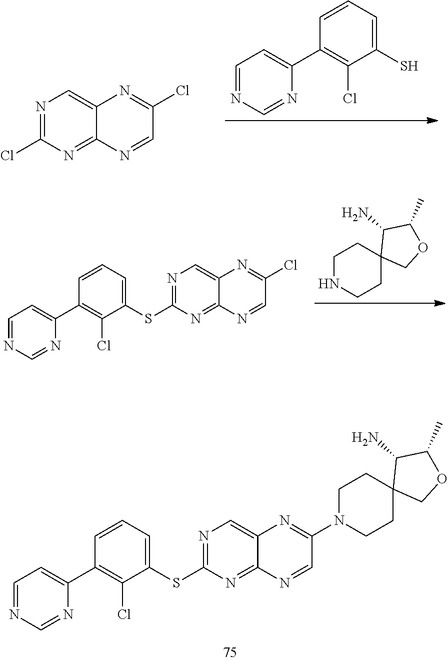 [SMILES: Clc1cnc2nc(Cl)ncc2n1 ; Sc1cccc(-c2ccncn2)c1Cl ; Clc1cnc2nc(Sc3cccc(-c4ccncn4)c3Cl)ncc2n1 ; C[C@@H]1OCC2(CCNCC2)[C@@H]1N ; C[C@@H]1OCC2(CCN(c3cnc4nc(Sc5cccc(-c6ccncn6)c5Cl)ncc4n3)CC2)[C@@H]1N]

Compound 75 (320 mg, yield 41%) is obtained in a similar manner to Embodiment 63. ¹H NMR (400 MHz, DMSO-d₆) δ: 9.24 (s, 1H), 8.85 (d, 1H), 8.43 (s, 1H), 8.35 (s, 1H), 8.12 (d, 1H), 7.50 (d, 1H), 7.21 (t, 1H), 6.86 (d, 1H), 4.13-4.09 (m, 1H), 3.94-3.75 (m, 2H), 3.65 (d, 1H), 3.47 (d, 1H), 3.32-3.25 (m, 2H), 2.91 (d, 1H), 1.77-1.33 (m, 6H), 1.14 (d, 3H); LC/MS(ESI): m/z=521.2[M+H]⁺.

### Embodiment 76

### Preparation of (3S,4S)-8-(6-((2-chloro-3-(pyrimidine-4-yl)phenyl)mercapto)pteridine-2-yl)-3-methyl-2-oxa-8-azaspiro[4.5]decane-4-amine

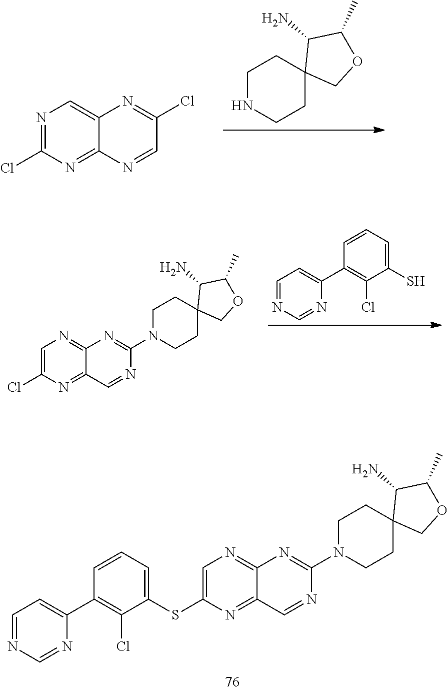

Compound 76 (295 mg, yield 37%) is obtained in a similar manner to Embodiment 2. LC/MS(ESI): m/z=521.2[M+H]⁺.

### Embodiment 77

### Preparation of (3S,4S)-8-(2-((2-chloro-3-(pyrimidine-4-yl)phenyl)mercapto)pyridinyl[3,2-d]pyrimid ine-6-yl)-3-methyl-2-oxa-8-azaspiro[4.5]decane-4-amine

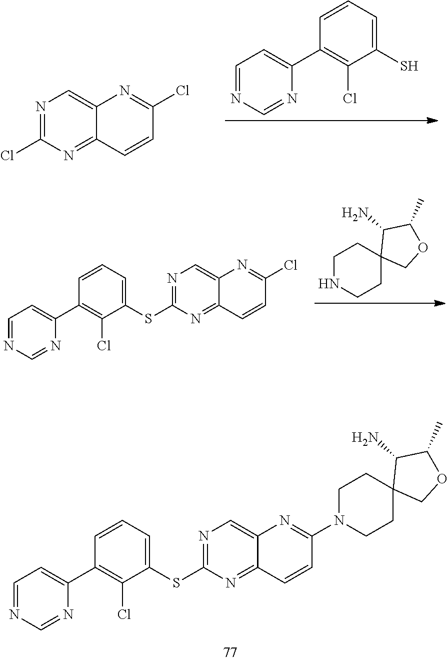 [SMILES: Clc1ccc2nc(Cl)ncc2n1 ; Sc1cccc(-c2ccncn2)c1Cl ; Clc1ccc2nc(Sc3cccc(-c4ccncn4)c3Cl)ncc2n1 ; C[C@@H]1OCC2(CCNCC2)[C@@H]1N ; C[C@@H]1OCC2(CCN(c3ccc4nc(Sc5cccc(-c6ccncn6)c5Cl)ncc4n3)CC2)[C@@H]1N]

Compound 77 (350 mg, yield 45%) is obtained in a similar manner to Embodiment 63. LC/MS(ESI): m/z=520.2[M+H]⁺.

### Embodiment 78

### Preparation of (3S,4S)-8-(2-((2-chloro-3-(pyrimidine-3-yl)phenyl)mercapto)pteridine-6-yl)-3-methyl-2-oxa-8-azaspiro[4.5]decane-4-amine

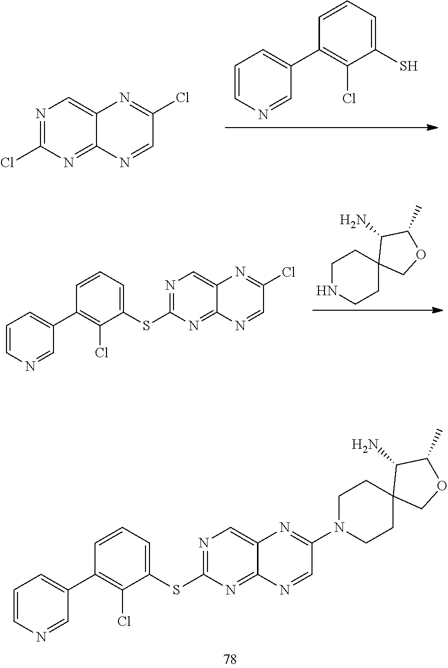 [SMILES: Clc1cnc2nc(Cl)ncc2n1 ; Sc1cccc(-c2cccnc2)c1Cl ; Clc1cnc2nc(Sc3cccc(-c4cccnc4)c3Cl)ncc2n1 ; C[C@@H]1OCC2(CCNCC2)[C@@H]1N ; C[C@@H]1OCC2(CCN(c3cnc4nc(Sc5cccc(-c6cccnc6)c5Cl)ncc4n3)CC2)[C@@H]1N]

Compound 78 (327 mg, yield 42%) is obtained in a similar manner to Embodiment 63. ¹H NMR (400 MHZ, DMSO-d₆) δ: 8.87 (s, 1H), 8.71 (d, 1H), 8.34-8.30 (m, 3H), 7.55-7.51 (m, 2H), 7.22 (t, 1H), 7.04 (d, 1H), 4.13-4.08 (m, 1H), 3.94-3.45 (m, 4H), 3.31-3.25 (m, 2H), 2.91 (d, 1H), 1.78-1.31 (m, 6H), 1.15 (d, 3H); LC/MS(ESI): m/z=520.2[M+H]⁺.

### Embodiment 79

### Preparation of (3S,4S)-8-(6-((2-chloro-3-(pyrimidine-4-yl)phenyl)mercapto)pteridine-2-yl)-3-methyl-2-oxa-8-azaspiro[4.5]decane-4-amine

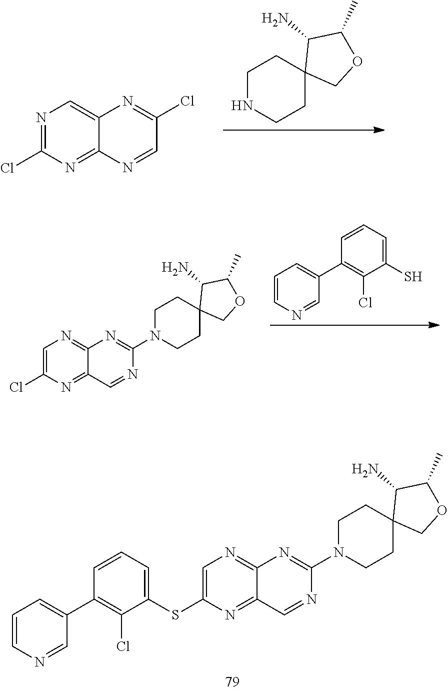

Compound 79 (273 mg, yield 35%) is obtained in a similar manner to Embodiment 64. LC/MS(ESI): m/z=520.2[M+H]⁺.

### Embodiment 80

### Preparation of (3S,4S)-8-(2-((2-chloro-3-(pyrimidine-4-yl)phenyl)mercapto)pyridinyl[3,2-d]pyrimid ine-6-yl)-3-methyl-2-oxa-8-azaspiro[4.5]decane-4-amine

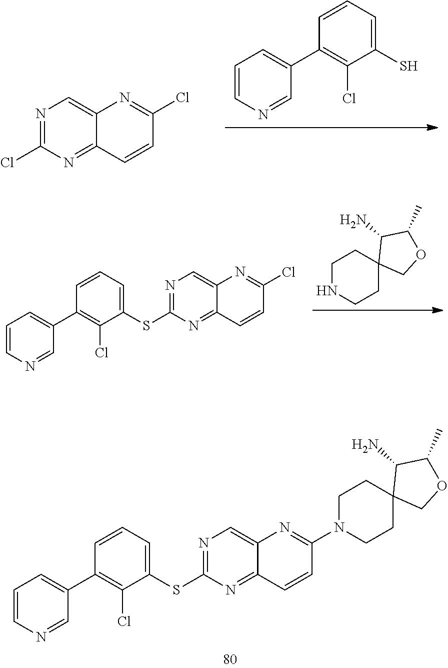 [SMILES: Clc1ccc2nc(Cl)ncc2n1 ; Sc1cccc(-c2cccnc2)c1Cl ; Clc1ccc2nc(Sc3cccc(-c4cccnc4)c3Cl)ncc2n1 ; C[C@@H]1OCC2(CCNCC2)[C@@H]1N ; C[C@@H]1OCC2(CCN(c3ccc4nc(Sc5cccc(-c6cccnc6)c5Cl)ncc4n3)CC2)[C@@H]1N]

Compound 80 (295 mg, yield 38%) is obtained in a similar manner to Embodiment 63. LC/MS(ESI): m/z=519.2[M+H]⁺.

### Embodiment 81

### Preparation of N-(3-((6-((3S,4S)-4-amino-3-methyl-2-oxa-8-azaspiro[4.5]decane-8-yl)pteridine-2-yl) mercapto)-2-chlorophenyl)pyrimidine-4-carboxamide

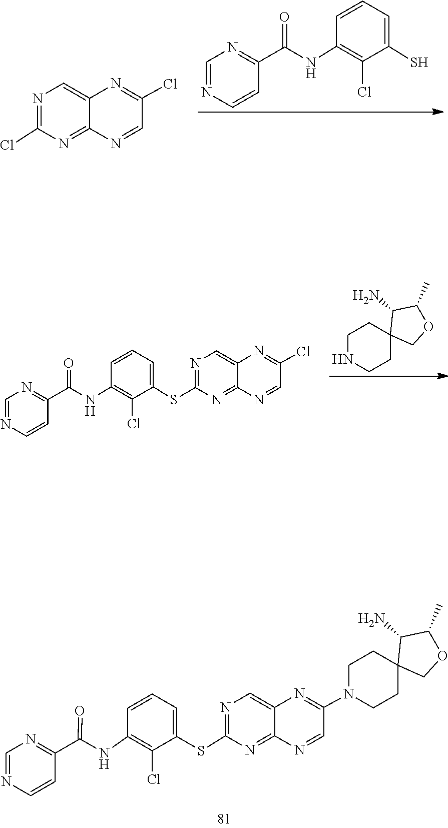

Compound 81 (320 mg, yield 41%) is obtained in a similar manner to Embodiment 63. ¹H NMR (400 MHz, DMSO-d₆) δ: 9.88 (s, 1H), 9.31 (s, 1H), 9.01 (d, 1H), 8.43 (s, 1H), 8.35 (s, 1H), 8.31 (s, 2H), 8.21 (d, 1H), 7.75 (d, 1H), 7.42 (t, 1H), 7.15 (d, 1H), 4.15-4.11 (m, 1H), 3.93-3.45 (m, 4H), 3.33-3.21 (m, 2H), 2.90 (d, 1H), 1.75-1.31 (m, 6H), 1.15 (d, 3H); LC/MS(ESI): m/z=564.2[M+H]⁺.

### Embodiment 82

### Preparation of N-(3-((2-((3S,4S)-4-amino-3-methyl-2-oxa-8-azaspiro[4.5]decane-8-yl)pteridine-6-yl) mercapto)-2-chlorophenyl)pyrimidine-4-carboxamide

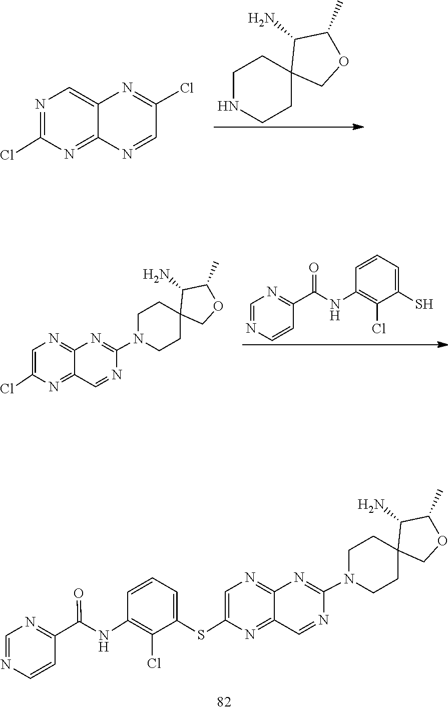

Compound 82 (295 mg, yield 37%) is obtained in a similar manner to Embodiment 64. LC/MS(ESI): m/z=564.2[M+H]⁺.

### Embodiment 83

### Preparation of N-(3-((6-((3S,4S)-4-amino-3-methyl-2-oxa-8-azaspiro[4.5]decane-8-yl)pyridinyl[3,2-d]pyrimidine-2-yl)mercapto)-2-chlorophenyl)pyrimidine-4-carboxamide

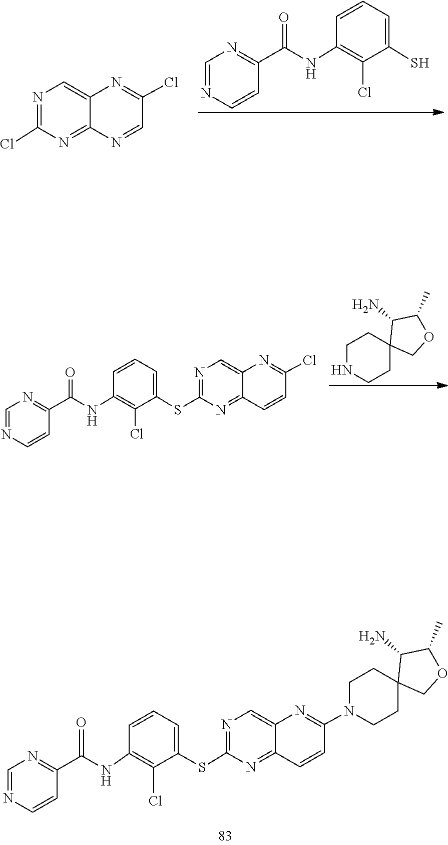

Compound 83 (350 mg, yield 45%) is obtained in a similar manner to Embodiment 63. LC/MS(ESI): m/z=563.2[M+H]⁺.

### Embodiment 84

### Preparation of N-(3-((6-((3S,4S)-4-amino-3-methyl-2-oxa-8-azaspiro[4.5]decane-8-yl)pteridine-2-yl) mercapto)-2-chlorophenyl)pyrazine-2-carboxamide

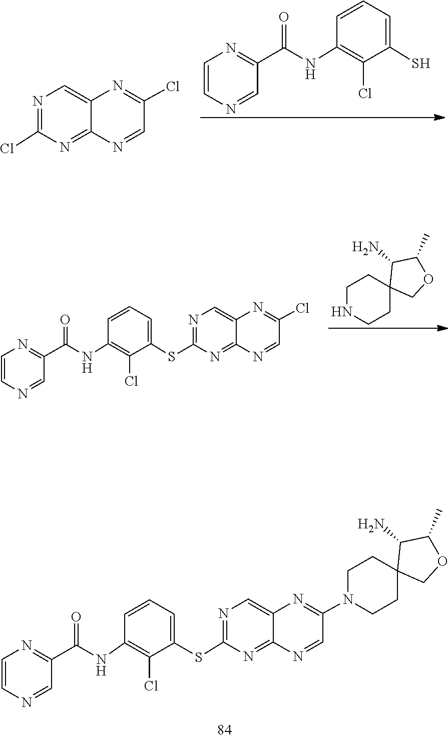

Compound 84 (313 mg, yield 37%) is obtained in a similar manner to Embodiment 63. ¹H NMR (400 MHZ, DMSO-d₆) δ: 9.70 (s, 1H), 9.51 (s, 1H), 8.81 (d, 1H), 8.55-8.52 (m, 1H), 8.43 (s, 1H), 8.35 (s, 1H), 7.75 (d, 1H), 7.42 (t, 1H), 7.14 (d, 1H), 4.15-4.11 (m, 1H), 3.93-3.45 (m, 4H), 3.33-3.21 (m, 2H), 2.90 (d, 1H), 1.75-1.31 (m, 6H), 1.15 (d, 3H); LC/MS(ESI): m/z=564.2[M+H]⁺.

### Embodiment 85

### Preparation of N-(3-((2-((3S,4S)-4-amino-3-methyl-2-oxa-8-azaspiro[4.5]decane-8-yl)pteridine-6-yl) mercapto)-2-chlorophenyl)pyrazine-2-carboxamide

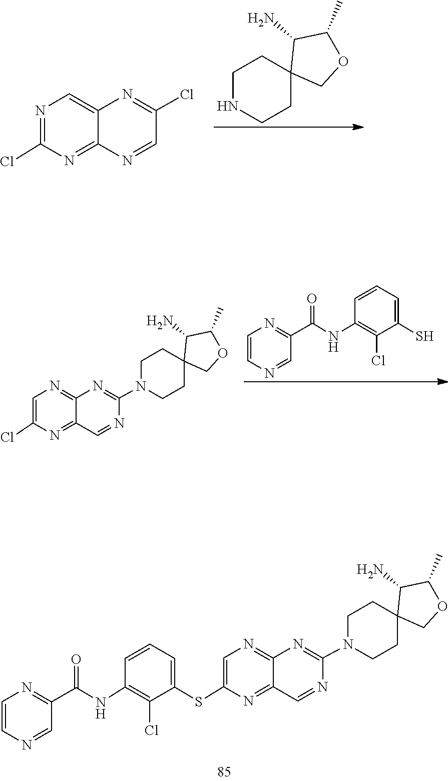

Compound 85 (337 mg, yield 40%) is obtained in a similar manner to Embodiment 64. LC/MS(ESI): m/z=564.2[M+H]⁺.

### Embodiment 86

### Preparation of N-(3-((6-((3S,4S)-4-amino-3-methyl-2-oxa-8-azaspiro[4.5]decane-8-yl)pyridinyl[3,2-d]pyrimidine-2-yl)mercapto)-2-chlorophenyl)pyrazine-2-carboxamide

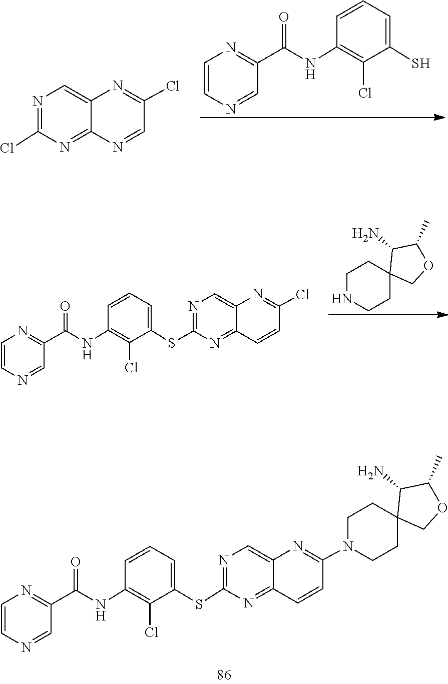

Compound 76 (346 mg, yield 41%) is obtained in a similar manner to Embodiment 63. LC/MS(ESI): m/z=563.2[M+H]⁺.

### Embodiment 87

### Preparation of N-(3-((6-((3S,4S)-4-amino-3-methyl-2-oxa-8-azaspiro[4.5]decane-8-yl)pteridine-2-yl) mercapto)-2-chlorophenyl)-2-hydroxy-4-oxo-6,7,8,9-tetrahydro-4H-pyridinyl[1,2-a]p yrimidine-3-carboxamide

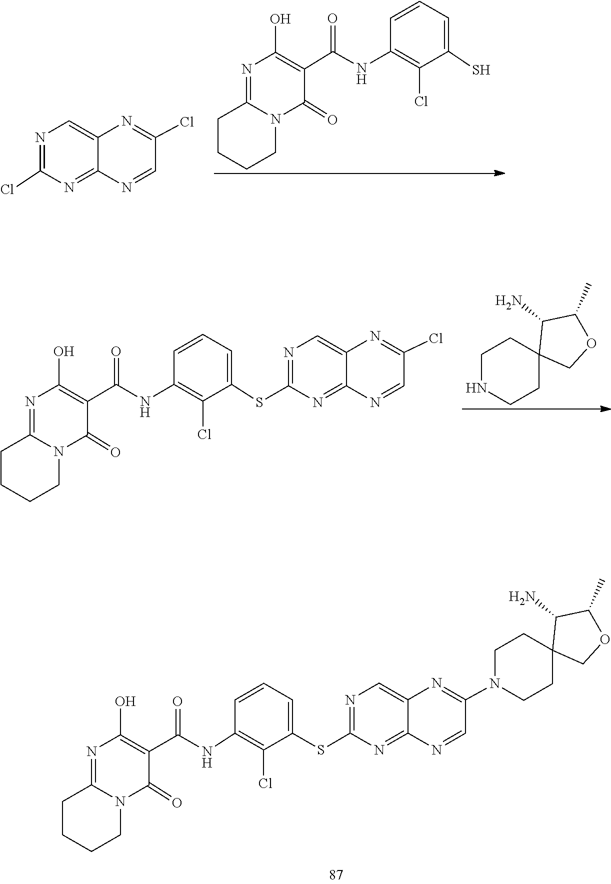

Compound 87 (341 mg, yield 35%) is obtained in a similar manner to Embodiment 63. ¹H NMR (400 MHZ, DMSO-d₆) δ: 12.3 (br s, 1H), 8.42 (s, 1H), 8.35 (s, 1H), 8.24 (s, 1H), 7.23 (s, 1H), 6.63 (d, 1H), 5.51 (br s, 3H), 4.13-4.10 (m, 1H), 3.93-3.45 (m, 8H), 2.95 (d, 1H), 2.74 (s, 2H), 1.85-1.41 (m, 8H), 1.14 (d, 3H); LC/MS(ESI): m/z=650.2[M+H]⁺.

### Embodiment 88

### Preparation of N-(3-((2-((3S,4S)-4-amino-3-methyl-2-oxa-8-azaspiro[4.5]decane-8-yl)pteridine-6-yl) mercapto)-2-chlorophenyl)-2-hydroxy-4-oxo-6,7,8,9-tetrahydro-4H-pyridinyl[1,2-a]p yrimidine-3-carboxamide

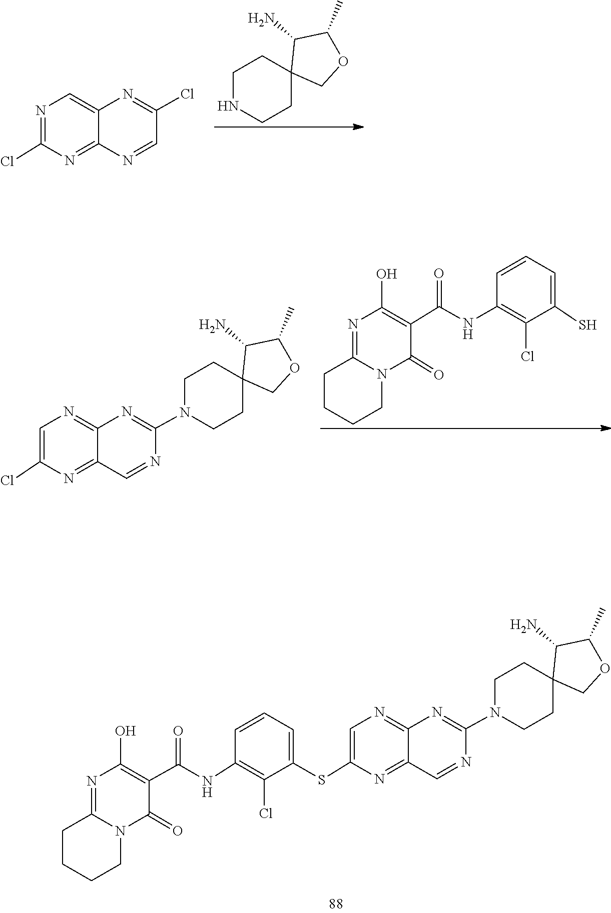

Compound 88 (389 mg, yield 40%) is obtained in a similar manner to Embodiment 64. LC/MS(ESI): m/z=650.2[M+H]⁺.

### Embodiment 89

### Preparation of N-(3-((6-((3S,4S)-4-amino-3-methyl-2-oxa-8-azaspiro[4.5]decane-8-yl)pyridinyl[3,2-d]pyrimidine-2-yl)mercapto)-2-chlorophenyl)-2-hydroxy-4-oxo-6,7,8,9-tetrahydro-4H-pyridinyl[1,2-a]pyrimidine-3-carboxamide

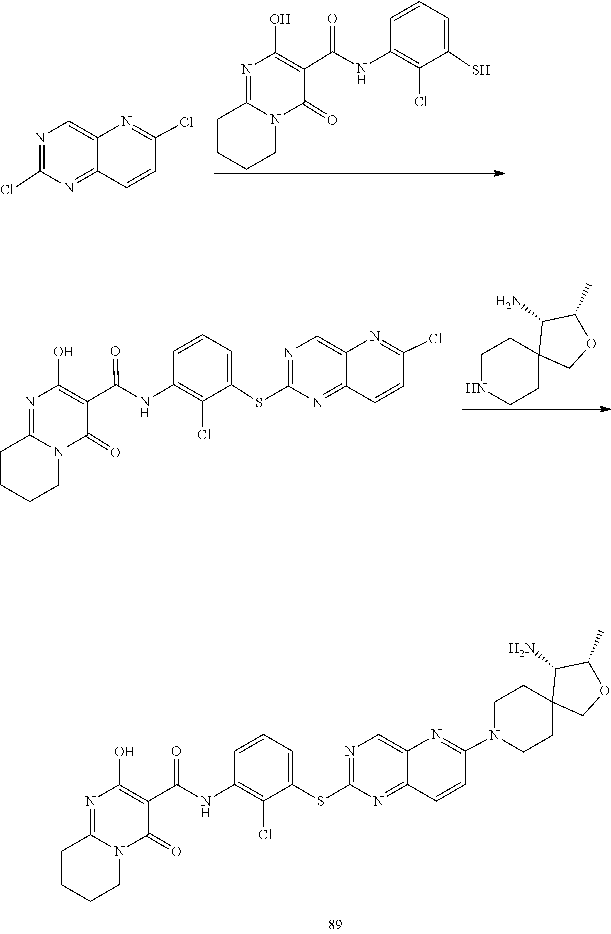

Compound 79 (369 mg, yield 38%) is obtained in a similar manner to Embodiment 63. LC/MS(ESI): m/z=649.2[M+H]⁺.

### Embodiment 90

### Preparation of (3S,4S)-8-(6-((2-chloro-3-(1-methyl-1H-pyrazole-3-yl)phenyl)mercapto)pyridinyl[3,2-b]pyrimidine-2-yl)-3-methyl-2-oxa-8-azaspiro[4.5]decane-4-amine

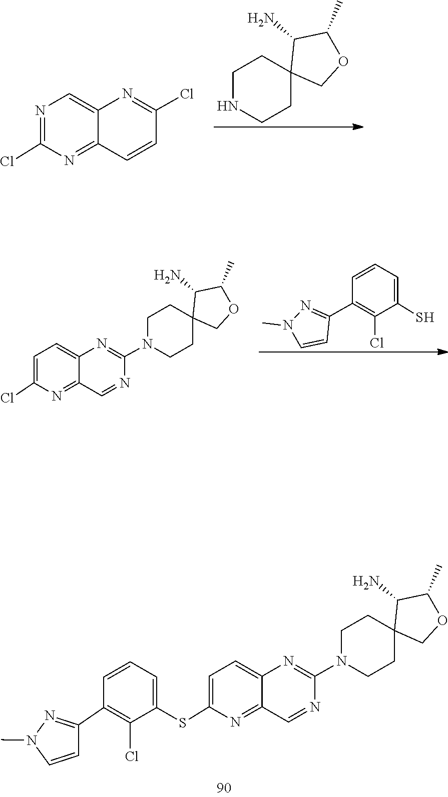

Compound 90 (281 mg, yield 36%) is obtained in a similar manner to Embodiment 64. ¹H NMR (400 MHZ, DMSO-d₆) δ: 8.43 (s, 1H), 7.96 (d, 1H), 7.78 (d, 1H), 7.49-7.23 (m, 3H), 6.88 (d, 1H), 6.59 (d, 1H), 4.13-3.45 (m, 8H), 3.30-3.25 (m, 2H), 2.91 (d, 1H), 1.75-1.30 (m, 6H), 1.15 (d, 3H); LC/MS(ESI): m/z=522.2[M+H]⁺.

### Embodiment 91

### Preparation of (3S,4S)-8-(2-((2-chloro-3-(1-methyl-1H-pyrazole-3-yl)phenyl)mercapto)pyridinyl[2,3-b]pyrazine-6-yl)-3-methyl-2-oxa-8-azaspiro[4.5]decane-4-amine

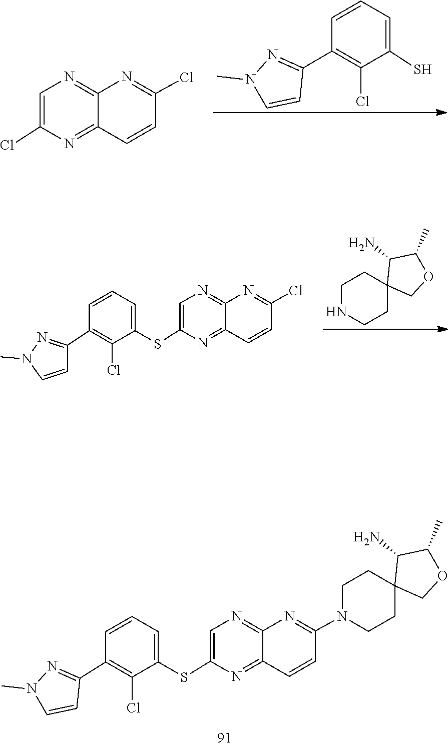

Compound 91 (235 mg, yield 30%) is obtained in a similar manner to Embodiment 63. LC/MS(ESI): m/z=522.2[M+H]⁺.

### Embodiment 92

### Preparation of (3S,4S)-8-(6-((2-chloro-3-(1-methyl-1H-pyrazole-3-yl)phenyl)mercapto)pyridinyl[2,3-b]pyrazine-2-yl)-3-methyl-2-oxa-8-azaspiro[4.5]decane-4-amine

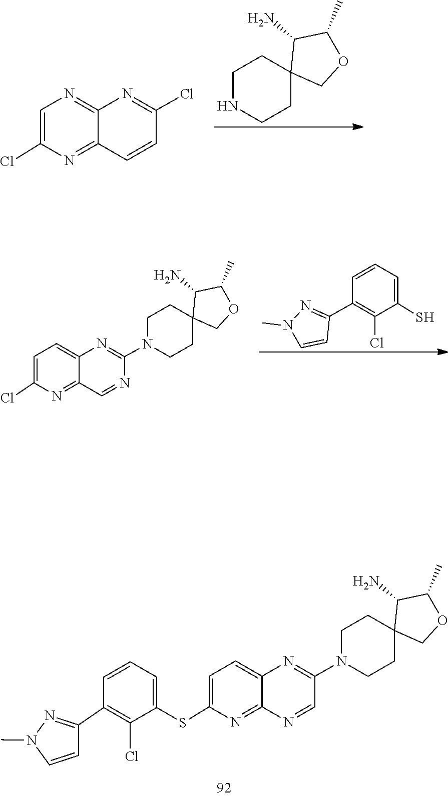

Compound 92 (211 mg, yield 27%) is obtained in a similar manner to Embodiment 64. LC/MS(ESI): m/z=522.2[M+H]⁺.

### Embodiment 93

### Preparation of (3S,4S)-8-(6-((2-chloro-3-(1-methyl-1H-pyrazole-3-yl)hydroxy)phenyl)mercapto)pter idine-2-yl)-3-methyl-2-oxa-8-azaspiro[4.5]decane-4-amine

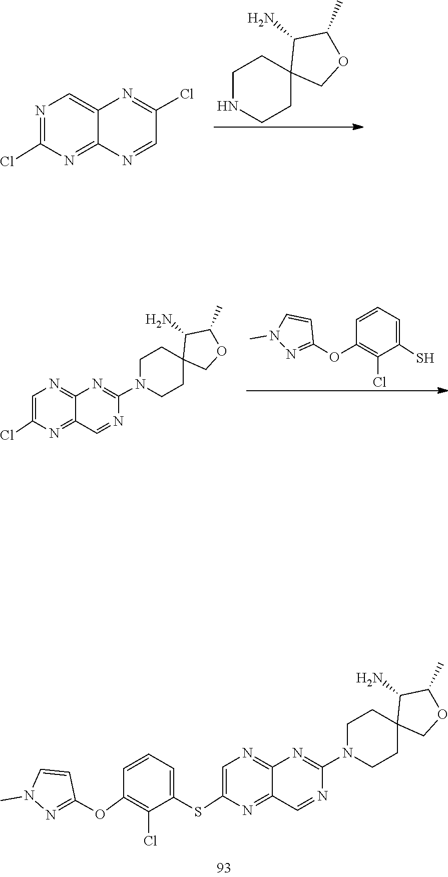

Compound 93 (315 mg, yield 39%) is obtained in a similar manner to Embodiment 65. ¹H NMR (400 MHz, DMSO-d₆) δ: 8.43-8.40 (m, 2H), 7.78 (d, 1H), 7.03-6.55 (m, 4H), 4.14-3.45 (m, 8H), 3.32-3.25 (m, 2H), 2.91 (d, 1H), 1.75-1.30 (m, 6H), 1.14 (d, 3H); LC/MS(ESI): m/z=539.2[M+H]⁺.

### Embodiment 94

### Preparation of (3S,4S)-8-(2-((2-chloro-3-(1-methyl-1H-pyrazole-3-yl)hydroxy)phenyl)mercapto)pter idine-6-yl)-3-methyl-2-oxa-8-azaspiro[4.5]decane-4-amine

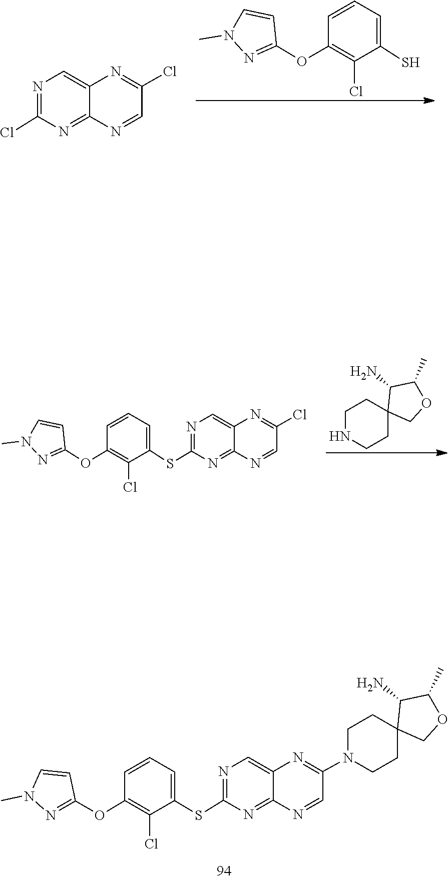 [SMILES: Clc1cnc2nc(Cl)ncc2n1 ; Cn1ccc(Oc2cccc(S)c2Cl)n1 ; Cn1ccc(Oc2cccc(Sc3ncc4nc(Cl)cnc4n3)c2Cl)n1 ; C[C@@H]1OCC2(CCNCC2)[C@@H]1N ; C[C@@H]1OCC2(CCN(c3cnc4nc(Sc5cccc(Oc6ccn(C)n6)c5Cl)ncc4n3)CC2)[C@@H]1N]

Compound 94 (331 mg, yield 41%) is obtained in a similar manner to Embodiment 63. LC/MS(ESI): m/z=539.2[M+H]⁺.

### Embodiment 95

### Preparation of (3S,4S)-8-(2-((2-chloro-3-(1-methyl-1H-pyrazole-3-yl)hydroxy)phenyl)mercapto)pyri dinyl[3,2-d]pyrimidine-6-yl)-3-methyl-2-oxa-8-azaspiro[4.5]decane-4-amine

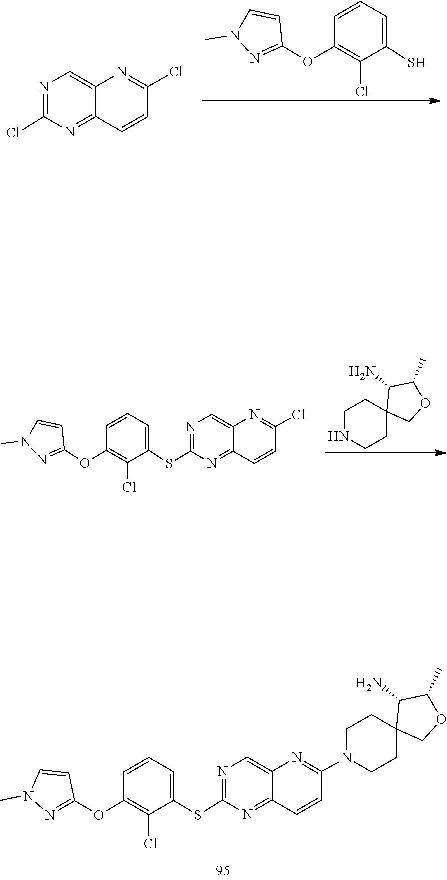 [SMILES: Clc1ccc2nc(Cl)ncc2n1 ; Cn1ccc(Oc2cccc(S)c2Cl)n1 ; Cn1ccc(Oc2cccc(Sc3ncc4nc(Cl)ccc4n3)c2Cl)n1 ; C[C@@H]1OCC2(CCNCC2)[C@@H]1N ; C[C@@H]1OCC2(CCN(c3ccc4nc(Sc5cccc(Oc6ccn(C)n6)c5Cl)ncc4n3)CC2)[C@@H]1N]

Compound 95 (282 mg, yield 35%) is obtained in a similar manner to Embodiment 63. LC/MS(ESI): m/z=538.2[M+H]⁺.

### Embodiment 96

### Preparation of N-(3-((2-((3S,4S)-4-amino-3-methyl-2-oxa-8-azaspiro[4.5]decane-8-yl)pyridinyl[3,2-d]pyrimidine-6-yl)mercapto)-2-chlorophenyl)-2-hydroxy-4-oxo-6,7,8,9-tetrahydro-4H-pyridinyl[1,2-a]pyrimidine-3-carboxamide

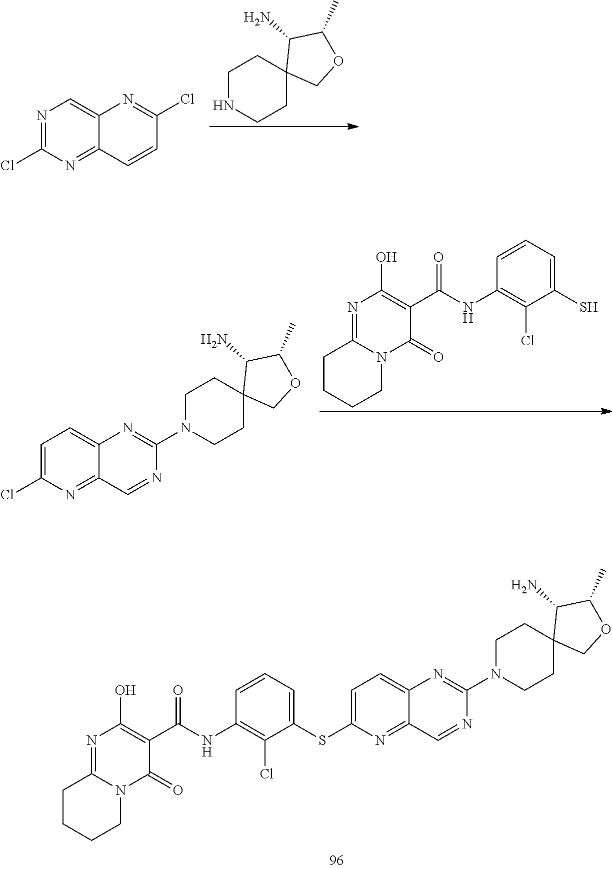

Compound 96 (379 mg, yield 39%) is obtained in a similar manner to Embodiment 64. ¹H NMR (400 MHZ, DMSO-d₆) δ: 12.1 (br s, 1H), 8.43 (s, 1H), 7.94-7.76 (m, 2H), 7.33-7.24 (m, 2H), 6.67 (d, 1H), 5.52 (br s, 3H), 4.13-4.10 (m, 1H), 3.93-3.45 (m, 8H), 2.93 (d, 1H), 2.71 (s, 2H), 1.83-1.35 (m, 8H), 1.14 (d, 3H); LC/MS(ESI): m/z=649.2[M+H]⁺.

### Embodiment 97

### Preparation of N-(3-((6-((3S,4S)-4-amino-3-methyl-2-oxa-8-azaspiro[4.5]decane-8-yl)pyridinyl[2,3-b]pyrazine-2-yl)mercapto)-2-chlorophenyl)-2-hydroxy-4-oxo-6,7,8,9-tetrahydro-4H-p yridinyl[1,2-a]pyrimidine-3-carboxamide

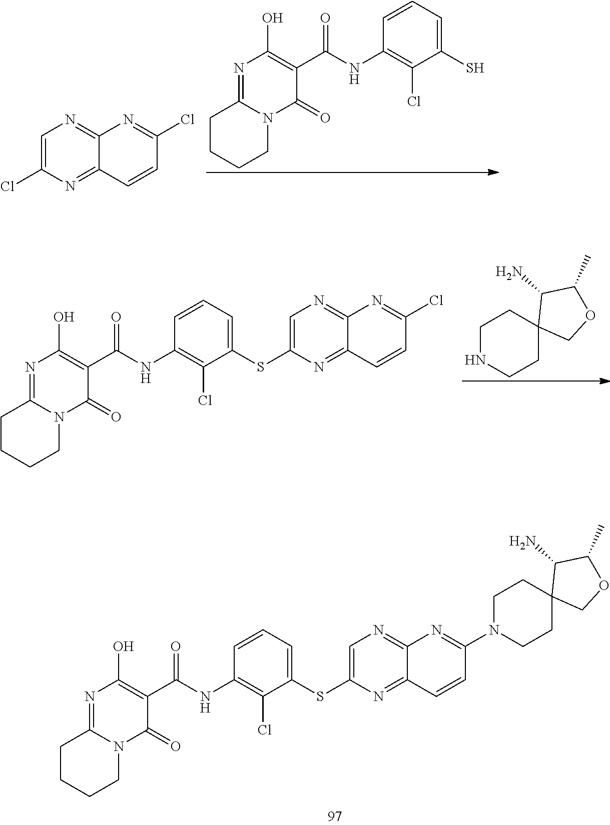

Compound 97 (340 mg, yield 35%) is obtained in a similar manner to Embodiment 63. LC/MS(ESI): m/z=649.2[M+H]⁺.

### Embodiment 98

### Preparation of N-(3-((2-((3S,4S)-4-amino-3-methyl-2-oxa-8-azaspiro[4.5]decane-8-yl)pyridinyl[2,3-b]pyrazine-6-yl)mercapto)-2-chlorophenyl)-2-hydroxy-4-oxo-6,7,8,9-tetrahydro-4H-p yridinyl[1,2-a]pyrimidine-3-carboxamide

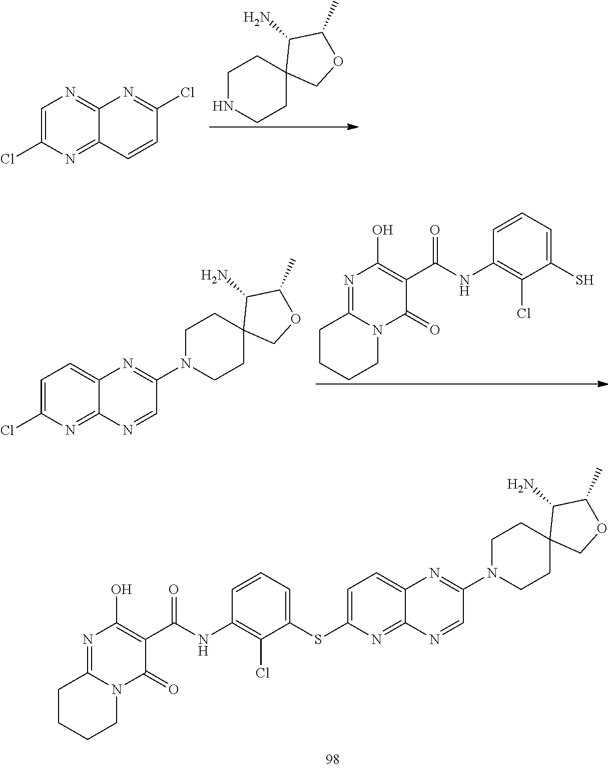

Compound 98 (315 mg, yield 33%) is obtained in a similar manner to Embodiment 64. LC/MS(ESI): m/z=649.2[M+H]⁺.

### Embodiment 99

### Preparation of (3S,4S)-8-(2-((2-chloro-3-(1-methyl-1H-pyrazole-3-yl)phenyl)mercapto)pyrimidine-5-yl)-3-methyl-2-oxa-8-azaspiro[4.5]decane-4-amine

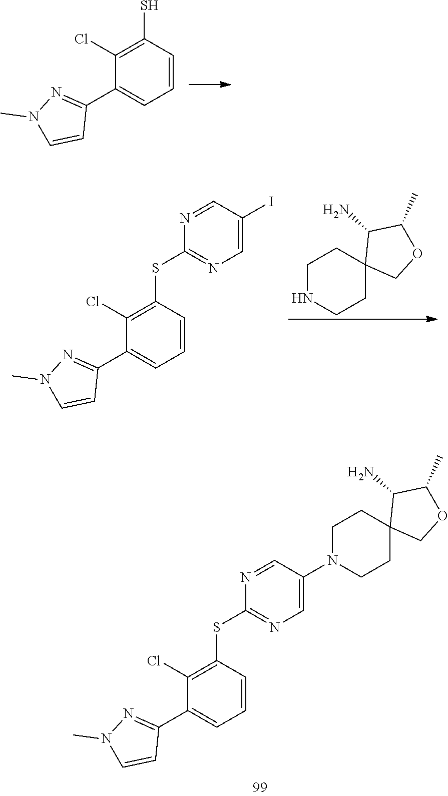 [SMILES: Cn1ccc(-c2cccc(S)c2Cl)n1 ; Cn1ccc(-c2cccc(Sc3ncc(I)cn3)c2Cl)n1 ; C[C@@H]1OCC2(CCNCC2)[C@@H]1N ; C[C@@H]1OCC2(CCN(c3cnc(Sc4cccc(-c5ccn(C)n5)c4Cl)nc3)CC2)[C@@H]1N]

Dissolve compound 3-(1-methyl-1H-pyrazole-3-yl)-2-chloro-thiophenol (674 mg, 3 mmol) in 8 mL of ethanol, add 2-chloro-iodopyrimidine (864 mg, 3.6 mmol) and sodium ethoxide (408 mg, 6.0 mmol). Stir the reaction mixture and reflux overnight. After cooling to room temperature, the reaction solution is diluted with water and extracted with ethyl acetate. The obtained organic phase is washed with water and saturated brine, dried with anhydrous sodium sulfate, and the organic phase is evaporated to dryness under reduced pressure to give the intermediate 2-((2-chloro-3-(1-methyl-1H-pyrazole-3-yl)phenyl)mercapto)-5-iodopyrimidine (304 mg, 43% yield). LC/MS(ESI): m/z=471.2[M+H]⁺.

Dissolve the compound 2-((2-chloro-3-(1-methyl-1H-pyrazole-3-yl)phenyl) mercapto)-5-iodopyrimidine (506 mg, 1.5 mmol) in 10 mL of N,N-dimethylformamide, add (3S,4S)-4-amino-3-methyl-2-oxa-8-azaspiro[4.5]decane dihydrochloride (438 mg, 1.8 mmol) and potassium carbonate (829 mg, 6.0 mmol), stir at 120° ° C. for 6 hours. After cooling to room temperature, the reaction solution is diluted with water and extracted with ethyl acetate. The obtained organic phase is washed with water and saturated brine, dried with anhydrous sodium sulfate, and the organic phase is evaporated to dryness under reduced pressure. The residue is purified by column chromatography to give the target product 99 (304 mg, 43% yield). 1H NMR (400 MHZ, DMSO-d₆) δ: 8.31 (s, 2H), 7.78 (d, 1H), 7.51 (d, 1H), 7.23 (t, 1H), 6.83 (d, 1H), 6.60 (d, 1H), 4.13-4.09 (m, 1H), 3.92-3.75 (m, 5H), 3.65 (d, 1H), 3.47 (d, 1H), 3.32-3.25 (m, 2H), 2.90 (d, 1H), 1.77-1.32 (m, 6H), 1.13 (d, 3H); LC/MS(ESI): m/z=471.2[M+H]⁺.

### Embodiment 100

### Preparation of (3S,4S)-8-(2-((2-chloro-3-(1-ethyl-1H-pyrazole-3-yl)phenyl)mercapto)pyrimidine-5-y l)-3-methyl-2-oxa-8-azaspiro[4.5]decane-4-amine

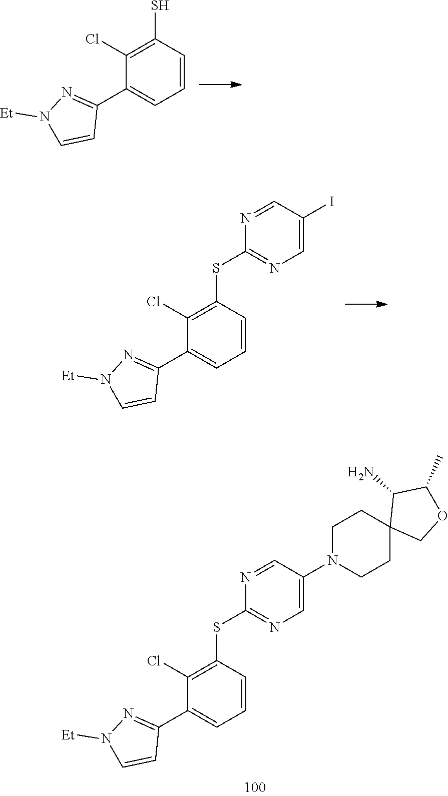 [SMILES: CCn1ccc(-c2cccc(S)c2Cl)n1 ; CCn1ccc(-c2cccc(Sc3ncc(I)cn3)c2Cl)n1 ; CCn1ccc(-c2cccc(Sc3ncc(N4CCC5(CC4)CO[C@@H](C)[C@H]5N)cn3)c2Cl)n1]

Compound 100 (327 mg, 45% yield, which is the yield of the last step, the same below) is obtained by a method similar to that of Embodiment 99. ¹H NMR (400 MHZ, DMSO-d₆) δ: 8.31 (s, 2H), 7.72 (d, 1H), 7.51 (d, 1H), 7.23 (t, 1H), 6.83 (d, 1H), 6.58 (d, 1H), 4.13-4.09 (m, 1H), 3.94-3.77 (m, 5H), 3.65 (d, 1H), 3.47 (d, 1H), 3.32-3.25 (m, 2H), 2.90 (d, 1H), 1.77-1.33 (m, 6H), 1.28 (t, 3H), 1.13 (d, 3H); LC/MS(ESI): m/z=485.2[M+H]⁺.

### Embodiment 101

### Preparation of (3S,4S)-8-(2-((2-chloro-3-(1-isopropyl-1H-pyrazole-3-yl)phenyl)mercapto)pyrimidine-5-yl)-3-methyl-2-oxa-8-azaspiro[4.5]decane-4-amine

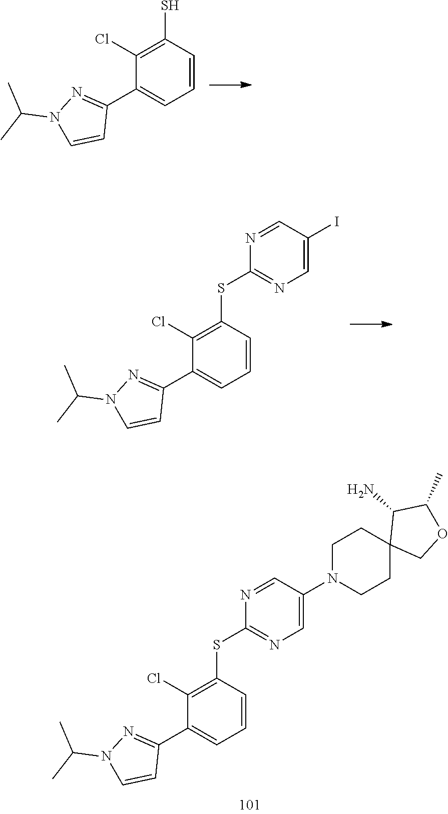 [SMILES: CC(C)n1ccc(-c2cccc(S)c2Cl)n1 ; CC(C)n1ccc(-c2cccc(Sc3ncc(I)cn3)c2Cl)n1 ; CC(C)n1ccc(-c2cccc(Sc3ncc(N4CCC5(CC4)CO[C@@H](C)[C@H]5N)cn3)c2Cl)n1]

Compound 101 (381 mg, yield 51%) is obtained in a similar manner to Embodiment 99. ¹H NMR (400 MHZ, DMSO-d₆) δ: 8.31 (s, 2H), 7.70 (s, 1H), 7.51 (d, 1H), 7.23 (t, 1H), 6.83 (d, 1H), 6.54 (d, 1H), 4.14-4.07 (m, 2H), 3.92-3.75 (m, 5H), 3.65 (d, 1H), 3.47 (d, 1H), 3.32-3.25 (m, 2H), 2.90 (d, 1H), 1.77-1.32 (m, 12H), 1.13 (d, 3H); LC/MS(ESI): m/z=499.2[M+H]⁺.

### Embodiment 102

### Preparation of N-(3-((5-((3S,4S)-4-amino-3-methyl-2-oxa-8-azaspiro[4.5]decane-8-yl)pyrimidine-2-y l)mercapto)-2-chlorobenzene)-2-hydroxyl-4-oxa-6,7,8,9-tetrahydro-4H-pyridinyl[1,2-a]pyrimidine-3-carboxamide

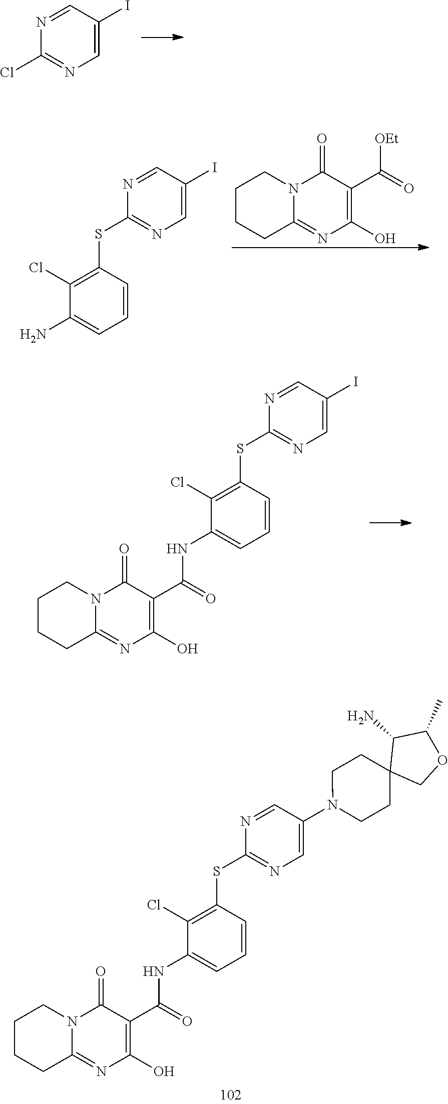

Dissolve the compound 3-amino-2-chloro-thiophenol (3.19 g, 20 mmol) in 60 mL of dimethyl sulfoxide, add 2-chloro-5-iodopyrimidine (4.8 g, 20 mmol) and carbonic acid Cesium (13.0 g, 40 mmol), heat to 80° C. and stir for 6 hours. After cooling to room temperature, the reaction solution is diluted with water and extracted with ethyl acetate. The obtained organic phase is washed with water and saturated brine, dried with anhydrous sodium sulfate, and the organic phase is evaporated to dryness under reduced pressure. The residue is purified by column chromatography to give the compound 2-chloro-3-((5-iodopyrimidine-2-yl)mercapto)aniline (2.45 g, 45% yield). LC/MS(ESI): m/z=272.0[M+H]⁺.

Dissolve compound 2-hydroxy-4-oxo-pyridinyl[1,2-a]pyrimidine-3-carboxylic acid ethyl ester (408 mg, 1.5 mmol) and compound 2-chloro-3-((5-iodopyrimidine-2-yl) mercapto)aniline (429 mg, 1.8 mmol) in 5 mL of chlorobenzene, heat to 130° C. and stir for 5 hours, then cool to room temperature, filter. After drying, compound N-(2-chloro-3-((5-iodopyrimidine-2-yl)mercapto)phenyl)-2-hydroxyl-4-oxa-6,7,8,9-tetrahydro-4H-pyridinyl[1,2-a]pyrimidine-3-carboxamide is obtained(474 mg, 68% yield). LC/MS(ESI): m/z=464.0[M+H]⁺.

Dissolve the compound N-(2-chloro-3-((5-iodopyrimidine-2-yl)mercapto)phenyl)-2-hydroxyl-4-oxa-6,7,8,9-tetrah ydro-4H-pyridinyl[1,2-a]pyrimidine-3-carboxamide (464 mg, 1 mmol) in 4 mL of N,N-dimethylformamide, add (3S,4S)-4-amino-3-methyl-2-oxa-8-azaspiro[4.5]decane dihydrochloride (292 mg, 1.2 mmol) and potassium carbonate (553 mg, 4.0 mmol), stir at 120° C. for 8 hours. After cooling to room temperature, the reaction solution is diluted with water and extracted with ethyl acetate. The obtained organic phase is washed with water and saturated brine, dried with anhydrous sodium sulfate, and the organic phase is evaporated to dryness under reduced pressure. The residue is then purified by column chromatography to give the target product 102 (191 mg, 32% yield). 1H NMR (400 MHZ, DMSO-d₆) δ: 12.5 (br s, 1H), 8.35-8.23 (m, 3H), 7.23 (s, 1H), 6.58 (d, 1H), 5.54 (br s, 3H), 4.13-4.09 (m, 1H), 3.92-3.55 (m, 7H), 3.32-3.25 (m, 3H), 2.92 (d, 1H), 1.82-1.43 (m, 8H), 1.13 (d, 3H); LC/MS(ESI): m/z=598.2[M+H]⁺.

### Embodiment 103

### Preparation of (3S,4S)-8-(2-((2-chloro-3-(pyrimidine-5-yl)phenyl)mercapto)pyrimidine-5-yl)-3-meth yl-2-oxa-8-azaspiro[4.5]decane-4-amine

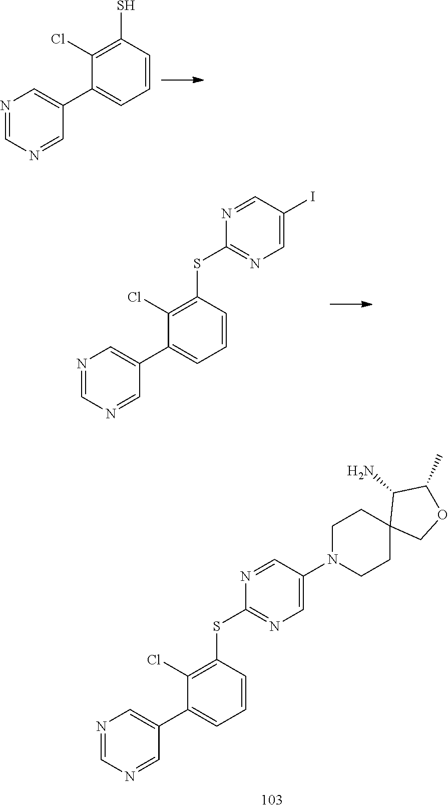 [SMILES: Sc1cccc(-c2cncnc2)c1Cl ; Clc1c(Sc2ncc(I)cn2)cccc1-c1cncnc1 ; C[C@@H]1OCC2(CCN(c3cnc(Sc4cccc(-c5cncnc5)c4Cl)nc3)CC2)[C@@H]1N]

Compound 103 (205 mg, yield 38%) is obtained in a similar manner to Embodiment 99. ¹H NMR (400 MHZ, DMSO-d₆) δ: 9.22 (s, 1H), 8.89 (s, 2H), 8.31 (s, 2H), 7.50 (d, 1H), 7.21 (t, 1H), 6.86 (d, 1H), 4.13-4.09 (m, 1H), 3.94-3.75 (m, 5H), 3.65 (d, 1H), 3.47 (d, 1H), 3.32-3.25 (m, 2H), 2.90 (d, 1H), 1.77-1.33 (m, 3H), 1.13 (d, 3H); LC/MS(ESI): m/z=469.1 [M+H]⁺.

### Embodiment 104

### Preparation of (3S,4S)-8-(2-((2-chloro-3-(pyrazine-2-yl)phenyl)mercapto)pyrimidine-5-yl)-3-methyl-2-oxa-8-azaspiro[4.5]decane-4-amine

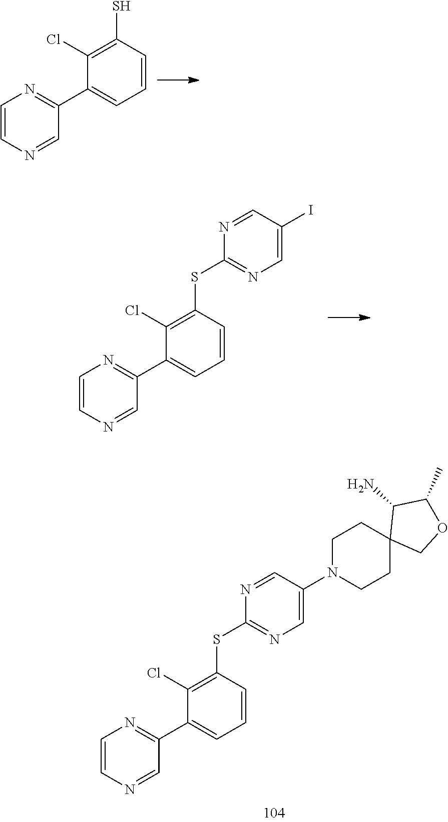 [SMILES: Sc1cccc(-c2cnccn2)c1Cl ; Clc1c(Sc2ncc(I)cn2)cccc1-c1cnccn1 ; C[C@@H]1OCC2(CCN(c3cnc(Sc4cccc(-c5cnccn5)c4Cl)nc3)CC2)[C@@H]1N]

Compound 104 (189 mg, yield 35%) is obtained in a similar manner to Embodiment 102. ¹H NMR (400 MHZ, DMSO-d₆) δ: 8.81 (s, 1H), 8.75 (s, 2H), 8.31 (s, 2H), 7.50 (d, 1H), 7.21 (t, 1H), 6.86 (d, 1H), 4.13-4.09 (m, 1H), 3.94-3.75 (m, 5H), 3.65 (d, 1H), 3.47 (d, 1H), 3.32-3.25 (m, 2H), 2.90 (d, 1H), 1.77-1.33 (m, 3H), 1.13 (d, 3H); LC/MS(ESI): m/z=469.1 [M+H]⁺.

### Embodiment 105

### Preparation of (3S,4S)-8-(2-((2-chloro-3-(pyridine-3-yl)phenyl)mercapto)pyrimidine-5-yl)-3-methyl-2-oxa-8-azaspiro[4.5]decane-4-amine

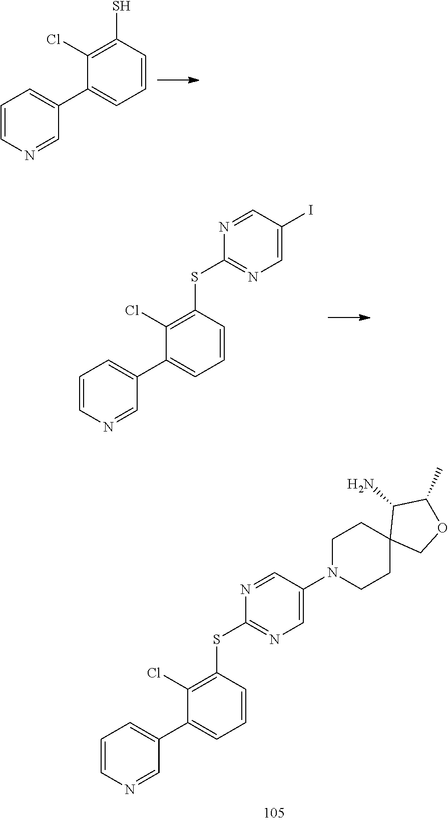 [SMILES: Sc1cccc(-c2cccnc2)c1Cl ; Clc1c(Sc2ncc(I)cn2)cccc1-c1cccnc1 ; C[C@@H]1OCC2(CCN(c3cnc(Sc4cccc(-c5cccnc5)c4Cl)nc3)CC2)[C@@H]1N]

Compound 105 (327 mg, yield 42%) is obtained in a similar manner to Embodiment 93. ¹H NMR (400 MHZ, DMSO-d₆) δ: 8.85 (s, 1H), 8.70 (d, 1H), 8.32-8.30 (m, 3H), 7.53-7.50 (m, 2H), 7.21 (t, 1H), 6.86 (d, 1H), 4.13-4.09 (m, 1H), 3.94-3.75 (m, 5H), 3.65 (d, 1H), 3.47 (d, 1H), 3.32-3.25 (m, 2H), 2.90 (d, 1H), 1.77-1.33 (m, 3H), 1.13 (d, 3H); LC/MS(ESI): m/z=468.2[M+H]⁺.

### Embodiment 106

### Preparation of (3S,4S)-8-(2-((2-chloro-3-(pyrimidine-4-yl)phenyl)mercapto)pyrimidine-5-yl)-3-meth yl-2-oxa-8-azaspiro[4.5]decane-4-amine

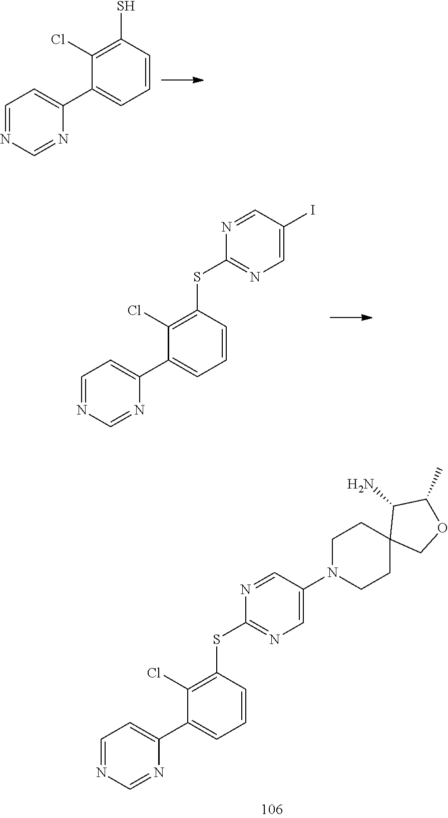 [SMILES: Sc1cccc(-c2ccncn2)c1Cl ; Clc1c(Sc2ncc(I)cn2)cccc1-c1ccncn1 ; C[C@@H]1OCC2(CCN(c3cnc(Sc4cccc(-c5ccncn5)c4Cl)nc3)CC2)[C@@H]1N]

Compound 106 (216 mg, yield 40%) is obtained in a similar manner to Embodiment 102. ¹H NMR (400 MHZ, DMSO-d₆) δ: 9.23 (s, 1H), 8.85 (d, 1H), 8.31 (s, 2H), 8.12 (d, 1H), 7.50 (d, 1H), 7.21 (t, 1H), 6.86 (d, 1H), 4.13-4.09 (m, 1H), 3.94-3.75 (m, 5H), 3.65 (d, 1H), 3.47 (d, 1H), 3.32-3.25 (m, 2H), 2.90 (d, 1H), 1.77-1.33 (m, 3H), 1.13 (d, 3H); LC/MS(ESI): m/z=469.1[M+H]⁺.

### Embodiment 107

### Preparation of (3S,4S)-8-(2-((2-chloro-3-(pyridine-3-amino)phenyl)mercapto)pyrimidine-5-yl)-3-me thyl-2-oxa-8-azaspiro[4.5]decane-4-amine

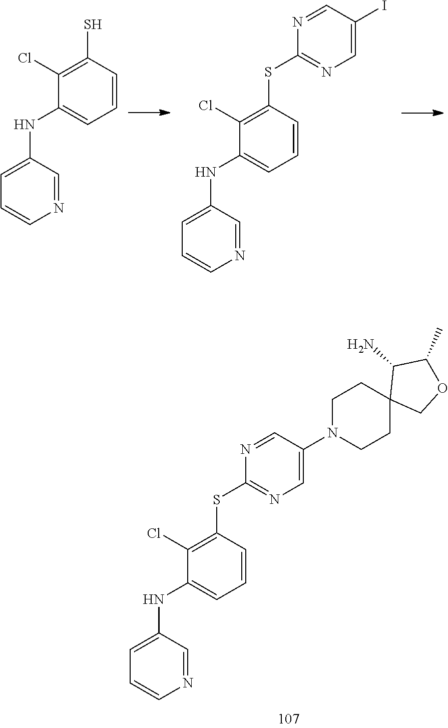 [SMILES: Sc1cccc(Nc2cccnc2)c1Cl ; Clc1c(Nc2cccnc2)cccc1Sc1ncc(I)cn1 ; C[C@@H]1OCC2(CCN(c3cnc(Sc4cccc(Nc5cccnc5)c4Cl)nc3)CC2)[C@@H]1N]

Dissolve compound 3-(pyridine-4-amino)-2-chloro-thiophenol (592 mg, 2.5 mmol) in 6 mL of methanol, add 2-chloro-5-iodopyrimidine (720 mg, 3 mmol) and potassium carbonate (830 mg, 6.0 mmol), stir and reflux for 5 hours. After cooling to room temperature, the reaction solution is evaporated to dryness under reduced pressure. The residue is purified by column chromatography to give the compound N-(2-chloro-3-((5-iodopyrimidine-2-yl) mercapto) phenyl) pyridine-3-amine (463 mg, 53% yield). LC/MS(ESI): m/z=349.0[M+H]⁺.

Dissolve compound N-(2-chloro-3-((5-iodopyrimidine-2-yl)mercapto)phenyl)pyridine-3-amine(419 mg, 1.2 mmol) in 5 mL of N-methylpyrrolidone, add (3S,4S)-4-amino-3-methyl-2-oxa-8-azaspiro[4.5]decane dihydrochloride (365 mg, 1.5 mmol) and triethylamine (486 mg, 4.8 mmol), stir and react overnight at 120° C. After cooling to room temperature, the reaction solution is diluted with water and extracted with ethyl acetate. The obtained organic phase is washed with water and saturated brine, dried with anhydrous sodium sulfate, and the organic phase is evaporated to dryness under reduced pressure. The residue is then purified by column chromatography to give the target product 107 (203 mg, 35% yield). 1H NMR (400 MHZ, DMSO-d₆) δ: 8.43 (s, 1H), 8.32-8.25 (m, 3H), 7.63-7.03 (m, 5H), 6.05 (s, 1H), 4.13-4.09 (m, 1H), 3.94-3.75 (m, 5H), 3.65 (d, 1H), 3.46 (d, 1H), 3.33-3.25 (m, 2H), 2.90 (d, 1H), 1.77-1.34 (m, 3H), 1.13 (d, 3H); LC/MS(ESI): m/z=483.2 [M+H]⁺.

### Embodiment 108

### Preparation of (3S,4S)-8-(2-((2-chloro-3-(pyrimidine-2-amino)phenyl)mercapto)pyrimidine-5-yl)-3-methyl-2-oxa-8-azaspiro[4.5]decane-4-amine

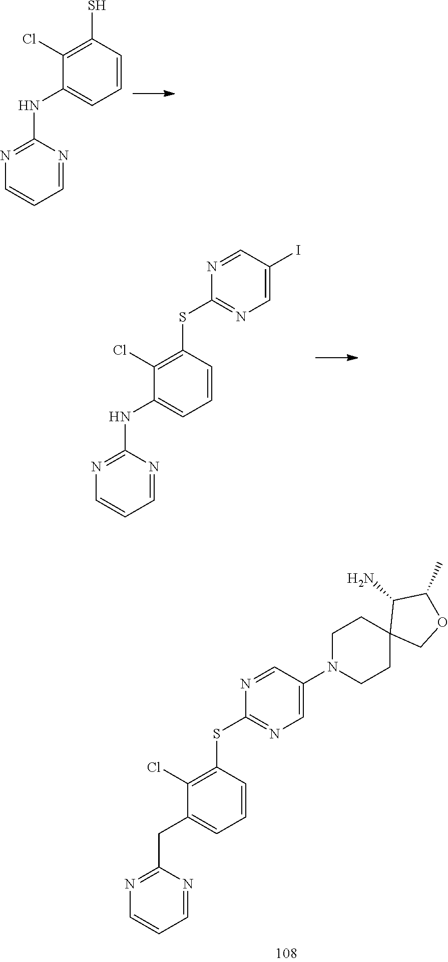 [SMILES: Sc1cccc(Nc2ncccn2)c1Cl ; Clc1c(Nc2ncccn2)cccc1Sc1ncc(I)cn1 ; C[C@@H]1OCC2(CCN(c3cnc(Sc4cccc(Cc5ncccn5)c4Cl)nc3)CC2)[C@@H]1N]

Compound 108 (186 mg, yield 32%) is obtained in a similar manner to Embodiment 107. ¹H NMR (400 MHZ, DMSO-d₆) δ: 8.45 (d, 2H), 8.30-8.22 (m, 3H), 7.52-7.03 (m, 3H), 6.17 (s, 1H), 4.14-4.09 (m, 1H), 3.95-3.75 (m, 5H), 3.65 (d, 1H), 3.46 (d, 1H), 3.33-3.25 (m, 2H), 2.92 (d, 1H), 1.77-1.34 (m, 3H), 1.13 (d, 3H); LC/MS(ESI): m/z=484.2 [M+H]⁺.

### Embodiment 109

### Preparation of (3S,4S)-8-(2-((2-chloro-3-(pyrazine-2-amino)phenyl)mercapto)pyrimidine-5-yl)-3-me thyl-2-oxa-8-azaspiro[4.5]decane-4-amine

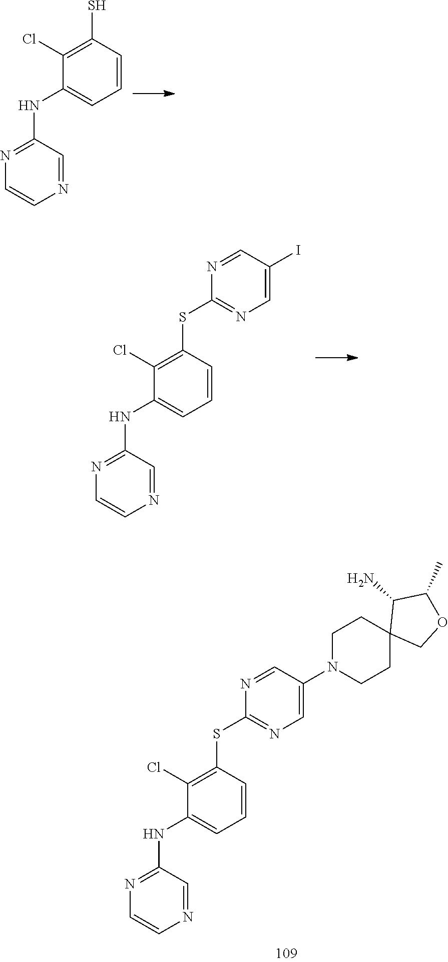 [SMILES: Sc1cccc(Nc2cnccn2)c1Cl ; Clc1c(Nc2cnccn2)cccc1Sc1ncc(I)cn1 ; C[C@@H]1OCC2(CCN(c3cnc(Sc4cccc(Nc5cnccn5)c4Cl)nc3)CC2)[C@@H]1N]

Compound 109 (174 mg, yield 30%) is obtained in a similar manner to Embodiment 107. ¹H NMR (400 MHZ, DMSO-d₆) δ: 8.40-8.35 (m, 3H), 8.28 (s, 2H), 7.52-7.09 (m, 3H), 6.23 (s, 1H), 4.14-4.09 (m, 1H), 3.92-3.75 (m, 5H), 3.64 (d, 1H), 3.46 (d, 1H), 3.35-3.25 (m, 2H), 2.92 (d, 1H), 1.77-1.34 (m, 3H), 1.13 (d, 3H); LC/MS(ESI): m/z=484.2 [M+H]⁺.

### Embodiment 110

### Preparation of (3S,4S)-8-(2-((2-chloro-3-(pyrimidine-4-amino)phenyl)mercapto)pyrimidine-5-yl)-3-methyl-2-oxa-8-azaspiro[4.5]decane-4-amine

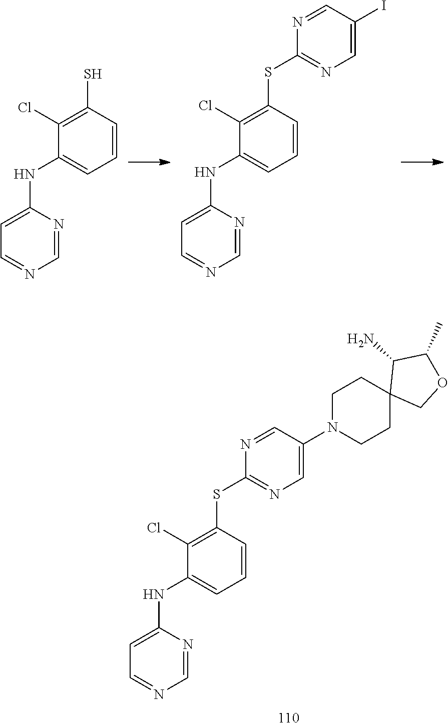 [SMILES: Sc1cccc(Nc2ccncn2)c1Cl ; Clc1c(Nc2ccncn2)cccc1Sc1ncc(I)cn1 ; C[C@@H]1OCC2(CCN(c3cnc(Sc4cccc(Nc5ccncn5)c4Cl)nc3)CC2)[C@@H]1N]

Compound 110 (186 mg, yield 32%) is obtained in a similar manner to Embodiment 107. ¹H NMR (400 MHZ, DMSO-d₆) δ: 8.43 (s, 1H), 8.40 (d, 1H), 8.29 (s, 2H), 7.52-6.93 (m, 4H), 6.05 (s, 1H), 4.13-4.09 (m, 1H), 3.94-3.75 (m, 5H), 3.65 (d, 1H), 3.46 (d, 1H), 3.33-3.24 (m, 2H), 2.88 (d, 1H), 1.79-1.34 (m, 3H), 1.13 (d, 3H); LC/MS(ESI): m/z=484.2 [M+H]⁺.

### Embodiment 111

### Preparation of (3S,4S)-8-(5-((2-chloro-3-(1-methyl-1H-pyrazole-3-yl)phenyl)mercapto)pyrimidine-2-yl)-3-methyl-2-oxa-8-azaspiro[4.5]decane-4-amine

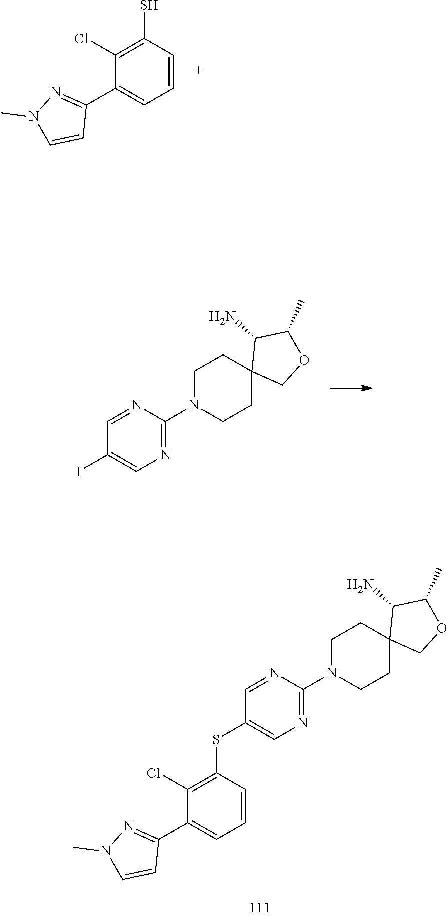 [SMILES: Cn1ccc(-c2cccc(S)c2Cl)n1 ; C[C@@H]1OCC2(CCN(c3ncc(I)cn3)CC2)[C@@H]1N ; C[C@@H]1OCC2(CCN(c3ncc(Sc4cccc(-c5ccn(C)n5)c4Cl)cn3)CC2)[C@@H]1N]

Dissolve compound (3S,4S)-8-(5-chloropyrimidine-2-yl)-3-methyl-2-oxa-8-azaspiro[4.5]decane-4-amine (1.13 g, 4 mmol) in 10 mL of 1,4-dioxane, add compound 3-(1-methyl-1H-pyrazole-3-yl)-2-chloro-thiophenol (1.08 g, 4.8 mmol), potassium tert-butoxide (0.67 g, 6.0 mmol), and cuprous iodide (76 mg, 0.4 mmol). Perform nitrogen substitution 3 times. Stir the reaction mixture and reflux for 24 hours. After cooling to room temperature, the reaction solution is passed through a silica gel short column, rinse the column with ethyl acetate, and evaporate the solution to dryness under reduced pressure. The residue is purified by column chromatography to give compound 111 (810 mg, 43% yield). 1H NMR (400 MHZ, DMSO-d₆) δ: 8.33 (s, 2H), 7.77 (d, 1H), 7.51 (d, 1H), 7.23 (t, 1H), 6.83 (d, 1H), 6.58 (d, 1H), 4.13-4.09 (m, 1H), 3.94-3.75 (m, 5H), 3.63 (d, 1H), 3.47 (d, 1H), 3.34-3.25 (m, 2H), 2.92 (d, 1H), 1.77-1.32 (m, 6H), 1.15 (d, 3H); LC/MS(ESI): m/z=471.2[M+H]⁺.

### Embodiment 112

### Preparation of (3S,4S)-8-(5-((2-chloro-3-(1-ethyl-1H-pyrazole-3-yl)phenyl)mercapto)pyrimidine-2-y l)-3-methyl-2-oxa-8-azaspiro[4.5]decane-4-amine

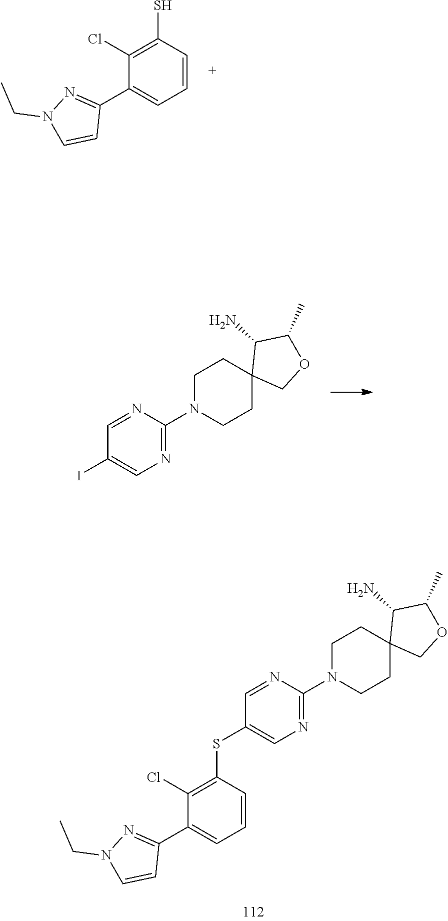 [SMILES: CCn1ccc(-c2cccc(S)c2Cl)n1 ; C[C@@H]1OCC2(CCN(c3ncc(I)cn3)CC2)[C@@H]1N ; CCn1ccc(-c2cccc(Sc3cnc(N4CCC5(CC4)CO[C@@H](C)[C@H]5N)nc3)c2Cl)n1]

Compound 112 (912 mg, yield 47%) is obtained in a similar manner to Embodiment 111. ¹H NMR (400 MHZ, DMSO-d₆) δ: 8.32 (s, 2H), 7.77 (d, 1H), 7.52 (d, 1H), 7.23 (t, 1H), 6.85 (d, 1H), 6.57 (d, 1H), 4.13-4.09 (m, 1H), 3.98-3.77 (m, 5H), 3.65 (d, 1H), 3.49 (d, 1H), 3.34-3.25 (m, 2H), 2.90 (d, 1H), 1.78-1.33 (m, 6H), 1.28 (t, 3H), 1.15 (d, 3H); LC/MS(ESI): m/z=485.2[M+H]⁺.

### Embodiment 113

### Preparation of (3S,4S)-8-(5-((2-chloro-3-(1-isopropyl-1H-pyrazole-3-yl)phenyl)mercapto)pyrimidine-2-yl)-3-methyl-2-oxa-8-azaspiro[4.5]decane-4-amine

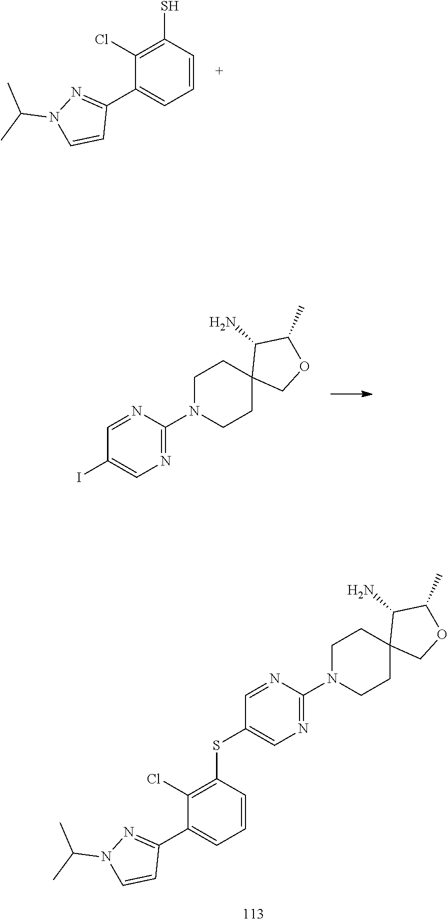 [SMILES: CC(C)n1ccc(-c2cccc(S)c2Cl)n1 ; C[C@@H]1OCC2(CCN(c3ncc(I)cn3)CC2)[C@@H]1N ; CC(C)n1ccc(-c2cccc(Sc3cnc(N4CCC5(CC4)CO[C@@H](C)[C@H]5N)nc3)c2Cl)n1]

Compound 113 (799 mg, yield 40%) is obtained in a similar manner to Embodiment 111. ¹H NMR (400 MHZ, DMSO-d₆) δ: 8.32 (s, 2H), 7.78 (s, 1H), 7.51 (d, 1H), 7.23 (t, 1H), 6.83 (d, 1H), 6.57 (d, 1H), 4.14-4.07 (m, 2H), 3.97-3.74 (m, 5H), 3.64 (d, 1H), 3.47 (d, 1H), 3.34-3.25 (m, 2H), 2.90 (d, 1H), 1.79-1.32 (m, 12H), 1.15 (d, 3H); LC/MS(ESI): m/z=499.2[M+H]⁺.

### Embodiment 114

### Preparation of N-(3-((2-((3S,4S)-4-amino-3-methyl-2-oxa-8-azaspiro[4.5]decane-8-yl)pyrimidine-5-y l)mercapto)-2-chlorophenyl)-2-hydroxyl-4-oxo-6,7,8,9-tetrahydro-4H-pyridinyl[1,2-a]pyrimidine-3-carboxamide

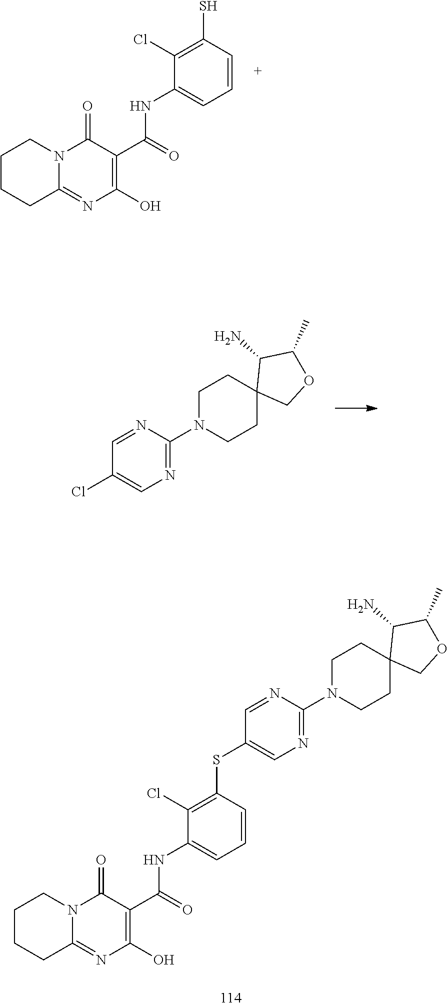 [SMILES: O=C(Nc1cccc(S)c1Cl)c1c(O)nc2n(c1=O)CCCC2 ; C[C@@H]1OCC2(CCN(c3ncc(Cl)cn3)CC2)[C@@H]1N ; C[C@@H]1OCC2(CCN(c3ncc(Sc4cccc(NC(=O)c5c(O)nc6n(c5=O)CCCC6)c4Cl)cn3)CC2)[C@@H]1N]

Dissolve compound (3S,4S)-8-(5-chloropyrimidine-2-yl)-3-methyl-2-oxa-8-azaspiro[4.5]decane-4-amine (565 mg, 2 mmol) in 6 mL of 1,4-dioxane, add the compound N-(2-chloro-3-mercaptophenyl)-2-hydroxyl-4-oxo-6,7,8,9-tetrahydro-4H-pyridinyl[1,2-a]pyrimidine-3-carboxamide (504 mg, 2.4 mmol), potassium tert-butoxide (335 mg, 3 mmol), and cuprous iodide (38 mg, 0.2 mmol). Perform nitrogen substitution 3 times. Stir the mixture and reflux for 24 hours. The reaction mixture is refluxed and stirred for 24 hours. After cooling to room temperature, the reaction solution is passed through a silica gel short column, rinse the column with ethyl acetate, and evaporate the solution to dryness under reduced pressure. The residue is purified by column chromatography to give compound 114 (538 mg, yield 45%). 1H NMR (400 MHz, DMSO-d₆) δ: 12.7(br s, 1H), 8.35-8.23 (m, 3H), 7.24 (s, 1H), 6.59 (d, 1H), 5.52 (br s, 3H), 4.13-4.09 (m, 1H), 3.92-3.55 (m, 7H), 3.32-3.25 (m, 3H), 2.93 (d, 1H), 1.87-1.35 (m, 8H), 1.15 (d, 3H); LC/MS(ESI): m/z=598.2[M+H]⁺.

### Embodiment 115

### Preparation of (3S,4S)-8-(5-((2-chloro-3-(pyrimidine-5-yl)phenyl)mercapto)pyrimidine-2-yl)-3-meth yl-2-oxa-8-azaspiro[4.5]decane-4-amine

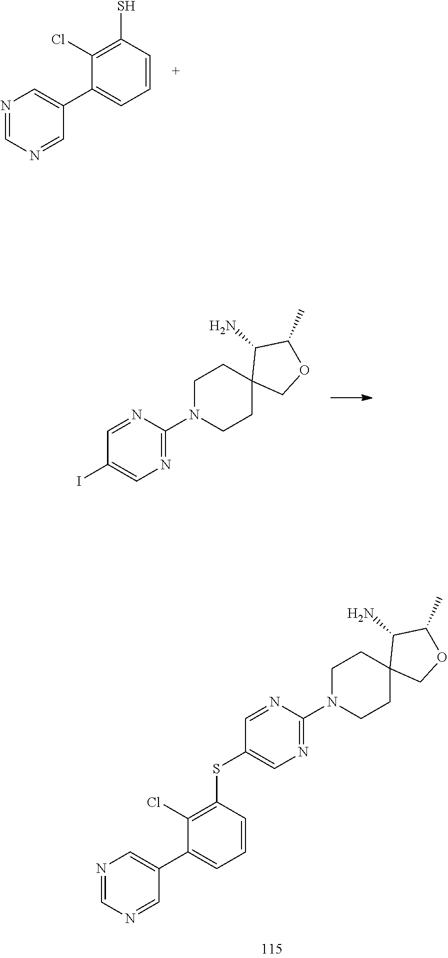 [SMILES: Sc1cccc(-c2cncnc2)c1Cl ; C[C@@H]1OCC2(CCN(c3ncc(I)cn3)CC2)[C@@H]1N ; C[C@@H]1OCC2(CCN(c3ncc(Sc4cccc(-c5cncnc5)c4Cl)cn3)CC2)[C@@H]1N]

Compound 115 (441 mg, yield 47%) is obtained in a similar manner to the last step of Embodiment 114. ¹H NMR (400 MHZ, DMSO-d₆) δ: 9. 22 (s, 1H), 8. 90 (s, 2H), 8. 33 (s, 2H), 7.51 (d, 1H), 7.21 (t, 1H), 7. 06 (d, 1H), 4. 13-4.09 (m, 1H), 3.9 4-3.45 (m, 7H), 3.3 4-3.25 (m, 2H), 2.90 (d, 1H), 1. 81-1.33 (m, 3H), 1. 13 (d, 3H); LC/MS(ESI): m/z=469.1[M+H]⁺.

### Embodiment 116

### Preparation of (3S,4S)-8-(5-((2-chloro-3-(pyrazine-2-yl)phenyl)mercapto)pyrimidine-2-yl)-3-methyl-2-oxa-8-azaspiro[4.5]decane-4-amine

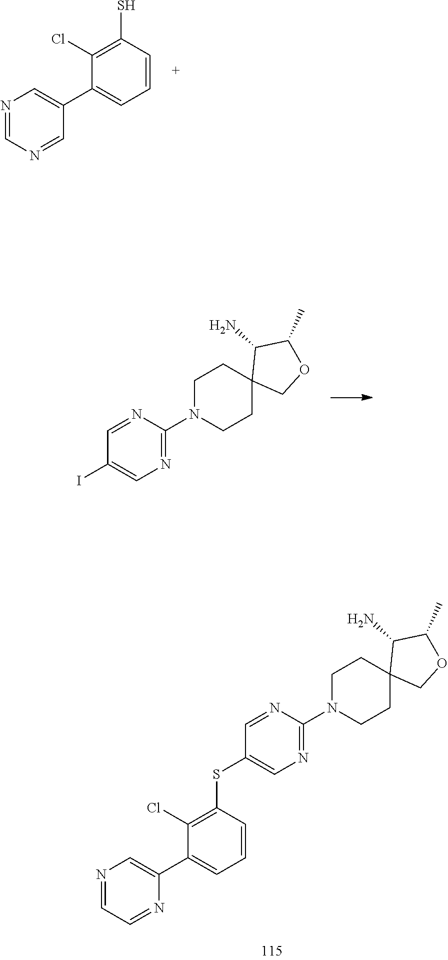 [SMILES: Sc1cccc(-c2cncnc2)c1Cl ; C[C@@H]1OCC2(CCN(c3ncc(I)cn3)CC2)[C@@H]1N ; C[C@@H]1OCC2(CCN(c3ncc(Sc4cccc(-c5cnccn5)c4Cl)cn3)CC2)[C@@H]1N]

Compound 116 (422 mg, yield 45%) is obtained in a similar manner to the last step of Embodiment 114. ¹H NMR (400 MHZ, DMSO-d₆) δ: 8.80 (s, 1H), 8.75 (s, 2H), 8.32 (s, 2H), 7.51 (d, 1H), 7.23 (t, 1H), 7.05 (d, 1H), 4.13-4.09 (m, 1H), 3.94-3.43 (m, 7H), 3.32-3.23 (m, 2H), 2.91 (d, 1H), 1.79-1.33 (m, 3H), 1.15 (d, 3H); LC/MS(ESI): m/z=469.1[M+H]⁺.

### Embodiment 117

### Preparation of (3S,4S)-8-(5-((2-chloro-3-(pyridine-3-yl)phenyl)mercapto)pyrimidine-2-yl)-3-methyl-2-oxa-8-azaspiro[4.5]decane-4-amine

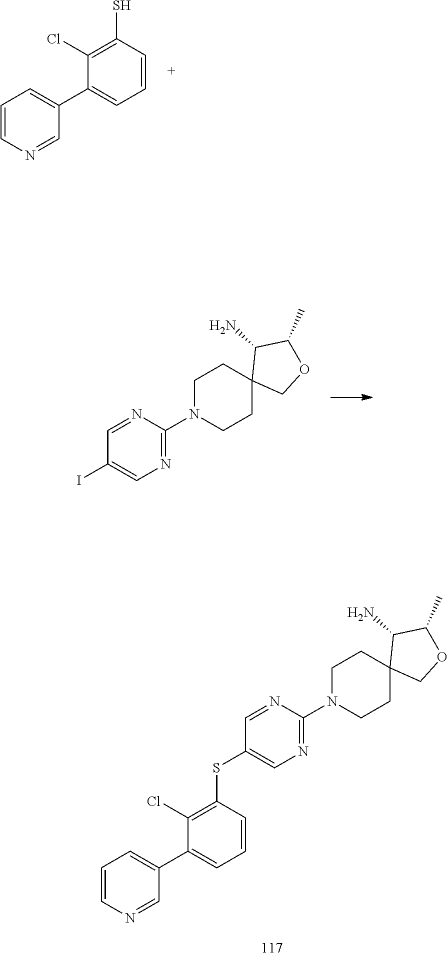 [SMILES: Sc1cccc(-c2cccnc2)c1Cl ; C[C@@H]1OCC2(CCN(c3ncc(I)cn3)CC2)[C@@H]1N ; C[C@@H]1OCC2(CCN(c3ncc(Sc4cccc(-c5cccnc5)c4Cl)cn3)CC2)[C@@H]1N]

Compound 117 (393 mg, yield 42%) is obtained in a similar manner to the last step of Embodiment 114. ¹H NMR (400 MHZ, DMSO-d₆) δ: 8.87 (s, 1H), 8.71 (d, 1H), 8.34-8.30 (m, 3H), 7.55-7.51 (m, 2H), 7.22 (t, 1H), 7.04 (d, 1H), 4.13-4.08 (m, 1H), 3.94-3.45 (m, 7H), 3.31-3.25 (m, 2H), 2.91 (d, 1H), 1.78-1.31 (m, 3H), 1.15 (d, 3H); LC/MS(ESI): m/z=468.2[M+H]⁺.

### Embodiment 118

### Preparation of (3S,4S)-8-(5-((2-chloro-3-(pyrimidine-4-yl)phenyl)mercapto)pyrimidine-2-yl)-3-meth yl-2-oxa-8-azaspiro[4.5]decane-4-amine

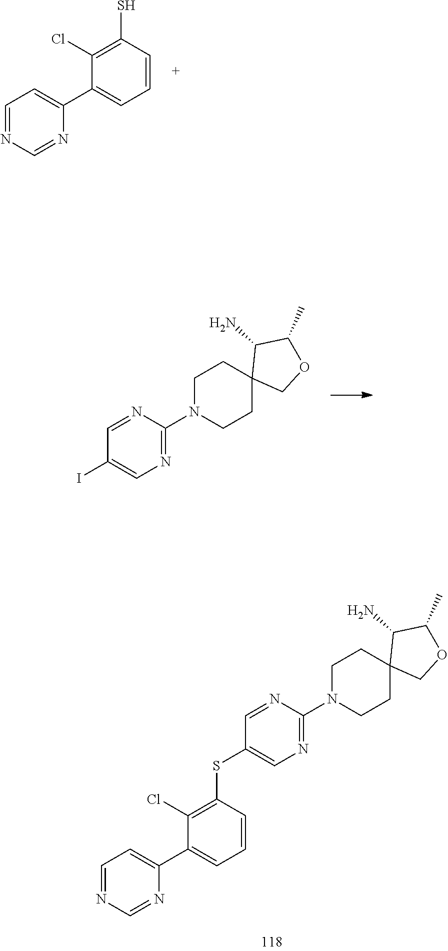 [SMILES: Sc1cccc(-c2ccncn2)c1Cl ; C[C@@H]1OCC2(CCN(c3ncc(I)cn3)CC2)[C@@H]1N ; C[C@@H]1OCC2(CCN(c3ncc(Sc4cccc(-c5ccncn5)c4Cl)cn3)CC2)[C@@H]1N]

Compound 118 (431 mg, yield 46%) is obtained in a similar manner to the last step of Embodiment 114. ¹H NMR (400 MHZ, DMSO-d₆) δ: 9.25 (s, 1H), 8.86 (d, 1H), 8.32 (s, 2H), 8.12 (d, 1H), 7.51 (d, 1H), 7.22 (t, 1H), 7.06 (d, 1H), 4.13-4.10 (m, 1H), 3.94-3.45 (m, 7H), 3.32-3.25 (m, 2H), 2.90 (d, 1H), 1.78-1.31 (m, 3H), 1.15 (d, 3H);

LC/MS(ESI): m/z=469.1 [M+H]⁺.

### Embodiment 119

### Preparation of (3S,4S)-8-(5-((2-chloro-3-(pyridine-3-amino)phenyl)mercapto)pyrimidine-2-yl)-3-me thyl-2-oxa-8-azaspiro[4.5]decane-4-amine

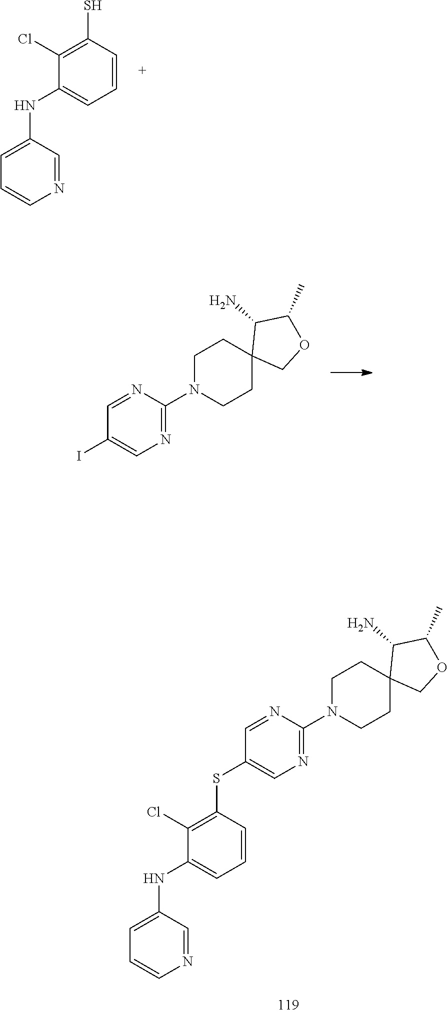 [SMILES: Sc1cccc(Nc2cccnc2)c1Cl ; C[C@@H]1OCC2(CCN(c3ncc(I)cn3)CC2)[C@@H]1N ; C[C@@H]1OCC2(CCN(c3ncc(Sc4cccc(Nc5cccnc5)c4Cl)cn3)CC2)[C@@H]1N]

Compound 119 (347 mg, yield 36%) is obtained in a similar manner to the last step of Embodiment 114. ¹H NMR (400 MHZ, DMSO-d₆) δ: 8.43 (s, 1H), 8.35-8.25 (m, 3H), 7.58-6.89 (m, 5H), 6.11 (s, 1H), 4.13-4.09 (m, 1H), 3.94-3.45 (m, 7H), 3.33-3.25 (m, 2H), 2.91 (d, 1H), 1.76-1.32 (m, 3H), 1.15 (d, 3H); LC/MS(ESI): m/z=483.2 [M+H]⁺.

### Embodiment 120

### Preparation of (3S,4S)-8-(5-((2-chloro-3-(pyrimidine-2-amino)phenyl)mercapto)pyrimidine-2-yl)-3-methyl-2-oxa-8-azaspiro[4.5]decane-4-amine

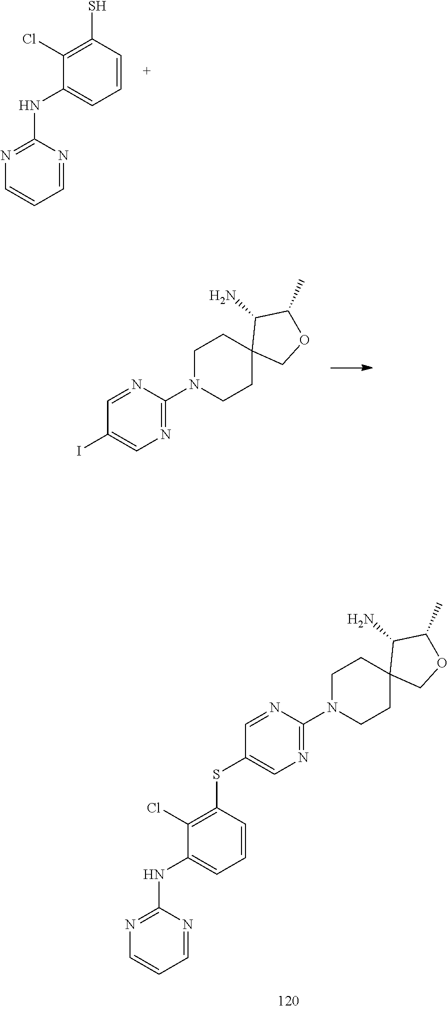 [SMILES: Sc1cccc(Nc2ncccn2)c1Cl ; C[C@@H]1OCC2(CCN(c3ncc(I)cn3)CC2)[C@@H]1N ; C[C@@H]1OCC2(CCN(c3ncc(Sc4cccc(Nc5ncccn5)c4Cl)cn3)CC2)[C@@H]1N]

Compound 120 (386 mg, yield 40%) is obtained in a similar manner to the last step of Embodiment 114. ¹H NMR (400 MHZ, DMSO-d₆) δ: 8.46 (d, 2H), 8.33 (s, 2H), 8.05 (d, 1H), 7.32-6.95 (m, 3H), 6.15 (s, 1H), 4.14-4.10 (m, 1H), 3.95-3.45 (m, 7H), 3.33-3.25 (m, 2H), 2.93 (d, 1H), 1.75-1.31 (m, 3H), 1.15 (d, 3H); LC/MS(ESI): m/z=484.2 [M+H]⁺.

### Embodiment 121

### Preparation of (3S,4S)-8-(5-((2-chloro-3-(pyrazine-2-amino)phenyl)mercapto)pyrimidine-2-yl)-3-me thyl-2-oxa-8-azaspiro[4.5]decane-4-amine

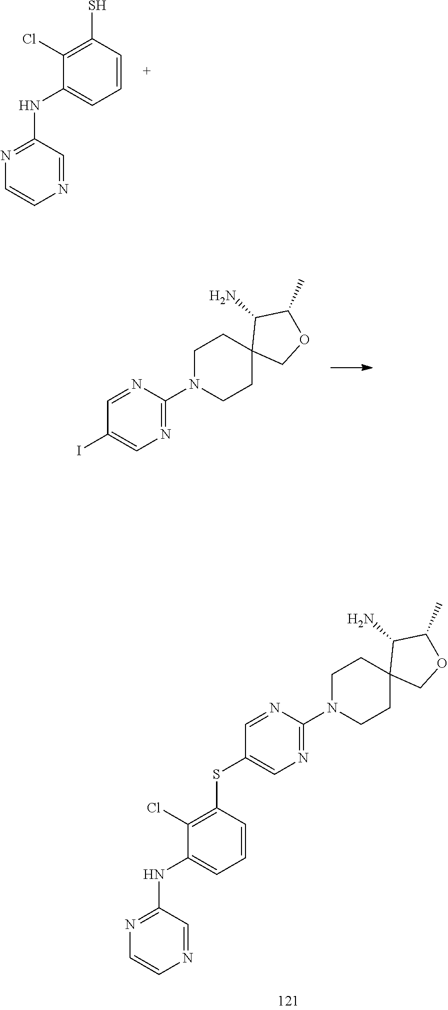 [SMILES: Sc1cccc(Nc2cnccn2)c1Cl ; C[C@@H]1OCC2(CCN(c3ncc(I)cn3)CC2)[C@@H]1N ; C[C@@H]1OCC2(CCN(c3ncc(Sc4cccc(Nc5cnccn5)c4Cl)cn3)CC2)[C@@H]1N]

Compound 121 (405 mg, yield 42%) is obtained in a similar manner to the last step of Embodiment 114. ¹H NMR (400 MHZ, DMSO-d₆) δ: 8.41-8.35 (m, 3H), 8.31 (s, 2H), 7.41-6.94 (m, 3H), 6.21 (s, 1H), 4.14-4.10 (m, 1H), 3.92-3.75 (m, 5H), 3.64 (d, 1H), 3.46 (d, 1H), 3.35-3.25 (m, 2H), 2.92 (d, 1H), 1.75-1.31 (m, 3H), 1.15 (d, 3H); LC/MS(ESI): m/z=484.2 [M+H]⁺.

### Embodiment 122

### Preparation of (3S,4S)-8-(5-((2-chloro-3-(pyrimidine-4-amino)phenyl)mercapto)pyrimidine-2-yl)-3-methyl-2-oxa-8-azaspiro[4.5]decane-4-amine

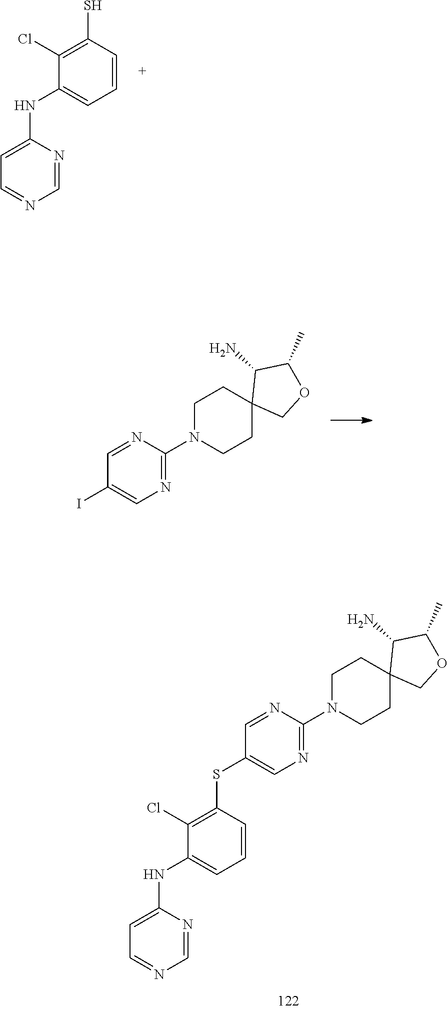 [SMILES: Sc1cccc(Nc2ccncn2)c1Cl ; C[C@@H]1OCC2(CCN(c3ncc(I)cn3)CC2)[C@@H]1N ; C[C@@H]1OCC2(CCN(c3ncc(Sc4cccc(Nc5ccncn5)c4Cl)cn3)CC2)[C@@H]1N]

Compound 122 (424 mg, yield 44%) is obtained in a similar manner to the last step of Embodiment 114. ¹H NMR (400 MHZ, DMSO-d₆) δ: 8.43 (s, 1H), 8.41 (d, 1H), 8.31 (s, 2H), 7.64-6.93 (m, 4H), 6.12 (s, 1H), 4.15-4.11 (m, 1H), 3.93-3.45 (m, 7H), 3.33-3.23 (m, 2H), 2.90 (d, 1H), 1.74-1.31 (m, 3H), 1.15 (d, 3H); LC/MS(ESI): m/z=484.2 [M+H]⁺.

### Embodiment 123

Preparation N-(3-((2-((3S,4S)-4-amino-3-methyl-2-oxa-8-azaspiro[4.5]decane-8-yl I)pyrimidine-5-yl)mercapto)-2-chlorophenyl)pyrimidine-2-carboxamide of

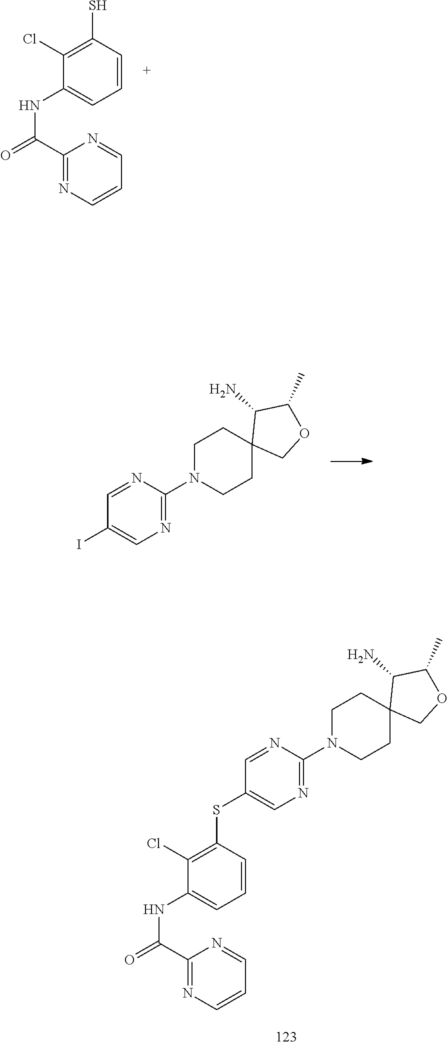 [SMILES: O=C(Nc1cccc(S)c1Cl)c1ncccn1 ; C[C@@H]1OCC2(CCN(c3ncc(I)cn3)CC2)[C@@H]1N ; C[C@@H]1OCC2(CCN(c3ncc(Sc4cccc(NC(=O)c5ncccn5)c4Cl)cn3)CC2)[C@@H]1N]

The subsequent two steps are similar to the last two steps of Embodiment 114 and performed to give compound 123 (266 mg, yield 52%). 1H NMR (400 MHZ, DMSO-d₆) δ: 9.93 (s, 1H), 8.91 (d, 2H), 8.31 (s, 2H), 7.64-6.93 (m, 4H), 4.15-4.11 (m, 1H), 3.93-3.45 (m, 7H), 3.33-3.23 (m, 2H), 2.90 (d, 1H), 1.74-1.31 (m, 3H), 1.15 (d, 3H); LC/MS(ESI): m/z=512.2 [M+H]⁺.

### Embodiment 124

Preparation N-(3-((2-((3S,4S)-4-amino-3-methyl-2-oxa-8-azaspiro[4.5]decane-8-yl of 1)pyrimidine-5-yl)mercapto)-2-chlorophenyl)pyrimidine-4-carboxamide

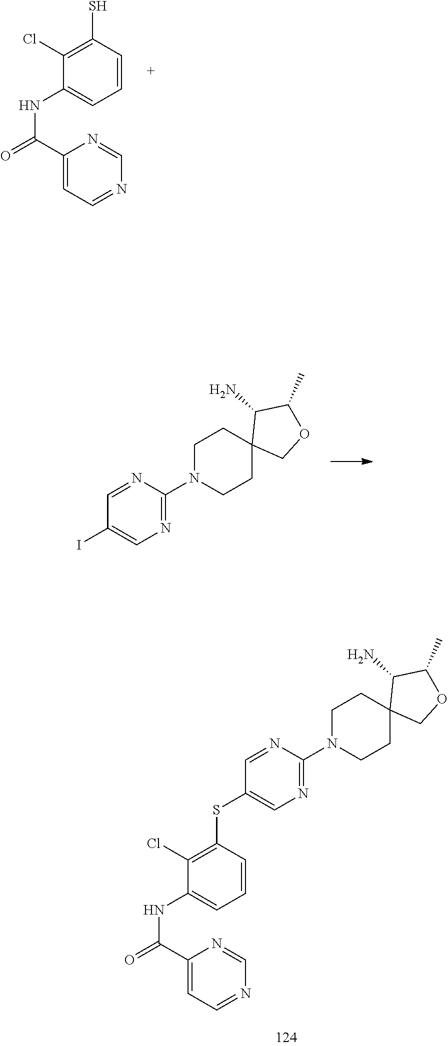 [SMILES: O=C(Nc1cccc(S)c1Cl)c1ccncn1 ; C[C@@H]1OCC2(CCN(c3ncc(I)cn3)CC2)[C@@H]1N ; C[C@@H]1OCC2(CCN(c3ncc(Sc4cccc(NC(=O)c5ccncn5)c4Cl)cn3)CC2)[C@@H]1N]

Compound 124 (282 mg, yield 55%) is obtained in a similar manner to Embodiment 123 (the raw material is changed to pyrimidine-4-carboxylic acid). 1H NMR (400 MHZ, DMSO-d₆) δ: 9.88 (s, 1H), 9.31 (s, 1H), 9.01 (d, 1H), 8.31 (s, 2H), 8.21 (d, 1H), 7.75 (d, 1H), 7.42 (t, 1H), 7.15 (d, 1H), 4.15-4.11 (m, 1H), 3.93-3.45 (m, 7H), 3.33-3.23 (m, 2H), 2.90 (d, 1H), 1.74-1.31 (m, 3H), 1.15 (d, 3H); LC/MS(ESI): m/z=512.2 [M+H]⁺.

### Embodiment 125

### Preparation of N-(3-((2-((3S,4S)-4-amino-3-methyl-2-oxa-8-azaspiro[4.5]decane-8-yl)pyrimidine-5-yl)mercapto)-2-chlorophenyl)pyridine-2-carboxamide

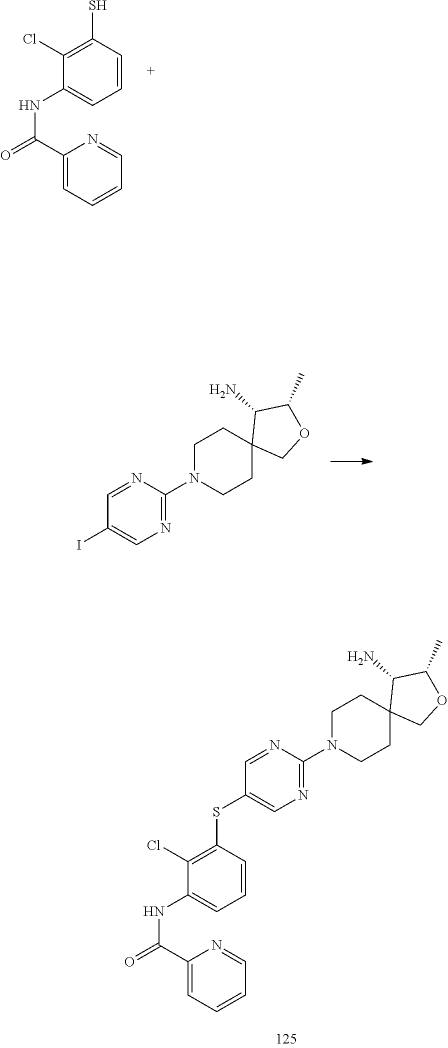 [SMILES: O=C(Nc1cccc(S)c1Cl)c1ccccn1 ; C[C@@H]1OCC2(CCN(c3ncc(I)cn3)CC2)[C@@H]1N ; C[C@@H]1OCC2(CCN(c3ncc(Sc4cccc(NC(=O)c5ccccn5)c4Cl)cn3)CC2)[C@@H]1N]

Compound 125 (245 mg, yield 48%) is obtained in a similar manner to Embodiment 123 (the raw material is changed to pyridine-2-carboxylic acid). 1H NMR (400 MHZ, DMSO-d₆) δ: 10.02 (s, 1H), 8.61-8.59 (m, 1H), 8.32-8.30 (m, 3H), 7.75-7.55 (m, 3H), 7.44-7.39 (m, 1H), 7.14-7.03 (m, 1H), 4.15-4.11 (m, 1H), 3.93-3.45 (m, 7H), 3.32-3.21 (m, 2H), 2.90 (d, 1H), 1.73-1.30 (m, 3H), 1.15 (d, 3H); LC/MS(ESI): m/z=511.2 [M+H]⁺.

### Embodiment 126

### Preparation of N-(3-((2-((3S,4S)-4-amino-3-methyl-2-oxa-8-azaspiro[4.5]decane-8-yl)pyrimidine-5-yl)mercapto)-2-chlorophenyl)pyrazine-2-carboxamide

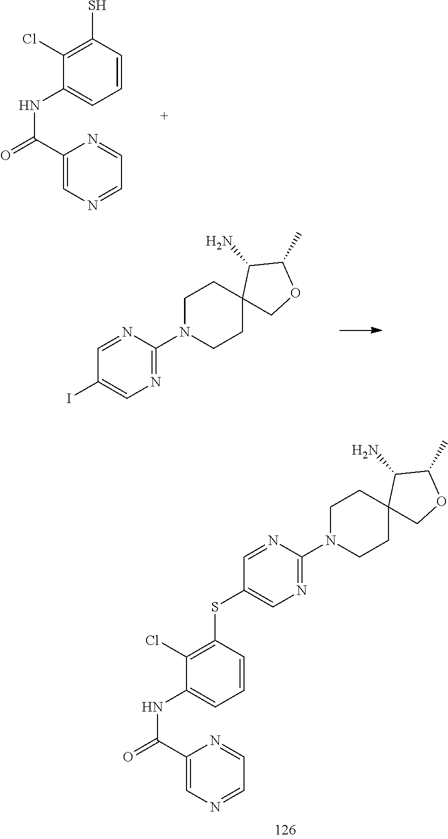 [SMILES: O=C(Nc1cccc(S)c1Cl)c1cnccn1 ; C[C@@H]1OCC2(CCN(c3ncc(I)cn3)CC2)[C@@H]1N ; C[C@@H]1OCC2(CCN(c3ncc(Sc4cccc(NC(=O)c5cnccn5)c4Cl)cn3)CC2)[C@@H]1N]

Compound 126 (261 mg, yield 51%) is obtained in a similar manner to Embodiment 123 (the raw material is changed to pyrazine-2-carboxylic acid). 1H NMR (400 MHZ, DMSO-d₆) δ: 9.68 (s, 1H), 9.51 (s, 1H), 8.81 (d, 1H), 8.55-8.52 (m, 1H), 8.31 (s, 2H), 7.71-7.62 (m, 1H), 7.43-7.39 (m, 1H), 7.15 (d, 1H), 4.15-4.11 (m, 1H), 3.93-3.45 (m, 7H), 3.33-3.23 (m, 2H), 2.90 (d, 1H), 1.74-1.31 (m, 3H), 1.15 (d, 3H); LC/MS(ESI): m/z=512.2 [M+H]⁺.

### Embodiment 127

### Preparation of (3S,4S)-8-(3-((2-chloro-3-(1-ethyl-1H-pyrazole-3-yl)phenyl)mercapto)pyridazine-6-yl)-3-methyl-2-oxa-8-azaspiro[4.5]decane-4-amine

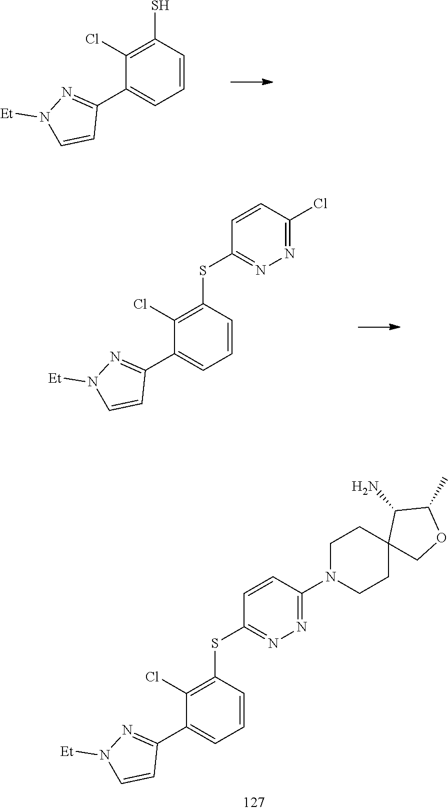 [SMILES: CCn1ccc(-c2cccc(S)c2Cl)n1 ; CCn1ccc(-c2cccc(Sc3ccc(Cl)nn3)c2Cl)n1 ; CCn1ccc(-c2cccc(Sc3ccc(N4CCC5(CC4)CO[C@@H](C)[C@H]5N)nn3)c2Cl)n1]

Compound 127 (320 mg, 44% yield, which is the yield of the last step, the same below) is obtained by a method similar to that of Embodiment 114. LC/MS(ESI): m/z=486.2[M+H]⁺.

### Embodiment 128

### Preparation of (3S,4S)-8-(3-((2-chloro-3-(1-ethyl-1H-pyrazole-3-yl)phenyl)mercapto)-tetrazine-6-yl)-3-methyl-2-oxa-8-azaspiro[4.5]decane-4-amine

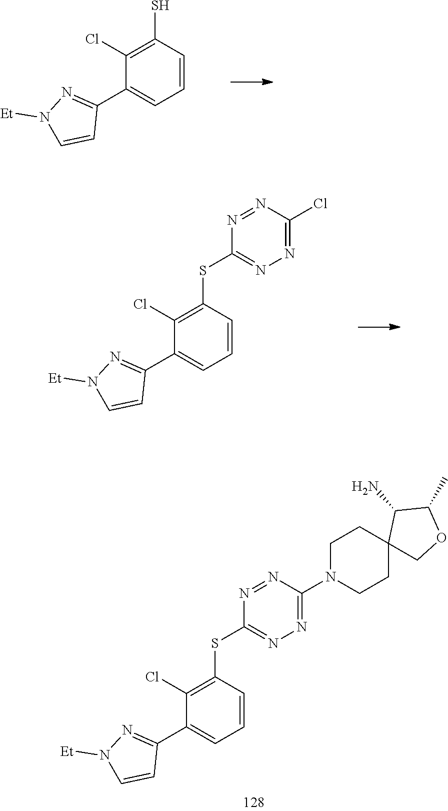 [SMILES: CCn1ccc(-c2cccc(S)c2Cl)n1 ; CCn1ccc(-c2cccc(Sc3nnc(Cl)nn3)c2Cl)n1 ; CCn1ccc(-c2cccc(Sc3nnc(N4CCC5(CC4)CO[C@@H](C)[C@H]5N)nn3)c2Cl)n1]

Compound 128 (358 mg, 49% yield, which is the yield of the last step, the same below) is obtained by a method similar to that of Embodiment 114. LC/MS(ESI): m/z=488.2[M+H]⁺.

### Embodiment 129

### Preparation of (3S,4S)-8-(3-((2-chloro-3-(1-ethyl-1H-pyrazole-3-yl)phenyl)mercapto)-1,2,4-triazine-6-yl)-3-methyl-2-oxa-8-azaspiro[4.5]decane-4-amine

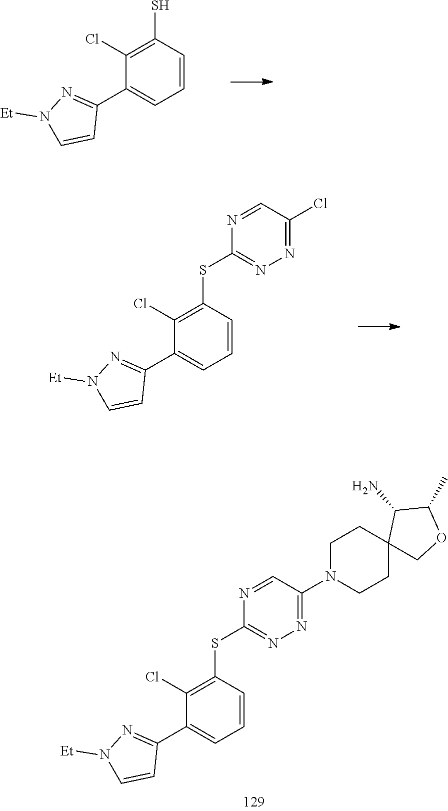 [SMILES: CCn1ccc(-c2cccc(S)c2Cl)n1 ; CCn1ccc(-c2cccc(Sc3ncc(Cl)nn3)c2Cl)n1 ; CCn1ccc(-c2cccc(Sc3ncc(N4CCC5(CC4)CO[C@@H](C)[C@H]5N)nn3)c2Cl)n1]

Compound 129 (350 mg, 48% yield, which is the yield of the last step, the same below) is obtained by a method similar to that of Embodiment 114. LC/MS(ESI): m/z=487.2[M+H]⁺.

### Embodiment 130

### Preparation of N-(3-((2-((3S,4S)-4-amino-3-methyl-2-oxa-8-azaspiro[4.5]decane-8-yl)-1,2,4-triazine-6-yl)mercapto)-2-chlorophenyl)-2-hydroxy-4-oxo-6,7,8,9-tetrahydro-4H-pyridinyl[1,2-a]pyrimidine-3-carboxamide

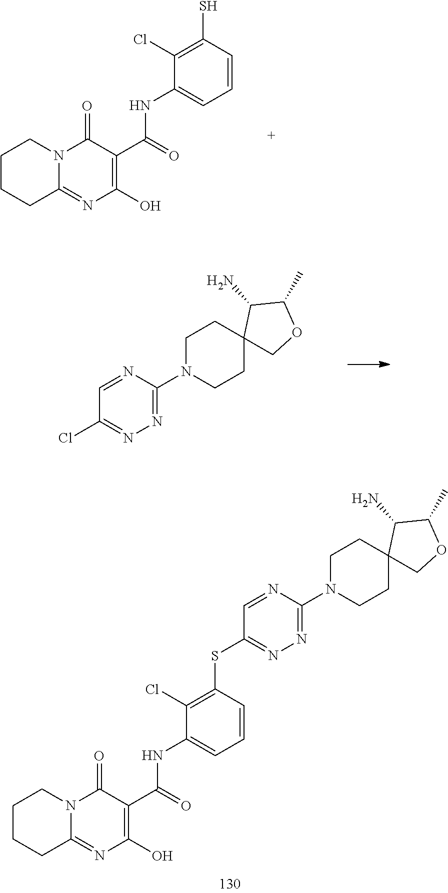 [SMILES: O=C(Nc1cccc(S)c1Cl)c1c(O)nc2n(c1=O)CCCC2 ; C[C@@H]1OCC2(CCN(c3ncc(Cl)nn3)CC2)[C@@H]1N ; C[C@@H]1OCC2(CCN(c3ncc(Sc4cccc(NC(=O)c5c(O)nc6n(c5=O)CCCC6)c4Cl)nn3)CC2)[C@@H]1N]

Compound 130 (299 mg, 41% yield, which is the yield of the last step, the same below) is obtained by a method similar to that of Embodiment 123. LC/MS(ESI): m/z=600.2[M+H]⁺.

### Embodiment 131

### Preparation of (3S,4S)-8-(6-((2-chloro-3-(1-ethyl-1H-pyrazole-3-yl)phenyl)mercapto)-1,2,4-triazine-3-yl)-3-methyl-2-oxa-8-azaspiro[4.5]decane-4-amine

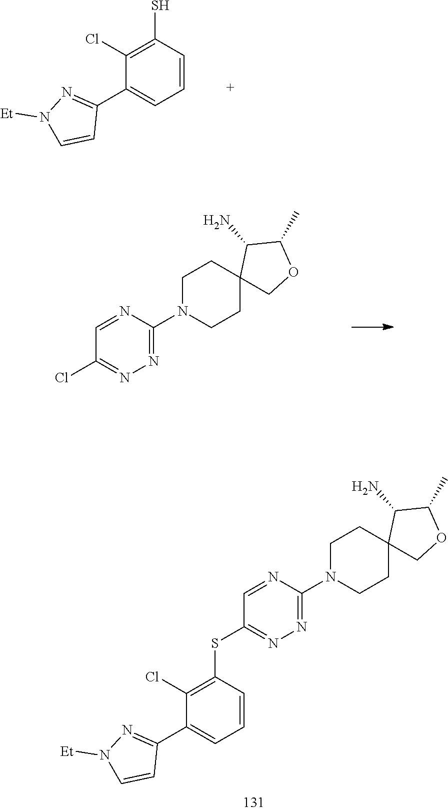 [SMILES: CCn1ccc(-c2cccc(S)c2Cl)n1 ; C[C@@H]1OCC2(CCN(c3ncc(Cl)nn3)CC2)[C@@H]1N ; CCn1ccc(-c2cccc(Sc3cnc(N4CCC5(CC4)CO[C@@H](C)[C@H]5N)nn3)c2Cl)n1]

Compound 131 (283 mg, yield 39%, this is the yield of the last step, the same below) is obtained by a method similar to that of Embodiment 123. LC/MS(ESI): m/z=487.2[M+H]⁺.

### Embodiment 132

### Preparation of (3S,4S)-8-(3-((2-chloro-3-(pyrazine-2-yl)phenyl)mercapto)pyridazine-6-yl)-3-methyl-2-oxa-8-azaspiro[4.5]decane-4-amine

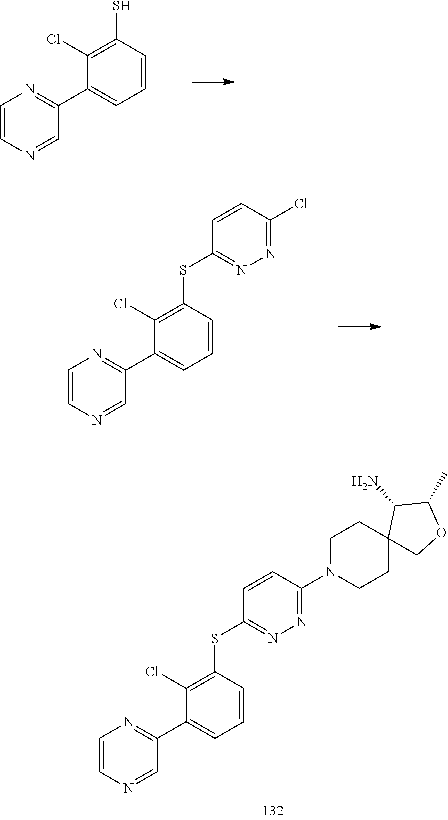 [SMILES: Sc1cccc(-c2cnccn2)c1Cl ; Clc1ccc(Sc2cccc(-c3cnccn3)c2Cl)nn1 ; C[C@@H]1OCC2(CCN(c3ccc(Sc4cccc(-c5cnccn5)c4Cl)nn3)CC2)[C@@H]1N]

Compound 132 (200 mg, yield 37%) is obtained in a similar manner to Embodiment 118. LC/MS(ESI): m/z=470.1[M+H]⁺.

### Embodiment 133

### Preparation of (3S,4S)-8-(3-((2-chloro-3-(pyrazine-2-yl)phenyl)mercapto)-tetrazine-6-yl)-3-methyl-2-oxa-8-azaspiro[4.5]decane-4-amine

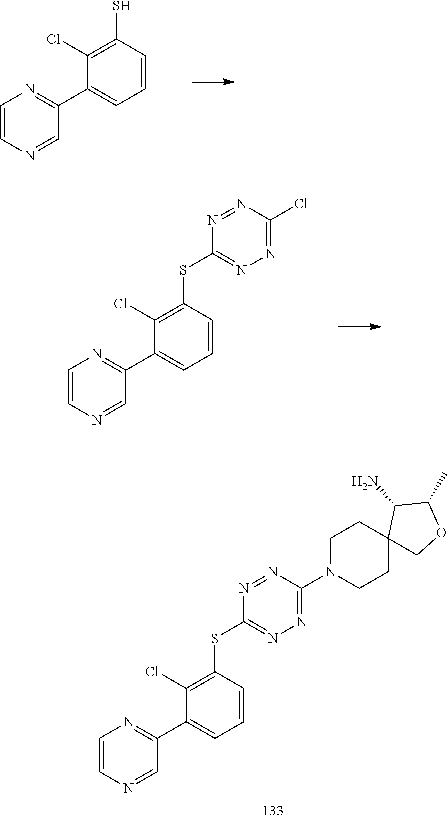 [SMILES: Sc1cccc(-c2cnccn2)c1Cl ; Clc1nnc(Sc2cccc(-c3cnccn3)c2Cl)nn1 ; C[C@@H]1OCC2(CCN(c3nnc(Sc4cccc(-c5cnccn5)c4Cl)nn3)CC2)[C@@H]1N]

Compound 133 (211 mg, yield 39%) is obtained in a similar manner to Embodiment 118. LC/MS(ESI): m/z=472.1[M+H]⁺.

### Embodiment 134

### Preparation of (3S,4S)-8-(3-((2-chloro-3-(pyrazine-2-yl)phenyl)mercapto)-1,2,4-triazine-6-yl)-3-methyl-2-oxa-8-azaspiro[4.5]decane-4-amine

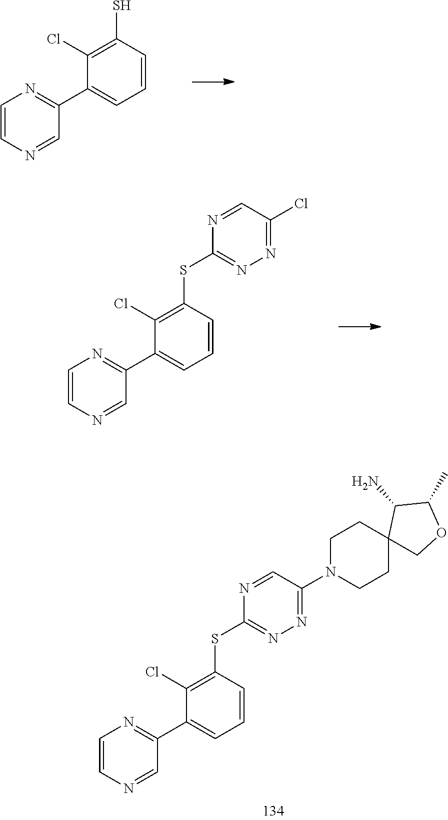 [SMILES: Sc1cccc(-c2cnccn2)c1Cl ; Clc1cnc(Sc2cccc(-c3cnccn3)c2Cl)nn1 ; C[C@@H]1OCC2(CCN(c3cnc(Sc4cccc(-c5cnccn5)c4Cl)nn3)CC2)[C@@H]1N]

Compound 134 (227 mg, yield 42%) is obtained in a similar manner to Embodiment 114. LC/MS(ESI): m/z=471.1[M+H]⁺.

### Embodiment 135

### Preparation of (3S,4S)-8-(6-((2-chloro-3-(pyrazine-2-yl)phenyl)mercapto)-1,2,4-triazine-3-yl)-3-methyl-2-oxa-8-azaspiro[4.5]decane-4-amine

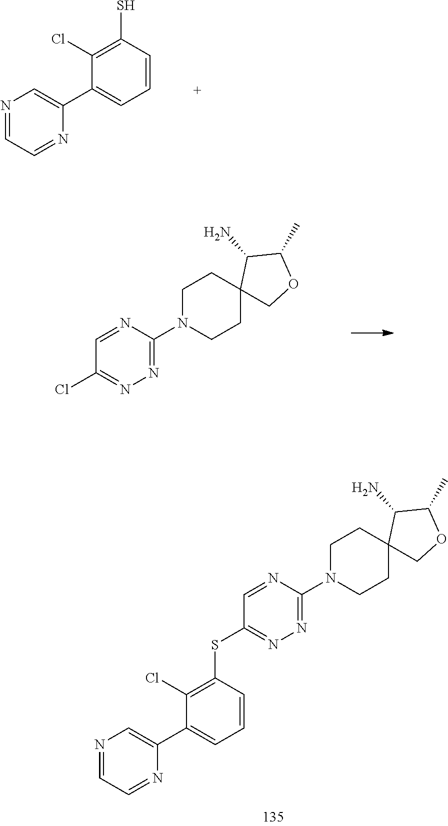 [SMILES: Sc1cccc(-c2cnccn2)c1Cl ; C[C@@H]1OCC2(CCN(c3ncc(Cl)nn3)CC2)[C@@H]1N ; C[C@@H]1OCC2(CCN(c3ncc(Sc4cccc(-c5cnccn5)c4Cl)nn3)CC2)[C@@H]1N]

Compound 135 (384 mg, yield 41%) is obtained in a similar manner to the last step of Embodiment 114. LC/MS(ESI): m/z=471.1[M+H]⁺.

### Embodiment 136

### Preparation of N-(3-((5-((3S,4S)-4-amino-3-methyl-2-oxa-8-azaspiro[4.5]decane-8-yl)pyridazine-3-yl) mercapto)-2-chlorophenyl)-2-hydroxy-4-oxa-6,7,8,9-tetrahydro-4H-pyridinyl[1,2-a]pyrimidine-3-carboxamide

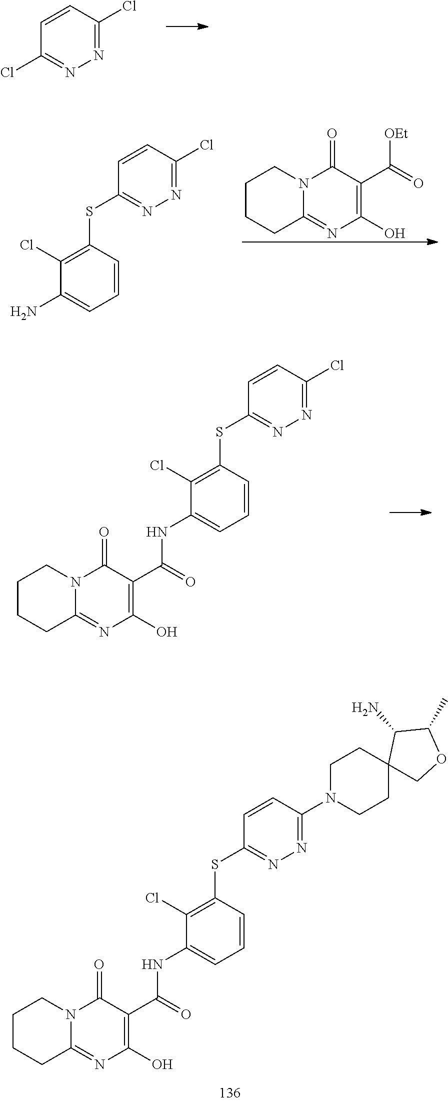 [SMILES: Clc1ccc(Cl)nn1 ; Nc1cccc(Sc2ccc(Cl)nn2)c1Cl ; CCOC(=O)c1c(O)nc2n(c1=O)CCCC2 ; O=C(Nc1cccc(Sc2ccc(Cl)nn2)c1Cl)c1c(O)nc2n(c1=O)CCCC2 ; C[C@@H]1OCC2(CCN(c3ccc(Sc4cccc(NC(=O)c5c(O)nc6n(c5=O)CCCC6)c4Cl)nn3)CC2)[C@@H]1N]

Compound 136 (197 mg, yield 32%) is obtained in a similar manner to Embodiment 117. LC/MS(ESI): m/z=499.1[M+H]⁺.

### Embodiment 137

### Preparation of N-(3-((5-((3S,4S)-4-amino-3-methyl-2-oxa-8-azaspiro[4.5]decane-8-yl)s-tetrazine-3-yl) mercapto)-2-chlorophenyl)-2-hydroxy-4-oxa-6,7,8,9-tetrahydro-4H-pyridinyl[1,2-a]pyrimidine-3-carboxamide

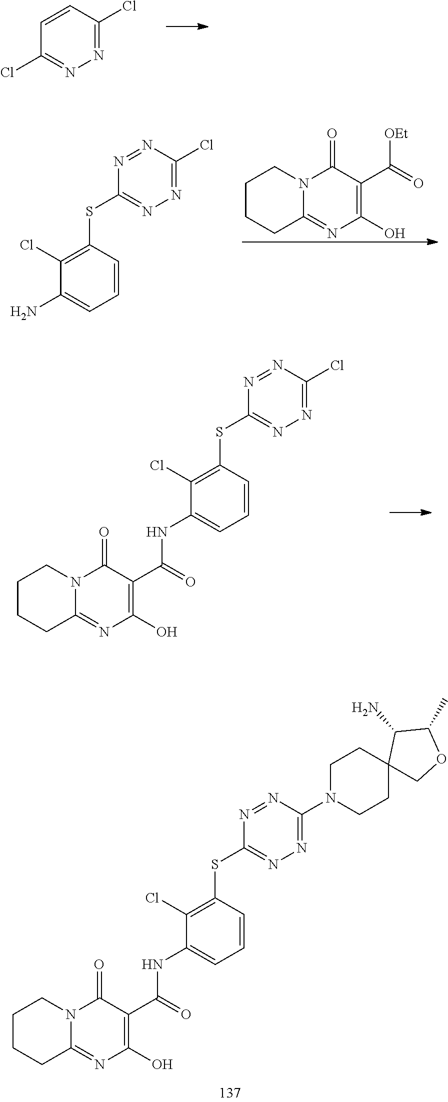 [SMILES: Clc1ccc(Cl)nn1 ; Nc1cccc(Sc2nnc(Cl)nn2)c1Cl ; CCOC(=O)c1c(O)nc2n(c1=O)CCCC2 ; O=C(Nc1cccc(Sc2nnc(Cl)nn2)c1Cl)c1c(O)nc2n(c1=O)CCCC2 ; C[C@@H]1OCC2(CCN(c3nnc(Sc4cccc(NC(=O)c5c(O)nc6n(c5=O)CCCC6)c4Cl)nn3)CC2)[C@@H]1N]

Compound 137 (159 mg, yield 25%) is obtained in a similar manner to Embodiment 117. LC/MS(ESI): m/z=601.1[M+H]⁺.

### Embodiment 138

### Preparation of N-(3-((5-((3S,4S)-4-amino-3-methyl-2-oxa-8-azaspiro[4.5]decane-8-yl)-1,2,4-triazine-3-yl)mercapto)-2-chlorobenzene)-2-hydroxy-4-oxa-6,7,8,9-tetrahydro-4H-pyridinyl[1,2-a]pyrimidine-3-carboxamide

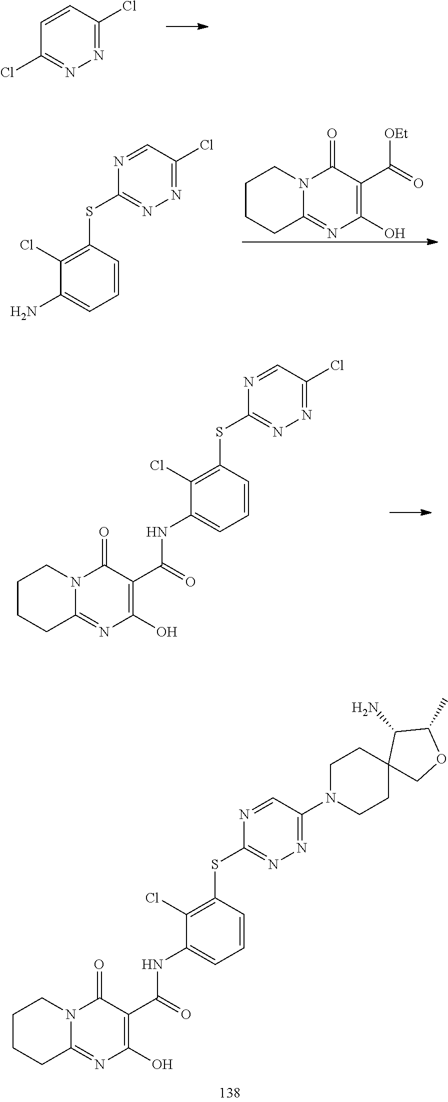 [SMILES: Clc1ccc(Cl)nn1 ; Nc1cccc(Sc2ncc(Cl)nn2)c1Cl ; CCOC(=O)c1c(O)nc2n(c1=O)CCCC2 ; O=C(Nc1cccc(Sc2ncc(Cl)nn2)c1Cl)c1c(O)nc2n(c1=O)CCCC2 ; C[C@@H]1OCC2(CCN(c3cnc(Sc4cccc(NC(=O)c5c(O)nc6n(c5=O)CCCC6)c4Cl)nn3)CC2)[C@@H]1N]

Compound 138 (178 mg, yield 29%) is obtained in a similar manner to Embodiment 117. LC/MS(ESI): m/z=600.1[M+H]⁺.

### Embodiment 139

### Biological Activity Test of Embodiment 139

The following further describes and explains the present invention in combination with test examples, but these examples are not meant to limit the scope of the present invention. SHP2 allosteric inhibition experiment

### Determination of Compound's Inhibitory Effect on SHP2 Kinase Activity

The purpose of this test is to measure the ability of the compounds to inhibit the allosteric activity of the SHP2 full-length protein. Experimental equipment: a centrifuge (5810R) is purchased from Eppendorf Company; pipettes are purchased from Eppendorf Company and Rainin Company, and microplate readers are purchased from BioTek Company of the United States, SynergyH1 multimode microplate reader.

Experimental method: Homogeneous Full Length SHP-2 Assay Kit (BPS Bioscience, #79330) is used to detect SHP2 activity in vitro. First, add 18 μL of Master Mix to 96-well low-adsorption microplate (NUNC, #267342), that is, 0.5 μL of SHP-2 activating Peptide and 5 mM DTT are included in the reaction buffer with a final concentration of 1×. After centrifugation, add 5 μL of the compound to be tested in DMSO (final DMSO content is 1% (V/V) in each well: dissolve the compound to be tested in DMSO to 1 mM, conduct triple dilution with a total of 10 concentration levels ranging from 1 μM to 0.05 nM in the reaction buffer. After diluting SHP2 in 1× reaction buffer to a final concentration of 0.06 nM, add it to the reaction microwell plate, 2 μL per well. Set a full activity control (compound plus DMSO only) and a full inhibition control (without SHP-2) on the reaction plate, and incubate the reaction mixture at room temperature for 60 minutes after centrifugation.

After incubation, add 25 μL of Substrate solution to each well, containing Substrate with a final concentration of 10 μM and DTT with a final concentration of 5 mM, and after centrifugation, continue to incubate at room temperature for 30 minutes. After the reaction, set the excitation wavelength at 340 nm, the emission wavelength at 455 nM, and the gain value to 75 on the Synergy H1 microplate reader (Biotek) for reading.

### Experimental Data Processing Method

Take the full activity control and full inhibition control as the values of Max and Min, and calculate the percentage inhibition rate of the wells treated with the compound based on the positive control wells (DMSO control wells) and negative control wells (no kinase added) on the reaction plate, (inhibition rate percentage=100−[(test compound−Min average value)/(Max average value−Min average value)]×100%. IC₅₀ values of test compounds are calculated by fitting percentage of inhibition and ten-point concentration data to a 4-parameter nonlinear logic formula using GraphPad prism.

### Experimental Conclusion

By following the above protocol, the tested compounds of the present invention exhibited the biological activities shown in Table 1 in the SHP2 kinase activity test. Where “A” represents IC₅₀≤10 nM; “B” represents 10<IC₅₀≤100 nM; “C” represents 100<IC_{50≤1000} nM; “D” represents 1000<IC₅₀ nM.

### IC₅₀ Values of Compounds Inhibiting SHP2

| No. | IC₅₀ |
|---|---|
| 1 | A |
| 2 | A |
| 3 | A |
| 4 | A |
| 5 | A |
| 6 | A |
| 7 | A |
| 8 | A |
| 9 | B |
| 10 | B |
| 11 | B |
| 12 | B |
| 13 | B |
| 14 | B |
| 15 | B |
| 16 | B |
| 17 | A |
| 18 | A |
| 19 | A |
| 20 | A |
| 21 | A |
| 22 | A |
| 23 | A |
| 24 | A |
| 25 | A |
| 26 | A |
| 27 | A |
| 28 | A |
| 63 | A |
| 64 | A |
| 65 | A |
| 66 | A |
| 67 | A |
| 68 | A |
| 69 | A |
| 70 | A |
| 71 | A |
| 72 | A |
| 73 | A |
| 74 | A |
| 75 | A |
| 76 | A |
| 77 | A |
| 78 | A |
| 79 | A |
| 80 | A |
| 81 | A |
| 82 | A |
| 83 | A |
| 84 | A |
| 85 | A |
| 86 | A |
| 87 | A |
| 88 | A |
| 89 | A |
| 90 | A |
| 91 | A |
| 92 | A |
| 93 | A |
| 94 | A |
| 95 | A |
| 96 | A |
| 97 | A |
| 98 | A |
| 99 | A |
| 100 | A |
| 101 | A |
| 102 | A |
| 103 | A |
| 104 | A |
| 105 | A |
| 106 | A |
| 107 | B |
| 108 | B |
| 109 | B |
| 110 | B |
| 111 | B |
| 112 | B |
| 113 | B |
| 114 | B |
| 115 | A |
| 116 | A |
| 117 | A |
| 118 | A |
| 119 | A |
| 120 | A |
| 121 | A |
| 122 | A |
| 123 | A |
| 124 | A |
| 125 | A |
| 126 | A |
| 127 | A |
| 128 | A |
| 129 | A |
| 130 | A |
| 131 | A |
| 132 | A |
| 133 | A |
| 134 | A |
| 135 | A |
| 136 | A |
| 137 | A |
| 138 | A |

1. A compound represented by general formula (I) and general formula (II) or its prodrug, stable isotope derivative, pharmaceutically acceptable salt, polymorph or isomer, 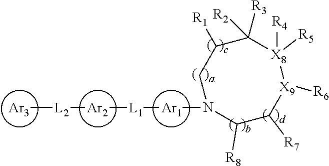
Wherein:
Each L₁ is, at each occurrence, independently selected from bond, O, CH₂, NH, CO, —S(O)ₘ—, or S;
Each L₂ is, at each occurrence, independently selected from bond, O, CH₂, NH, CONH₂, CO, —S(O)ₘ—, or S;
Each Ar₁, at each occurrence, is independently selected from a 6-membered heteroaryl or a 10-membered heteroaryl; Each Ar₁ at each occurrence is independently optionally substituted or unsubstituted by 1 or 2 R_{19S};
Each Ar₂ is, at each occurrence, independently selected from phenyl, naphthyl, 5-membered heteroaryl, 6-membered heteroaryl, 7-membered heteroaryl, 8-membered heteroaryl, 9-membered heteroaryl, or 10-membered heteroaryl, 3-membered to 10-membered cycloalkyl, 5-membered to 10-membered heterocyclic alkyl; and each heteroaryl and heterocyclic alkyl at each occurrence independently comprises, 1, 2, 3, or 4 heteroatoms selected from N, O, or S; Each Ar₂ at each occurrence is independently optionally substituted or unsubstituted by 1, 2, 3, 4, 5, or 6 R_{19S};
Each Ar₃ is, at each occurrence, independently selected from H, phenyl, naphthyl, 5-membered heteroaryl, 6-membered heteroaryl, 7-membered heteroaryl, 8-membered heteroaryl, 9-membered heteroaryl, or 10-membered heteroaryl, 3-membered to 10-membered cycloalkyl, 5-membered to 10-membered heterocyclic alkyl; and each heteroaryl and heterocyclic alkyl is, at each occurrence, independently comprises 1, 2, 3, or 4 heteroatoms selected from N, O, or S; Each Ar₃ at each occurrence is independently optionally substituted or unsubstituted by 1, 2, 3, 4, or 6 R_{19S};
Each R₁₉ is, at each occurrence, independently selected from deuterium, halogen, oxo, —C₁₋₆ alkyl, —C₁₋₆ alkylidene-(halogen)₁₋₃, C₁₋₆ heteroalkyl, —CN, —OR₁₀, —C₁₋₆ alkylidene-(OR₁₀)₁₋₃, —O—C₁₋₆ alkylidene-(halogen)₁₋₃, —SR₁₀, —S—C₁₋₆ alkylidene-(halogen)₁₋₃, —NR₁₀R₁₁, —C₁₋₆ alkylidene-NR₁₀R₁₁, —C(═O)R₁₀, —C(═O)OR₁₀, —OC(═O)R₁₀, —C(═O)NR₁₀R₁₁, —NR₁₀C(═O)R₁₁, —S(O)₂NR₁₀R₁₁, or —C₃₋₆ carbocyclyl; Each R₁₉ is independently optionally substituted or unsubstituted by 1, 2, 3, 4, 5 or 6 substituents selected from deuterium, halogen, —C₁₋₆ alkyl, —C₁₋₆ alkoxy, oxo, —OR₁₀, —NR₁₀R₁₁, —CN, —C(═O)R₁₀, —C(═O)OR₁₀, —OC(═O)R₁₀, —C(═O)NR₁₀R₁₁, —NR₁₀C(═O)R₁₁, or —S(O)₂NR₆R₁₁;
Each R₁₀ and R₁₁ is, at each occurrence, independently selected from hydrogen, deuterium, or —C₁₋₆ alkyl, and each R₁₀ and R₁₁ is independently optionally substituted or unsubstituted by 1, 2, 3, 4, 5, or 6 substituents of R₁₉; Or, R₁₀, R₁₁ and the N atoms attached to the R₁₀ and R₁₁ together form a 3-membered and 10-membered heterocyclic ring, which can further comprise 1, 2, 3, or 4 heteroatoms selected from N, O, S, S(═O), S(═O), or S(═O)₂, and the stated 3-membered and 10-membered heterocyclic ring is independently optionally substituted or unsubstituted by 1, 2, 3, 4, 5, or 6 R₂₀ₛ;
Each R₂₀ is, at each occurrence, independently selected from deuterium, halogen, oxo, —C₁₋₆ alkyl, —C₁₋₆ alkylidene-(halogen)₁₋₃, C₁₋₆ heteroalkyl, —CN, —OC₁₋₆, —C₁₋₆ alkylidene-(OC₁-6)₁₋₃, —O—C₁₋₆ alkylidene-(halogen)₁₋₃, —SC₁₋₆, —S—C₁₋₆ alkylidene-(halogen)₁₋₃, or —C₃₋₆ carbocyclyl;
Each X₈ is, at each occurrence, independently selected from CR₄R₅, SiR₄R₅, NH, or O;
Each X₉ is, at each occurrence, independently selected from CR₆ or NH, wherein, one of X₇ and X₈ must be carbon atom;
Each R₁ is, at each occurrence, independently selected from H, deuterium, or —C₁₋₆ alkyl;
Each R₂ is, at each occurrence, independently selected from H, deuterium, OH, or CH₂NH₂;
Each R₃, R₇ and R₈ is, at each occurrence, independently selected from H or deuterium;
Each R₄ is, at each occurrence, independently selected from H, deuterium, OH, or C₀₋₃NR₁₂R₁₃;
Each R₅ is, at each occurrence, independently selected from H, deuterium, OH, or C₁₋₆ alkyl; C₁₋₆ alkyl comprises 1, 2, 3, 4, 5, or 6 deuteriums, OH, methyl, OCH₃, and 5-membered to 10-membered heteroaryl;
Each R₆ is, at each occurrence, independently selected from H, deuterium, or NH₂;
Two of R₁, R₂, R₃, R₄, R₅, R₆, R₇, and R₈ can connect each other in the following manner:
R1 and R2 can be connected by CH₂NHCH₂ to form a fused bicyclic structure,
R₁ and R₆ can be connected by an alkylidene group to form a bridged bicyclic structure,
R₂ and R₃ can be connected by an NH₂-substituted alkylidene to form a spiro,
R₄ and R₅ can be connected to form a C₃₋₁₂ cycloalkyl, a C₃₋₁₂ heterocyclic alkyl, a C₃₋₁₂ bicyclic alkyl, or a C₃₋₁₂ heterobicyclic alkyl, wherein, the heterocyclic alkyl and the heterobicyclic alkyl of C₃₋₁₂, at each occurrence, independently comprise 1, 2, 3, or 4 heteroatoms selected from N, O, or S, and wherein each of C₃₋₁₂ cycloalkyl, C₃₋₁₂ heterocyclic alkyl, C₃₋₁₂ bicyclic alkyl, and C₃₋₁₂ heterobicyclic alkyl is, at each occurrence, independently optionally is substituted by deuterium, halogen, OH, CH₃, OCH₃, or NH₂ to form a spiro.
R₁ and R₇ can form a bridged bicyclic structure through the connection of alkylidene, O and NH,
R₂ and R₆ can form a bridged bicyclic structure through the connection of alkylidenes,
R₂ and R₇ can form a bridged bicyclic structure through the connection of alkylidene and O,
R₄ and R₆ can connect with each other to form a fused bicyclic structure through NHCH₂ and NH₂-substituted C₃₋₁₂ cycloalkyl,
Each of a, b, c, and d is, at each occurrence, independently selected from 0, or 1;

2. The compound of (I) according to claim 1, a pharmaceutically acceptable salt thereof or a stereoisomer thereof, wherein each Ar₁ is independently selected from 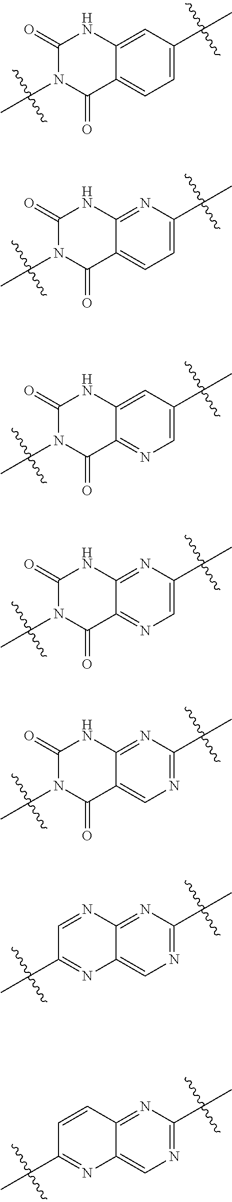 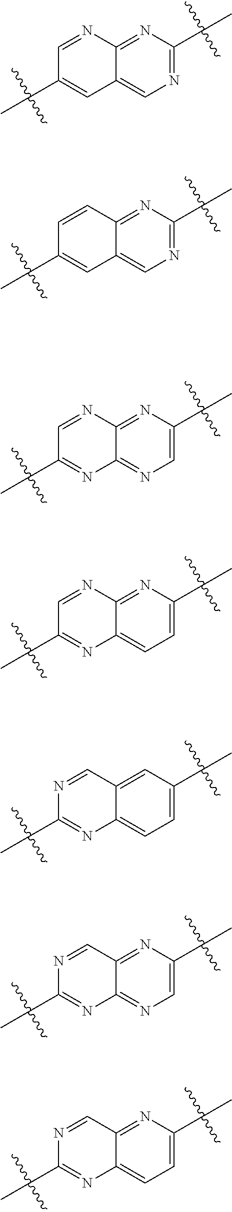 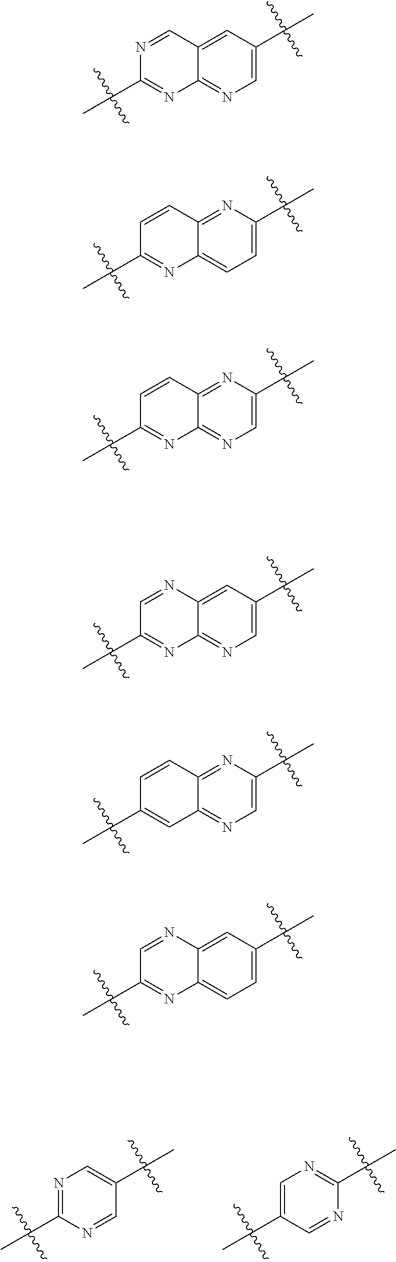 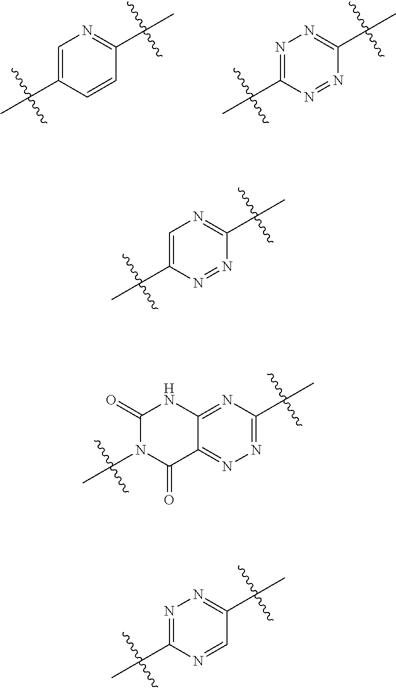 [SMILES: *c1ccc(*)nc1 ; *c1nnc(*)nn1 ; *c1cnc(*)nn1 ; *c1nnc2c(=O)n(*)c(=O)[nH]c2n1 ; *c1cnc(*)nn1]
Each Ar₁ is, at each occurrence, independently optionally substituted or unsubstituted by 1 or 2 R_{19S};
Each R₁₉ is independently selected, at each occurrence, from deuterium, halogen, oxo, —C₁₋₆ alkyl, —C₁₋₆ alkylidene-(halogen)₁₋₃, C₁₋₆ heteroalkyl, —CN, —OR₁₀, —C₁₋₆ alkylidene-(OR 10)₁₋₃, —O—C₁₋₆ alkylidene-(halogen)₁₋₃, —SR₁₀, —S—C₁₋₆ alkylidene-(halogen)₁₋₃, —NR₁₀R₁₁, —C₁₋₆ alkylidene-NR₁₀R₁₁, —C(═O)R₁₀, —C(═O)OR₁₀, —OC(═O)R₁₀, —C(═O)NR₁₀R₁₁, —NR₁₀C(═O)R₁₁, —S(O)₂NR₁₀R₁₁, or —C₃₋₆ carbocyclic; Each R₁₉ is independently optionally substituted by 1, 2, 3, 4, 5 or 6 substituents selected from deuterium, halogen, —C₁₋₆ alkyl, —C₁₋₆ alkoxy, oxo, —OR₁₀, —NR₁₀R₁₁, —CN, —C(═O)R₁₀, —C(═O)OR₁₀, —OC(═O)R₁₀, —C(═O)NR₁₀R₁₁, —NR₁₀C(═O)R₁₁, or —S(O)₂NR₆R₁₁;
Each of R₁₀ and R₁₁ is independently selected, at each occurrence, from hydrogen, deuterium, or —C₁₋₆ alkyl, and each of R₁₀ and R₁₁ is independently optionally substituted or unsubstituted by 1, 2, 3, 4, 5, or 6 R₁₉ₛ; or R₁₀, R₁₁, and the atoms attached thereto form a 3-membered to 10-membered heterocyclic ring, and the stated 3-membered to 10-membered heterocyclic ring can further comprise 1, 2, 3, or 4 heteroatoms selected from N, O, S, S(═O), S(═O), or S(═O)₂, and the stated 3-membered to 10-membered heterocyclic ring is independently optionally substituted or unsubstituted by 1, 2, 3, 4, 5, or 6 R_{20S};
Each R₂₀ is independently selected, at each occurrence, from deuterium, halogen, oxo, —C₁₋₆ alkyl, —C₁₋₆ alkylidene-(halogen)₁₋₃, C₁₋₆ heteroalkyl, .CN, —OC₁₋₆, —C₁₋₆ alkylidene-(OC₁-6)₁₋₃, —O—C₁₋₆ alkylidene-(halogen)₁₋₃, —SC₁₋₆, —S—C₁₋₆ alkylidene-(halogen)₁₋₃, or —C₃₋₆ carbocyclic;

3. In some embodiments, the compound of (I) described above, the pharmaceutically acceptable salt thereof, or the stereoisomer thereof, wherein the structure 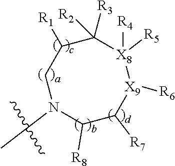
is selected from the following:

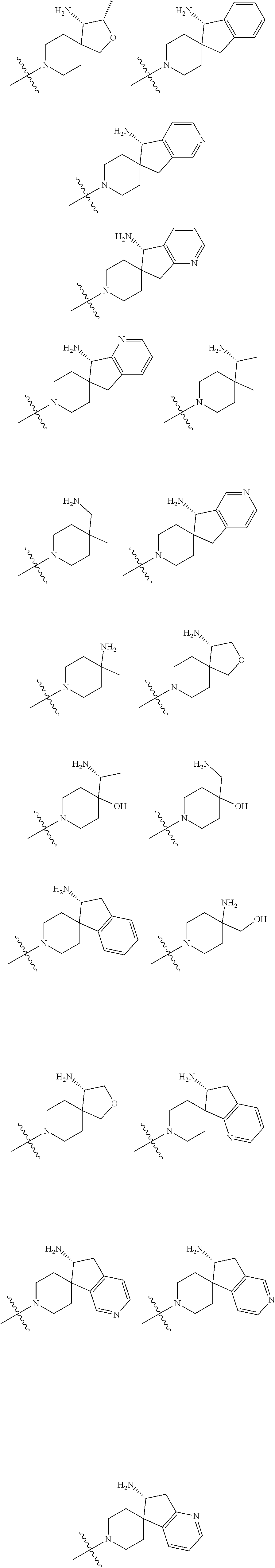

4. The compounds of (I) according to claim 1, pharmaceutically acceptable salts thereof, or a stereoisomers thereof, wherein Ar₂ is, at each occurrence, independently selected of phenyl, naphthyl, 5-membered heteroaryl, 6-membered heteroaryl, 7-membered heteroaryl, 8-membered heteroaryl, 9-membered heteroaryl or 10-membered heteroaryl, 3-membered to 10-membered cycloalkyl, 5-membered to 10-membered heterocycloalkyl; Each of heteroaryl and heterocycloalkyl at each occurrence independently comprises 1, 2, 3, or 4 heteroatoms selected from N, O, or S; Each Ar₃ is, at each occurrence, independently optionally substituted or unsubstituted by 1, 2, 3, 4, 5 or 6 R_{19S};
Each Ar₃ is independently selected, at each occurrence, from H, phenyl, naphthyl, 5-membered heteroaryl, 6-membered heteroaryl, 7-membered heteroaryl, 8-membered heteroaryl, 9-membered heteroaryl, or 10-membered heteroaryl, 3-membered to 10-membered cycloalkyl, and 5-membered to 10-membered heterocyclic alkyl; Each of heteroaryl and heterocycloalkyl at each occurrence independently comprises 1, 2, 3, or 4 heteroatoms selected from N, O, or S; Each Ar₂ is, at each occurrence, independently and optionally substituted or unsubstituted by 1, 2, 3, 4, 5, or 6 R_{19S};
Each R₁₉ is independently selected, at each occurrence, from deuterium, halogen, oxo, —C₁₋₆ alkyl, —C₁₋₆ alkylidene-(halogen)₁₋₃, C₁₋₆ heteroalkyl, —CN, —OR₁₀, —C₁₋₆ alkylidene-(OR₁₀)₁₋₃, —O—C₁₋₆ alkylidene-(halogen)₁₋₃, —SR₁₀, —S—C₁₋₆ alkylidene-(halogen)₁₋₃, —NR₁₀R₁₁, —C₁₋₆ alkylidene-NR₁₀R₁₁, —C(═O)R₁₀, —C(═O)OR₁₀, —OC(═O)R₁₀, —C(═O)NR₁₀R₁₁, —NR₁₀C(═O)R₁₁, —S(O)₂NR₁₀R₁₁, or —C₃₋₆ carbocyclic; Each R₁₉ is independently optionally substituted by 1, 2, 3, 4, 5 or 6 substituents selected from deuterium, halogen, —C₁₋₆ alkyl, —C₁₋₆ alkoxy, oxo, —OR₁₀, —NR₁₀R₁₁, —CN, —C(═O)R₁₀, —C(═O)OR₁₀, —OC(═O)R₁₀, —C(═O)NR₁₀R₁₁, —NR₁₀C(═O)R₁₁, or —S(O)₂NR₆R₁₁;
Each of R₁₀ and R₁₁ is independently selected, at each occurrence, from hydrogen, deuterium, or —C₁₋₆ alkyl, and each of R₁₀ and R₁₁ is independently optionally substituted or unsubstituted by 1, 2, 3, 4, 5, or 6 R₁₉ₛ; or R₁₀, R₁₁, and the atoms attached thereto form a 3-membered to 10-membered heterocyclic ring, and the stated 3-membered to 10-membered heterocyclic ring can further comprise 1, 2, 3, or 4 heteroatoms selected from N, O, S, S(═O), or S(═O)₂, and the stated 3-membered to 10-membered heterocyclic ring is independently optionally substituted or unsubstituted by 1, 2, 3, 4, 5, or 6 R_{20S};
Each R₂₀ is independently selected, at each occurrence, from deuterium, halogen, oxo, —C₁₋₆ alkyl, —C₁₋₆ alkylidene-(halogen)₁₋₃, C₁₋₆ heteroalkyl, .CN, —OC₁₋₆, —C₁₋₆ alkylidene-(OC₁-6)₁₋₃, —O—C₁₋₆ alkylidene-(halogen)₁₋₃, —SC₁₋₆, —S—C₁₋₆ alkylidene-(halogen)₁₋₃, or —C₃₋₆ carbocyclic;
Further preferably, each 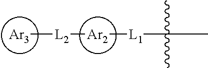
is selected from the following structure:

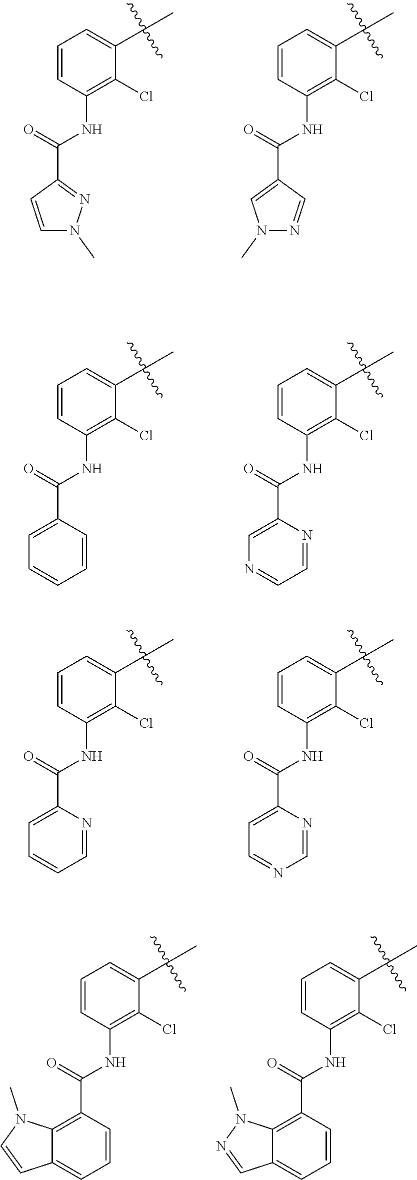

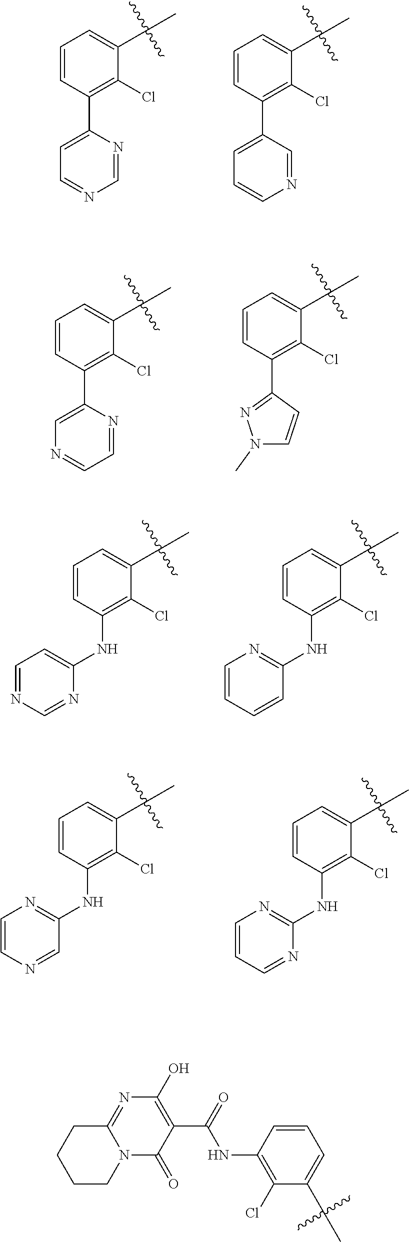

5. The compounds according to claim 1 or its prodrug, stable isotope derivatives, pharmaceutically acceptable salts, polymorphs or isomers and mixtures thereof, which are selected from the following compounds: 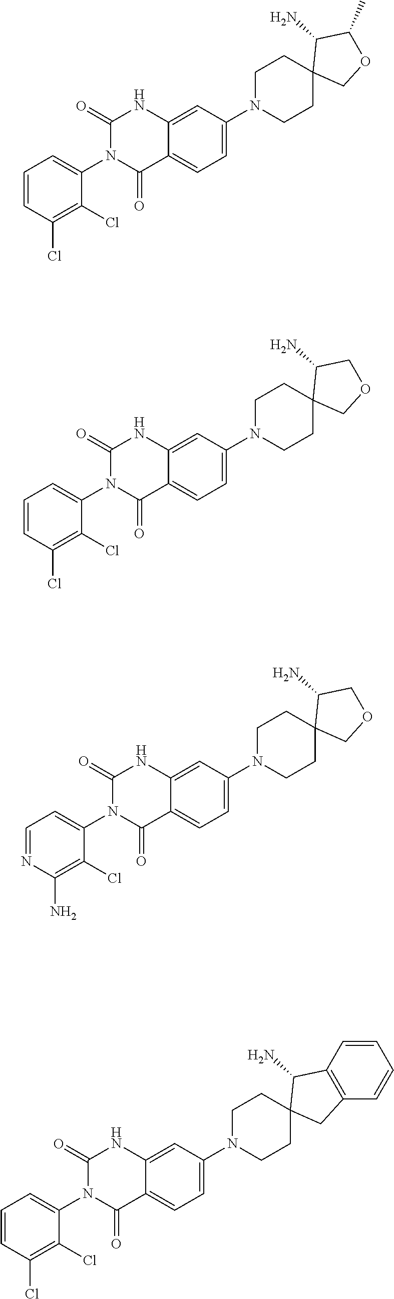 [SMILES: C[C@@H]1OCC2(CCN(c3ccc4c(=O)n(-c5cccc(Cl)c5Cl)c(=O)[nH]c4c3)CC2)[C@@H]1N ; N[C@@H]1COCC12CCN(c1ccc3c(=O)n(-c4cccc(Cl)c4Cl)c(=O)[nH]c3c1)CC2 ; Nc1nccc(-n2c(=O)[nH]c3cc(N4CCC5(CC4)COC[C@H]5N)ccc3c2=O)c1Cl ; N[C@@H]1c2ccccc2CC12CCN(c1ccc3c(=O)n(-c4cccc(Cl)c4Cl)c(=O)[nH]c3c1)CC2] 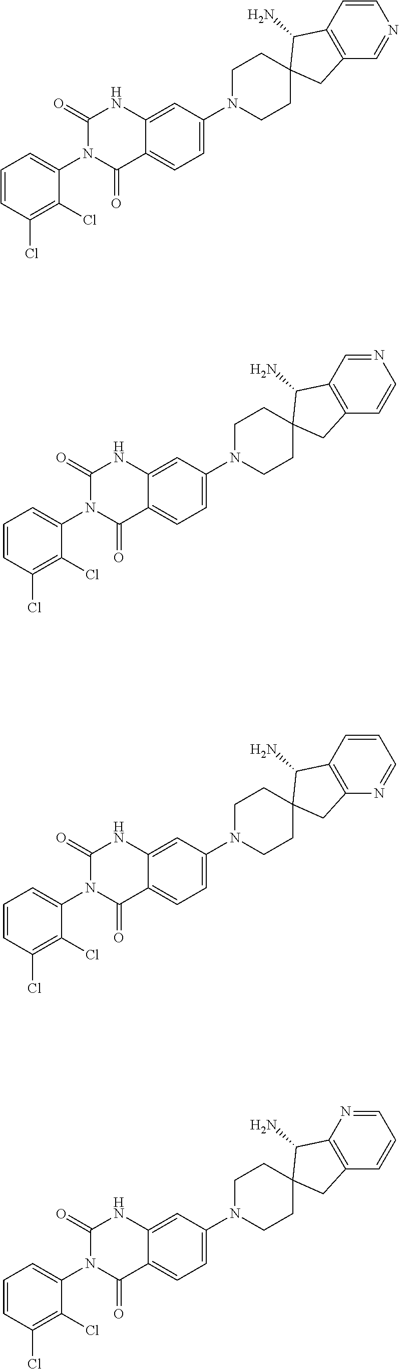 [SMILES: N[C@@H]1c2ccncc2CC12CCN(c1ccc3c(=O)n(-c4cccc(Cl)c4Cl)c(=O)[nH]c3c1)CC2 ; N[C@@H]1c2cnccc2CC12CCN(c1ccc3c(=O)n(-c4cccc(Cl)c4Cl)c(=O)[nH]c3c1)CC2 ; N[C@@H]1c2cccnc2CC12CCN(c1ccc3c(=O)n(-c4cccc(Cl)c4Cl)c(=O)[nH]c3c1)CC2 ; N[C@@H]1c2ncccc2CC12CCN(c1ccc3c(=O)n(-c4cccc(Cl)c4Cl)c(=O)[nH]c3c1)CC2] 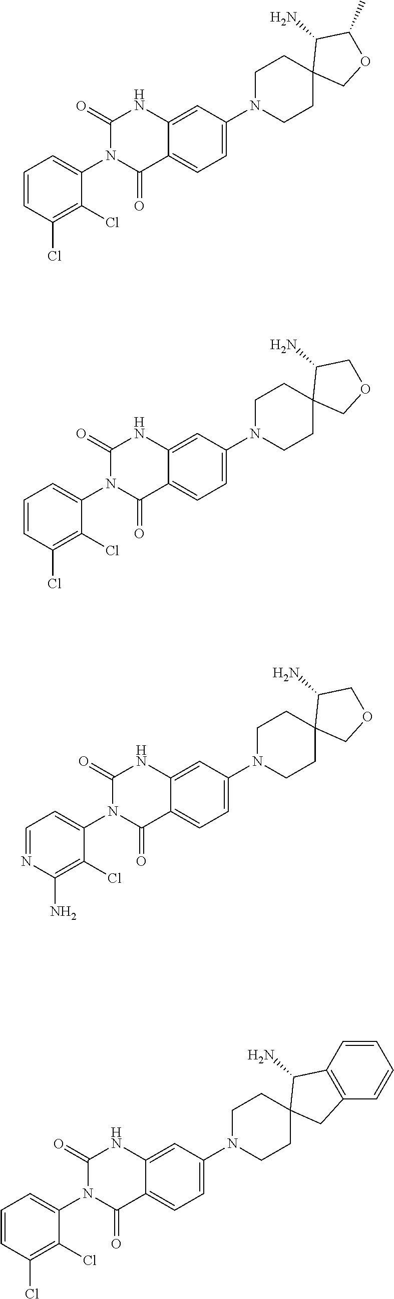 [SMILES: C[C@@H]1OCC2(CCN(c3ccc4c(=O)n(-c5cccc(Cl)c5Cl)c(=O)[nH]c4c3)CC2)[C@@H]1N ; N[C@@H]1COCC12CCN(c1ccc3c(=O)n(-c4cccc(Cl)c4Cl)c(=O)[nH]c3c1)CC2 ; Nc1nccc(-n2c(=O)[nH]c3cc(N4CCC5(CC4)COC[C@H]5N)ccc3c2=O)c1Cl ; N[C@@H]1c2ccccc2CC12CCN(c1ccc3c(=O)n(-c4cccc(Cl)c4Cl)c(=O)[nH]c3c1)CC2] 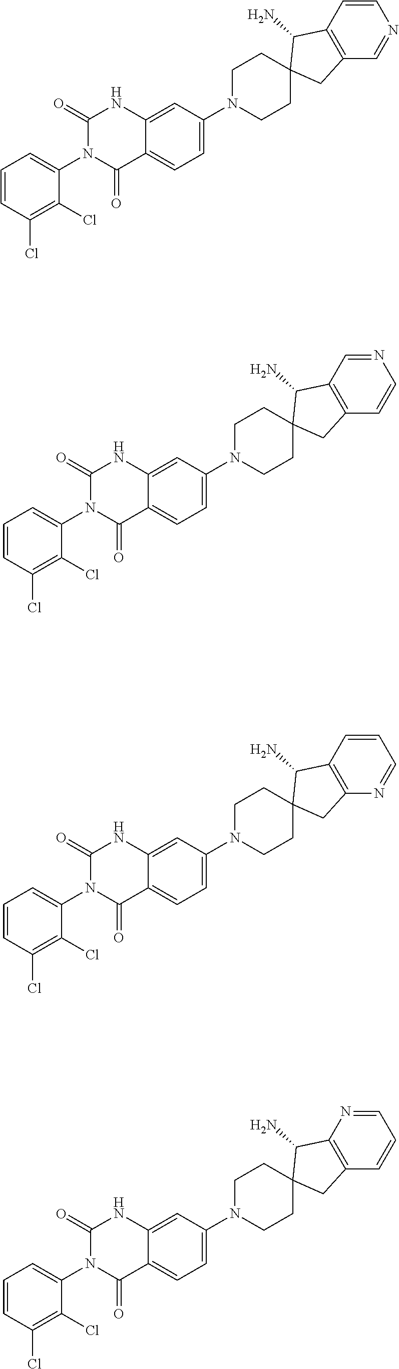 [SMILES: N[C@@H]1c2ccncc2CC12CCN(c1ccc3c(=O)n(-c4cccc(Cl)c4Cl)c(=O)[nH]c3c1)CC2 ; N[C@@H]1c2cnccc2CC12CCN(c1ccc3c(=O)n(-c4cccc(Cl)c4Cl)c(=O)[nH]c3c1)CC2 ; N[C@@H]1c2cccnc2CC12CCN(c1ccc3c(=O)n(-c4cccc(Cl)c4Cl)c(=O)[nH]c3c1)CC2 ; N[C@@H]1c2ncccc2CC12CCN(c1ccc3c(=O)n(-c4cccc(Cl)c4Cl)c(=O)[nH]c3c1)CC2] 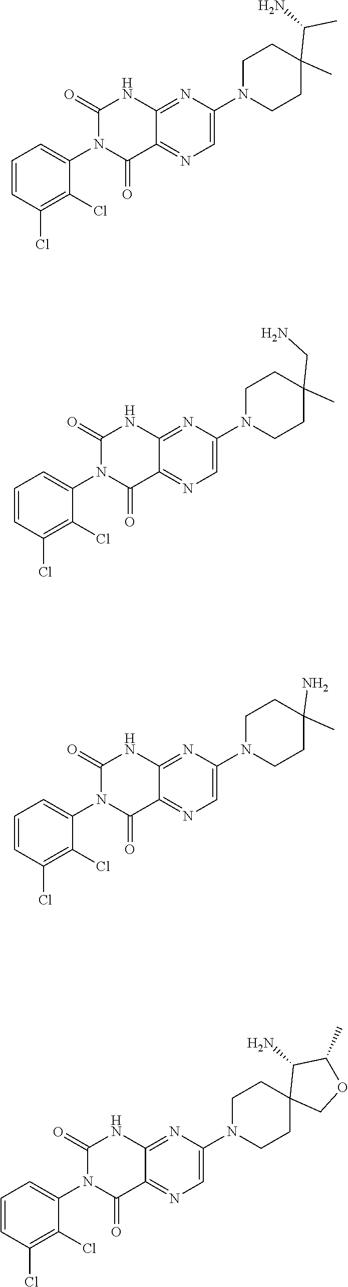 [SMILES: C[C@@H](N)C1(C)CCN(c2cnc3c(=O)n(-c4cccc(Cl)c4Cl)c(=O)[nH]c3n2)CC1 ; CC1(CN)CCN(c2cnc3c(=O)n(-c4cccc(Cl)c4Cl)c(=O)[nH]c3n2)CC1 ; CC1(N)CCN(c2cnc3c(=O)n(-c4cccc(Cl)c4Cl)c(=O)[nH]c3n2)CC1 ; C[C@@H]1OCC2(CCN(c3cnc4c(=O)n(-c5cccc(Cl)c5Cl)c(=O)[nH]c4n3)CC2)[C@@H]1N] 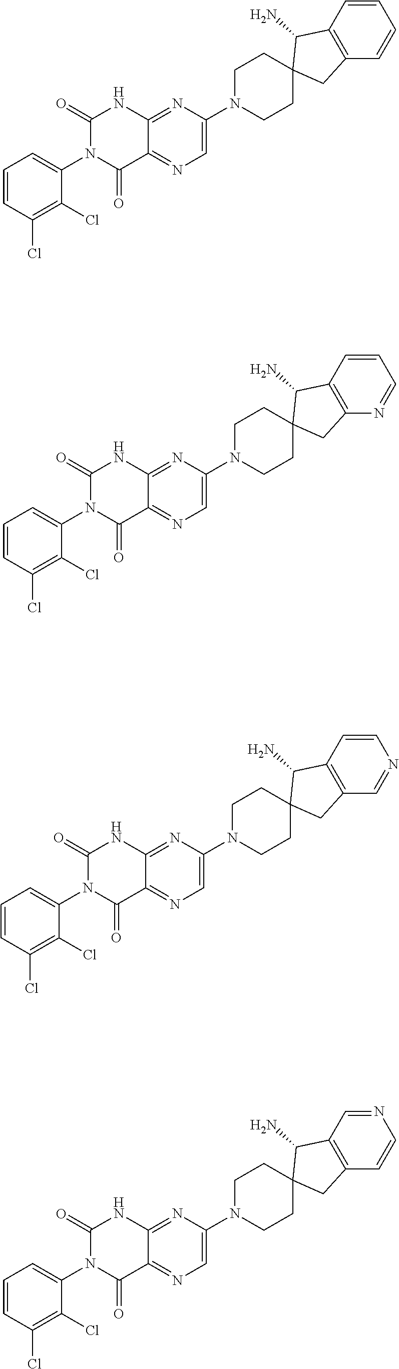 [SMILES: N[C@@H]1c2ccccc2CC12CCN(c1cnc3c(=O)n(-c4cccc(Cl)c4Cl)c(=O)[nH]c3n1)CC2 ; N[C@@H]1c2cccnc2CC12CCN(c1cnc3c(=O)n(-c4cccc(Cl)c4Cl)c(=O)[nH]c3n1)CC2 ; N[C@@H]1c2ccncc2CC12CCN(c1cnc3c(=O)n(-c4cccc(Cl)c4Cl)c(=O)[nH]c3n1)CC2 ; N[C@@H]1c2cnccc2CC12CCN(c1cnc3c(=O)n(-c4cccc(Cl)c4Cl)c(=O)[nH]c3n1)CC2] 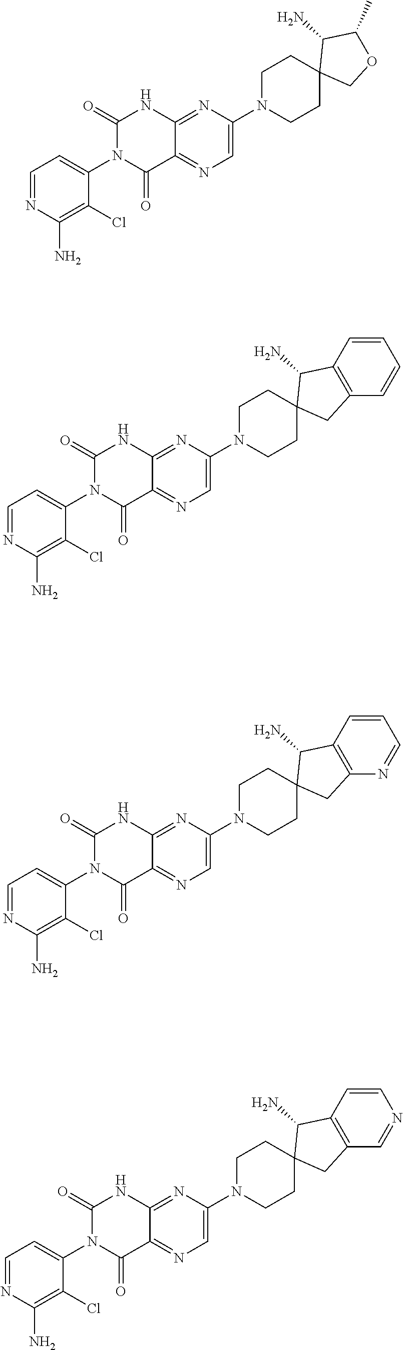 [SMILES: C[C@@H]1OCC2(CCN(c3cnc4c(=O)n(-c5ccnc(N)c5Cl)c(=O)[nH]c4n3)CC2)[C@@H]1N ; Nc1nccc(-n2c(=O)[nH]c3nc(N4CCC5(CC4)Cc4ccccc4[C@H]5N)cnc3c2=O)c1Cl ; Nc1nccc(-n2c(=O)[nH]c3nc(N4CCC5(CC4)Cc4ncccc4[C@H]5N)cnc3c2=O)c1Cl ; Nc1nccc(-n2c(=O)[nH]c3nc(N4CCC5(CC4)Cc4cnccc4[C@H]5N)cnc3c2=O)c1Cl] 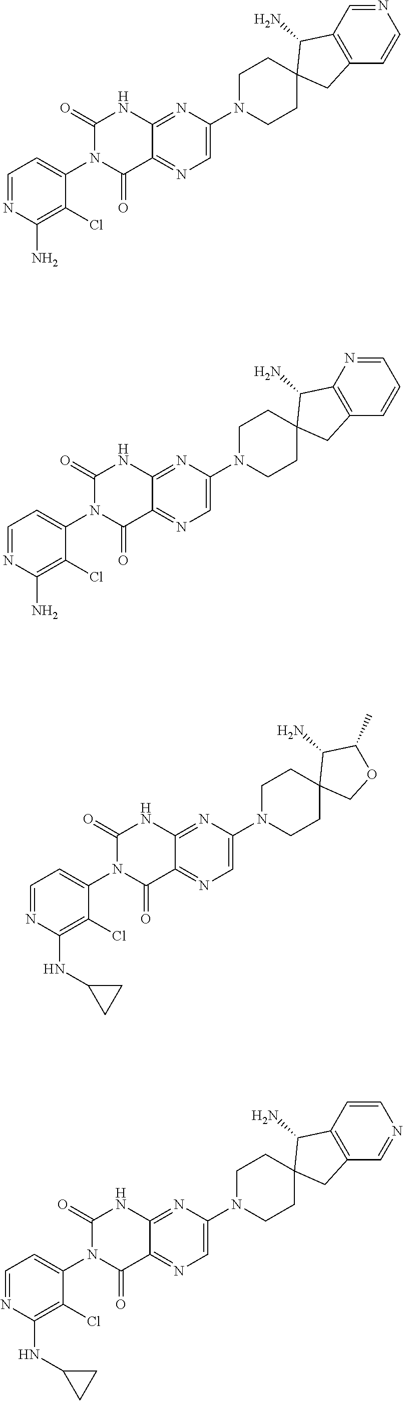 [SMILES: Nc1nccc(-n2c(=O)[nH]c3nc(N4CCC5(CC4)Cc4ccncc4[C@H]5N)cnc3c2=O)c1Cl ; Nc1nccc(-n2c(=O)[nH]c3nc(N4CCC5(CC4)Cc4cccnc4[C@H]5N)cnc3c2=O)c1Cl ; C[C@@H]1OCC2(CCN(c3cnc4c(=O)n(-c5ccnc(NC6CC6)c5Cl)c(=O)[nH]c4n3)CC2)[C@@H]1N ; N[C@@H]1c2ccncc2CC12CCN(c1cnc3c(=O)n(-c4ccnc(NC5CC5)c4Cl)c(=O)[nH]c3n1)CC2] 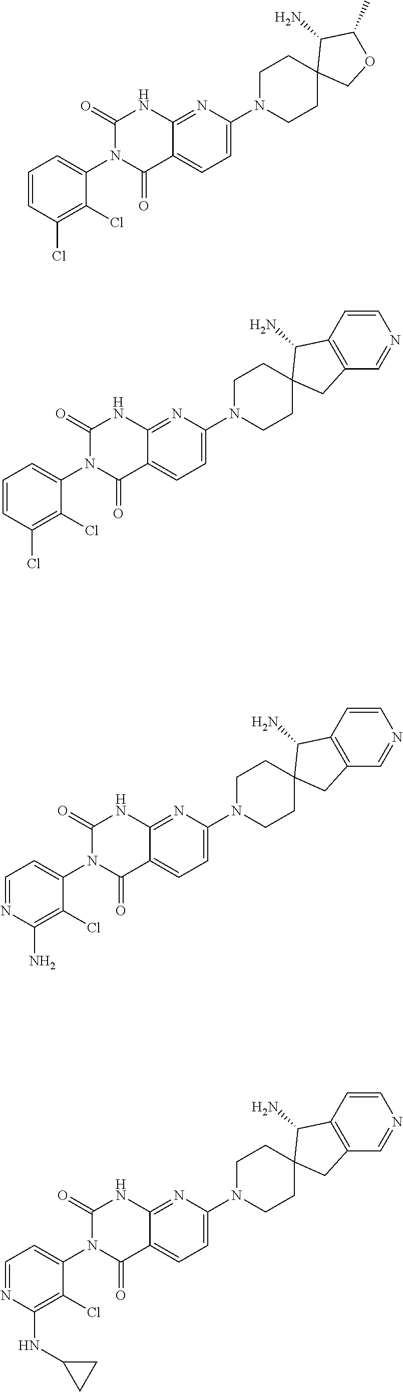 [SMILES: C[C@@H]1OCC2(CCN(c3ccc4c(=O)n(-c5cccc(Cl)c5Cl)c(=O)[nH]c4n3)CC2)[C@@H]1N ; N[C@@H]1c2ccncc2CC12CCN(c1ccc3c(=O)n(-c4cccc(Cl)c4Cl)c(=O)[nH]c3n1)CC2 ; Nc1nccc(-n2c(=O)[nH]c3nc(N4CCC5(CC4)Cc4cnccc4[C@H]5N)ccc3c2=O)c1Cl ; N[C@@H]1c2ccncc2CC12CCN(c1ccc3c(=O)n(-c4ccnc(NC5CC5)c4Cl)c(=O)[nH]c3n1)CC2] 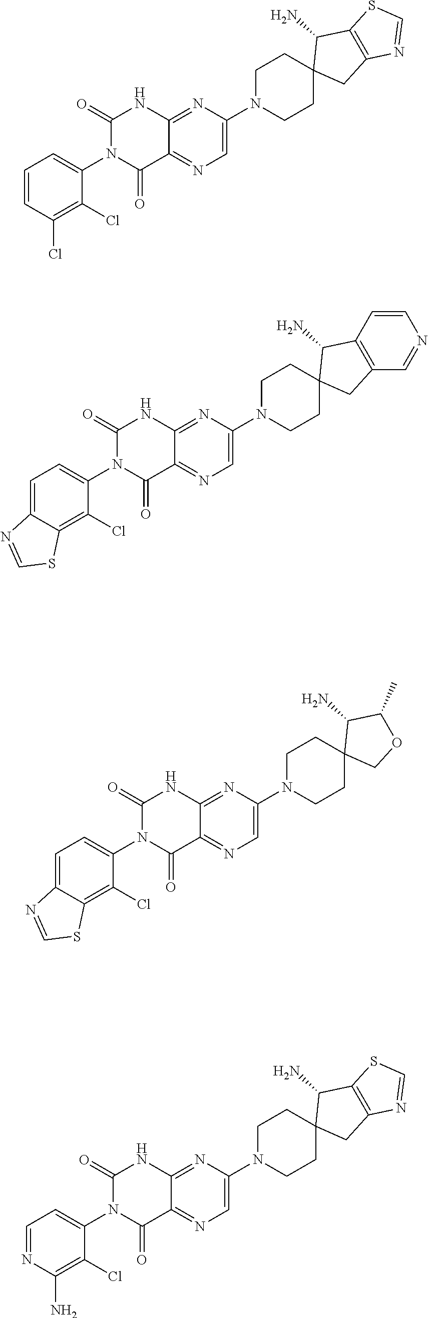 [SMILES: N[C@@H]1c2scnc2CC12CCN(c1cnc3c(=O)n(-c4cccc(Cl)c4Cl)c(=O)[nH]c3n1)CC2 ; N[C@@H]1c2ccncc2CC12CCN(c1cnc3c(=O)n(-c4ccc5ncsc5c4Cl)c(=O)[nH]c3n1)CC2 ; C[C@@H]1OCC2(CCN(c3cnc4c(=O)n(-c5ccc6ncsc6c5Cl)c(=O)[nH]c4n3)CC2)[C@@H]1N ; Nc1nccc(-n2c(=O)[nH]c3nc(N4CCC5(CC4)Cc4ncsc4[C@H]5N)cnc3c2=O)c1Cl] 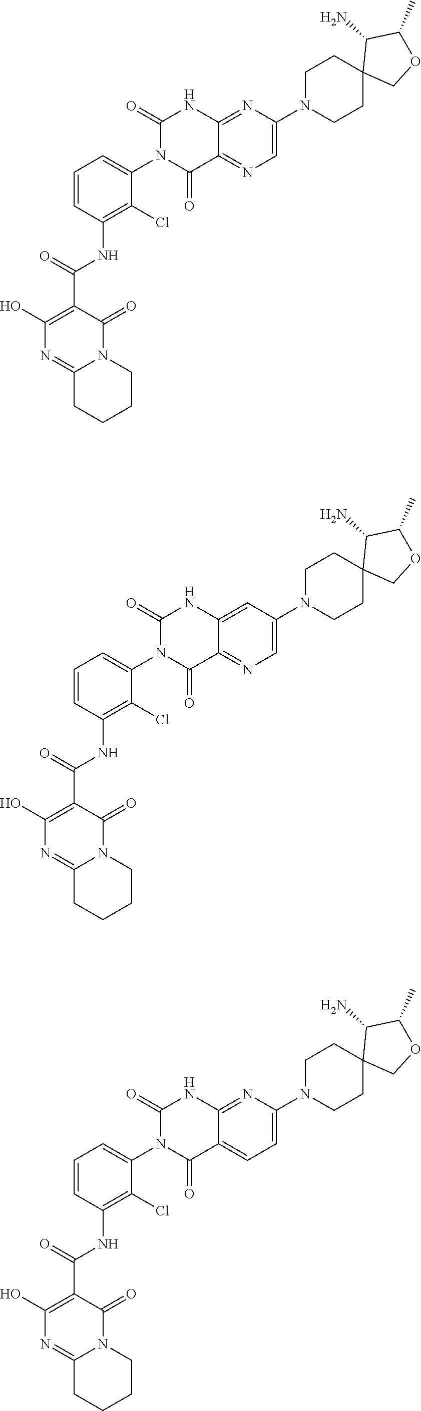 [SMILES: C[C@@H]1OCC2(CCN(c3cnc4c(=O)n(-c5cccc(NC(=O)c6c(O)nc7n(c6=O)CCCC7)c5Cl)c(=O)[nH]c4n3)CC2)[C@@H]1N ; C[C@@H]1OCC2(CCN(c3cnc4c(=O)n(-c5cccc(NC(=O)c6c(O)nc7n(c6=O)CCCC7)c5Cl)c(=O)[nH]c4c3)CC2)[C@@H]1N ; C[C@@H]1OCC2(CCN(c3ccc4c(=O)n(-c5cccc(NC(=O)c6c(O)nc7n(c6=O)CCCC7)c5Cl)c(=O)[nH]c4n3)CC2)[C@@H]1N] 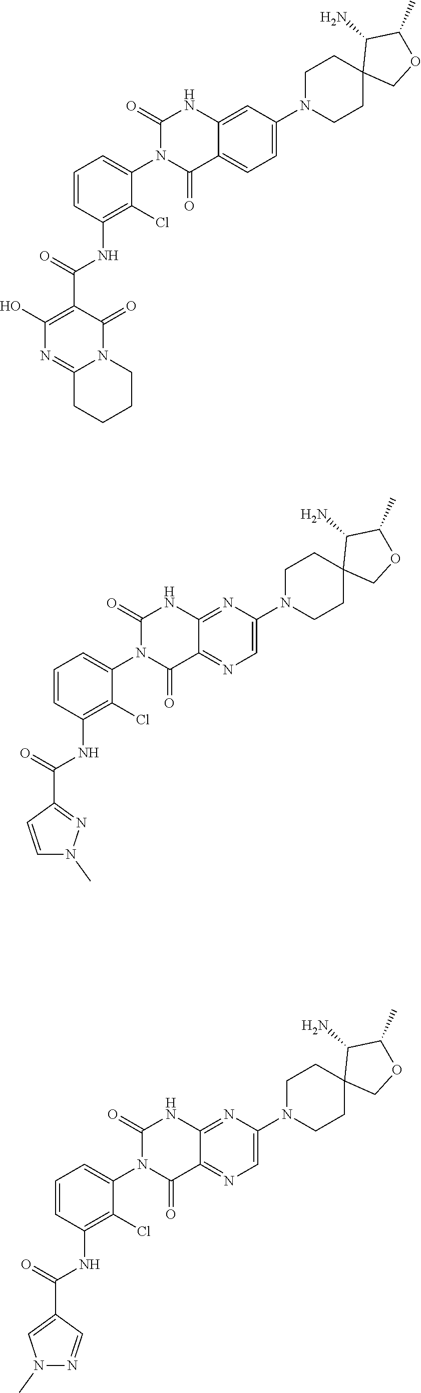 [SMILES: C[C@@H]1OCC2(CCN(c3ccc4c(=O)n(-c5cccc(NC(=O)c6c(O)nc7n(c6=O)CCCC7)c5Cl)c(=O)[nH]c4c3)CC2)[C@@H]1N ; C[C@@H]1OCC2(CCN(c3cnc4c(=O)n(-c5cccc(NC(=O)c6ccn(C)n6)c5Cl)c(=O)[nH]c4n3)CC2)[C@@H]1N ; C[C@@H]1OCC2(CCN(c3cnc4c(=O)n(-c5cccc(NC(=O)c6cnn(C)c6)c5Cl)c(=O)[nH]c4n3)CC2)[C@@H]1N] 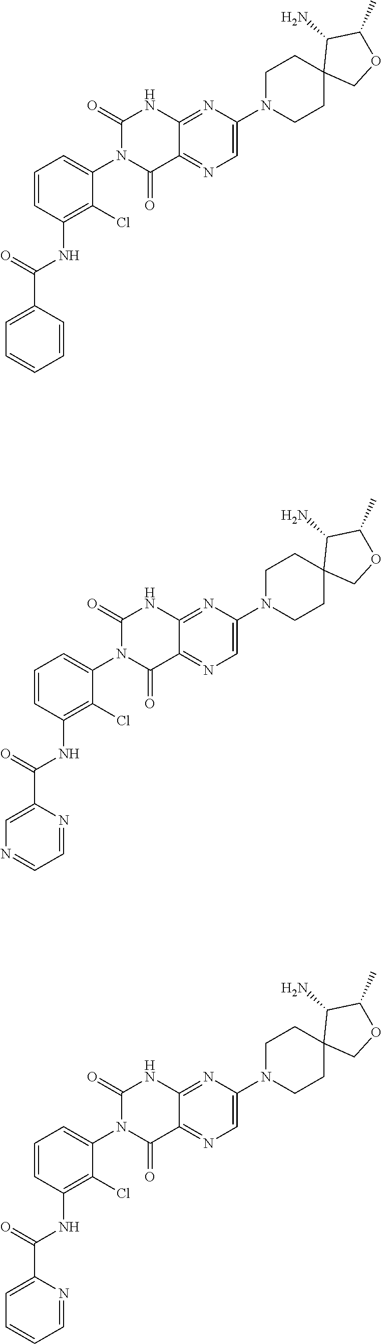 [SMILES: C[C@@H]1OCC2(CCN(c3cnc4c(=O)n(-c5cccc(NC(=O)c6ccccc6)c5Cl)c(=O)[nH]c4n3)CC2)[C@@H]1N ; C[C@@H]1OCC2(CCN(c3cnc4c(=O)n(-c5cccc(NC(=O)c6cnccn6)c5Cl)c(=O)[nH]c4n3)CC2)[C@@H]1N ; C[C@@H]1OCC2(CCN(c3cnc4c(=O)n(-c5cccc(NC(=O)c6ccccn6)c5Cl)c(=O)[nH]c4n3)CC2)[C@@H]1N] 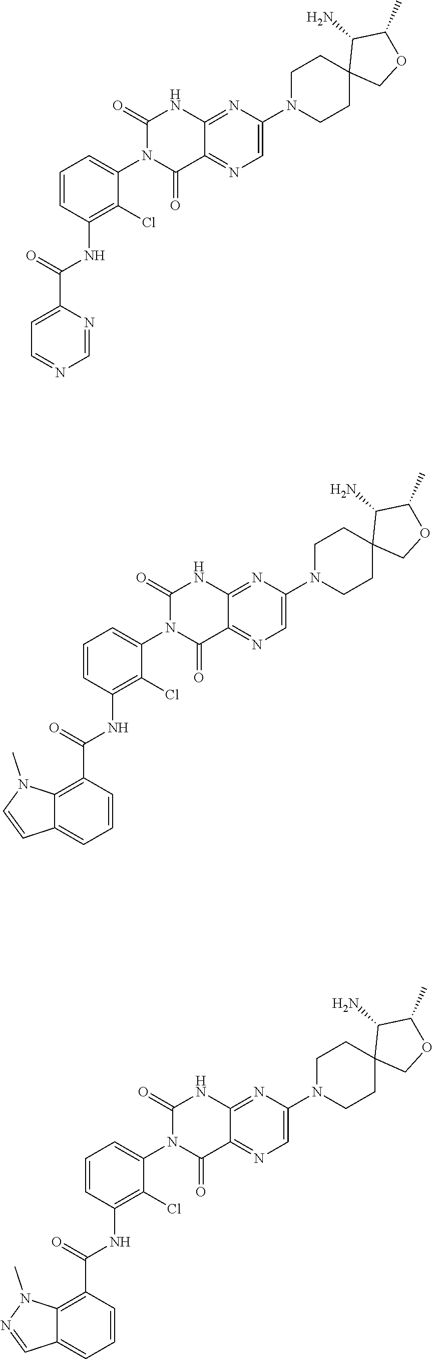 [SMILES: C[C@@H]1OCC2(CCN(c3cnc4c(=O)n(-c5cccc(NC(=O)c6ccncn6)c5Cl)c(=O)[nH]c4n3)CC2)[C@@H]1N ; C[C@@H]1OCC2(CCN(c3cnc4c(=O)n(-c5cccc(NC(=O)c6cccc7ccn(C)c67)c5Cl)c(=O)[nH]c4n3)CC2)[C@@H]1N ; C[C@@H]1OCC2(CCN(c3cnc4c(=O)n(-c5cccc(NC(=O)c6cccc7cnn(C)c67)c5Cl)c(=O)[nH]c4n3)CC2)[C@@H]1N] 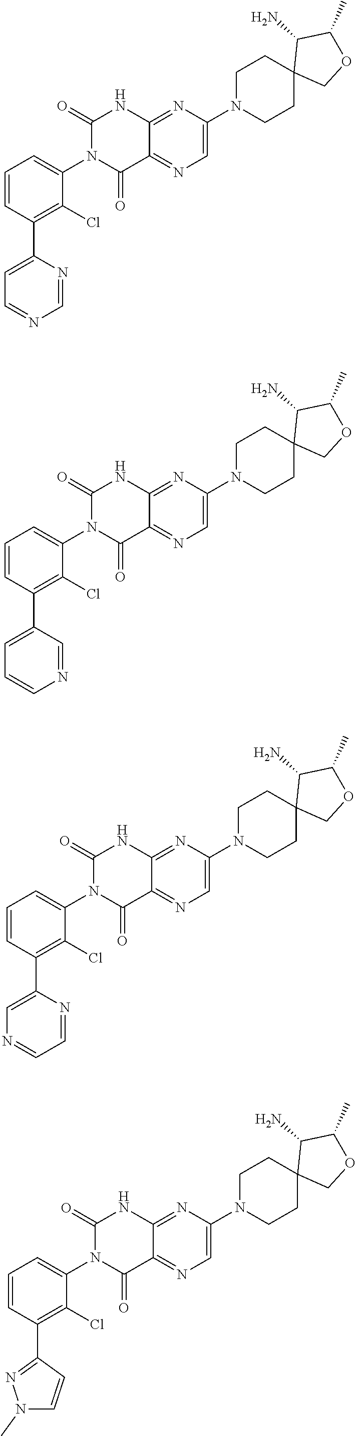 [SMILES: C[C@@H]1OCC2(CCN(c3cnc4c(=O)n(-c5cccc(-c6ccncn6)c5Cl)c(=O)[nH]c4n3)CC2)[C@@H]1N ; C[C@@H]1OCC2(CCN(c3cnc4c(=O)n(-c5cccc(-c6cccnc6)c5Cl)c(=O)[nH]c4n3)CC2)[C@@H]1N ; C[C@@H]1OCC2(CCN(c3cnc4c(=O)n(-c5cccc(-c6cnccn6)c5Cl)c(=O)[nH]c4n3)CC2)[C@@H]1N ; C[C@@H]1OCC2(CCN(c3cnc4c(=O)n(-c5cccc(-c6ccn(C)n6)c5Cl)c(=O)[nH]c4n3)CC2)[C@@H]1N] 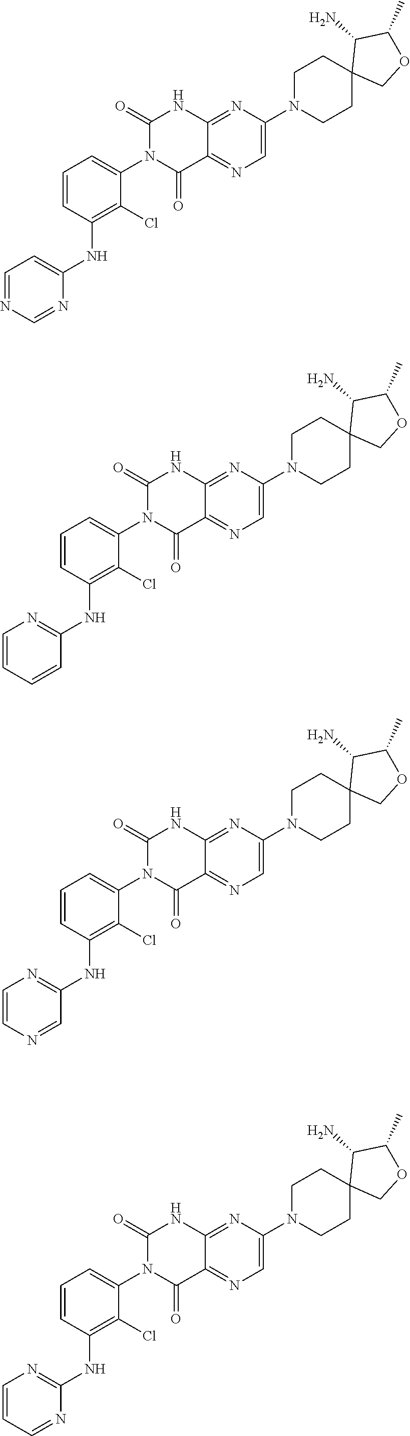 [SMILES: C[C@@H]1OCC2(CCN(c3cnc4c(=O)n(-c5cccc(Nc6ccncn6)c5Cl)c(=O)[nH]c4n3)CC2)[C@@H]1N ; C[C@@H]1OCC2(CCN(c3cnc4c(=O)n(-c5cccc(Nc6ccccn6)c5Cl)c(=O)[nH]c4n3)CC2)[C@@H]1N ; C[C@@H]1OCC2(CCN(c3cnc4c(=O)n(-c5cccc(Nc6cnccn6)c5Cl)c(=O)[nH]c4n3)CC2)[C@@H]1N ; C[C@@H]1OCC2(CCN(c3cnc4c(=O)n(-c5cccc(Nc6ncccn6)c5Cl)c(=O)[nH]c4n3)CC2)[C@@H]1N] 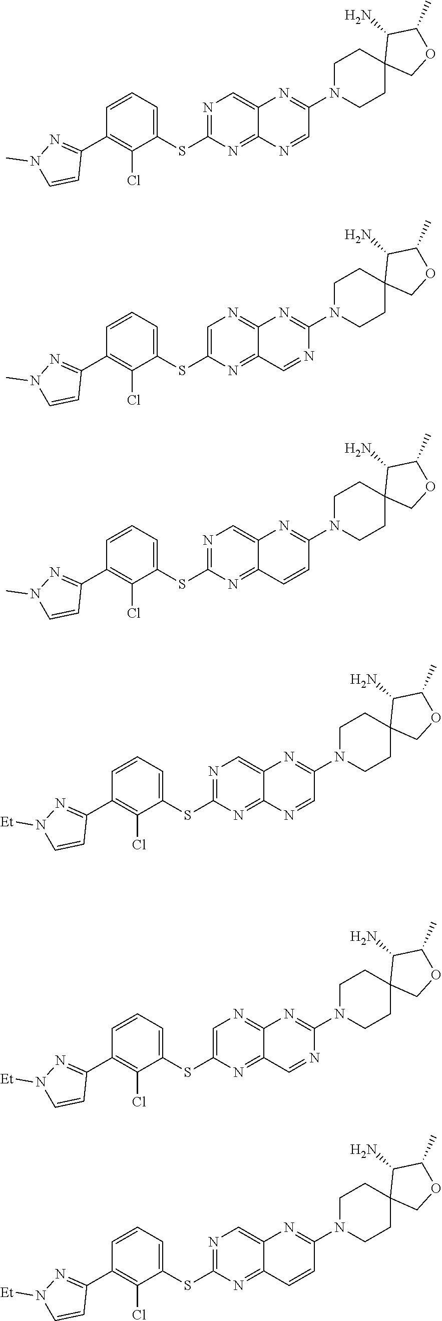 [SMILES: C[C@@H]1OCC2(CCN(c3cnc4nc(Sc5cccc(-c6ccn(C)n6)c5Cl)ncc4n3)CC2)[C@@H]1N ; C[C@@H]1OCC2(CCN(c3ncc4nc(Sc5cccc(-c6ccn(C)n6)c5Cl)cnc4n3)CC2)[C@@H]1N ; C[C@@H]1OCC2(CCN(c3ccc4nc(Sc5cccc(-c6ccn(C)n6)c5Cl)ncc4n3)CC2)[C@@H]1N ; CCn1ccc(-c2cccc(Sc3ncc4nc(N5CCC6(CC5)CO[C@@H](C)[C@H]6N)cnc4n3)c2Cl)n1 ; CCn1ccc(-c2cccc(Sc3cnc4nc(N5CCC6(CC5)CO[C@@H](C)[C@H]6N)ncc4n3)c2Cl)n1 ; CCn1ccc(-c2cccc(Sc3ncc4nc(N5CCC6(CC5)CO[C@@H](C)[C@H]6N)ccc4n3)c2Cl)n1] 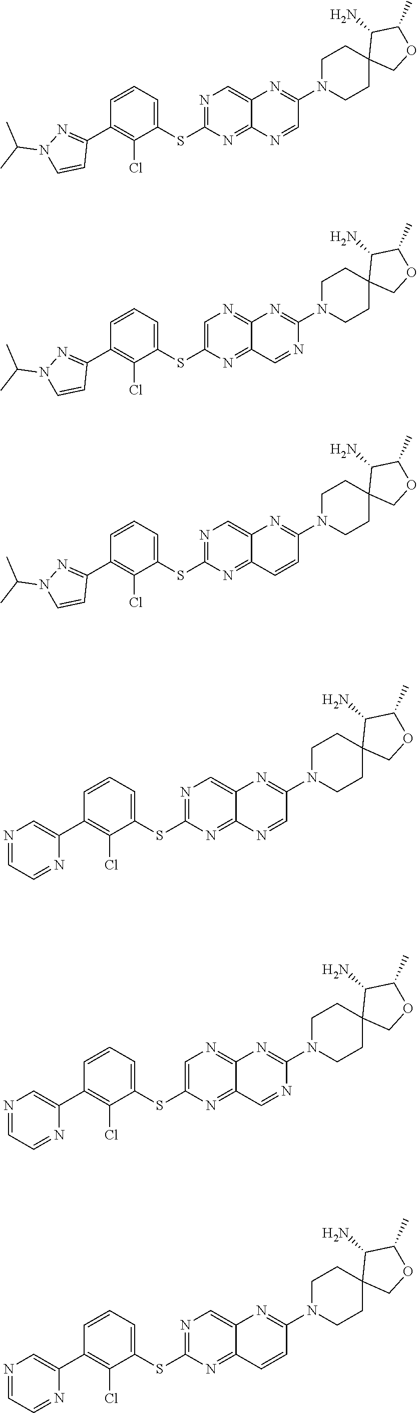 [SMILES: CC(C)n1ccc(-c2cccc(Sc3ncc4nc(N5CCC6(CC5)CO[C@@H](C)[C@H]6N)cnc4n3)c2Cl)n1 ; CC(C)n1ccc(-c2cccc(Sc3cnc4nc(N5CCC6(CC5)CO[C@@H](C)[C@H]6N)ncc4n3)c2Cl)n1 ; CC(C)n1ccc(-c2cccc(Sc3ncc4nc(N5CCC6(CC5)CO[C@@H](C)[C@H]6N)ccc4n3)c2Cl)n1 ; C[C@@H]1OCC2(CCN(c3cnc4nc(Sc5cccc(-c6cnccn6)c5Cl)ncc4n3)CC2)[C@@H]1N ; C[C@@H]1OCC2(CCN(c3ncc4nc(Sc5cccc(-c6cnccn6)c5Cl)cnc4n3)CC2)[C@@H]1N ; C[C@@H]1OCC2(CCN(c3ccc4nc(Sc5cccc(-c6cnccn6)c5Cl)ncc4n3)CC2)[C@@H]1N] 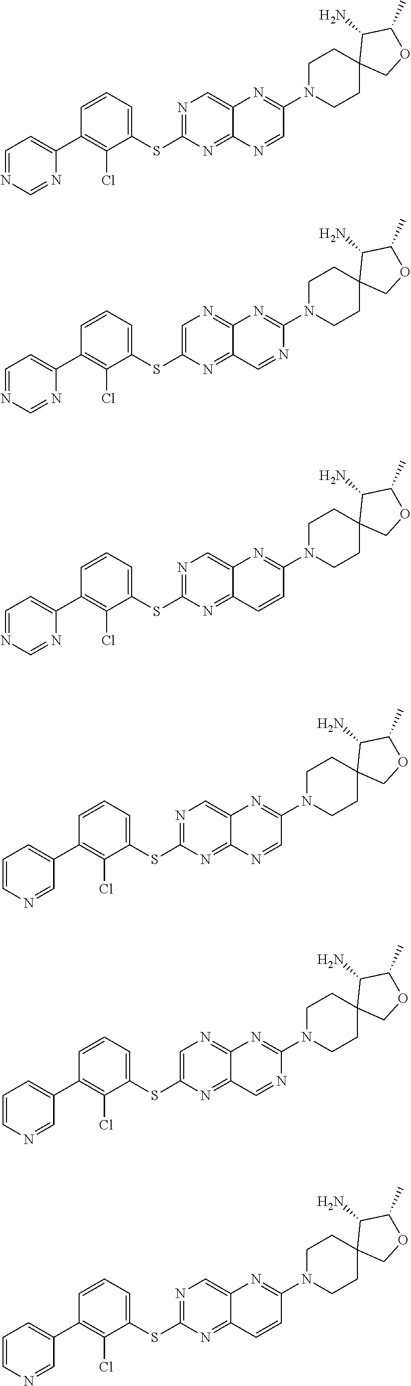 [SMILES: C[C@@H]1OCC2(CCN(c3cnc4nc(Sc5cccc(-c6ccncn6)c5Cl)ncc4n3)CC2)[C@@H]1N ; C[C@@H]1OCC2(CCN(c3ncc4nc(Sc5cccc(-c6ccncn6)c5Cl)cnc4n3)CC2)[C@@H]1N ; C[C@@H]1OCC2(CCN(c3ccc4nc(Sc5cccc(-c6ccncn6)c5Cl)ncc4n3)CC2)[C@@H]1N ; C[C@@H]1OCC2(CCN(c3cnc4nc(Sc5cccc(-c6cccnc6)c5Cl)ncc4n3)CC2)[C@@H]1N ; C[C@@H]1OCC2(CCN(c3ncc4nc(Sc5cccc(-c6cccnc6)c5Cl)cnc4n3)CC2)[C@@H]1N ; C[C@@H]1OCC2(CCN(c3ccc4nc(Sc5cccc(-c6cccnc6)c5Cl)ncc4n3)CC2)[C@@H]1N] 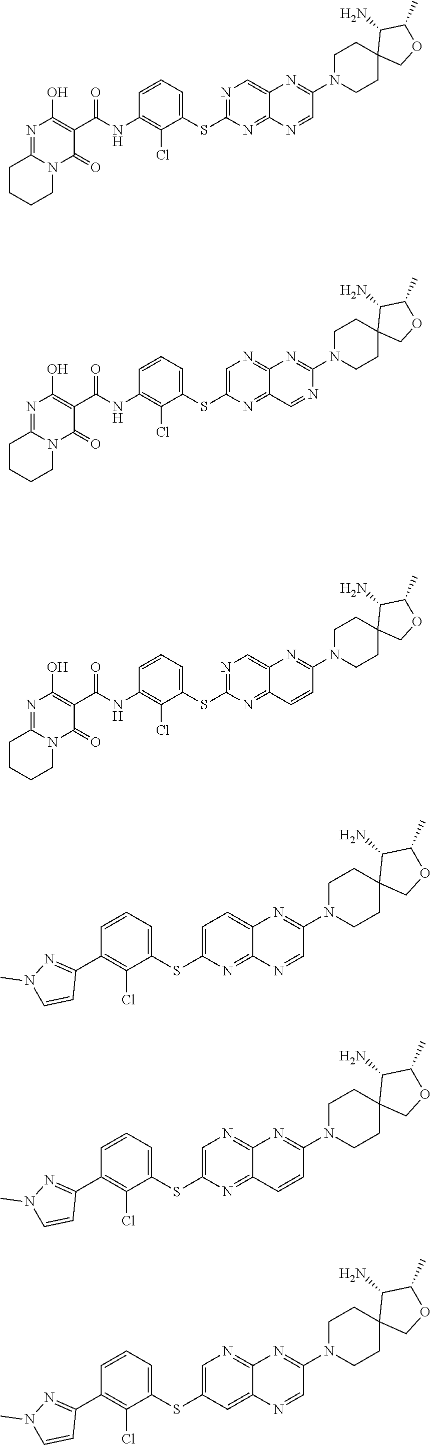 [SMILES: C[C@@H]1OCC2(CCN(c3cnc4nc(Sc5cccc(NC(=O)c6c(O)nc7n(c6=O)CCCC7)c5Cl)ncc4n3)CC2)[C@@H]1N ; C[C@@H]1OCC2(CCN(c3ncc4nc(Sc5cccc(NC(=O)c6c(O)nc7n(c6=O)CCCC7)c5Cl)cnc4n3)CC2)[C@@H]1N ; C[C@@H]1OCC2(CCN(c3ccc4nc(Sc5cccc(NC(=O)c6c(O)nc7n(c6=O)CCCC7)c5Cl)ncc4n3)CC2)[C@@H]1N ; C[C@@H]1OCC2(CCN(c3cnc4nc(Sc5cccc(-c6ccn(C)n6)c5Cl)ccc4n3)CC2)[C@@H]1N ; C[C@@H]1OCC2(CCN(c3ccc4nc(Sc5cccc(-c6ccn(C)n6)c5Cl)cnc4n3)CC2)[C@@H]1N ; C[C@@H]1OCC2(CCN(c3cnc4cc(Sc5cccc(-c6ccn(C)n6)c5Cl)cnc4n3)CC2)[C@@H]1N] 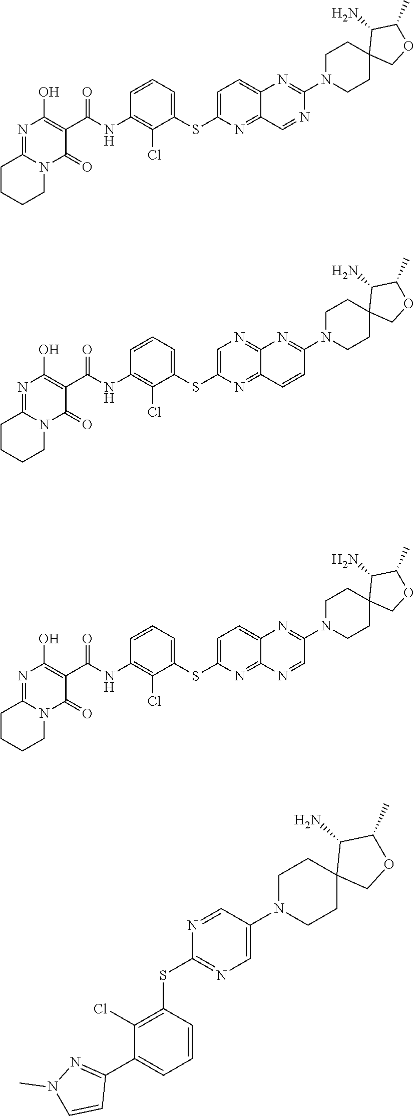 [SMILES: C[C@@H]1OCC2(CCN(c3ncc4nc(Sc5cccc(NC(=O)c6c(O)nc7n(c6=O)CCCC7)c5Cl)ccc4n3)CC2)[C@@H]1N ; C[C@@H]1OCC2(CCN(c3ccc4nc(Sc5cccc(NC(=O)c6c(O)nc7n(c6=O)CCCC7)c5Cl)cnc4n3)CC2)[C@@H]1N ; C[C@@H]1OCC2(CCN(c3cnc4nc(Sc5cccc(NC(=O)c6c(O)nc7n(c6=O)CCCC7)c5Cl)ccc4n3)CC2)[C@@H]1N ; C[C@@H]1OCC2(CCN(c3cnc(Sc4cccc(-c5ccn(C)n5)c4Cl)nc3)CC2)[C@@H]1N] 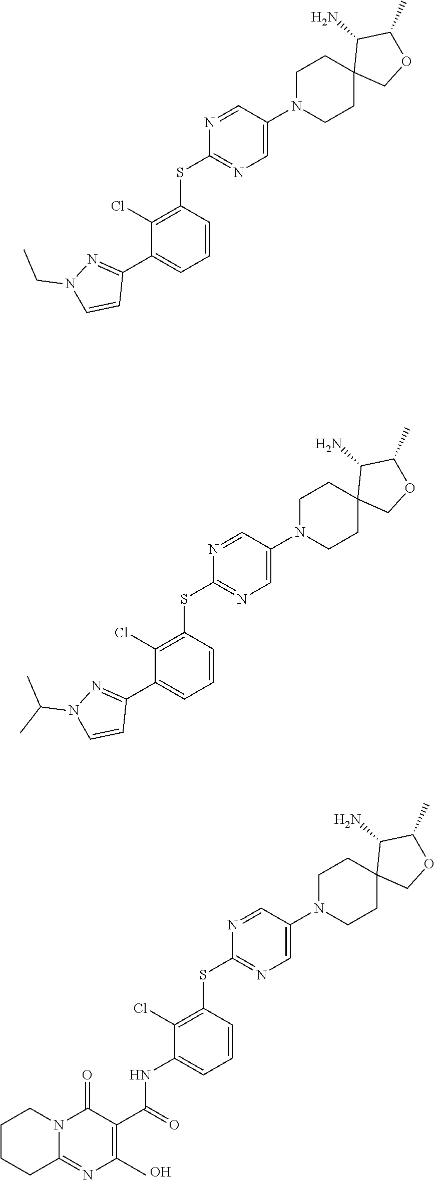 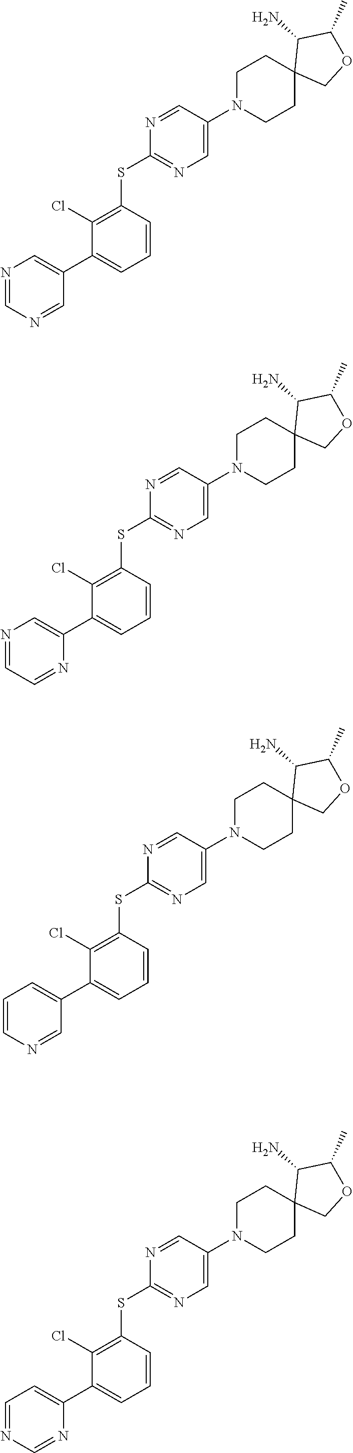 [SMILES: C[C@@H]1OCC2(CCN(c3cnc(Sc4cccc(-c5cncnc5)c4Cl)nc3)CC2)[C@@H]1N ; C[C@@H]1OCC2(CCN(c3cnc(Sc4cccc(-c5cnccn5)c4Cl)nc3)CC2)[C@@H]1N ; C[C@@H]1OCC2(CCN(c3cnc(Sc4cccc(-c5cccnc5)c4Cl)nc3)CC2)[C@@H]1N ; C[C@@H]1OCC2(CCN(c3cnc(Sc4cccc(-c5ccncn5)c4Cl)nc3)CC2)[C@@H]1N] 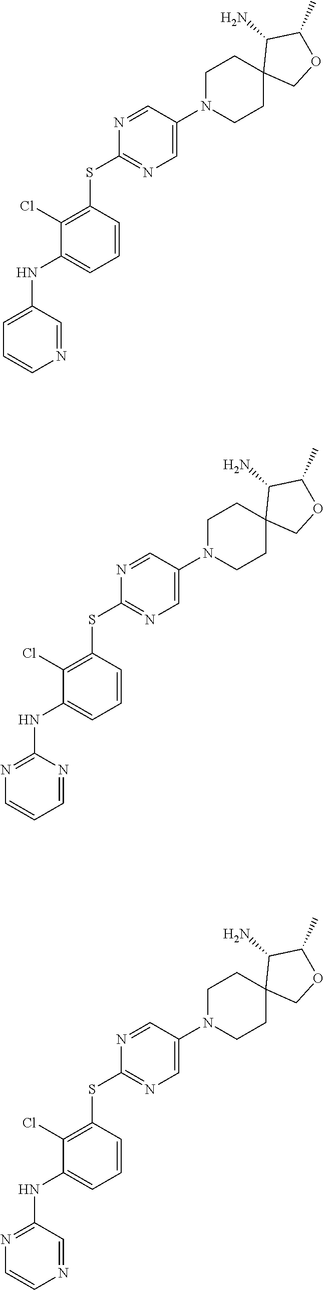 [SMILES: C[C@@H]1OCC2(CCN(c3cnc(Sc4cccc(Nc5cccnc5)c4Cl)nc3)CC2)[C@@H]1N ; C[C@@H]1OCC2(CCN(c3cnc(Sc4cccc(Nc5ncccn5)c4Cl)nc3)CC2)[C@@H]1N ; C[C@@H]1OCC2(CCN(c3cnc(Sc4cccc(Nc5cnccn5)c4Cl)nc3)CC2)[C@@H]1N] 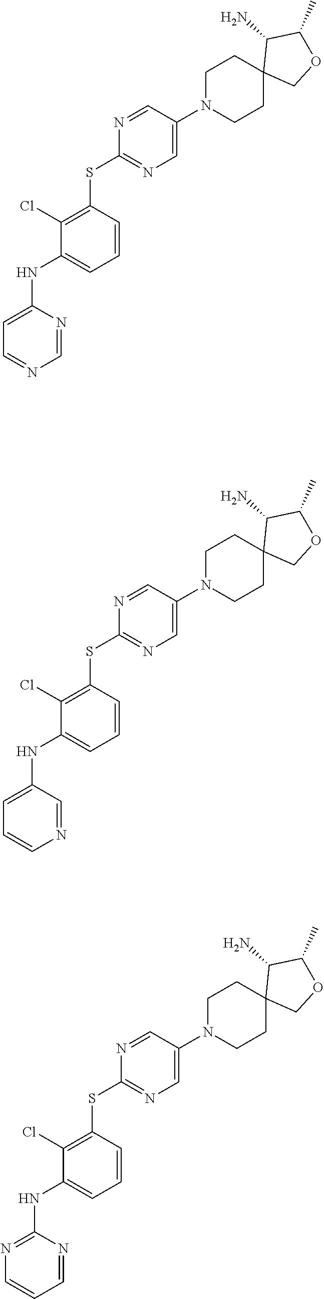 [SMILES: C[C@@H]1OCC2(CCN(c3cnc(Sc4cccc(Nc5ccncn5)c4Cl)nc3)CC2)[C@@H]1N ; C[C@@H]1OCC2(CCN(c3cnc(Sc4cccc(Nc5cccnc5)c4Cl)nc3)CC2)[C@@H]1N ; C[C@@H]1OCC2(CCN(c3cnc(Sc4cccc(Nc5ncccn5)c4Cl)nc3)CC2)[C@@H]1N] 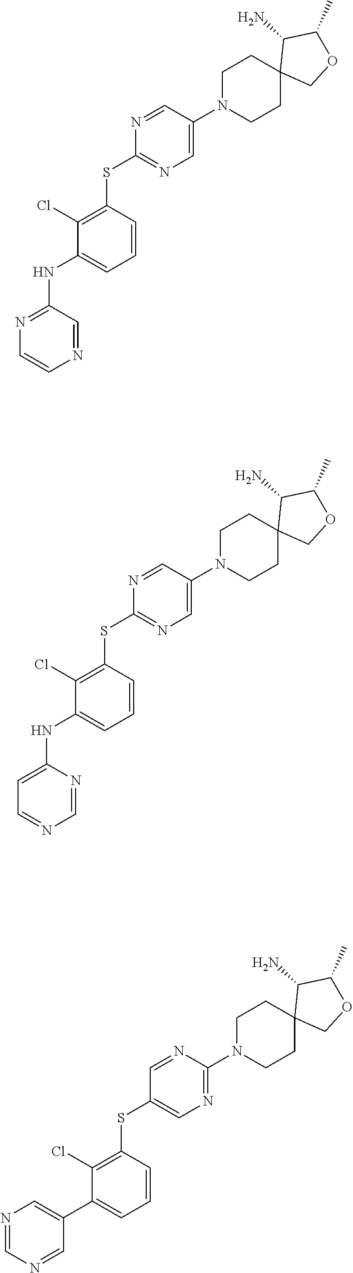 [SMILES: C[C@@H]1OCC2(CCN(c3cnc(Sc4cccc(Nc5cnccn5)c4Cl)nc3)CC2)[C@@H]1N ; C[C@@H]1OCC2(CCN(c3cnc(Sc4cccc(Nc5ccncn5)c4Cl)nc3)CC2)[C@@H]1N ; C[C@@H]1OCC2(CCN(c3ncc(Sc4cccc(-c5cncnc5)c4Cl)cn3)CC2)[C@@H]1N] 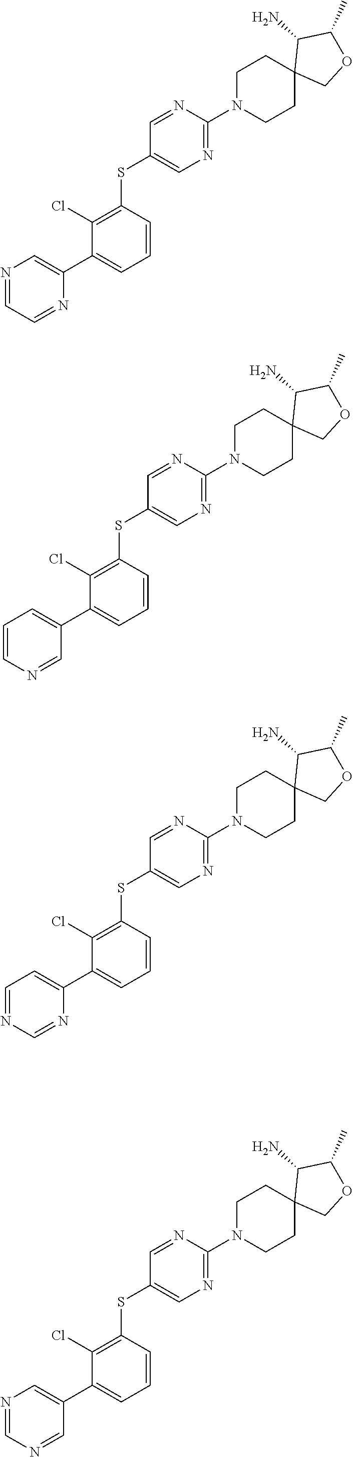 [SMILES: C[C@@H]1OCC2(CCN(c3ncc(Sc4cccc(-c5cnccn5)c4Cl)cn3)CC2)[C@@H]1N ; C[C@@H]1OCC2(CCN(c3ncc(Sc4cccc(-c5cccnc5)c4Cl)cn3)CC2)[C@@H]1N ; C[C@@H]1OCC2(CCN(c3ncc(Sc4cccc(-c5ccncn5)c4Cl)cn3)CC2)[C@@H]1N ; C[C@@H]1OCC2(CCN(c3ncc(Sc4cccc(-c5cncnc5)c4Cl)cn3)CC2)[C@@H]1N] 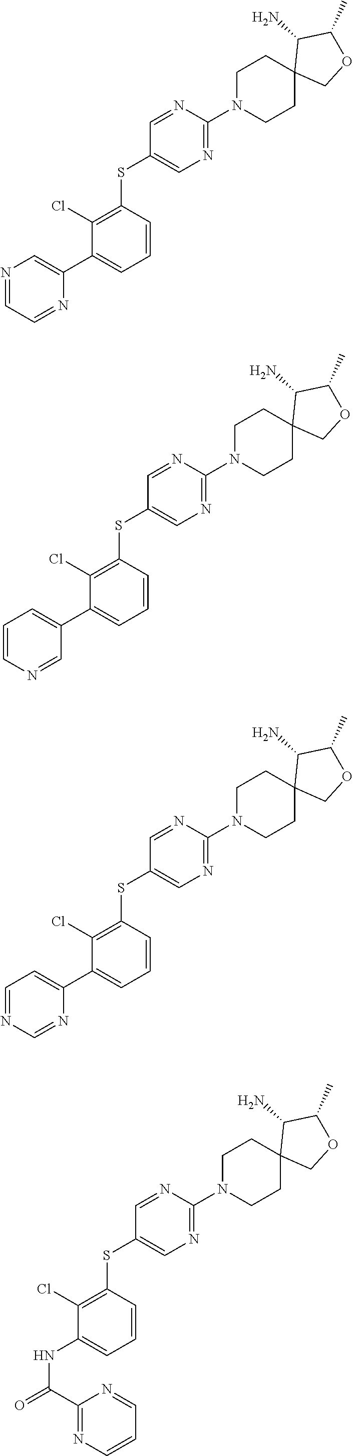 [SMILES: C[C@@H]1OCC2(CCN(c3ncc(Sc4cccc(-c5cnccn5)c4Cl)cn3)CC2)[C@@H]1N ; C[C@@H]1OCC2(CCN(c3ncc(Sc4cccc(-c5cccnc5)c4Cl)cn3)CC2)[C@@H]1N ; C[C@@H]1OCC2(CCN(c3ncc(Sc4cccc(-c5ccncn5)c4Cl)cn3)CC2)[C@@H]1N ; C[C@@H]1OCC2(CCN(c3ncc(Sc4cccc(NC(=O)c5ncccn5)c4Cl)cn3)CC2)[C@@H]1N] 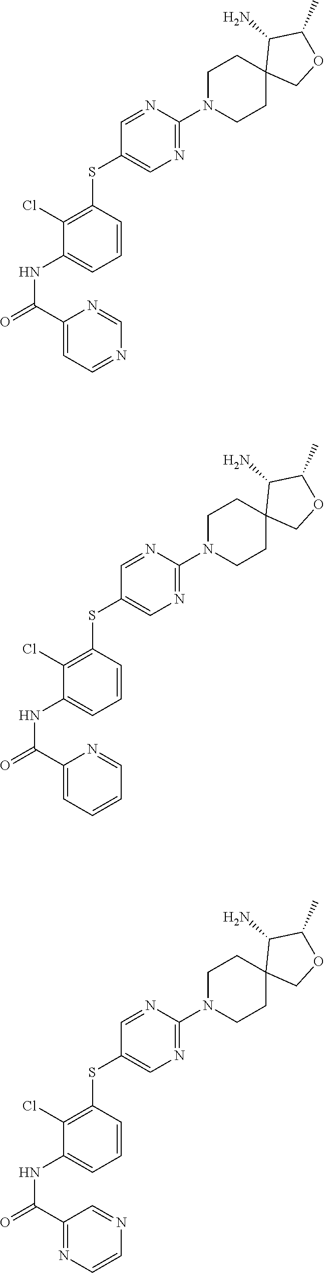 [SMILES: C[C@@H]1OCC2(CCN(c3ncc(Sc4cccc(NC(=O)c5ccncn5)c4Cl)cn3)CC2)[C@@H]1N ; C[C@@H]1OCC2(CCN(c3ncc(Sc4cccc(NC(=O)c5ccccn5)c4Cl)cn3)CC2)[C@@H]1N ; C[C@@H]1OCC2(CCN(c3ncc(Sc4cccc(NC(=O)c5cnccn5)c4Cl)cn3)CC2)[C@@H]1N] 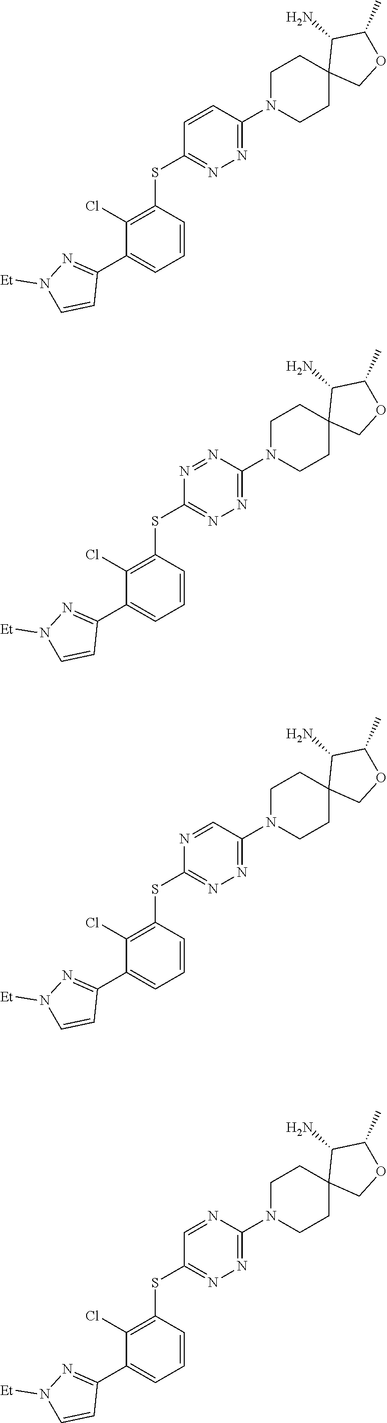 [SMILES: CCn1ccc(-c2cccc(Sc3ccc(N4CCC5(CC4)CO[C@@H](C)[C@H]5N)nn3)c2Cl)n1 ; CCn1ccc(-c2cccc(Sc3nnc(N4CCC5(CC4)CO[C@@H](C)[C@H]5N)nn3)c2Cl)n1 ; CCn1ccc(-c2cccc(Sc3ncc(N4CCC5(CC4)CO[C@@H](C)[C@H]5N)nn3)c2Cl)n1 ; CCn1ccc(-c2cccc(Sc3cnc(N4CCC5(CC4)CO[C@@H](C)[C@H]5N)nn3)c2Cl)n1] 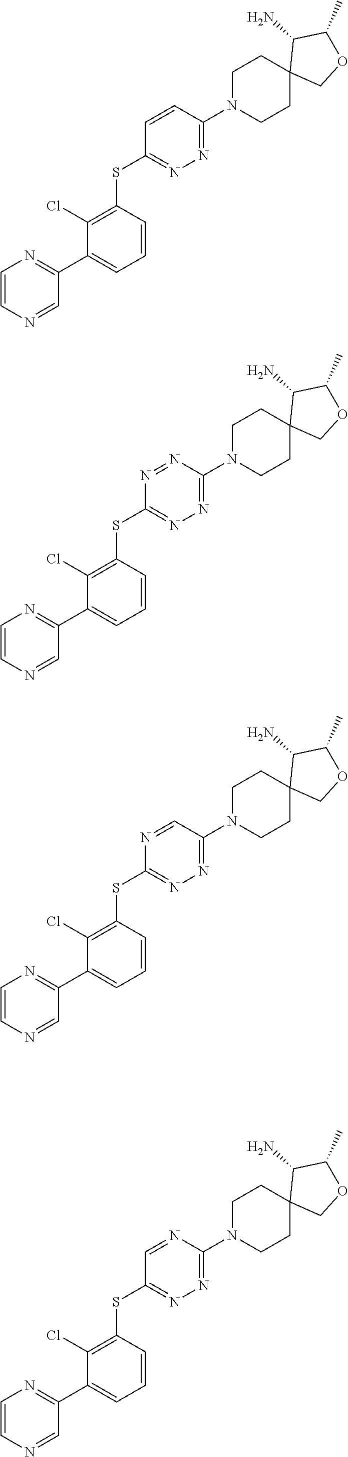 [SMILES: C[C@@H]1OCC2(CCN(c3ccc(Sc4cccc(-c5cnccn5)c4Cl)nn3)CC2)[C@@H]1N ; C[C@@H]1OCC2(CCN(c3nnc(Sc4cccc(-c5cnccn5)c4Cl)nn3)CC2)[C@@H]1N ; C[C@@H]1OCC2(CCN(c3cnc(Sc4cccc(-c5cnccn5)c4Cl)nn3)CC2)[C@@H]1N ; C[C@@H]1OCC2(CCN(c3ncc(Sc4cccc(-c5cnccn5)c4Cl)nn3)CC2)[C@@H]1N] 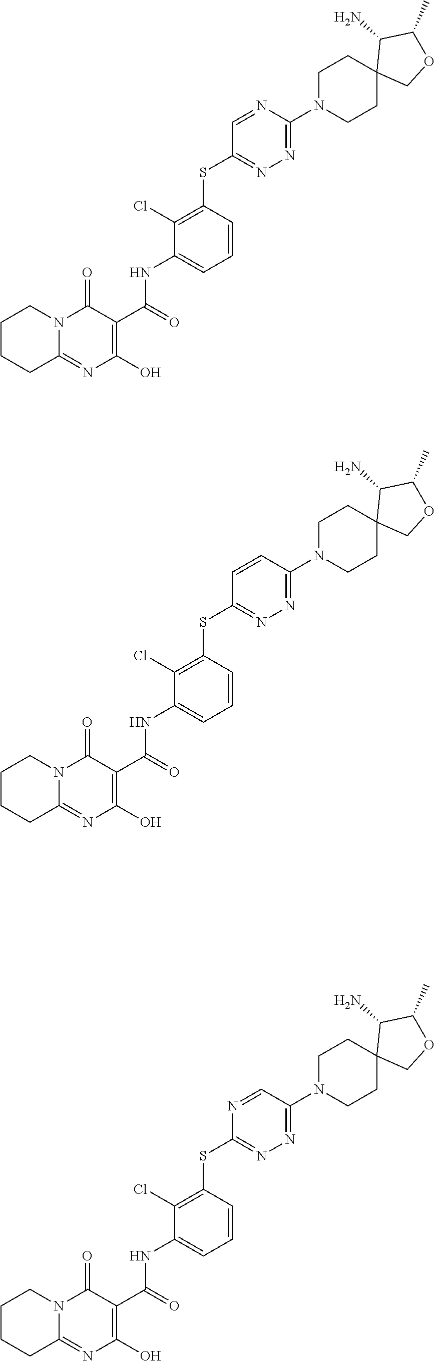 [SMILES: C[C@@H]1OCC2(CCN(c3ncc(Sc4cccc(NC(=O)c5c(O)nc6n(c5=O)CCCC6)c4Cl)nn3)CC2)[C@@H]1N ; C[C@@H]1OCC2(CCN(c3ccc(Sc4cccc(NC(=O)c5c(O)nc6n(c5=O)CCCC6)c4Cl)nn3)CC2)[C@@H]1N ; C[C@@H]1OCC2(CCN(c3cnc(Sc4cccc(NC(=O)c5c(O)nc6n(c5=O)CCCC6)c4Cl)nn3)CC2)[C@@H]1N] 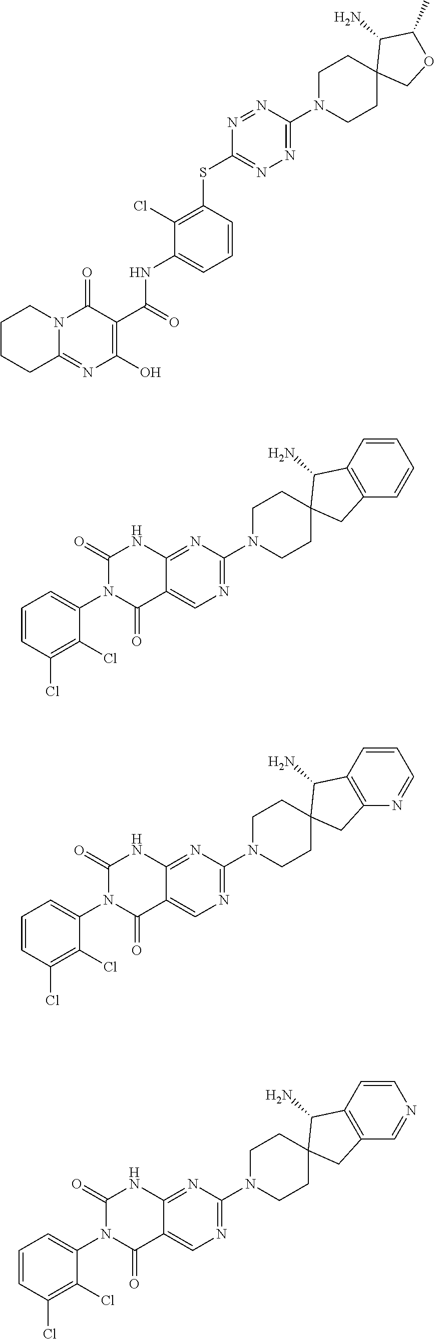 [SMILES: C[C@@H]1OCC2(CCN(c3nnc(Sc4cccc(NC(=O)c5c(O)nc6n(c5=O)CCCC6)c4Cl)nn3)CC2)[C@@H]1N ; N[C@@H]1c2ccccc2CC12CCN(c1ncc3c(=O)n(-c4cccc(Cl)c4Cl)c(=O)[nH]c3n1)CC2 ; N[C@@H]1c2cccnc2CC12CCN(c1ncc3c(=O)n(-c4cccc(Cl)c4Cl)c(=O)[nH]c3n1)CC2 ; N[C@@H]1c2ccncc2CC12CCN(c1ncc3c(=O)n(-c4cccc(Cl)c4Cl)c(=O)[nH]c3n1)CC2] 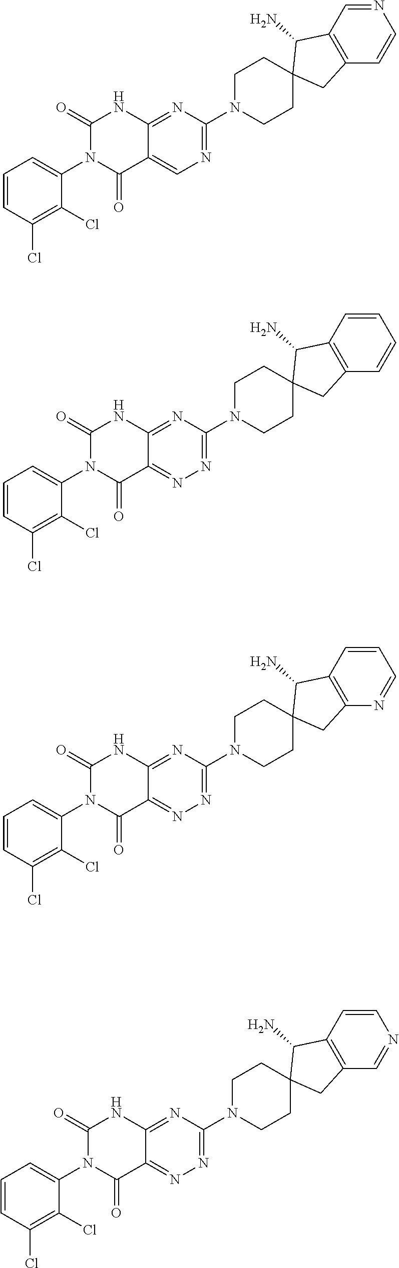 [SMILES: N[C@@H]1c2cnccc2CC12CCN(c1ncc3c(=O)n(-c4cccc(Cl)c4Cl)c(=O)[nH]c3n1)CC2 ; N[C@@H]1c2ccccc2CC12CCN(c1nnc3c(=O)n(-c4cccc(Cl)c4Cl)c(=O)[nH]c3n1)CC2 ; N[C@@H]1c2cccnc2CC12CCN(c1nnc3c(=O)n(-c4cccc(Cl)c4Cl)c(=O)[nH]c3n1)CC2 ; N[C@@H]1c2ccncc2CC12CCN(c1nnc3c(=O)n(-c4cccc(Cl)c4Cl)c(=O)[nH]c3n1)CC2] 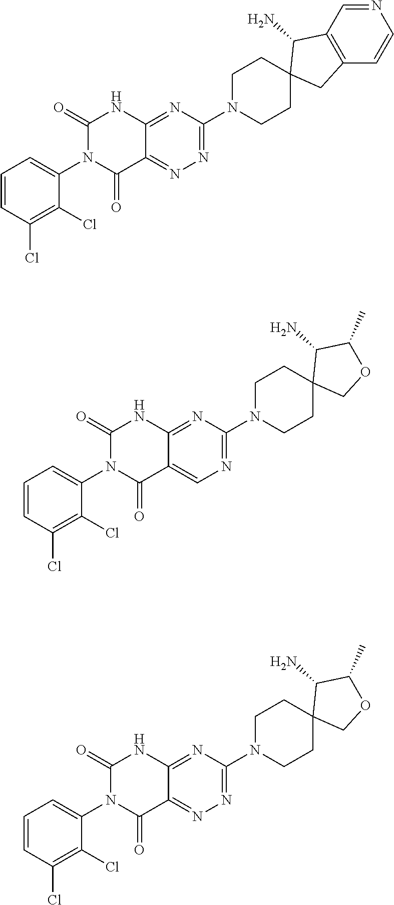 [SMILES: N[C@@H]1c2cnccc2CC12CCN(c1nnc3c(=O)n(-c4cccc(Cl)c4Cl)c(=O)[nH]c3n1)CC2 ; C[C@@H]1OCC2(CCN(c3ncc4c(=O)n(-c5cccc(Cl)c5Cl)c(=O)[nH]c4n3)CC2)[C@@H]1N ; C[C@@H]1OCC2(CCN(c3nnc4c(=O)n(-c5cccc(Cl)c5Cl)c(=O)[nH]c4n3)CC2)[C@@H]1N]
Or their prodrugs, stable isotope derivatives, pharmaceutically acceptable salts, solvates, isomers and mixtures thereof.

6. A pharmaceutical composition, which comprises a compound of formula (I) stated in claim 1, its pharmaceutically acceptable prodrug, stable isotope derivative, pharmaceutically acceptable salt, solvate, or isomer and mixture thereof.

7. A pharmaceutical preparation, which comprises a compound of formula (I) stated in claim 1, its pharmaceutically acceptable prodrug, stable isotope derivative, pharmaceutically acceptable salt, solvate, or isomer and its mixture thereof, or the pharmaceutical compositions stated in claim 5; wherein, the described preparation is any of tablet, capsule, injection, granule, powder, suppository, pill, cream, paste, gel, pulvis, oral solution, inhalation, suspension, dry suspension, patch, and lotion.

8. A compound of formula (I) stated in claim 1, or its pharmaceutically acceptable prodrug, stable isotope derivative, pharmaceutically acceptable salt, solvate, or its isomer and mixture thereof; Or, the pharmaceutical composition stated in claim 6; Or, the pharmaceutical preparation stated in claim 7; all above are used for the prevention and treatment of non-receptor protein tyrosine phosphatase-mediated or -dependent diseases or symptoms.

9. A compound of formula (I) stated in claim 1, or its pharmaceutically acceptable prodrug, stable isotope derivative, pharmaceutically acceptable salt, solvate, or its isomer and mixture thereof; Or, the pharmaceutical composition stated in claim 6; Or, the pharmaceutical preparation stated in claim 7; all above are used for the prevention and treatment of non-receptor protein tyrosine phosphatase-mediated or -dependent diseases or symptoms.

10. A compound of formula (I) stated in claim 1, or its pharmaceutically acceptable prodrug, stable isotope derivative, pharmaceutically acceptable salt, solvate, or its isomer and mixture thereof; Or, the pharmaceutical preparation stated in claim 4 which is used for the prevention and treatment of non-receptor protein tyrosine phosphatase-mediated or -dependent diseases or symptoms.

11. The compound of general formula (I) stated in claim 1, or its tautomer, mesomer, racemate, enantiomer, diastereoisomer, or atropisomer or mixture thereof; Or, its pharmaceutically acceptable salt thereof; Or, the use of the pharmaceutical compositions containing it in the prevention or treatment of Noonan syndrome, Leopard syndrome, juvenile myelomonocytic leukemia, neuroblastoma, melanoma, acute myeloid leukemia, breast cancer, esophageal cancer, lung cancer, colon cancer, head cancer, pancreatic cancer, head and neck squamous cell carcinoma, gastric cancer, liver cancer, anaplastic large cell lymphoma and glioblastoma.
